SEQUENCE LISTING (504 sequences)

SEQ ID NO: 1 - protein, 116 aa, synthetic construct
  REGION 1..116: note = artificial sequence
QVQLVESGGGLVQPGGSLRLSCAASGFKYDVYAMSWVRQAPGKGLEWVGEISPRGDSKYYNTKFIDRFTISRDNEKNTLYLQMNSLRAEDTAVYYCARDWEYFDNWGQGTLVTVSS

SEQ ID NO: 2 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSHEEYTLTISSLQPEDFATYYCQQYRETPYTFGQGTKLEIK

SEQ ID NO: 3 - protein, 116 aa, synthetic construct
  REGION 1..116: note = artificial sequence
KVQLVESGGGLVQPGGSLRLSCAASGFKWEHYAMSWVRQAPGKGLEWVGSISPRGDHKYYNTKFIDRFTISRDNDKNTLYLQMNSLRAEDTAVYYCARDWDYFDRWGQGTLVTVSS

SEQ ID NO: 4 - protein, 106 aa, synthetic construct
  REGION 1..106: note = artificial sequence
AIQMTQSPSSLSASVGDRVTITCQSQFWLSNYLAWYQQKPGKAPKLLIFDGDLPYERPSRFSGSGSHDEYTLTISSLQPEDFATYYCQQYREEPYTFGQGTKLEIK

SEQ ID NO: 5 - protein, 116 aa, synthetic construct
  REGION 1..116: note = artificial sequence
EVQLVESGGGLVQPGGSLRLSCAASGFRFDIYAMSWVRQAPGKGLEWVGEISPDGRTKYYNTKFIDRFTISRDNEKNTLYLQMNSLRAEDTAVYYCARDWEYFDNWGQGTLVTVSS

SEQ ID NO: 6 - protein, 106 aa, synthetic construct; identical to SEQ ID NO: 4
  REGION 1..106: note = artificial sequence

SEQ ID NO: 7 - protein, 116 aa, synthetic construct
  REGION 1..116: note = artificial sequence
RVQLVESGGGLVQPGGSLRLSCAASGFRYDHYAMSWVRQAPGKGLEWVGSISPRGNHKYYNTKFIDRFTISRDDEKNTLYLQMNSLRAEDTAVYYCARDWDYFDHWGQGTLVTVSS

SEQ ID NO: 8 - protein, 106 aa, synthetic construct; identical to SEQ ID NO: 2
  REGION 1..106: note = artificial sequence

SEQ ID NO: 9 - protein, 116 aa, synthetic construct; identical to SEQ ID NO: 1
  REGION 1..116: note = artificial sequence

SEQ ID NO: 10 - protein, 106 aa, synthetic construct
  REGION 1..106: note = artificial sequence
AIQMTQSPSSLSASVGDRVTITCQASYWLSNYLAWYQQKPGKAPKLLIFDGDIPWEIPSRFSGSGSHEEYTLTISSLQPEDFATYYCQQYRDTPYTFGQGTKLEIK

SEQ ID NO: 11 - protein, 116 aa, synthetic construct; identical to SEQ ID NO: 3
  REGION 1..116: note = artificial sequence

SEQ ID NO: 12 - protein, 106 aa, synthetic construct; identical to SEQ ID NO: 4
  REGION 1..106: note = artificial sequence

SEQ ID NO: 13 - protein, 116 aa, synthetic construct; identical to SEQ ID NO: 1
  REGION 1..116: note = artificial sequence

SEQ ID NO: 14 - protein, 106 aa, synthetic construct; identical to SEQ ID NO: 10
  REGION 1..106: note = artificial sequence

SEQ ID NO: 15 - protein, 116 aa, synthetic construct
  REGION 1..116: note = artificial sequence
KLQLVESGGGLVKPGGSLRLSCAASGRRFGVQDMSWVRQAPGKGLEWVGSISPKGDHKYLNTKFIGRFTISRDNSKNTLYLQMNSLRAEDTAVYYCARDWGYFDQWGQGTLVTVSS

SEQ ID NO: 16 - protein, 107 aa, synthetic construct
  REGION 1..107: note = artificial sequence
AIQMTQSPSSLSASVGDRVTITCHGSYWLSSEVAWYQQKPGKAPKLLIYDGNGIIGNVPSRFSGSGSHEDYTLTISSLQPEDFATYYCQQYRYHPYTFGHGTKVEIK

SEQ ID NO: 17 - protein, 116 aa, synthetic construct
  REGION 1..116: note = artificial sequence
YGQLVESGGGLVKPGGSLRLSCAATGTLFSKQDMSWVRQAPGKGLEWVGSISPKGDHKYLNTKFIGRFTISRDNSKNTLYLQMNSLRAEDTAVYYCAKDWGYFDHWGQGTLVTVSS

SEQ ID NO: 18 - protein, 107 aa, synthetic construct
  REGION 1..107: note = artificial sequence
AIQMTQSPSSLSASVGDRVTITCHGSYWLSSEVAWYQQKPGKAPKLLIYDGNGVQGSVPSRFSGSGSHEDYTLTISSLQQEDFATYYCQQYRYHPYTFGHGTKVEIK

SEQ ID NO: 19 - protein, 116 aa, synthetic construct
  REGION 1..116: note = artificial sequence
DPQLVESGGGLVKPGGSLRLSCAARGLHFNNTDMSWVRQAPGKGLEWVGSISPKGDHKYLNTKFIGRFTISRDNSKNTLYLQMNSLRAEDTAVYYCARDWGYFDWWGQGTLVTVSS

SEQ ID NO: 20 - protein, 107 aa, synthetic construct
  REGION 1..107: note = artificial sequence
AIQMTQSPSSLSASVGDRVTITCHGSYWLSSEVAWYQQKPGKAPKLLIYDGNGLKGGVPSRFSGSGSHEDYTLTISSLQPEDFATYYCQQYRYHPYTFGHGTKVEIK

SEQ ID NO: 21 - protein, 115 aa, synthetic construct
  REGION 1..115: note = artificial sequence
GQLVESGGGLVKPGGSLRLSCKASGFYWGPGAMSWVRQAPGKGLEWVGSISPKSGSKGYAQNVPGRFTISRDNNQDTLYLQMNSLRAEDTAVYYCARDIGFFDTWGQGTLVTVSS

SEQ ID NO: 22 - protein, 108 aa, synthetic construct
  REGION 1..108: note = artificial sequence
HDIQMTQSPSSLSASVGDRVTITCDADFGISNYLAWYQQKPGKAPKLLIFDAKYRAPEVPSRFSGSGSGGSFTLTISSLQPEDFATYYCQQYGVHPFTFGQGTKVEIK

SEQ ID NO: 23 - protein, 115 aa, synthetic construct
  REGION 1..115: note = artificial sequence
GQLVESGGGLVKPGGSLRLSCKASGFYWGPGAMSWVRQAPGKGLEWVGSISPKGGSTDINPKIGGRFTISRDNNQDTLYLQMNSLRAEDTAVYYCARDIGFFDTWGQGTLVTVSS

SEQ ID NO: 24 - protein, 107 aa, synthetic construct
  REGION 1..107: note = artificial sequence
AIQMTQSPSSLSASVGDRVTITCQGSWLISNYLAWYQQKPGKAPKLLIFDAKYRAPEVPSRFSGSGSGTDFTLTISSLQPEDFATYYCQQYGGRLLTFGQGTKVEIK

SEQ ID NO: 25 - protein, 115 aa, synthetic construct
  REGION 1..115: note = artificial sequence
GQLVESGGGLVKPGGSLRLSCKASGFYWGPGAMSWVRQAPGKGLEWVGSISPKGGSTEYNTKVYGRFTISRDNNQDTLYLQMNSLRAEDTAVYYCARDIGFFDTWGQGTLVTVSS

SEQ ID NO: 26 - protein, 106 aa, synthetic construct
  REGION 1..106: note = artificial sequence
HQMTQSPSSLSASVGDRVTITCDGEWDISNYLAWYQQKPGKAPKLLIFDAKYRAPEVPSRFSGSGSGRDFTLTISSLQPEDFATYYCQQYNRMYFTFGQGTKVEIK

SEQ ID NO: 27 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAWYNNNFTGRFTISRDNNQDTLYLQMNSLRAEDTAVYYCARDIGFFDTWGQGTLVTVSS

SEQ ID NO: 28 - protein, 108 aa, synthetic construct
  REGION 1..108: note = artificial sequence
HINQMTQSPSSLSASVGDRVTITCQAKWVISNYLAWYQQKPGKAPKLLIFDAKYRAPEVPSRFSGSGSDGTFTLTISSLQPEDFATYYCQQYDWWPYTFGQGTKVEIK

SEQ ID NO: 29 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAWYNNKFHGRFTISRDNNQDTLYLQMNSLRAEDTAVYYCARDIGFFDTWGQGTLVTVSS

SEQ ID NO: 30 - protein, 108 aa, synthetic construct
  REGION 1..108: note = artificial sequence
HYIQMTQSPSSLSASVGDRVTITCQGNAFISNYLAWYQQKPGKAPKLLIFDAKYRAPEVPSRFSGSGSGDDFTLTISSLQPEDFATYYCQQYDWWPYTFGQGTKVEIK

SEQ ID NO: 31 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAWFNRNFAGRFTISRDNNQDTLYLQMNSLRAEDTAVYYCARDIGFFDTWGQGTLVTVSS

SEQ ID NO: 32 - protein, 108 aa, synthetic construct
  REGION 1..108: note = artificial sequence
GHVQMTQSPSSLSASVGDRVTITCQAEDEISNYLAWYQQKPGKAPKLLIFDAKYRAPEVPSRFSGSGSDASFTLTISSLQPEDFATYYCQQYDWWPYTFGQGTKVEIK

SEQ ID NO: 33 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAWYNDKFKGRFTISRDNNQDTLYLQMNSLRAEDTAVYYCARDIGFFDTWGQGTLVTVSS

SEQ ID NO: 34 - protein, 108 aa, synthetic construct
  REGION 1..108: note = artificial sequence
HINQMTQSPSSLSASVGDRVTITCNSTEFISNYLAWYQQKPGKAPKLLIFDAKYRAPEVPSRFSGSGSDQEFTLTISSLQPEDFATYYCQQYDWWPYTFGQGTKVEIK

SEQ ID NO: 35 - protein, 116 aa, synthetic construct
  REGION 1..116: note = artificial sequence
RKQLVESGGGLVQPGGSLRLSCAAHGSLVSRNDMSWVRQAPGKGLEWVGSISPKGDHKYYNTKFIDRFTISRDNSKNTLYLQMNSLRAEDTAVYYCAKDVDYMDLWGQGTLVTVSS

SEQ ID NO: 36 - protein, 107 aa, synthetic construct
  REGION 1..107: note = artificial sequence
AIQMTQSPSSLSASVGDRVTITCQASYWLNSELAWYQQKPGKAPKLLIFDGDQTWFGVPSRFSGSGSHEDYTLTISSLQPEDFATYYCQQYRYTPYTFGQGTKLEIK

SEQ ID NO: 37 - protein, 115 aa, synthetic construct
  REGION 1..115: note = artificial sequence
GQLVESGGGLVKPGGSLRLSCKASGFYWGPGAMSWVRQAPGKGLEWVGSISPKGGSAEYASKVYGRFTISRDNNQDTLYLQMNSLRAEDTAVYYCARDIGFFDTWGQGTLVTVSS

SEQ ID NO: 38 - protein, 107 aa, synthetic construct
  REGION 1..107: note = artificial sequence
DYQMTQSPSSLSASVGDRVTITCEAHSQISNYLAWYQQKPGKAPKLLIFDAKYRAPEVPSRFSGSGSGPDFTLTISSLQPEDFATYYCQQYNDFWLTFGQGTKVEIK

SEQ ID NO: 39 - protein, 116 aa, synthetic construct
  REGION 1..116: note = artificial sequence
EVQLVESGGGLVQPGGSLRLSCAASGFSWEHYAMTWVRQAPGKGLEWVGSISPKGDHKYYNTKFIDRFTISRDNSKNTLYLQMNSLRAEDTAVYYCARDWDYFDEWGQGTLVTVSS

SEQ ID NO: 40 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYRYTPYTFGQGTKLEIK

SEQ ID NO: 41 - protein, 115 aa, synthetic construct
  REGION 1..115: note = artificial sequence
GQLVESGGGLVQPGGSLRLSCAASGFTWDHYAMTWVRQAPGKGLEWVGSISPKGDHKYYNTKFIDRFTISRDNSKNTLYLQMNSLRAEDTAVYYCARDWDYFDEWGQGTLVTVSS

SEQ ID NO: 42 - protein, 106 aa, synthetic construct; identical to SEQ ID NO: 40
  REGION 1..106: note = artificial sequence

SEQ ID NO: 43 - protein, 116 aa, synthetic construct; identical to SEQ ID NO: 39
  REGION 1..116: note = artificial sequence

SEQ ID NO: 44 - protein, 106 aa, synthetic construct; identical to SEQ ID NO: 40
  REGION 1..106: note = artificial sequence

SEQ ID NO: 45 - protein, 116 aa, synthetic construct; identical to SEQ ID NO: 5
  REGION 1..116: note = artificial sequence

SEQ ID NO: 46 - protein, 106 aa, synthetic construct; identical to SEQ ID NO: 40
  REGION 1..106: note = artificial sequence

SEQ ID NO: 47 - protein, 116 aa, synthetic construct; identical to SEQ ID NO: 1
  REGION 1..116: note = artificial sequence

SEQ ID NO: 48 - protein, 106 aa, synthetic construct; identical to SEQ ID NO: 40
  REGION 1..106: note = artificial sequence

SEQ ID NO: 49 - protein, 116 aa, synthetic construct
  REGION 1..116: note = artificial sequence
KVQLVESGGGLVQPGGSLRLSCAASGFRWEHYAMSWVRQAPGKGLEWVGEISPDGDTKYYNTKFIDRFTISRDDSKNTLYLQMNSLRAEDTAVYYCARDWEYFDNWGQGTLVTVSS

SEQ ID NO: 50 - protein, 106 aa, synthetic construct; identical to SEQ ID NO: 40
  REGION 1..106: note = artificial sequence

SEQ ID NO: 51 - protein, 116 aa, synthetic construct; identical to SEQ ID NO: 7
  REGION 1..116: note = artificial sequence

SEQ ID NO: 52 - protein, 106 aa, synthetic construct; identical to SEQ ID NO: 40
  REGION 1..106: note = artificial sequence

SEQ ID NO: 53 - protein, 116 aa, synthetic construct
  REGION 1..116: note = artificial sequence
RVQLVESGGGLVQPGGSLRLSCAASGFTWDHYAMSWVRQAPGKGLEWVGSISPKGDHKYYNTKFIDRFTISRDNSKNTLYLQMNSLRAEDTAVYYCARDWDYFDRWGQGTLVTVSS

SEQ ID NO: 54 - protein, 106 aa, synthetic construct; identical to SEQ ID NO: 40
  REGION 1..106: note = artificial sequence

SEQ ID NO: 55 - protein, 116 aa, synthetic construct
  REGION 1..116: note = artificial sequence
RVQLVESGGGLVQPGGSLRLSCAASGFTYDHYAMSWVRQAPGKGLEWVGEISPRGNTKYYNTKFIDRFTISRDNSKNTLYLQMNSLRAEDTAVYYCARDWEYFDYWGQGTLVTVSS

SEQ ID NO: 56 - protein, 106 aa, synthetic construct; identical to SEQ ID NO: 40
  REGION 1..106: note = artificial sequence

SEQ ID NO: 57 - protein, 116 aa, synthetic construct
  REGION 1..116: note = artificial sequence
KVQLVESGGGLVQPGGSLRLSCAASGYRYDKYAMSWVRQAPGKGLEWVGEISPDGRTKYYNTKFIDRFTISRDNDKNTLYLQMNSLRAEDTAVYYCARDWEYFDNWGQGTLVTVSS

SEQ ID NO: 58 - protein, 106 aa, synthetic construct; identical to SEQ ID NO: 40
  REGION 1..106: note = artificial sequence

SEQ ID NO: 59 - protein, 116 aa, synthetic construct; identical to SEQ ID NO: 3
  REGION 1..116: note = artificial sequence

SEQ ID NO: 60 - protein, 106 aa, synthetic construct; identical to SEQ ID NO: 40
  REGION 1..106: note = artificial sequence

SEQ ID NO: 61 - protein, 116 aa, synthetic construct
  REGION 1..116: note = artificial sequence
RVQLVESGGGLVQPGGSLRLSCAASGYRWDHYAMSWVRQAPGKGLEWVGSISPKGDHKYYNTKFIDRFTISRDNSKNTLYLQMNSLRAEDTAVYYCARDWDYFDYWGQGTLVTVSS

SEQ ID NO: 62 - protein, 106 aa, synthetic construct; identical to SEQ ID NO: 40
  REGION 1..106: note = artificial sequence

SEQ ID NO: 63 - protein, 116 aa, synthetic construct
  REGION 1..116: note = artificial sequence
KVQLVESGGGLVQPGGSLRLSCAASGFTYDAYAMSWVRQAPGKGLEWVGEISPRGESKYYNTKFIDRFTISRDNSKNTLYLQMNSLRAEDTAVYYCARDWEYFDNWGQGTLVTVSS

SEQ ID NO: 64 - protein, 106 aa, synthetic construct; identical to SEQ ID NO: 40
  REGION 1..106: note = artificial sequence

SEQ ID NO: 65 - protein, 116 aa, synthetic construct
  REGION 1..116: note = artificial sequence
EVQLVESGGGLVQPGGSLRLSCAASGFQWDHYAMSWVRQAPGKGLEWVGEISPRGDTKYYNTKFIDRFTISRDDEKNTLYLQMNSLRAEDTAVYYCARDWEYFDYWGQGTLVTVSS

SEQ ID NO: 66 - protein, 106 aa, synthetic construct; identical to SEQ ID NO: 40
  REGION 1..106: note = artificial sequence

SEQ ID NO: 67 - protein, 116 aa, synthetic construct
  REGION 1..116: note = artificial sequence
RVQLVESGGGLVQPGGSLRLSCAASGFKFEHYAMSWVRQAPGKGLEWVGEISPKGDHKYYNTKFIDRFTISRDNEKNTLYLQMNSLRAEDTAVYYCARDWEYFDRWGQGTLVTVSS

SEQ ID NO: 68 - protein, 106 aa, synthetic construct; identical to SEQ ID NO: 40
  REGION 1..106: note = artificial sequence

SEQ ID NO: 69 - protein, 115 aa, synthetic construct; identical to SEQ ID NO: 41
  REGION 1..115: note = artificial sequence

SEQ ID NO: 70 - protein, 106 aa, synthetic construct
  REGION 1..106: note = artificial sequence
AIQMTQSPSSLSASVGDRVTITCQAQYWLSNYLAWYQQKPGKAPKLLIFDGDLPYEVPSRFSGSGSHDEYTLTISSLQPEDFATYYCQQYRNTPYTFGQGTKLEIK

SEQ ID NO: 71 - protein, 115 aa, synthetic construct; identical to SEQ ID NO: 41
  REGION 1..115: note = artificial sequence

SEQ ID NO: 72 - protein, 106 aa, synthetic construct; identical to SEQ ID NO: 4
  REGION 1..106: note = artificial sequence

SEQ ID NO: 73 - protein, 115 aa, synthetic construct; identical to SEQ ID NO: 41
  REGION 1..115: note = artificial sequence

SEQ ID NO: 74 - protein, 106 aa, synthetic construct; identical to SEQ ID NO: 10
  REGION 1..106: note = artificial sequence

SEQ ID NO: 75 - protein, 115 aa, synthetic construct; identical to SEQ ID NO: 41
  REGION 1..115: note = artificial sequence

SEQ ID NO: 76 - protein, 106 aa, synthetic construct; identical to SEQ ID NO: 2
  REGION 1..106: note = artificial sequence

SEQ ID NO: 77 - protein, 115 aa, synthetic construct; identical to SEQ ID NO: 41
  REGION 1..115: note = artificial sequence

SEQ ID NO: 78 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSEDEYTLTISSLQPEDFATYYCQQYRETPYTFGQGTKLEIK

SEQ ID NO: 79 - protein, 115 aa, synthetic construct; identical to SEQ ID NO: 41
  REGION 1..115: note = artificial sequence

SEQ ID NO: 80 - protein, 106 aa, synthetic construct
  REGION 1..106: note = artificial sequence
AIQMTQSPSSLSASVGDRVTITCQSSHWLSNYLAWYQQKPGKAPKLLIFDGDLPWEVPSRFSGSGSEDEYTLTISSLQPEDFATYYCQQYRYTPYTFGQGTKLEIK

SEQ ID NO: 81 - protein, 115 aa, synthetic construct; identical to SEQ ID NO: 41
  REGION 1..115: note = artificial sequence

SEQ ID NO: 82 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYRNTPYTFGQGTKLEIK

SEQ ID NO: 83 - protein, 115 aa, synthetic construct; identical to SEQ ID NO: 41
  REGION 1..115: note = artificial sequence

SEQ ID NO: 84 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEEYTLTISSLQPEDFATYYCQQYRSTPYTFGQGTKLEIK

SEQ ID NO: 85 - protein, 115 aa, synthetic construct; identical to SEQ ID NO: 41
  REGION 1..115: note = artificial sequence

SEQ ID NO: 86 - protein, 106 aa, synthetic construct
  REGION 1..106: note = artificial sequence
AIQMTQSPSSLSASVGDRVTITCQSTHWLSAYLAWYQQKPGKAPKLLIFDGDIPYERPSRFSGSGSHDEYTLTISSLQPEDFATYYCQQYRNEPYTFGQGTKLEIK

SEQ ID NO: 87 - protein, 116 aa, synthetic construct
  REGION 1..116: note = artificial sequence
EVQLVESGGGLVQPGGSLRLSCAASGFRWDKYAMSWVRQAPGKGLEWVGEISPRGDSKYYNTKFIDRFTISRDDEKNTLYLQMNSLRAEDTAVYYCARDWEYFDNWGQGTLVTVSS

SEQ ID NO: 88 - protein, 106 aa, synthetic construct; identical to SEQ ID NO: 40
  REGION 1..106: note = artificial sequence

SEQ ID NO: 89 - protein, 116 aa, synthetic construct
  REGION 1..116: note = artificial sequence
RVQLVESGGGLVQPGGSLRLSCAASGFTYEHYAMSWVRQAPGKGLEWVGSISPRGDHKYYNTKFIDRFTISRDNSKNTLYLQMNSLRAEDTAVYYCARDWDYFDTWGQGTLVTVSS

SEQ ID NO: 90 - protein, 106 aa, synthetic construct; identical to SEQ ID NO: 40
  REGION 1..106: note = artificial sequence

SEQ ID NO: 91 - protein, 116 aa, synthetic construct
  REGION 1..116: note = artificial sequence
GVQLVESGGGLVQPGGSLRLSCAASGFRYDIYAMSWVRQAPGKGLEWVGEISPEGNHKYYNTKFIDRFTISRDNEKNTLYLQMNSLRAEDTAVYYCARDWEYFDTWGQGTLVTVSS

SEQ ID NO: 92 - protein, 106 aa, synthetic construct; identical to SEQ ID NO: 40
  REGION 1..106: note = artificial sequence

SEQ ID NO: 93 - protein, 116 aa, synthetic construct
  REGION 1..116: note = artificial sequence
KVQLVESGGGLVQPGGSLRLSCAASGYTYDIYAMSWVRQAPGKGLEWVGEISPDGNTKYYNTKFIDRFTISRDNSKNTLYLQMNSLRAEDTAVYYCARDWEYFDHWGQGTLVTVSS

SEQ ID NO: 94 - protein, 106 aa, synthetic construct; identical to SEQ ID NO: 40
  REGION 1..106: note = artificial sequence

SEQ ID NO: 95 - protein, 116 aa, synthetic construct
  REGION 1..116: note = artificial sequence
KVQLVESGGGLVQPGGSLRLSCAASGYRYEHYAMSWVRQAPGKGLEWVGSISPKGQHKYYNTKFIDRFTISRDNEKNTLYLQMNSLRAEDTAVYYCARDWEYFDVWGQGTLVTVSS

SEQ ID NO: 96 - protein, 106 aa, synthetic construct; identical to SEQ ID NO: 40
  REGION 1..106: note = artificial sequence

SEQ ID NO: 97 - protein, 116 aa, synthetic construct
  REGION 1..116: note = artificial sequence
KVQLVESGGGLVQPGGSLRLSCAASGFRFDIYAMSWVRQAPGKGLEWVGEISPKGEHKYYNTKFIDRFTISRDNSKNTLYLQMNSLRAEDTAVYYCARDWEYFDGWGQGTLVTVSS

SEQ ID NO: 98 - protein, 106 aa, synthetic construct; identical to SEQ ID NO: 40
  REGION 1..106: note = artificial sequence

SEQ ID NO: 99 - protein, 116 aa, synthetic construct
  REGION 1..116: note = artificial sequence
KVQLVESGGGLVQPGGSLRLSCAASGFHYDIYAMSWVRQAPGKGLEWVGEISPDGRHKYYNTKFIDRFTISRDNSKNTLYLQMNSLRAEDTAVYYCARDWEYFDNWGQGTLVTVSS

SEQ ID NO: 100 - protein, 106 aa, synthetic construct; identical to SEQ ID NO: 40
  REGION 1..106: note = artificial sequence

SEQ ID NO: 101 - protein, 116 aa, synthetic construct
  REGION 1..116: note = artificial sequence
GVQLVESGGGLVQPGGSLRLSCAASGFKYEHYAMSWVRQAPGKGLEWVGEISPKGDHKYYNTKFIDRFTISRDNEKNTLYLQMNSLRAEDTAVYYCARDWEYFDYWGQGTLVTVSS

SEQ ID NO: 102 - protein, 106 aa, synthetic construct; identical to SEQ ID NO: 40
  REGION 1..106: note = artificial sequence

SEQ ID NO: 103 - protein, 116 aa, synthetic construct
  REGION 1..116: note = artificial sequence
KVQLVESGGGLVQPGGSLRLSCAASGFRWEHYAMSWVRQAPGKGLEWVGSISPRGDHKYYNTKFIDRFTISRDNSKNTLYLQMNSLRAEDTAVYYCARDWDYFDQWGQGTLVTVSS

SEQ ID NO: 104 - protein, 106 aa, synthetic construct; identical to SEQ ID NO: 40
  REGION 1..106: note = artificial sequence

SEQ ID NO: 105 - protein, 116 aa, synthetic construct
  REGION 1..116: note = artificial sequence
RVQLVESGGGLVQPGGSLRLSCAASGFKWDHYAMSWVRQAPGKGLEWVGSISPDGNHKYYNTKFIDRFTISRDDSKNTLYLQMNSLRAEDTAVYYCARDWDYFDYWGQGTLVTVSS

SEQ ID NO: 106 - protein, 106 aa, synthetic construct; identical to SEQ ID NO: 40
  REGION 1..106: note = artificial sequence

SEQ ID NO: 107 - protein, 116 aa, synthetic construct
  REGION 1..116: note = artificial sequence
KVQLVESGGGLVQPGGSLRLSCAASGYKYDHYAMSWVRQAPGKGLEWVGEISPEGNSKYYNTKFIDRFTISRDNSKNTLYLQMNSLRAEDTAVYYCARDWEYFDNWGQGTLVTVSS

SEQ ID NO: 108 - protein, 106 aa, synthetic construct; identical to SEQ ID NO: 40
  REGION 1..106: note = artificial sequence

SEQ ID NO: 109 - protein, 116 aa, synthetic construct
  REGION 1..116: note = artificial sequence
KVQLVESGGGLVQPGGSLRLSCAASGFKYEVYAMSWVRQAPGKGLEWVGEISPKGDTKYYNTKFIDRFTISRDNDKNTLYLQMNSLRAEDTAVYYCARDWEYFDNWGQGTLVTVSS

SEQ ID NO: 110 - protein, 106 aa, synthetic construct; identical to SEQ ID NO: 40
  REGION 1..106: note = artificial sequence

SEQ ID NO: 111 - protein, 116 aa, synthetic construct
  REGION 1..116: note = artificial sequence
RVQLVESGGGLVQPGGSLRLSCAASGFKWEIYAMSWVRQAPGKGLEWVGDISPDGQTKYYNTKFIDRFTISRDDEKNTLYLQMNSLRAEDTAVYYCARDWEYFDHWGQGTLVTVSS

SEQ ID NO: 112 - protein, 106 aa, synthetic construct; identical to SEQ ID NO: 40
  REGION 1..106: note = artificial sequence

SEQ ID NO: 113 - protein, 116 aa, synthetic construct
  REGION 1..116: note = artificial sequence
QVQLVESGGGLVQPGGSLRLSCAASGFTWDHYAMSWVRQAPGKGLEWVGEISPRGDTKYYNTKFIDRFTISRDNEKNTLYLQMNSLRAEDTAVYYCARDWDYFDNWGQGTLVTVSS

SEQ ID NO: 114 - protein, 106 aa, synthetic construct; identical to SEQ ID NO: 40
  REGION 1..106: note = artificial sequence

SEQ ID NO: 115 - protein, 116 aa, synthetic construct
  REGION 1..116: note = artificial sequence
RVQLVESGGGLVQPGGSLRLSCAASGFKWEHYAMSWVRQAPGKGLEWVGSISPRGDHKYYNTKFIDRFTISRDDDKNTLYLQMNSLRAEDTAVYYCARDWDYFDSWGQGTLVTVSS

SEQ ID NO: 116 - protein, 106 aa, synthetic construct; identical to SEQ ID NO: 40
  REGION 1..106: note = artificial sequence

SEQ ID NO: 117 - protein, 116 aa, synthetic construct
  REGION 1..116: note = artificial sequence
KVQLVESGGGLVQPGGSLRLSCAASGYRWDIYAMSWVRQAPGKGLEWVGSISPDGNHKYYNTKFIDRFTISRDDSKNTLYLQMNSLRAEDTAVYYCARDWDYFDTWGQGTLVTVSS

SEQ ID NO: 118 - protein, 106 aa, synthetic construct; identical to SEQ ID NO: 40
  REGION 1..106: note = artificial sequence

SEQ ID NO: 119 - protein, 116 aa, synthetic construct
  REGION 1..116: note = artificial sequence
KVQLVESGGGLVQPGGSLRLSCAASGFRYDYYAMSWVRQAPGKGLEWVGSISPRGDHKYYNTKFIDRFTISRDNDKNTLYLQMNSLRAEDTAVYYCARDWDYFDYWGQGTLVTVSS

SEQ ID NO: 120 - protein, 106 aa, synthetic construct; identical to SEQ ID NO: 40
  REGION 1..106: note = artificial sequence

SEQ ID NO: 121 - protein, 116 aa, synthetic construct
  REGION 1..116: note = artificial sequence
KVQLVESGGGLVQPGGSLRLSCAASGFTWEHYAMSWVRQAPGKGLEWVGSISPDGQHKYYNTKFIDRFTISRDDSKNTLYLQMNSLRAEDTAVYYCARDWDYFDYWGQGTLVTVSS

SEQ ID NO: 122 - protein, 106 aa, synthetic construct; identical to SEQ ID NO: 40
  REGION 1..106: note = artificial sequence

SEQ ID NO: 123 - protein, 116 aa, synthetic construct
  REGION 1..116: note = artificial sequence
QVQLVESGGGLVQPGGSLRLSCAASGYRWDHYAMSWVRQAPGKGLEWVGSISPDGRHKYYNTKFIDRFTISRDNSKNTLYLQMNSLRAEDTAVYYCARDWDYFDRWGQGTLVTVSS

SEQ ID NO: 124 - protein, 106 aa, synthetic construct; identical to SEQ ID NO: 40
  REGION 1..106: note = artificial sequence

SEQ ID NO: 125 - protein, 116 aa, synthetic construct
  REGION 1..116: note = artificial sequence
RVQLVESGGGLVQPGGSLRLSCAASGFRFDIYAMSWVRQAPGKGLEWVGEISPDGRHKYYNTKFIDRFTISRDDEKNTLYLQMNSLRAEDTAVYYCARDWEYFDEWGQGTLVTVSS

SEQ ID NO: 126 - protein, 106 aa, synthetic construct; identical to SEQ ID NO: 40
  REGION 1..106: note = artificial sequence

SEQ ID NO: 127 - protein, 116 aa, synthetic construct
  REGION 1..116: note = artificial sequence
RVQLVESGGGLVQPGGSLRLSCAASGFKWEHYAMSWVRQAPGKGLEWVGEISPDGRTKYYNTKFIDRFTISRDDSKNTLYLQMNSLRAEDTAVYYCARDWDYFDHWGQGTLVTVSS

SEQ ID NO: 128 - protein, 106 aa, synthetic construct; identical to SEQ ID NO: 40
  REGION 1..106: note = artificial sequence

SEQ ID NO: 129 - protein, 116 aa, synthetic construct
  REGION 1..116: note = artificial sequence
RVQLVESGGGLVQPGGSLRLSCAASGFTYDHYAMSWVRQAPGKGLEWVGEISPKGDTKYYNTKFIDRFTISRDDDKNTLYLQMNSLRAEDTAVYYCARDWDYFDYWGQGTLVTVSS

SEQ ID NO: 130 - protein, 106 aa, synthetic construct; identical to SEQ ID NO: 40
  REGION 1..106: note = artificial sequence

SEQ ID NO: 131 - protein, 116 aa, synthetic construct
  REGION 1..116: note = artificial sequence
KVQLVESGGGLVQPGGSLRLSCAASGFTWDHYAMSWVRQAPGKGLEWVGSISPRGDHKYYNTKFIDRFTISRDDSKNTLYLQMNSLRAEDTAVYYCARDWDYFDSWGQGTLVTVSS

SEQ ID NO: 132 - protein, 106 aa, synthetic construct; identical to SEQ ID NO: 40
  REGION 1..106: note = artificial sequence

SEQ ID NO: 133 - protein, 116 aa, synthetic construct
  REGION 1..116: note = artificial sequence
RVQLVESGGGLVQPGGSLRLSCAASGFHFDKYAMSWVRQAPGKGLEWVGSISPKGRTKYYNTKFIDRFTISRDNSKNTLYLQMNSLRAEDTAVYYCARDWDYFDNWGQGTLVTVSS

SEQ ID NO: 134 - protein, 106 aa, synthetic construct; identical to SEQ ID NO: 40
  REGION 1..106: note = artificial sequence

SEQ ID NO: 135 - protein, 116 aa, synthetic construct
  REGION 1..116: note = artificial sequence
RVQLVESGGGLVQPGGSLRLSCAASGFRWDYYAMSWVRQAPGKGLEWVGSISPDGQHKYYNTKFIDRFTISRDDDKNTLYLQMNSLRAEDTAVYYCARDWDYFDHWGQGTLVTVSS

SEQ ID NO: 136 - protein, 106 aa, synthetic construct; identical to SEQ ID NO: 40
  REGION 1..106: note = artificial sequence

SEQ ID NO: 137 - protein, 116 aa, synthetic construct
  REGION 1..116: note = artificial sequence
KVQLVESGGGLVQPGGSLRLSCAASGYRWDHYAMSWVRQAPGKGLEWVGEISPDGQNKYYNTKFIDRFTISRDNDKNTLYLQMNSLRAEDTAVYYCARDWEYFDNWGQGTLVTVSS

SEQ ID NO: 138 - protein, 106 aa, synthetic construct; identical to SEQ ID NO: 40
  REGION 1..106: note = artificial sequence

SEQ ID NO: 139 - protein, 116 aa, synthetic construct
  REGION 1..116: note = artificial sequence
KVQLVESGGGLVQPGGSLRLSCAASGFKWEHYAMSWVRQAPGKGLEWVGSISPEGNHKYYNTKFIDRFTISRDNSKNTLYLQMNSLRAEDTAVYYCARDWDYFDRWGQGTLVTVSS

SEQ ID NO: 140 - protein, 106 aa, synthetic construct; identical to SEQ ID NO: 40
  REGION 1..106: note = artificial sequence

SEQ ID NO: 141 - protein, 116 aa, synthetic construct
  REGION 1..116: note = artificial sequence
GVQLVESGGGLVQPGGSLRLSCAASGFKWEHYAMSWVRQAPGKGLEWVGEISPDGRHKYYNTKFIDRFTISRDDSKNTLYLQMNSLRAEDTAVYYCARDWEYFDYWGQGTLVTVSS

SEQ ID NO: 142 - protein, 106 aa, synthetic construct; identical to SEQ ID NO: 40
  REGION 1..106: note = artificial sequence

SEQ ID NO: 143 - protein, 116 aa, synthetic construct
  REGION 1..116: note = artificial sequence
RVQLVESGGGLVQPGGSLRLSCAASGYRWDHYAMSWVRQAPGKGLEWVGSISPDGRTKYYNTKFIDRFTISRDDEKNTLYLQMNSLRAEDTAVYYCARDWEYFDTWGQGTLVTVSS

SEQ ID NO: 144 - protein, 106 aa, synthetic construct; identical to SEQ ID NO: 40
  REGION 1..106: note = artificial sequence

SEQ ID NO: 145 - protein, 116 aa, synthetic construct
  REGION 1..116: note = artificial sequence
RVQLVESGGGLVQPGGSLRLSCAASGFKFDIYAMSWVRQAPGKGLEWVGEISPDGQSKYYNTKFIDRFTISRDDSKNTLYLQMNSLRAEDTAVYYCARDWDYFDNWGQGTLVTVSS

SEQ ID NO: 146 - protein, 106 aa, synthetic construct; identical to SEQ ID NO: 40
  REGION 1..106: note = artificial sequence

SEQ ID NO: 147 - protein, 116 aa, synthetic construct
  REGION 1..116: note = artificial sequence
KVQLVESGGGLVQPGGSLRLSCAASGFKWEHYAMSWVRQAPGKGLEWVGEISPKGDNKYYNTKFIDRFTISRDDEKNTLYLQMNSLRAEDTAVYYCARDWEYFDNWGQGTLVTVSS

SEQ ID NO: 148 - protein, 106 aa, synthetic construct; identical to SEQ ID NO: 40
  REGION 1..106: note = artificial sequence

SEQ ID NO: 149 - protein, 116 aa, synthetic construct
  REGION 1..116: note = artificial sequence
RVQLVESGGGLVQPGGSLRLSCAASGYRWDHYAMSWVRQAPGKGLEWVGEISPKGDSKYYNTKFIDRFTISRDDEKNTLYLQMNSLRAEDTAVYYCARDWEYFDNWGQGTLVTVSS

SEQ ID NO: 150 - protein, 106 aa, synthetic construct; identical to SEQ ID NO: 40
  REGION 1..106: note = artificial sequence

SEQ ID NO: 151 - protein, 116 aa, synthetic construct
  REGION 1..116: note = artificial sequence
KVQLVESGGGLVQPGGSLRLSCAASGFRWEHYAMSWVRQAPGKGLEWVGEISPDGQDKYYNTKFIDRFTISRDDSKNTLYLQMNSLRAEDTAVYYCARDWEYFDTWGQGTLVTVSS

SEQ ID NO: 152 - protein, 106 aa, synthetic construct; identical to SEQ ID NO: 40
  REGION 1..106: note = artificial sequence

SEQ ID NO: 153 - protein, 116 aa, synthetic construct
  REGION 1..116: note = artificial sequence
KVQLVESGGGLVQPGGSLRLSCAASGFTWDHYAMSWVRQAPGKGLEWVGSISPRGNHKYYNTKFIDRFTISRDDDKNTLYLQMNSLRAEDTAVYYCARDWDYFDHWGQGTLVTVSS

SEQ ID NO: 154 - protein, 106 aa, synthetic construct; identical to SEQ ID NO: 40
  REGION 1..106: note = artificial sequence

SEQ ID NO: 155 - protein, 116 aa, synthetic construct
  REGION 1..116: note = artificial sequence
KVQLVESGGGLVQPGGSLRLSCAASGFKYDIYAMSWVRQAPGKGLEWVGDISPKGETKYYNTKFIDRFTISRDNSKNTLYLQMNSLRAEDTAVYYCARDWDYFDAWGQGTLVTVSS

SEQ ID NO: 156 - protein, 106 aa, synthetic construct; identical to SEQ ID NO: 40
  REGION 1..106: note = artificial sequence

SEQ ID NO: 157 - protein, 116 aa, synthetic construct
  REGION 1..116: note = artificial sequence
EVQLVESGGGLVQPGGSLRLSCAASGYKFDKYAMSWVRQAPGKGLEWVGEISPRGDHKYYNTKFIDRFTISRDDDKNTLYLQMNSLRAEDTAVYYCARDWEYFDSWGQGTLVTVSS

SEQ ID NO: 158 - protein, 106 aa, synthetic construct; identical to SEQ ID NO: 40
  REGION 1..106: note = artificial sequence

SEQ ID NO: 159 - protein, 116 aa, synthetic construct
  REGION 1..116: note = artificial sequence
KVQLVESGGGLVQPGGSLRLSCAASGFRWDIYAMSWVRQAPGKGLEWVGEISPDGRTKYYNTKFIDRFTISRDDSKNTLYLQMNSLRAEDTAVYYCARDWDYFDTWGQGTLVTVSS

SEQ ID NO: 160 - protein, 106 aa, synthetic construct; identical to SEQ ID NO: 40
  REGION 1..106: note = artificial sequence

SEQ ID NO: 161 - protein, 116 aa, synthetic construct
  REGION 1..116: note = artificial sequence
RVQLVESGGGLVQPGGSLRLSCAASGFRYDHYAMSWVRQAPGKGLEWVGSISPKGNHKYYNTKFIDRFTISRDNSKNTLYLQMNSLRAEDTAVYYCARDWDYFDHWGQGTLVTVSS

SEQ ID NO: 162 - protein, 106 aa, synthetic construct; identical to SEQ ID NO: 40
  REGION 1..106: note = artificial sequence

SEQ ID NO: 163 - protein, 116 aa, synthetic construct
  REGION 1..116: note = artificial sequence
EVQLVESGGGLVQPGGSLRLSCAASGFQFDHYAMSWVRQAPGKGLEWVGSISPRGDHKYYNTKFIDRFTISRDNSKNTLYLQMNSLRAEDTAVYYCARDWDYFDQWGQGTLVTVSS

SEQ ID NO: 164 - protein, 106 aa, synthetic construct; identical to SEQ ID NO: 40
  REGION 1..106: note = artificial sequence

SEQ ID NO: 165 - protein, 116 aa, synthetic construct
  REGION 1..116: note = artificial sequence
KVQLVESGGGLVQPGGSLRLSCAASGYRYDYYAMSWVRQAPGKGLEWVGSISPKGDHKYYNTKFIDRFTISRDNSKNTLYLQMNSLRAEDTAVYYCARDWDYFDGWGQGTLVTVSS

SEQ ID NO: 166 - protein, 106 aa, synthetic construct; identical to SEQ ID NO: 40
  REGION 1..106: note = artificial sequence

SEQ ID NO: 167 - protein, 116 aa, synthetic construct
  REGION 1..116: note = artificial sequence
KVQLVESGGGLVQPGGSLRLSCAASGYTWEHYAMSWVRQAPGKGLEWVGSISPDGRHKYYNTKFIDRFTISRDDDKNTLYLQMNSLRAEDTAVYYCARDWDYFDYWGQGTLVTVSS

SEQ ID NO: 168 - protein, 106 aa, synthetic construct; identical to SEQ ID NO: 40
  REGION 1..106: note = artificial sequence

SEQ ID NO: 169 - protein, 116 aa, synthetic construct
  REGION 1..116: note = artificial sequence
KVQLVESGGGLVQPGGSLRLSCAASGFRYDHYAMSWVRQAPGKGLEWVGSISPDGDHKYYNTKFIDRFTISRDDSKNTLYLQMNSLRAEDTAVYYCARDWDYFDHWGQGTLVTVSS

SEQ ID NO: 170 - protein, 106 aa, synthetic construct; identical to SEQ ID NO: 40
  REGION 1..106: note = artificial sequence

SEQ ID NO: 171 - protein, 116 aa, synthetic construct
  REGION 1..116: note = artificial sequence
RVQLVESGGGLVQPGGSLRLSCAASGFTWDHYAMTWVRQAPGKGLEWVGEISPRGDSKYYNTKFIDRFTISRDNSKNTLYLQMNSLRAEDTAVYYCARDWDYFDHWGQGTLVTVSS

SEQ ID NO: 172 - protein, 106 aa, synthetic construct; identical to SEQ ID NO: 40
  REGION 1..106: note = artificial sequence

SEQ ID NO: 173 - protein, 116 aa, synthetic construct
  REGION 1..116: note = artificial sequence
EVQLVESGGGLVQPGGSLRLSCAASGWSYDHYAMSWVRQAPGKGLEWVGSISPKGDHKYYNTKFIDRFTISRDNEKNTLYLQMNSLRAEDTAVYYCARDWDYFDYWGQGTLVTVSS

SEQ ID NO: 174 - protein, 106 aa, synthetic construct; identical to SEQ ID NO: 40
  REGION 1..106: note = artificial sequence

SEQ ID NO: 175 - protein, 116 aa, synthetic construct
  REGION 1..116: note = artificial sequence
RVQLVESGGGLVQPGGSLRLSCAASGFRWDYYAMSWVRQAPGKGLEWVGSISPDGEHKYYNTKFIDRFTISRDDSKNTLYLQMNSLRAEDTAVYYCARDWDYFDTWGQGTLVTVSS

SEQ ID NO: 176 - protein, 106 aa, synthetic construct; identical to SEQ ID NO: 40
  REGION 1..106: note = artificial sequence

SEQ ID NO: 177 - protein, 116 aa, synthetic construct
  REGION 1..116: note = artificial sequence
RVQLVESGGGLVQPGGSLRLSCAASGFRYDYYAMSWVRQAPGKGLEWVGSISPKGDTKYYNTKFIDRFTISRDDEKNTLYLQMNSLRAEDTAVYYCARDWDYFDIWGQGTLVTVSS

SEQ ID NO: 178 - protein, 106 aa, synthetic construct; identical to SEQ ID NO: 40
  REGION 1..106: note = artificial sequence

SEQ ID NO: 179 - protein, 115 aa, synthetic construct; identical to SEQ ID NO: 41
  REGION 1..115: note = artificial sequence

SEQ ID NO: 180 - protein, 106 aa, synthetic construct
  REGION 1..106: note = artificial sequence
AIQMTQSPSSLSASVGDRVTITCQASYWLSNYLAWYQQKPGKAPKLLIFDGDLPYERPSRFSGSGSHDEYTLTISSLQPEDFATYYCQQYRNEPYTFGQGTKLEIK

SEQ ID NO: 181 - protein, 115 aa, synthetic construct; identical to SEQ ID NO: 41
  REGION 1..115: note = artificial sequence

SEQ ID NO: 182 - protein, 106 aa, synthetic construct
  REGION 1..106: note = artificial sequence
AIQMTQSPSSLSASVGDRVTITCQSQYWLSNYLAWYQQKPGKAPKLLIFDGDIPYEIPSRFSGSGSHEEYTLTISSLQPEDFATYYCQQYRDEPYTFGQGTKLEIK

SEQ ID NO: 183 - protein, 115 aa, synthetic construct; identical to SEQ ID NO: 41
  REGION 1..115: note = artificial sequence

SEQ ID NO: 184 - protein, 106 aa, synthetic construct
  REGION 1..106: note = artificial sequence
AIQMTQSPSSLSASVGDRVTITCQASYWLSNYLAWYQQKPGKAPKLLIFDGDLPYEIPSRFSGSGSHEEYTLTISSLQPEDFATYYCQQYREEPYTFGQGTKLEIK

SEQ ID NO: 185 - protein, 115 aa, synthetic construct; identical to SEQ ID NO: 41
  REGION 1..115: note = artificial sequence

SEQ ID NO: 186 - protein, 106 aa, synthetic construct
  REGION 1..106: note = artificial sequence
AIQMTQSPSSLSASVGDRVTITCQSTLWLSNYLAWYQQKPGKAPKLLIFDGDLPYEIPSRFSGSGSHEEYTLTISSLQPEDFATYYCQQYKETPYTFGQGTKLEIK

SEQ ID NO: 187 - protein, 115 aa, synthetic construct; identical to SEQ ID NO: 41
  REGION 1..115: note = artificial sequence

SEQ ID NO: 188 - protein, 106 aa, synthetic construct
  REGION 1..106: note = artificial sequence
AIQMTQSPSSLSASVGDRVTITCQASYWLSNYLAWYQQKPGKAPKLLIFDGDIPWERPSRFSGSGSHDEYTLTISSLQPEDFATYYCQQYRDTPYTFGQGTKLEIK

SEQ ID NO: 189 - protein, 115 aa, synthetic construct; identical to SEQ ID NO: 41
  REGION 1..115: note = artificial sequence

SEQ ID NO: 190 - protein, 106 aa, synthetic construct
  REGION 1..106: note = artificial sequence
AIQMTQSPSSLSASVGDRVTITCQASYWLSNYLAWYQQKPGKAPKLLIFDGDIPYERPSRFSGSGSHEEYTLTISSLQPEDFATYYCQQYRETPYTFGQGTKLEIK

SEQ ID NO: 191 - protein, 115 aa, synthetic construct; identical to SEQ ID NO: 41
  REGION 1..115: note = artificial sequence

SEQ ID NO: 192 - protein, 106 aa, synthetic construct
  REGION 1..106: note = artificial sequence
AIQMTQSPSSLSASVGDRVTITCQASYWLSNYLAWYQQKPGKAPKLLIFDGDLPYERPSRFSGSGSEEEYTLTISSLQPEDFATYYCQQYRNTPYTFGQGTKLEIK

SEQ ID NO: 193 - protein, 115 aa, synthetic construct; identical to SEQ ID NO: 41
  REGION 1..115: note = artificial sequence

SEQ ID NO: 194 - protein, 106 aa, synthetic construct
  REGION 1..106: note = artificial sequence
AIQMTQSPSSLSASVGDRVTITCQASYWLSNYLAWYQQKPGKAPKLLIFDGDLPYEIPSRFSGSGSHEEYTLTISSLQPEDFATYYCQQYKETPYTFGQGTKLEIK

SEQ ID NO: 195 - protein, 115 aa, synthetic construct; identical to SEQ ID NO: 41
  REGION 1..115: note = artificial sequence

SEQ ID NO: 196 - protein, 106 aa, synthetic construct
  REGION 1..106: note = artificial sequence
AIQMTQSPSSLSASVGDRVTITCQASYWLSNYLAWYQQKPGKAPKLLIFDGDLPYEVPSRFSGSGSHDEYTLTISSLQPEDFATYYCQQYRDTPYTFGQGTKLEIK

SEQ ID NO: 197 - protein, 115 aa, synthetic construct; identical to SEQ ID NO: 41
  REGION 1..115: note = artificial sequence

SEQ ID NO: 198 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYRETPYTFGQGTKLEIK

SEQ ID NO: 199 - protein, 115 aa, synthetic construct; identical to SEQ ID NO: 41
  REGION 1..115: note = artificial sequence

SEQ ID NO: 200 - protein, 106 aa, synthetic construct
  REGION 1..106: note = artificial sequence
AIQMTQSPSSLSASVGDRVTITCQSQLWLSNYLAWYQQKPGKAPKLLIFDGDIPYERPSRFSGSGSHEEYTLTISSLQPEDFATYYCQQYRSEPYTFGQGTKLEIK

SEQ ID NO: 201 - protein, 115 aa, synthetic construct; identical to SEQ ID NO: 41
  REGION 1..115: note = artificial sequence

SEQ ID NO: 202 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYREDPYTFGQGTKLEIK

SEQ ID NO: 203 - protein, 115 aa, synthetic construct; identical to SEQ ID NO: 41
  REGION 1..115: note = artificial sequence

SEQ ID NO: 204 - protein, 106 aa, synthetic construct
  REGION 1..106: note = artificial sequence
AIQMTQSPSSLSASVGDRVTITCQVSHWLSNYLAWYQQKPGKAPKLLIFDGDLPYEIPSRFSGSGSHDEYTLTISSLQPEDFATYYCQQYRYTPYTFGQGTKLEIK

SEQ ID NO: 205 - protein, 115 aa, synthetic construct; identical to SEQ ID NO: 41
  REGION 1..115: note = artificial sequence

SEQ ID NO: 206 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSHEEYTLTISSLQPEDFATYYCQQYRQTPYTFGQGTKLEIK

SEQ ID NO: 207 - protein, 115 aa, synthetic construct; identical to SEQ ID NO: 41
  REGION 1..115: note = artificial sequence

SEQ ID NO: 208 - protein, 106 aa, synthetic construct
  REGION 1..106: note = artificial sequence
AIQMTQSPSSLSASVGDRVTITCQSQFWLSNYLAWYQQKPGKAPKLLIFDGDLPYERPSRFSGSGSHDEYTLTISSLQPEDFATYYCQQYESEPYTFGQGTKLEIK

SEQ ID NO: 209 - protein, 115 aa, synthetic construct; identical to SEQ ID NO: 41
  REGION 1..115: note = artificial sequence

SEQ ID NO: 210 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSWDEYTLTISSLQPEDFATYYCQQYRETPYTFGQGTKLEIK

SEQ ID NO: 211 - protein, 115 aa, synthetic construct; identical to SEQ ID NO: 41
  REGION 1..115: note = artificial sequence

SEQ ID NO: 212 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYRDTPYTFGQGTKLEIK

SEQ ID NO: 213 - protein, 115 aa, synthetic construct; identical to SEQ ID NO: 41
  REGION 1..115: note = artificial sequence

SEQ ID NO: 214 - protein, 106 aa, synthetic construct
  REGION 1..106: note = artificial sequence
AIQMTQSPSSLSASVGDRVTITCQASYWLSNYLAWYQQKPGKAPKLLIFDGDIPWEIPSRFSGSGSHDEYTLTISSLQPEDFATYYCQQYRDTPYTFGQGTKLEIK

SEQ ID NO: 215 - protein, 115 aa, synthetic construct; identical to SEQ ID NO: 41
  REGION 1..115: note = artificial sequence

SEQ ID NO: 216 - protein, 106 aa, synthetic construct
  REGION 1..106: note = artificial sequence
AIQMTQSPSSLSASVGDRVTITCQSTLWLSNYLAWYQQKPGKAPKLLIFDGDIPWEIPSRFSGSGSWDEYTLTISSLQPEDFATYYCQQYRYTPYTFGQGTKLEIK

SEQ ID NO: 217 - protein, 115 aa, synthetic construct; identical to SEQ ID NO: 41
  REGION 1..115: note = artificial sequence

SEQ ID NO: 218 - protein, 106 aa, synthetic construct
  REGION 1..106: note = artificial sequence
AIQMTQSPSSLSASVGDRVTITCQSQYWLSNYLAWYQQKPGKAPKLLIFDGDLPWEVPSRFSGSGSHDEYTLTISSLQPEDFATYYCQQYKDTPYTFGQGTKLEIK

SEQ ID NO: 219 - protein, 115 aa, synthetic construct; identical to SEQ ID NO: 41
  REGION 1..115: note = artificial sequence

SEQ ID NO: 220 - protein, 106 aa, synthetic construct
  REGION 1..106: note = artificial sequence
AIQMTQSPSSLSASVGDRVTITCQAQHWLSNYLAWYQQKPGKAPKLLIFDGDIPYERPSRFSGSGSHDEYTLTISSLQPEDFATYYCQQYRETPYTFGQGTKLEIK

SEQ ID NO: 221 - protein, 115 aa, synthetic construct; identical to SEQ ID NO: 41
  REGION 1..115: note = artificial sequence

SEQ ID NO: 222 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEDYTLTISSLQPEDFATYYCQQYRSEPYTFGQGTKLEIK

SEQ ID NO: 223 - protein, 115 aa, synthetic construct; identical to SEQ ID NO: 41
  REGION 1..115: note = artificial sequence

SEQ ID NO: 224 - protein, 106 aa, synthetic construct
  REGION 1..106: note = artificial sequence
AIQMTQSPSSLSASVGDRVTITCQAHYWLSNYLAWYQQKPGKAPKLLIFDGDIPYEIPSRFSGSGSEDEYTLTISSLQPEDFATYYCQQYRSTPYTFGQGTKLEIK

SEQ ID NO: 225 - protein, 115 aa, synthetic construct; identical to SEQ ID NO: 41
  REGION 1..115: note = artificial sequence

SEQ ID NO: 226 - protein, 106 aa, synthetic construct
  REGION 1..106: note = artificial sequence
AIQMTQSPSSLSASVGDRVTITCQASYWLSNYLAWYQQKPGKAPKLLIFDGDLPYEIPSRFSGSGSHDEYTLTISSLQPEDFATYYCQQYRETPYTFGQGTKLEIK

SEQ ID NO: 227 - protein, 115 aa, synthetic construct; identical to SEQ ID NO: 41
  REGION 1..115: note = artificial sequence

SEQ ID NO: 228 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHDEYTLTISSLQPEDFATYYCQQYREEPYTFGQGTKLEIK

SEQ ID NO: 229 - protein, 115 aa, synthetic construct; identical to SEQ ID NO: 41
  REGION 1..115: note = artificial sequence

SEQ ID NO: 230 - protein, 106 aa, synthetic construct
  REGION 1..106: note = artificial sequence
AIQMTQSPSSLSASVGDRVTITCQSQLWLSNYLAWYQQKPGKAPKLLIFDGDLPYEIPSRFSGSGSHEEYTLTISSLQPEDFATYYCQQYRETPYTFGQGTKLEIK

SEQ ID NO: 231 - protein, 115 aa, synthetic construct; identical to SEQ ID NO: 41
  REGION 1..115: note = artificial sequence

SEQ ID NO: 232 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSHDEYTLTISSLQPEDFATYYCQQYREDPYTFGQGTKLEIK

SEQ ID NO: 233 - protein, 115 aa, synthetic construct; identical to SEQ ID NO: 41
  REGION 1..115: note = artificial sequence

SEQ ID NO: 234 - protein, 106 aa, synthetic construct
  REGION 1..106: note = artificial sequence
AIQMTQSPSSLSASVGDRVTITCQASYWLSNYLAWYQQKPGKAPKLLIFDGDIPWEVPSRFSGSGSHEEYTLTISSLQPEDFATYYCQQYREEPYTFGQGTKLEIK

SEQ ID NO: 235 - protein, 115 aa, synthetic construct; identical to SEQ ID NO: 41
  REGION 1..115: note = artificial sequence

SEQ ID NO: 236 - protein, 106 aa, synthetic construct
  REGION 1..106: note = artificial sequence
AIQMTQSPSSLSASVGDRVTITCQSQYWLSNYLAWYQQKPGKAPKLLIFDGDLPYERPSRFSGSGSHDEYTLTISSLQPEDFATYYCQQYRSEPYTFGQGTKLEIK

SEQ ID NO: 237 - protein, 115 aa, synthetic construct; identical to SEQ ID NO: 41
  REGION 1..115: note = artificial sequence

SEQ ID NO: 238 - protein, 106 aa, synthetic construct
  REGION 1..106: note = artificial sequence
AIQMTQSPSSLSASVGDRVTITCQASYWLSNYLAWYQQKPGKAPKLLIFDGDLPYERPSRFSGSGSHDEYTLTISSLQPEDFATYYCQQYRDTPYTFGQGTKLEIK

SEQ ID NO: 239 - protein, 115 aa, synthetic construct; identical to SEQ ID NO: 41
  REGION 1..115: note = artificial sequence

SEQ ID NO: 240 - protein, 106 aa, synthetic construct
  REGION 1..106: note = artificial sequence
AIQMTQSPSSLSASVGDRVTITCQASYWLSNYLAWYQQKPGKAPKLLIFDGDIPYEIPSRFSGSGSHDEYTLTISSLQPEDFATYYCQQYRETPYTFGQGTKLEIK

SEQ ID NO: 241 - protein, 115 aa, synthetic construct; identical to SEQ ID NO: 41
  REGION 1..115: note = artificial sequence

SEQ ID NO: 242 - protein, 106 aa, synthetic construct
  REGION 1..106: note = artificial sequence
AIQMTQSPSSLSASIGDRVTITCQSQYWLSNYLAWYQQKPGKAPKLLIFDGDIPWELPSRFSGSGSHDEYTLTISSLQPEDFATYYCQQYREEPYTFGQGTKLEIK

SEQ ID NO: 243 - protein, 115 aa, synthetic construct; identical to SEQ ID NO: 41
  REGION 1..115: note = artificial sequence

SEQ ID NO: 244 - protein, 106 aa, synthetic construct
  REGION 1..106: note = artificial sequence
AIQMTQSPSSLSASVGDRVTITCQASYWLSNYLAWYQQKPGKAPKLLIFDGDLPYETPSRFSGSGSHDEYTLTISSLQPEDFATYYCQQYRETPYTFGQGTKLEIK

SEQ ID NO: 245 - protein, 116 aa, synthetic construct; identical to SEQ ID NO: 5
  REGION 1..116: note = artificial sequence

SEQ ID NO: 246 - protein, 106 aa, synthetic construct; identical to SEQ ID NO: 70
  REGION 1..106: note = artificial sequence

SEQ ID NO: 247 - protein, 116 aa, synthetic construct; identical to SEQ ID NO: 49
  REGION 1..116: note = artificial sequence

SEQ ID NO: 248 - protein, 106 aa, synthetic construct; identical to SEQ ID NO: 70
  REGION 1..106: note = artificial sequence

SEQ ID NO: 249 - protein, 116 aa, synthetic construct; identical to SEQ ID NO: 1
  REGION 1..116: note = artificial sequence

SEQ ID NO: 250 - protein, 106 aa, synthetic construct; identical to SEQ ID NO: 70
  REGION 1..106: note = artificial sequence

SEQ ID NO: 251 - protein, 116 aa, synthetic construct; identical to SEQ ID NO: 53
  REGION 1..116: note = artificial sequence

SEQ ID NO: 252 - protein, 106 aa, synthetic construct; identical to SEQ ID NO: 70
  REGION 1..106: note = artificial sequence

SEQ ID NO: 253 - protein, 116 aa, synthetic construct; identical to SEQ ID NO: 7
  REGION 1..116: note = artificial sequence

SEQ ID NO: 254 - protein, 106 aa, synthetic construct; identical to SEQ ID NO: 70
  REGION 1..106: note = artificial sequence

SEQ ID NO: 255 - protein, 116 aa, synthetic construct; identical to SEQ ID NO: 55
  REGION 1..116: note = artificial sequence

SEQ ID NO: 256 - protein, 106 aa, synthetic construct; identical to SEQ ID NO: 70
  REGION 1..106: note = artificial sequence

SEQ ID NO: 257 - protein, 116 aa, synthetic construct; identical to SEQ ID NO: 3
  REGION 1..116: note = artificial sequence

SEQ ID NO: 258 - protein, 106 aa, synthetic construct; identical to SEQ ID NO: 70
  REGION 1..106: note = artificial sequence

SEQ ID NO: 259 - protein, 116 aa, synthetic construct; identical to SEQ ID NO: 61
  REGION 1..116: note = artificial sequence

SEQ ID NO: 260 - protein, 106 aa, synthetic construct; identical to SEQ ID NO: 70
  REGION 1..106: note = artificial sequence

SEQ ID NO: 261 - protein, 116 aa, synthetic construct; identical to SEQ ID NO: 65
  REGION 1..116: note = artificial sequence

SEQ ID NO: 262 - protein, 106 aa, synthetic construct; identical to SEQ ID NO: 70
  REGION 1..106: note = artificial sequence

SEQ ID NO: 263 - protein, 116 aa, synthetic construct; identical to SEQ ID NO: 63
  REGION 1..116: note = artificial sequence

SEQ ID NO: 264 - protein, 106 aa, synthetic construct; identical to SEQ ID NO: 70
  REGION 1..106: note = artificial sequence

SEQ ID NO: 265 - protein, 116 aa, synthetic construct; identical to SEQ ID NO: 67
  REGION 1..116: note = artificial sequence

SEQ ID NO: 266 - protein, 106 aa, synthetic construct; identical to SEQ ID NO: 70
  REGION 1..106: note = artificial sequence

SEQ ID NO: 267 - protein, 116 aa, synthetic construct; identical to SEQ ID NO: 49
  REGION 1..116: note = artificial sequence

SEQ ID NO: 268 - protein, 106 aa, synthetic construct; identical to SEQ ID NO: 4
  REGION 1..106: note = artificial sequence

SEQ ID NO: 269 - protein, 116 aa, synthetic construct; identical to SEQ ID NO: 1
  REGION 1..116: note = artificial sequence

SEQ ID NO: 270 - protein, 106 aa, synthetic construct; identical to SEQ ID NO: 4
  REGION 1..106: note = artificial sequence

SEQ ID NO: 271 - protein, 116 aa, synthetic construct; identical to SEQ ID NO: 53
  REGION 1..116: note = artificial sequence

SEQ ID NO: 272 - protein, 106 aa, synthetic construct; identical to SEQ ID NO: 4
  REGION 1..106: note = artificial sequence

SEQ ID NO: 273 - protein, 116 aa, synthetic construct; identical to SEQ ID NO: 7
  REGION 1..116: note = artificial sequence

SEQ ID NO: 274 - protein, 106 aa, synthetic construct; identical to SEQ ID NO: 4
  REGION 1..106: note = artificial sequence

SEQ ID NO: 275 - protein, 116 aa, synthetic construct; identical to SEQ ID NO: 55
  REGION 1..116: note = artificial sequence

SEQ ID NO: 276 - protein, 106 aa, synthetic construct; identical to SEQ ID NO: 4
  REGION 1..106: note = artificial sequence

SEQ ID NO: 277 - protein, 116 aa, synthetic construct; identical to SEQ ID NO: 57
  REGION 1..116: note = artificial sequence

SEQ ID NO: 278 - protein, 106 aa, synthetic construct; identical to SEQ ID NO: 4
  REGION 1..106: note = artificial sequence

SEQ ID NO: 279 - protein, 116 aa, synthetic construct; identical to SEQ ID NO: 61
  REGION 1..116: note = artificial sequence

SEQ ID NO: 280 - protein, 106 aa, synthetic construct; identical to SEQ ID NO: 4
  REGION 1..106: note = artificial sequence

SEQ ID NO: 281 - protein, 116 aa, synthetic construct; identical to SEQ ID NO: 65
  REGION 1..116: note = artificial sequence

SEQ ID NO: 282 - protein, 106 aa, synthetic construct; identical to SEQ ID NO: 4
  REGION 1..106: note = artificial sequence

SEQ ID NO: 283 - protein, 116 aa, synthetic construct; identical to SEQ ID NO: 63
  REGION 1..116: note = artificial sequence

SEQ ID NO: 284 - protein, 106 aa, synthetic construct; identical to SEQ ID NO: 4
  REGION 1..106: note = artificial sequence

SEQ ID NO: 285 - protein, 116 aa, synthetic construct; identical to SEQ ID NO: 67
  REGION 1..116: note = artificial sequence

SEQ ID NO: 286 - protein, 106 aa, synthetic construct; identical to SEQ ID NO: 4
  REGION 1..106: note = artificial sequence

SEQ ID NO: 287 - protein, 116 aa, synthetic construct; identical to SEQ ID NO: 5
  REGION 1..116: note = artificial sequence

SEQ ID NO: 288 - protein, 106 aa, synthetic construct; identical to SEQ ID NO: 10
  REGION 1..106: note = artificial sequence

SEQ ID NO: 289 - protein, 116 aa, synthetic construct; identical to SEQ ID NO: 49
  REGION 1..116: note = artificial sequence

SEQ ID NO: 290 - protein, 106 aa, synthetic construct; identical to SEQ ID NO: 10
  REGION 1..106: note = artificial sequence

SEQ ID NO: 291 - protein, 116 aa, synthetic construct; identical to SEQ ID NO: 7
  REGION 1..116: note = artificial sequence

SEQ ID NO: 292 - protein, 106 aa, synthetic construct; identical to SEQ ID NO: 10
  REGION 1..106: note = artificial sequence

SEQ ID NO: 293 - protein, 116 aa, synthetic construct; identical to SEQ ID NO: 61
  REGION 1..116: note = artificial sequence

SEQ ID NO: 294 - protein, 106 aa, synthetic construct; identical to SEQ ID NO: 10
  REGION 1..106: note = artificial sequence

SEQ ID NO: 295 - protein, 116 aa, synthetic construct; identical to SEQ ID NO: 65
  REGION 1..116: note = artificial sequence

SEQ ID NO: 296 - protein, 106 aa, synthetic construct; identical to SEQ ID NO: 10
  REGION 1..106: note = artificial sequence

SEQ ID NO: 297 - protein, 116 aa, synthetic construct; identical to SEQ ID NO: 63
  REGION 1..116: note = artificial sequence

SEQ ID NO: 298 - protein, 106 aa, synthetic construct; identical to SEQ ID NO: 10
  REGION 1..106: note = artificial sequence

SEQ ID NO: 299 - protein, 116 aa, synthetic construct; identical to SEQ ID NO: 53
  REGION 1..116: note = artificial sequence

SEQ ID NO: 300 - protein, 106 aa, synthetic construct; identical to SEQ ID NO: 2
  REGION 1..106: note = artificial sequence

SEQ ID NO: 301 - protein, 116 aa, synthetic construct; identical to SEQ ID NO: 55
  REGION 1..116: note = artificial sequence

SEQ ID NO: 302 - protein, 106 aa, synthetic construct; identical to SEQ ID NO: 2
  REGION 1..106: note = artificial sequence

SEQ ID NO: 303 - protein, 116 aa, synthetic construct; identical to SEQ ID NO: 3
  REGION 1..116: note = artificial sequence

SEQ ID NO: 304 - protein, 106 aa, synthetic construct; identical to SEQ ID NO: 2
  REGION 1..106: note = artificial sequence

SEQ ID NO: 305 - protein, 116 aa, synthetic construct; identical to SEQ ID NO: 57
  REGION 1..116: note = artificial sequence

SEQ ID NO: 306 - protein, 106 aa, synthetic construct; identical to SEQ ID NO: 2
  REGION 1..106: note = artificial sequence

SEQ ID NO: 307 - protein, 116 aa, synthetic construct; identical to SEQ ID NO: 61
  REGION 1..116: note = artificial sequence

SEQ ID NO: 308 - protein, 106 aa, synthetic construct; identical to SEQ ID NO: 2
  REGION 1..106: note = artificial sequence

SEQ ID NO: 309 - protein, 116 aa, synthetic construct; identical to SEQ ID NO: 67
  REGION 1..116: note = artificial sequence

SEQ ID NO: 310 - protein, 106 aa, synthetic construct; identical to SEQ ID NO: 2
  REGION 1..106: note = artificial sequence

SEQ ID NO: 311 - protein, 116 aa, synthetic construct; identical to SEQ ID NO: 5
  REGION 1..116: note = artificial sequence

SEQ ID NO: 312 - protein, 106 aa, synthetic construct; identical to SEQ ID NO: 78
  REGION 1..106: note = artificial sequence

SEQ ID NO: 313 - protein, 116 aa, synthetic construct; identical to SEQ ID NO: 49
  REGION 1..116: note = artificial sequence

SEQ ID NO: 314 - protein, 106 aa, synthetic construct; identical to SEQ ID NO: 78
  REGION 1..106: note = artificial sequence

SEQ ID NO: 315 - protein, 116 aa, synthetic construct; identical to SEQ ID NO: 1
  REGION 1..116: note = artificial sequence

SEQ ID NO: 316 - protein, 106 aa, synthetic construct; identical to SEQ ID NO: 78
  REGION 1..106: note = artificial sequence

SEQ ID NO: 317 - protein, 116 aa, synthetic construct; identical to SEQ ID NO: 53
  REGION 1..116: note = artificial sequence

SEQ ID NO: 318 - protein, 106 aa, synthetic construct; identical to SEQ ID NO: 78
  REGION 1..106: note = artificial sequence

SEQ ID NO: 319 - protein, 116 aa, synthetic construct; identical to SEQ ID NO: 7
  REGION 1..116: note = artificial sequence

SEQ ID NO: 320 - protein, 106 aa, synthetic construct; identical to SEQ ID NO: 78
  REGION 1..106: note = artificial sequence

SEQ ID NO: 321 - protein, 116 aa, synthetic construct; identical to SEQ ID NO: 55
  REGION 1..116: note = artificial sequence

SEQ ID NO: 322 - protein, 106 aa, synthetic construct; identical to SEQ ID NO: 78
  REGION 1..106: note = artificial sequence

SEQ ID NO: 323 - protein, 116 aa, synthetic construct; identical to SEQ ID NO: 3
  REGION 1..116: note = artificial sequence

SEQ ID NO: 324 - protein, 106 aa, synthetic construct; identical to SEQ ID NO: 78
  REGION 1..106: note = artificial sequence

SEQ ID NO: 325 - protein, 116 aa, synthetic construct; identical to SEQ ID NO: 57
  REGION 1..116: note = artificial sequence

SEQ ID NO: 326 - protein, 106 aa, synthetic construct; identical to SEQ ID NO: 78
  REGION 1..106: note = artificial sequence

SEQ ID NO: 327 - protein, 116 aa, synthetic construct; identical to SEQ ID NO: 61
  REGION 1..116: note = artificial sequence

SEQ ID NO: 328 - protein, 106 aa, synthetic construct; identical to SEQ ID NO: 78
  REGION 1..106: note = artificial sequence

SEQ ID NO: 329 - protein, 116 aa, synthetic construct; identical to SEQ ID NO: 65
  REGION 1..116: note = artificial sequence

SEQ ID NO: 330 - protein, 106 aa, synthetic construct; identical to SEQ ID NO: 78
  REGION 1..106: note = artificial sequence

SEQ ID NO: 331 - protein, 116 aa, synthetic construct; identical to SEQ ID NO: 63
  REGION 1..116: note = artificial sequence

SEQ ID NO: 332 - protein, 106 aa, synthetic construct; identical to SEQ ID NO: 78
  REGION 1..106: note = artificial sequence

SEQ ID NO: 333 - protein, 116 aa, synthetic construct; identical to SEQ ID NO: 67
  REGION 1..116: note = artificial sequence

SEQ ID NO: 334 - protein, 106 aa, synthetic construct; identical to SEQ ID NO: 78
  REGION 1..106: note = artificial sequence

SEQ ID NO: 335 - protein, 116 aa, synthetic construct; identical to SEQ ID NO: 5
  REGION 1..116: note = artificial sequence

SEQ ID NO: 336 - protein, 106 aa, synthetic construct; identical to SEQ ID NO: 80
  REGION 1..106: note = artificial sequence

SEQ ID NO: 337 - protein, 116 aa, synthetic construct; identical to SEQ ID NO: 49
  REGION 1..116: note = artificial sequence

SEQ ID NO: 338 - protein, 106 aa, synthetic construct; identical to SEQ ID NO: 80
  REGION 1..106: note = artificial sequence

SEQ ID NO: 339 - protein, 116 aa, synthetic construct; identical to SEQ ID NO: 1
  REGION 1..116: note = artificial sequence

SEQ ID NO: 340 - protein, 106 aa, synthetic construct; identical to SEQ ID NO: 80
  REGION 1..106: note = artificial sequence

SEQ ID NO: 341 - protein, 116 aa, synthetic construct; identical to SEQ ID NO: 53
  REGION 1..116: note = artificial sequence

SEQ ID NO: 342 - protein, 106 aa, synthetic construct; identical to SEQ ID NO: 80
  REGION 1..106: note = artificial sequence

SEQ ID NO: 343 - protein, 116 aa, synthetic construct; identical to SEQ ID NO: 7
  REGION 1..116: note = artificial sequence

SEQ ID NO: 344 - protein, 106 aa, synthetic construct; identical to SEQ ID NO: 80
  REGION 1..106: note = artificial sequence

SEQ ID NO: 345 - protein, 116 aa, synthetic construct; identical to SEQ ID NO: 55
  REGION 1..116: note = artificial sequence

SEQ ID NO: 346 - protein, 106 aa, synthetic construct; identical to SEQ ID NO: 80
  REGION 1..106: note = artificial sequence

SEQ ID NO: 347 - protein, 116 aa, synthetic construct; identical to SEQ ID NO: 3
  REGION 1..116: note = artificial sequence

SEQ ID NO: 348 - protein, 106 aa, synthetic construct; identical to SEQ ID NO: 80
  REGION 1..106: note = artificial sequence

SEQ ID NO: 349 - protein, 116 aa, synthetic construct; identical to SEQ ID NO: 57
  REGION 1..116: note = artificial sequence

SEQ ID NO: 350 - protein, 106 aa, synthetic construct; identical to SEQ ID NO: 80
  REGION 1..106: note = artificial sequence

SEQ ID NO: 351 - protein, 116 aa, synthetic construct; identical to SEQ ID NO: 61
  REGION 1..116: note = artificial sequence

SEQ ID NO: 352 - protein, 106 aa, synthetic construct; identical to SEQ ID NO: 80
  REGION 1..106: note = artificial sequence

SEQ ID NO: 353 - protein, 116 aa, synthetic construct; identical to SEQ ID NO: 65
  REGION 1..116: note = artificial sequence

SEQ ID NO: 354 - protein, 106 aa, synthetic construct; identical to SEQ ID NO: 80
  REGION 1..106: note = artificial sequence

SEQ ID NO: 355 - protein, 116 aa, synthetic construct; identical to SEQ ID NO: 63
  REGION 1..116: note = artificial sequence

SEQ ID NO: 356 - protein, 106 aa, synthetic construct; identical to SEQ ID NO: 80
  REGION 1..106: note = artificial sequence

SEQ ID NO: 357 - protein, 116 aa, synthetic construct; identical to SEQ ID NO: 67
  REGION 1..116: note = artificial sequence

SEQ ID NO: 358 - protein, 106 aa, synthetic construct; identical to SEQ ID NO: 80
  REGION 1..106: note = artificial sequence

SEQ ID NO: 359 - protein, 116 aa, synthetic construct; identical to SEQ ID NO: 5
  REGION 1..116: note = artificial sequence

SEQ ID NO: 360 - protein, 106 aa, synthetic construct; identical to SEQ ID NO: 82
  REGION 1..106: note = artificial sequence

SEQ ID NO: 361 - protein, 116 aa, synthetic construct; identical to SEQ ID NO: 49
  REGION 1..116: note = artificial sequence

SEQ ID NO: 362 - protein, 106 aa, synthetic construct; identical to SEQ ID NO: 82
  REGION 1..106: note = artificial sequence

SEQ ID NO: 363 - protein, 116 aa, synthetic construct; identical to SEQ ID NO: 1
  REGION 1..116: note = artificial sequence

SEQ ID NO: 364 - protein, 106 aa, synthetic construct; identical to SEQ ID NO: 82
  REGION 1..106: note = artificial sequence

SEQ ID NO: 365 - protein, 116 aa, synthetic construct; identical to SEQ ID NO: 53
  REGION 1..116: note = artificial sequence

SEQ ID NO: 366 - protein, 106 aa, synthetic construct; identical to SEQ ID NO: 82
  REGION 1..106: note = artificial sequence

SEQ ID NO: 367 - protein, 116 aa, synthetic construct; identical to SEQ ID NO: 7
  REGION 1..116: note = artificial sequence

SEQ ID NO: 368 - protein, 106 aa, synthetic construct; identical to SEQ ID NO: 82
  REGION 1..106: note = artificial sequence

SEQ ID NO: 369 - protein, 116 aa, synthetic construct; identical to SEQ ID NO: 55
  REGION 1..116: note = artificial sequence

SEQ ID NO: 370 - protein, 106 aa, synthetic construct; identical to SEQ ID NO: 82
  REGION 1..106: note = artificial sequence

SEQ ID NO: 371 - protein, 116 aa, synthetic construct; identical to SEQ ID NO: 3
  REGION 1..116: note = artificial sequence

SEQ ID NO: 372 - protein, 106 aa, synthetic construct; identical to SEQ ID NO: 82
  REGION 1..106: note = artificial sequence

SEQ ID NO: 373 - protein, 116 aa, synthetic construct; identical to SEQ ID NO: 57
  REGION 1..116: note = artificial sequence

SEQ ID NO: 374 - protein, 106 aa, synthetic construct; identical to SEQ ID NO: 82
  REGION 1..106: note = artificial sequence

SEQ ID NO: 375 - protein, 116 aa, synthetic construct; identical to SEQ ID NO: 61
  REGION 1..116: note = artificial sequence

SEQ ID NO: 376 - protein, 106 aa, synthetic construct; identical to SEQ ID NO: 82
  REGION 1..106: note = artificial sequence

SEQ ID NO: 377 - protein, 116 aa, synthetic construct; identical to SEQ ID NO: 65
  REGION 1..116: note = artificial sequence

SEQ ID NO: 378 - protein, 106 aa, synthetic construct; identical to SEQ ID NO: 82
  REGION 1..106: note = artificial sequence

SEQ ID NO: 379 - protein, 116 aa, synthetic construct; identical to SEQ ID NO: 63
  REGION 1..116: note = artificial sequence

SEQ ID NO: 380 - protein, 106 aa, synthetic construct; identical to SEQ ID NO: 82
  REGION 1..106: note = artificial sequence

SEQ ID NO: 381 - protein, 116 aa, synthetic construct; identical to SEQ ID NO: 67
  REGION 1..116: note = artificial sequence

SEQ ID NO: 382 - protein, 106 aa, synthetic construct; identical to SEQ ID NO: 82
  REGION 1..106: note = artificial sequence

SEQ ID NO: 383 - protein, 116 aa, synthetic construct; identical to SEQ ID NO: 5
  REGION 1..116: note = artificial sequence

SEQ ID NO: 384 - protein, 106 aa, synthetic construct; identical to SEQ ID NO: 84
  REGION 1..106: note = artificial sequence

SEQ ID NO: 385 - protein, 116 aa, synthetic construct; identical to SEQ ID NO: 49
  REGION 1..116: note = artificial sequence

SEQ ID NO: 386 - protein, 106 aa, synthetic construct; identical to SEQ ID NO: 84
  REGION 1..106: note = artificial sequence

SEQ ID NO: 387 - protein, 116 aa, synthetic construct; identical to SEQ ID NO: 1
  REGION 1..116: note = artificial sequence

SEQ ID NO: 388 - protein, 106 aa, synthetic construct; identical to SEQ ID NO: 84
  REGION 1..106: note = artificial sequence

SEQ ID NO: 389 - protein, 116 aa, synthetic construct; identical to SEQ ID NO: 53
  REGION 1..116: note = artificial sequence

SEQ ID NO: 390 - protein, 106 aa, synthetic construct; identical to SEQ ID NO: 84
  REGION 1..106: note = artificial sequence

SEQ ID NO: 391 - protein, 116 aa, synthetic construct; identical to SEQ ID NO: 7
  REGION 1..116: note = artificial sequence

SEQ ID NO: 392 - protein, 106 aa, synthetic construct; identical to SEQ ID NO: 84
  REGION 1..106: note = artificial sequence

SEQ ID NO: 393 - protein, 116 aa, synthetic construct; identical to SEQ ID NO: 55
  REGION 1..116: note = artificial sequence

SEQ ID NO: 394 - protein, 106 aa, synthetic construct; identical to SEQ ID NO: 84
  REGION 1..106: note = artificial sequence

SEQ ID NO: 395 - protein, 116 aa, synthetic construct; identical to SEQ ID NO: 3
  REGION 1..116: note = artificial sequence

SEQ ID NO: 396 - protein, 106 aa, synthetic construct; identical to SEQ ID NO: 84
  REGION 1..106: note = artificial sequence

SEQ ID NO: 397 - protein, 116 aa, synthetic construct; identical to SEQ ID NO: 57
  REGION 1..116: note = artificial sequence

SEQ ID NO: 398 - protein, 106 aa, synthetic construct; identical to SEQ ID NO: 84
  REGION 1..106: note = artificial sequence

SEQ ID NO: 399 - protein, 116 aa, synthetic construct; identical to SEQ ID NO: 61
  REGION 1..116: note = artificial sequence

SEQ ID NO: 400 - protein, 106 aa, synthetic construct; identical to SEQ ID NO: 84
  REGION 1..106: note = artificial sequence

SEQ ID NO: 401 - protein, 116 aa, synthetic construct; identical to SEQ ID NO: 65
  REGION 1..116: note = artificial sequence

SEQ ID NO: 402 - protein, 106 aa, synthetic construct; identical to SEQ ID NO: 84
  REGION 1..106: note = artificial sequence

SEQ ID NO: 403 - protein, 116 aa, synthetic construct; identical to SEQ ID NO: 63
  REGION 1..116: note = artificial sequence

SEQ ID NO: 404 - protein, 106 aa, synthetic construct; identical to SEQ ID NO: 84
  REGION 1..106: note = artificial sequence

SEQ ID NO: 405 - protein, 116 aa, synthetic construct; identical to SEQ ID NO: 67
  REGION 1..116: note = artificial sequence

SEQ ID NO: 406 - protein, 106 aa, synthetic construct; identical to SEQ ID NO: 84
  REGION 1..106: note = artificial sequence

SEQ ID NO: 407 - protein, 116 aa, synthetic construct; identical to SEQ ID NO: 5
  REGION 1..116: note = artificial sequence

SEQ ID NO: 408 - protein, 106 aa, synthetic construct; identical to SEQ ID NO: 86
  REGION 1..106: note = artificial sequence

SEQ ID NO: 409 - protein, 116 aa, synthetic construct; identical to SEQ ID NO: 1
  REGION 1..116: note = artificial sequence

SEQ ID NO: 410 - protein, 106 aa, synthetic construct; identical to SEQ ID NO: 86
  REGION 1..106: note = artificial sequence

SEQ ID NO: 411 - protein, 116 aa, synthetic construct; identical to SEQ ID NO: 49
  REGION 1..116: note = artificial sequence

SEQ ID NO: 412 - protein, 106 aa, synthetic construct; identical to SEQ ID NO: 86
  REGION 1..106: note = artificial sequence

SEQ ID NO: 413 - protein, 116 aa, synthetic construct; identical to SEQ ID NO: 7
  REGION 1..116: note = artificial sequence

SEQ ID NO: 414 - protein, 106 aa, synthetic construct; identical to SEQ ID NO: 86
  REGION 1..106: note = artificial sequence

SEQ ID NO: 415 - protein, 116 aa, synthetic construct; identical to SEQ ID NO: 55
  REGION 1..116: note = artificial sequence

SEQ ID NO: 416 - protein, 106 aa, synthetic construct; identical to SEQ ID NO: 86
  REGION 1..106: note = artificial sequence

SEQ ID NO: 417 - protein, 116 aa, synthetic construct; identical to SEQ ID NO: 3
  REGION 1..116: note = artificial sequence

SEQ ID NO: 418 - protein, 106 aa, synthetic construct; identical to SEQ ID NO: 86
  REGION 1..106: note = artificial sequence

SEQ ID NO: 419 - protein, 116 aa, synthetic construct; identical to SEQ ID NO: 57
  REGION 1..116: note = artificial sequence

SEQ ID NO: 420 - protein, 106 aa, synthetic construct; identical to SEQ ID NO: 86
  REGION 1..106: note = artificial sequence

SEQ ID NO: 421 - protein, 116 aa, synthetic construct; identical to SEQ ID NO: 63
  REGION 1..116: note = artificial sequence

SEQ ID NO: 422 - protein, 106 aa, synthetic construct; identical to SEQ ID NO: 86
  REGION 1..106: note = artificial sequence

SEQ ID NO: 423 - protein, 116 aa, synthetic construct; identical to SEQ ID NO: 65
  REGION 1..116: note = artificial sequence

SEQ ID NO: 424 - protein, 106 aa, synthetic construct; identical to SEQ ID NO: 86
  REGION 1..106: note = artificial sequence

SEQ ID NO: 425 - protein, 116 aa, synthetic construct; identical to SEQ ID NO: 67
  REGION 1..116: note = artificial sequence

SEQ ID NO: 426 - protein, 106 aa, synthetic construct; identical to SEQ ID NO: 86
  REGION 1..106: note = artificial sequence

SEQ ID NO: 427 - protein, 116 aa, synthetic construct; identical to SEQ ID NO: 57
  REGION 1..116: note = artificial sequence

SEQ ID NO: 428 - protein, 106 aa, synthetic construct; identical to SEQ ID NO: 70
  REGION 1..106: note = artificial sequence

SEQ ID NO: 429 - protein, 116 aa, synthetic construct; identical to SEQ ID NO: 53
  REGION 1..116: note = artificial sequence

SEQ ID NO: 430 - protein, 106 aa, synthetic construct; identical to SEQ ID NO: 10
  REGION 1..106: note = artificial sequence

SEQ ID NO: 431 - protein, 116 aa, synthetic construct; identical to SEQ ID NO: 3
  REGION 1..116: note = artificial sequence

SEQ ID NO: 432 - protein, 106 aa, synthetic construct; identical to SEQ ID NO: 10
  REGION 1..106: note = artificial sequence

SEQ ID NO: 433 - protein, 116 aa, synthetic construct; identical to SEQ ID NO: 63
  REGION 1..116: note = artificial sequence

SEQ ID NO: 434 - protein, 106 aa, synthetic construct; identical to SEQ ID NO: 2
  REGION 1..106: note = artificial sequence

SEQ ID NO: 435 - protein, 444 aa, synthetic construct
  REGION 1..444: note = artificial sequence
QVQLVESGGGLVQPGGSLRLSCAASGFKYDVYAMSWVRQAPGKGLEWVGEISPRGDSKYYNTKFIDRFTISRDNEKNTLYLQMNSLRAEDTAVYYCARDWEYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSP

SEQ ID NO: 436 - protein, 213 aa, synthetic construct
  REGION 1..213: note = artificial sequence
AIQMTQSPSSLSASVGDRVTITCQASYWLSNYLAWYQQKPGKAPKLLIFDGDIPYEIPSRFSGSGSHEEYTLTISSLQPEDFATYYCQQYRETPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 437 - protein, 444 aa, synthetic construct
  REGION 1..444: note = artificial sequence
KVQLVESGGGLVQPGGSLRLSCAASGFKWEHYAMSWVRQAPGKGLEWVGSISPRGDHKYYNTKFIDRFTISRDNDKNTLYLQMNSLRAEDTAVYYCARDWDYFDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSP

SEQ ID NO: 438 - protein, 213 aa, synthetic construct
  REGION 1..213: note = artificial sequence
AIQMTQSPSSLSASVGDRVTITCQSQFWLSNYLAWYQQKPGKAPKLLIFDGDLPYERPSRFSGSGSHDEYTLTISSLQPEDFATYYCQQYREEPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 439 - protein, 444 aa, synthetic construct
  REGION 1..444: note = artificial sequence
EVQLVESGGGLVQPGGSLRLSCAASGFRFDIYAMSWVRQAPGKGLEWVGEISPDGRTKYYNTKFIDRFTISRDNEKNTLYLQMNSLRAEDTAVYYCARDWEYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSP

SEQ ID NO: 440 - protein, 213 aa, synthetic construct; identical to SEQ ID NO: 438
  REGION 1..213: note = artificial sequence

SEQ ID NO: 441 - protein, 446 aa, synthetic construct
  REGION 1..446: note = artificial sequence
RVQLVESGGGLVQPGGSLRLSCAASGFRYDHYAMSWVRQAPGKGLEWVGSISPRGNHKYYNTKFIDRFTISRDDEKNTLYLQMNSLRAEDTAVYYCARDWDYFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 442 - protein, 213 aa, synthetic construct; identical to SEQ ID NO: 436
  REGION 1..213: note = artificial sequence

SEQ ID NO: 443 - protein, 444 aa, synthetic construct; identical to SEQ ID NO: 435
  REGION 1..444: note = artificial sequence

SEQ ID NO: 444 - protein, 213 aa, synthetic construct
  REGION 1..213: note = artificial sequence
AIQMTQSPSSLSASVGDRVTITCQASYWLSNYLAWYQQKPGKAPKLLIFDGDIPWEIPSRFSGSGSHEEYTLTISSLQPEDFATYYCQQYRDTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 220 aa, synthetic construct
  REGION 1..220: note = artificial sequence
KVQLVESGGGLVQPGGSLRLSCAASGFKWEHYAMSWVRQAPGKGLEWVGSISPRGDHKYYNTKFIDRFTISRDNDKNTLYLQMNSLRAEDTAVYYCARDWDYFDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 446 - protein, 213 aa, synthetic construct; identical to SEQ ID NO: 438
  REGION 1..213: note = artificial sequence

SEQ ID NO: 447 - protein, 220 aa, synthetic construct
  REGION 1..220: note = artificial sequence
QVQLVESGGGLVQPGGSLRLSCAASGFKYDVYAMSWVRQAPGKGLEWVGEISPRGDSKYYNTKFIDRFTISRDNEKNTLYLQMNSLRAEDTAVYYCARDWEYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 448 - protein, 213 aa, synthetic construct; identical to SEQ ID NO: 444
  REGION 1..213: note = artificial sequence

SEQ ID NO: 449 - protein, 10 aa, synthetic construct
  REGION 1..10: note = artificial sequence
GFKYDVYAMS

SEQ ID NO: 450 - protein, 17 aa, synthetic construct
  REGION 1..17: note = artificial sequence
EISPRGDSKYYNTKFID

SEQ ID NO: 451 - protein, 7 aa, synthetic construct
  REGION 1..7: note = artificial sequence
DWEYFDN

SEQ ID NO: 452 - protein, 11 aa, synthetic construct
  REGION 1..11: note = artificial sequence
QASYWLSNYLA

SEQ ID NO: 453 - protein, 6 aa, synthetic construct
  REGION 1..6: note = artificial sequence
DGDIPY

SEQ ID NO: 454 - protein, 9 aa, synthetic construct
  REGION 1..9: note = artificial sequence
QQYRETPYT

SEQ ID NO: 455 - protein, 10 aa, synthetic construct
  REGION 1..10: note = artificial sequence
GFKWEHYAMS

SEQ ID NO: 456 - protein, 17 aa, synthetic construct
  REGION 1..17: note = artificial sequence
SISPRGDHKYYNTKFID

SEQ ID NO: 457 - protein, 7 aa, synthetic construct
  REGION 1..7: note = artificial sequence
DWDYFDR

SEQ ID NO: 458 - protein, 11 aa, synthetic construct
  REGION 1..11: note = artificial sequence
QSQFWLSNYLA

SEQ ID NO: 459 - protein, 6 aa, synthetic construct
  REGION 1..6: note = artificial sequence
DGDLPY

SEQ ID NO: 460 - protein, 9 aa, synthetic construct
  REGION 1..9: note = artificial sequence
QQYREEPYT

SEQ ID NO: 461 - protein, 10 aa, synthetic construct
  REGION 1..10: note = artificial sequence
GFRFDIYAMS

SEQ ID NO: 462 - protein, 17 aa, synthetic construct
  REGION 1..17: note = artificial sequence
EISPDGRTKYYNTKFID

SEQ ID NO: 463 - protein, 7 aa, synthetic construct
  REGION 1..7: note = artificial sequence
DWEYFDN

SEQ ID NO: 464 - protein, 11 aa, synthetic construct
  REGION 1..11: note = artificial sequence
QSQFWLSNYLA

SEQ ID NO: 465 - protein, 6 aa, synthetic construct
  REGION 1..6: note = artificial sequence
DGDLPY

SEQ ID NO: 466 - protein, 9 aa, synthetic construct
  REGION 1..9: note = artificial sequence
QQYREEPYT

SEQ ID NO: 467 - protein, 10 aa, synthetic construct
  REGION 1..10: note = artificial sequence
GFRYDHYAMS

SEQ ID NO: 468 - protein, 17 aa, synthetic construct
  REGION 1..17: note = artificial sequence
SISPRGNHKYYNTKFID

SEQ ID NO: 469 - protein, 7 aa, synthetic construct
  REGION 1..7: note = artificial sequence
DWDYFDH

SEQ ID NO: 470 - protein, 11 aa, synthetic construct
  REGION 1..11: note = artificial sequence
QASYWLSNYLA

SEQ ID NO: 471 - protein, 6 aa, synthetic construct
  REGION 1..6: note = artificial sequence
DGDIPY

SEQ ID NO: 472 - protein, 9 aa, synthetic construct
  REGION 1..9: note = artificial sequence
QQYRETPYT

SEQ ID NO: 473 - protein, 10 aa, synthetic construct
  REGION 1..10: note = artificial sequence
GFKYDVYAMS

SEQ ID NO: 474 - protein, 17 aa, synthetic construct
  REGION 1..17: note = artificial sequence
EISPRGDSKYYNTKFID

SEQ ID NO: 475 - protein, 7 aa, synthetic construct
  REGION 1..7: note = artificial sequence
DWEYFDN

SEQ ID NO: 476 - protein, 11 aa, synthetic construct
  REGION 1..11: note = artificial sequence
QASYWLSNYLA

SEQ ID NO: 477 - protein, 6 aa, synthetic construct
  REGION 1..6: note = artificial sequence
DGDIPW

SEQ ID NO: 478 - protein, 9 aa, synthetic construct
  REGION 1..9: note = artificial sequence
QQYRDTPYT

SEQ ID NO: 479 - protein, 10 aa, synthetic construct
  REGION 1..10: note = artificial sequence
GFKWEHYAMS

SEQ ID NO: 480 - protein, 17 aa, synthetic construct
  REGION 1..17: note = artificial sequence
SISPRGDHKYYNTKFID

SEQ ID NO: 481 - protein, 7 aa, synthetic construct
  REGION 1..7: note = artificial sequence
DWDYFDR

SEQ ID NO: 482 - protein, 11 aa, synthetic construct
  REGION 1..11: note = artificial sequence
QSQFWLSNYLA

SEQ ID NO: 483 - protein, 6 aa, synthetic construct
  REGION 1..6: note = artificial sequence
DGDLPY

SEQ ID NO: 484 - protein, 9 aa, synthetic construct
  REGION 1..9: note = artificial sequence
QQYREEPYT

SEQ ID NO: 485 - protein, 10 aa, synthetic construct
  REGION 1..10: note = artificial sequence
GFKYDVYAMS

SEQ ID NO: 486 - protein, 17 aa, synthetic construct
  REGION 1..17: note = artificial sequence
EISPRGDSKYYNTKFID

SEQ ID NO: 487 - protein, 7 aa, synthetic construct
  REGION 1..7: note = artificial sequence
DWEYFDN

SEQ ID NO: 488 - protein, 11 aa, synthetic construct
  REGION 1..11: note = artificial sequence
QASYWLSNYLA

SEQ ID NO: 489 - protein, 6 aa, synthetic construct
  REGION 1..6: note = artificial sequence
DGDIPW

SEQ ID NO: 490 - protein, 9 aa, synthetic construct
  REGION 1..9: note = artificial sequence
QQYRDTPYT

SEQ ID NO: 491 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 492 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 493 - protein, 757 aa, synthetic construct
  REGION 1..757: note = artificial sequence
HHHHHHGSLEVLFQGPGGTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASNFTGAKKCLVNLIEKVASYGVKPRYGLVTYATYPKIWVKVSEADSSNADWVTKQLNEINYEDHKLNSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDIENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 494 - protein, 227 aa, synthetic construct
  REGION 1..227: note = artificial sequence
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVL

SEQ ID NO: 495 - protein, 1641 aa, synthetic construct
  REGION 1..1641: note = artificial sequence
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 496 - protein, 77 aa, synthetic construct
  REGION 1..77: note = artificial sequence
SVQLTEKRMDKVGKYPKELRKCCEDGMRENPMRFSCQRRTRFISLGEACKKVFLDCCNYITELRRQHARASHLGLAR

SEQ ID NO: 497 - protein, 1640 aa, synthetic construct
  REGION 1..1640: note = artificial sequence
PMYSMITPNVLRLESEETVVLEAHDANGDVPVTVTVHDFPGKKLVLSSEKTVLTPATSHMGSVTIRIPANKEFKSEKGHNKFVTVQATFGAQVVEKVVLVSLQSGYLFIQTDKTIYTPGSTVLCRIFTVNHKLLPVGRTVVVNIENPDGIPVKQDSLSSQNQFGILPLSWDIPELVNMGQWKIRAYYENSPQQVFSTEFEVKEYVLPSFEVIVEPTEKFYYIYNQKGLEVTITARFLYGKKVEGTAFVIFGIQDGEQRISLPESLKRIQIEDGSGDAVLSRKVLLDGVQNPRPEDLVGKSLYVSVTVILHSGSDMVQAERSGIPIVTSPYQIHFTKTPKYFKPGMPFDLMVFVTNPDGSPAYRVPVAVQGEDAVQSLTQGDGVAKLSINTHPSQKPLSITVRTKKRELSEAEQATRTMEAQPYSTVGNSNNYLHLSVPRAELRPGETLNVNFLLRMDRTQEAKIRYYTYLIMNKGKLLKVGRQVREPGQDLVVLPLSITTDFIPSFRLVAYYTLIGANGQREVVADSVWVDVKDSCVGSLVVKSGQSEDRQPLPGQQMTLKIEGDHGARVGLVAVDKGVFVLNKKNKLTQSKIWDVVEKADIGCTPGSGKDYAGVFSDAGLTFASSSGQQTAQRAELQCPQPATRRRRSVQLAEKRMDKVGQYPKELRKCCEHGMRENPMRFSCQRRTRYITLDEACKKAFLDCCNYITELRRQHARASHLGLARSNLDEDIIAEENIVSRSEFPESWLWKIEELKEAPKNGISTKLMNIFLKDSITTWEILAVSLSDKKGICVADPFEVTVMQDFFIDLRLPYSVVRNEQVEIRAVLYNYRQNQELKVRVELLHNPAFCSLATAKRRHQQTVTIPPKSSLSVPYVIVPLKTGQQEVEVKAAVYHFFISDGVRKSLKVVPEGIRMNKTVAVRTLDPERLGQEGVQREDVPPADLSDQVPDTESETRILLQGTPVAQMTEDAIDAERLKHLIVTPSGCGEQNMITMTPTVIAVHYLDETEQWEKFGPEKRQGALELIKKGYTQQLAFRQPSSAFAAFLNRAPSTWLTAYVVKVFSLAVNLIAIDSQVLCGAVKWLILEKQKPDGVFQEDAPVIHQEMTGGFRNTNEKDMALTAFVLISLQEAKEICEEQVNSLPGSITKAGDFLEANYMNLQRSYTVAIAAYALAQMGRLKGPLLNKFLTTAKDKNRWEEPGQQLYNVEATSYALLALLQLKDFDFVPPVVRWLNEQRYYGGGYGSTQATFMVFQALAQYQKDVPDHKELNLDVSLQLPSRSSKIIHRIHWESASLLRSEETKENEGFTVTAEGKGQGTLSVVTMYHAKAKGQLTCNKFDLKVTIKPAPETEKRPQDAKNTMILEICTRYRGDQDATMSILDISMMTGFVPDTDDLKQLANGVDRYISKYELDKAFSDRNTLIIYLDKVSHSEDDCIAFKVHQYFNVELIQPGAVKVYAYYNLAESCTRFYHPEKEDGKLNKLCRDELCRCAEENCFIQKLDDKVTLEERLDKACEPGVDYVYKTRLVKAQLSNDFDEYIMAIEQIIKSGSDEVQVGQQRTFISPIKCREALKLEERKHYLMWGLSSDFWGEKPNLSYIIGKDTWVEHWPEEDECQDEENQKQCQDLGTFTENMVVFGCPN

SEQ ID NO: 498 - protein, 241 aa, synthetic construct
  REGION 1..241: note = artificial sequence
ILGGREAEAHARPYMASVQVNGEHLCGGVLVAEQWVLSAAHCLEDAADGKVQVLLGAHSLSQPEPSKRLYDVLRAVPHPDSRPDTIDHDLLLLQLSEKATLGPAVRPLPWQRVDRDVEPGTLCDVAGWGIVSHAGRRPDRLQHVLLPVLDRATCNRRTHHDGAITQRMMCAESNRRDSCKGDSGGPLVCGGVLEGVVTSGSRVCGNRKKPGIYTRVASYAAWIDSLEVLFQGPGSHHHHHH

SEQ ID NO: 499 - protein, 756 aa, synthetic construct
  REGION 1..756: note = artificial sequence
HHHHHHGSLEVLFQGPGGTPLSSAQPQGSCSLEGVEIKGGSFRLLQEGQALEYVCPSGFYPYPVQTRTCRSTGSWSTLQTQDRKTVKKAECRAIRCPRPQDFENGEYRPRSPYYNVSDEISFHCYDGYTLRGSANRTCQVNGRWSGQTAICDNGAGYCSNPGIPIGTRKVGSRYRLEDSVTYHCSRGLTLRGSQRRTCQEGGSWSGTEPSCQDSFMYDTPQEVAEAFLSSLTETIEGVDAEDGHSPGEQQKRRIILDPSGSMNIYLVLDGSDSIGAGNFTGAKKCLVNLIEKVASYGVKPRYALVTYATYPRIWVKVSDQESSNADWVTKKLSEINYEDHKLKSGTNTKRALQAVYSMMSWPEDIPPEGWNRTRHVIILMTDGLHNMGGDPITVIDEIRDLLYIGKDRKNPREDYLDVYVFGVGPLVDQVNINALASKKDNEQHVFKVKDMENLEDVFFQMIDESQSLSLCGMVWEHTTGTDYHKQPWQAKISVTRPSKGHESCMGAVVSEYFVLTAAHCFTVDDKEHSIKVSVGKKRDLEIEKVLFHPDYNISGKKEAGIPEFYDYDVALIKLKKKLNYDPTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHVNLFQVLPWLKEKLQDEDLGFL

SEQ ID NO: 500 - protein, 756 aa, synthetic construct
  REGION 1..756: note = artificial sequence
HHHHHHGSLEVLFQGPGGTPLSSAQPQGSCSLEGVEIKGGSFRLLQEGQALEYVCPSGFYPYPVQTRTCRSTGSWSTLQTQDRKTVKKAECRAIRCPRPQDFENGEYRPRSPYYNVSDEISFHCYDGYTLRGSANRTCQVNGRWSGQTAICDNGAGYCSNPGIPIGTRKVGSRYRLEDSVTYHCSRGLTLRGSQRRTCQEGGSWSGTEPSCQDSFMYDTPQEVAEAFLSSLTETIEGVDAEDGHSPGEQQKRRIILDPSGSMNIYLVLDGSGSIGAGNFTGAKKCLVNLIEKVASYGVKPRYALVTYATYPRIWVKVSDQESSNADWVTKKLSEINYEDHKLNSGTNTKRALQAVYSMMSWPEDIPPEGWNRTRHVIILMTDGLHNMGGDPITVIDEIRDLLYIGKDRKNPREDYLDVYVFGVGPLVDQVNINALASKKDNEQHVFKVKDIENLEDVFFQMIDESQSLSLCGMVWEHTTGTDYHKQPWQAKISVTRPSKGHESCMGAVVSEYFVLTAAHCFTVDDKEHSIKVSVGKKRDLEIEKVLFHPDYNISGKKEAGIPEFYDYDVALIKLKKKLNYDPTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHVNLFQVLPWLKEKLQDEDLGFL

SEQ ID NO: 501 - protein, 739 aa, synthetic construct
  REGION 1..739: note = artificial sequence
TPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 502 - protein, 107 aa, synthetic construct [sequence omitted]

SEQ ID NO: 503 - protein, 104 aa, synthetic construct
  REGION 1..104: note = artificial sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 504 - protein, 764 aa, synthetic construct
  REGION 1..764: note = artificial sequence
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

(19) United States
(12) Patent Application Publication
(10) Pub. No.: US 2026/0308278 A1
(43) Pub. Date: Oct. 8, 2026

(54) ANTIBODY THAT BINDS TO C3bBb

(71) Applicant: Hoffmann-La Roche Inc., Little Falls, NJ (US)

(72) Inventors: Davide BERTOLDO, Darmstadt (DE); Joachim BUTZER, Penzberg (DE); Sebastian FENN, Achmühle (DE); Alexander FIDLER, Penzberg (DE); Anton JOCHNER, Oberammergau (DE); Eloed KOERTVELY, Riehen (CH); Jean-Luc MARY, Huningue (FR); Sebastian PIRNER, Illschwang (DE)

(73) Assignee: Hoffmann-La Roche Inc., Little Falls, NJ (US)

(21) Appl. No.: 19/713,603

(22) Filed: Jun. 18, 2026

Related U.S. Application Data
(63) Continuation of application No. 19/575,890, filed on Mar. 23, 2026.
Application No. 19/575,890 is a continuation of application No. PCT/EP2024/076826, filed on Sep. 25, 2024.

(30) Foreign Application Priority Data
Sep. 25, 2023 (EP) 23199373.4

(51) Int. Cl. C07K 16/40

(52) U.S. Cl. CPC C07K 16/40; C07K 2317/33; C07K 2317/565; C07K 2317/567; C07K 2317/76; C07K 2317/92; C07K 2317/94

(57) ABSTRACT

The present invention relates to anti-C3bBb antibodies and methods of using the same.

### CROSS REFERENCE TO RELATED APPLICATIONS

This application is a continuation of U.S. application Ser. No. 19/575,890, filed Mar. 23, 2026, which is a continuation of International Application No. PCT/EP2024/076826, filed Sep. 25, 2024, which claims priority to EP Application No 23199373.4, filed Sep. 25, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Jun. 16, 2026, is named “P38828-US-2_Sequence_Listing.xml” and is 687,620 bytes in size.

### FIELD OF THE INVENTION

The present invention relates to anti-C3bBb antibodies and methods of using the same.

### BACKGROUND OF THE INVENTION

The complement system is crucially involved in the disease biology of age related macular degeneration (AMD) (Cell Mol Life Sci 2021 May; 78(10):4487-4505). The alternative pathway C3 convertase (C3bBb) is a key amplifying factor of the complement cascade and it is formed by binding of Factor B (FB) to the C3b fragment of complement component C3 and subsequent cleavage by plasma serine protease Factor D (FD) under release of a fragment Ba. The remaining Bb fragment bound to C3b forms the functional C3 convertase C3bBb. C3bBb is relatively unstable with a half-life of a few minutes making it an unsuitable target for antibody generation.

Due to the central role of the alternative pathway in various pathologies, there is still a need for developing potent inhibitors acting at various complement components within this proteolytic cascade.

### SUMMARY OF THE INVENTION

The present invention relates to an antibody that specifically binds to human C3bBb.

The invention further relates to an antibody that binds to human C3bBb, wherein the antibody:
binds to wild-type human C3bBb, and/or
binds to recombinant human C3bBb, which comprises the Bb subunit of a recombinant Factor B (FB) protein, which comprises a D279G mutation and optionally mutations K350N and/or a M458I; and/or
inhibits the alternative pathway; and/or
is an agonist or antagonist of C3bBb activity; and/or
specifically binds to cynomolgus C3bBb and human C3bBb, and/or
specifically binds to African green monkey C3bBb and human C3bBb, and/or
binds with an affinity of ≤10 nM at 37° C. as measured by SPR, and/or
does not compete with properdin for C3bBb binding, and/or
does not stabilize C3 convertase.

In one aspect, the invention relates to an antibody that specifically binds to wild-type human C3bBb.

In another aspect, the invention relates to an antibody that specifically binds to recombinant human C3bBb, wherein the C3bBb comprises the Bb subunit of a recombinant FB protein that comprises a D279G and optionally further mutations K350N and/or M458I. In one embodiment the antibody specifically binds to human C3bBb comprising the Bb subunit of a recombinant FB protein according to SEQ ID NO: 492. In one embodiment the antibody specifically binds to human C3bBb comprising the Bb subunit of a recombinant FB protein according to SEQ ID NO: 493.

In another aspect, the invention relates to an antibody that specifically binds to an epitope of human C3bBb that includes amino acid residues of the C3b subunit and amino acid residues of the Bb subunit of human FB. In one embodiment the antibody of the invention specifically binds to human C3bBb, wherein the antibody does not specifically bind to the (isolated) C3b subunit of C3bBb, and wherein the antibody does not specifically bind to the (isolated) Bb subunit of C3bBb.

In another aspect, the invention relates to an antibody that binds to human C3bBb, wherein the antibody comprises the heavy and light chain CDRs of an antibody selected from Table D1. In one embodiment the antibody comprises the heavy and light chain variable domains of an antibody selected from Table D1.

In one embodiment, the antibody comprises a heavy chain variable domain (VH) (a) CDR-H1 comprising the amino acid sequence of SEQ ID NO:449; (b) CDR-H2 comprising the amino acid sequence of SEQ ID NO:450; and (c) CDR-H3 comprising the amino acid sequence of SEQ ID NO:451, and a light chain variable domain (VL) comprising (d) CDR-L1 comprising the amino acid sequence of SEQ ID NO:452; (e) CDR-L2 comprising the amino acid sequence of SEQ ID NO:453; and (f) CDR-L3 comprising the amino acid sequence of SEQ ID NO:454 (corresponding to antibody #1, P1AG9426); or a heavy chain variable domain (VH) (a) CDR-H1 comprising the amino acid sequence of SEQ ID NO:455; (b) CDR-H2 comprising the amino acid sequence of SEQ ID NO:456; and (c) CDR-H3 comprising the amino acid sequence of SEQ ID NO:457, and a light chain variable domain (VL) comprising (d) CDR-L1 comprising the amino acid sequence of SEQ ID NO:458; (e) CDR-L2 comprising the amino acid sequence of SEQ ID NO:459; and (f) CDR-L3 comprising the amino acid sequence of SEQ ID NO:460 (corresponding to antibody #2, P1AG9376); or a heavy chain variable domain (VH) (a) CDR-H1 comprising the amino acid sequence of SEQ ID NO:461; (b) CDR-H2 comprising the amino acid sequence of SEQ ID NO:462; and (c) CDR-H3 comprising the amino acid sequence of SEQ ID NO:463, and a light chain variable domain (VL) comprising (d) CDR-L1 comprising the amino acid sequence of SEQ ID NO:464; (e) CDR-L2 comprising the amino acid sequence of SEQ ID NO:465; and (f) CDR-L3 comprising the amino acid sequence of SEQ ID NO:466 (corresponding to antibody #3, P1AG9372); or a heavy chain variable domain (VH) (a) CDR-H1 comprising the amino acid sequence of SEQ ID NO:467; (b) CDR-H2 comprising the amino acid sequence of SEQ ID NO:468; and (c) CDR-H3 comprising the amino acid sequence of SEQ ID NO:469, and a light chain variable domain (VL) comprising (d) CDR-L1 comprising the amino acid sequence of SEQ ID NO:470; (e) CDR-L2 comprising the amino acid sequence of SEQ ID NO:471; and (f) CDR-L3 comprising the amino acid sequence of SEQ ID NO:472 (corresponding to antibody #4, P1AG9420); or a heavy chain variable domain (VH) (a) CDR-H1 comprising the amino acid sequence of SEQ ID NO:473; (b) CDR-H2 comprising the amino acid sequence of SEQ ID NO:474; and (c) CDR-H3 comprising the amino acid sequence of SEQ ID NO:475, and a light chain variable domain (VL) comprising (d) CDR-L1 comprising the amino acid sequence of SEQ ID NO:476; (e) CDR-L2 comprising the amino acid sequence of SEQ ID NO:477; and (f) CDR-L3 comprising the amino acid sequence of SEQ ID NO:478 (corresponding to antibody #5, P1AG9391); or a heavy chain variable domain (VH) (a) CDR-H1 comprising the amino acid sequence of SEQ ID NO:479; (b) CDR-H2 comprising the amino acid sequence of SEQ ID NO:480; and (c) CDR-H3 comprising the amino acid sequence of SEQ ID NO:481, and a light chain variable domain (VL) comprising (d) CDR-L1 comprising the amino acid sequence of SEQ ID NO:482; (e) CDR-L2 comprising the amino acid sequence of SEQ ID NO:483; and (f) CDR-L3 comprising the amino acid sequence of SEQ ID NO:484 (corresponding to antibody #6, P1AH1205); or a heavy chain variable domain (VH) (a) CDR-H1 comprising the amino acid sequence of SEQ ID NO:485; (b) CDR-H2 comprising the amino acid sequence of SEQ ID NO:486; and (c) CDR-H3 comprising the amino acid sequence of SEQ ID NO:487, and a light chain variable domain (VL) comprising (d) CDR-L1 comprising the amino acid sequence of SEQ ID NO:488; (e) CDR-L2 comprising the amino acid sequence of SEQ ID NO:489; and (f) CDR-L3 comprising the amino acid sequence of SEQ ID NO:490 (corresponding to antibody #7, P1AH 1199).

In one embodiment, the antibody comprises a VH domain comprising SEQ ID NO:1 and a VL domain comprising SEQ ID NO:2 (corresponding to antibody #1, P1AG9426); or a VH domain comprising SEQ ID NO:3 and a VL domain comprising SEQ ID NO:4 (corresponding to antibody #2, P1AG9376); or a VH domain comprising SEQ ID NO:5 and a VL domain comprising SEQ ID NO:6 (corresponding to antibody #3, P1AG9372); or a VH domain comprising SEQ ID NO:7 and a VL domain comprising SEQ ID NO:8 (corresponding to antibody #4, P1AG9420); or a VH domain comprising SEQ ID NO:9 and a VL domain comprising SEQ ID NO:10 (corresponding to antibody #5, P1AG9391); or a VH domain comprising SEQ ID NO:11 and a VL domain comprising SEQ ID NO:12 (corresponding to antibody #6, P1AH1205); or a VH domain comprising SEQ ID NO:13 and a VL domain comprising SEQ ID NO:14 (corresponding to to antibody #7, P1AH1199).

Also, the invention relates to an antibody that competes for binding to wild-type human C3bBb with the antibodies of the invention.

The invention also relates to an antibody that competes for binding to recombinant human C3bBb comprising the Bb subunit of a recombinant FB protein that comprises a D279G and optionally further mutations K350N and/or M458I with the antibody of the invention.

Another aspect of the invention is an isolated nucleic acid encoding the antibody of the invention.

The invention also relates to a host cell comprising the nucleic acid of the invention.

Further, the invention relates to a method of producing an antibody that binds to human C3bBb comprising culturing the host cell of the invention under conditions suitable for the expression of the antibody.

In another aspect, the invention relates to a pharmaceutical composition comprising the antibody of the invention.

In a further aspect, the invention relates to an antibody of the invention for use as a medicament.

Antibodies of the invention are suitable to inhibit the alternative pathway by binding to C3bBb. Antibodies of the invention may be capable of inhibiting the alternative pathway without stabilizing C3bBb upon binding, thus avoiding accumulation of C3bBb. Furthermore, antibodies of the invention may be specific for the C3bBb complex but not exhibit prominent antigen binding to its individual subunits.

The antibodies disclosed herein may be suitable for therapy, particularly treatment of ocular vascular diseases.

The antibodies of the invention provide several valuable properties that allow their therapeutic application, like high affinities, and a high stability advantageous for long duration. Also, the antibodies of the invention are advantageous to be provided in high concentrations liquid formulations with a viscosity suitable for ocular application.

### DESCRIPTION OF THE FIGURES

FIG. 1: The left image shows the trimeric complex formed between the P1AF8499 Fab fragment and C3bBb. The epitope bound by P1AF8499 Fab fragment comprises amino acids from both components, C3b and Bb (right image).

FIG. 2: Binding of antibody P1AG9426 (#1) to wild-type human C3bBb complex. Sensorgram generated as described in Example 14.

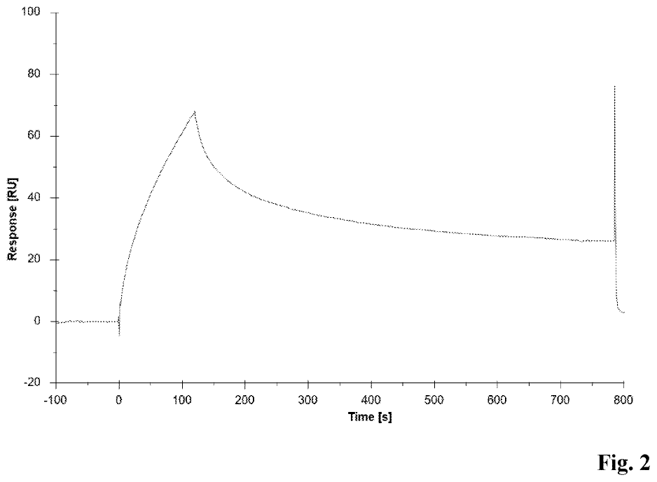

### DETAILED DESCRIPTION OF THE INVENTION

### 1. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Specific illustrative and exemplary methods for measuring binding affinity are described in the following. Antibodies that bind to human C3bBb have a dissociation constant (K_{D}) of ≤1 pM, ≤100 nM, ≤10 nM, ≤1 nM, ≤0.1 nM, ≤0.01 nM, or ≤0.001 nM, in one embodiment 10⁻⁶ M or less, in one embodiment 10⁻⁷ M or less, in one embodiment 10⁻⁸ M or less, in one embodiment from 10⁻⁸ M to 10⁻¹³ M, in one embodiment from 10⁻⁹ M to 10⁻¹³ M). An antibody is said to “specifically bind” to human C3bBb when the antibody has a KD of 1 μM or less. In certain aspects, an anti-human C3bBb antibody binds to an epitope of human C3bBb that is conserved among human C3bBb from different species.

The components of the innate immune system, i.e. the different elements from the classical and alternative pathways are referred to herein according to their standard meaning in the art and the termini used herein include recombinant variants of the respective natural components of the innate immune system that maintain functionality and may include variants comprising tags and/or labels.

In brief, the term “C3bBb” as used herein refers to the alternative pathway C3 convertase that is formed by binding of C3 fragment (“C3b”) to Factor B (“FB”) and subsequent cleavage by plasma serine protease Factor D (“FD”) under release of a fragment Ba. The term “C3b” as used herein refers to the larger of two elements resulting from the alternative pathway cleavage of complement component 3 (C3), the other one being C3a. The term “C3 convertase” refers to the serine proteases of the complement system and may refer to the alternative pathway C3 convertase (C3bBb) or the classical and lectin pathway C3 convertase (C4bC2b). The term “human Factor B” or “FB” refers to complement factor B encoded in humans by the CFB gene. The term “human Factor D” refers to complement factor D encoded in humans by the CFD gene. The term “C5 convertase” as used herein refers to the alternative pathway C5 convertase (C3bBbC3b).

The term “antibody screening” refers to techniques used to identify antibodies specifically binding to a target antigen. Various techniques for identifying antibodies are known in the art, such as administration of an immunogenic target antigen to a transgenic animal, hybridoma-based methods, or isolating variable domain sequences selected from phage-display libraries. The term “phage library panning” refers to an affinity selection technique used to identify phage display variants with desired binding properties towards a target antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “epitope” denotes the site on an antigen, either proteinaceous or non-proteinaceous, to which an anti-human C3bBb antibody binds. The epitope bound by antibodies of the invention comprises non-contiguous amino acids and hence is a conformational epitope.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), cross-blocking (see “Antibodies”, Harlow and Lane (Cold Spring Harbor Press, Cold Spring Harb., NY). In certain embodiments used herein, an epitope of an antibody is identified by cryogenic electron microscopy.

Also, competitive binding can be used to easily determine whether an antibody competes for binding to human C3bBb with a reference anti-human C3bBb antibody of the invention. For example, an “antibody that competes for binding with” a reference anti-human C3bBb antibody of the invention refers to an antibody that blocks binding of the reference anti-human C3bBb antibody to its antigen in a competition assay by 50% or more, and conversely, the reference antibody blocks binding of the antibody to its antigen in a competition assay by 50% or more.

In some aspects, two antibodies are deemed to compete for binding to human C3bBb if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain. The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-human C3bBb antibody” refers to one or more nucleic acid molecules encoding anti-human C3bBb antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### 2. Detailed Description of the Embodiments of the Invention

In one aspect, the invention is based, in part, on the provision of antibodies that specifically bind to human C3bBb that were obtained upon screening using a stabilized C3bBb complex. In certain aspects, antibodies that bind to human C3bBb are provided. Antibodies of the invention are useful, e.g., for the diagnosis, prevention or treatment of diseases, such as ocular diseases, e.g. age related macular degeneration.

### A. Exemplary Anti-C3bBb Antibodies

In one aspect, the invention provides an antibody that specifically binds to human C3bBb. In another aspect, the invention provides an antibody that specifically binds to human C3bBb comprising a Bb subunit of factor B (FB) with a D279G mutation. In one embodiment, the antibody binds to human C3bBb comprising a Bb subunit of factor B (FB) with an amino acid sequence of SEQ ID NO: 492.

In another aspect, the invention provides an antibody that specifically binds to human C3bBb comprising a Bb subunit of factor B (FB) with D279G, K350N and M458I mutations. In one embodiment, the antibody binds to human C3bBb comprising a Bb subunit of factor B (FB) with an amino acid sequence of SEQ ID NO: 493.

In another aspect, the invention provides an antibody that binds to human C3bBb, wherein the antibody:
binds to wild-type human C3bBb, and/or
binds to recombinant human C3bBb, which comprises the Bb subunit of a recombinant Factor B (FB) protein, which comprises a D279G mutation and optionally mutations K350N and/or a M458I; and/or
inhibits the alternative pathway; and/or
is an agonist or antagonist of C3bBb activity; and/or
specifically binds to cynomolgus C3bBb and human C3bBb, and/or
specifically binds to African green monkey C3bBb and human C3bBb, and/or
binds with an affinity of ≤10 nM at 37° C. as measured by SPR.

Antibodies of the invention may be provided by screening using a stabilized C3bBb that was provided using a recombinant FB protein with a D279G and optionally, further mutations K350N and/or M458I. Wild type C3bBb is a rapidly decaying molecule, which requires stabilization to be used for antibody screening purposes. The inventors of the present invention were able to increase the half life of the C3bBb complex from about 5 minutes to 11 hours, which allowed for using the stabilized complex for antibody generation in a phage library panning.

Hence, antibodies of the invention specifically bind to stabilized C3bBb, particularly to C3bBb comprising the Bb subunit of factor B (FB) with a D279G mutation and optionally mutations K350N and/or a M458I. In one embodiment, the antibodies of the invention specifically bind to stabilized C3bBb comprising the Bb subunit of factor B (FB) with D279G, K350N and M458I mutations (preferably of FB with an amino acid sequence of SEQ ID NO: 493).

Hence, one aspect of the invention is an antibody that binds to human C3bBb comprising the Bb subunit of recombinant factor B (FB) with D279G, K350N and M458I mutations (preferably of FB with an amino acid sequence of SEQ ID NO: 493) that inhibits the alternative pathway.

Also, antibodies of the invention specifically bind to wild-type human C3bBb (Example 14) and are capable of inhibiting the alternative pathway (Example 9).

The antibodies of the invention bind to an epitope on human C3bBb that includes amino acid residues of the C3b subunit and amino acid residues of the Bb subunit of human C3bBb. This may be achieved by screening antibodies using a stabilized C3bBb as target antigen, e.g. by a method as described herein.

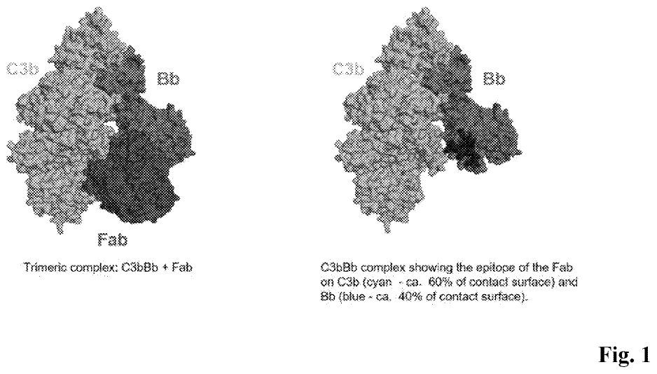

The epitope of antibodies of the invention may be identified by methods known in the art, e.g., by cryogenic electron microscopy. P1AF8499 (#20, see Examples 2 and 4) of the invention was analyzed by cryogenic electron microscopy of the trimeric complex of the antibody with C3bBb (Example 4). As indicated in FIG. 1, the epitope is a conformational epitope including amino acid residues of the C3b subunit and amino acid residues of the Bb subunit of human C3bBb, which results in that the antibodies specifically bind to C3bBb while showing no relevant binding activity to its subunits C3b and Bb. Cryogenic electron microscopy was used to identify amino acids that are bound by the antibody of the invention (Table E5 of Example 4).

In one aspect of the invention, the antibody binds to an epitope comprising the following amino acid residues on wild-type human C3bBb as detected by cryogenic electron microscopy, preferably with a method according to Example 4:
C3b subunit: Arg444, Lys534, Gly539, Ser540, Val524, Lys544, Gly546, Gln547, Ser548, Arg551, Gln557, Gln558, Thr560, Lys562, Glu564, Glu758, Pro759, Lys761, Asn762, Ile764, Leu768, Asn770, Asp797, and
Bb subunit: Tyr465, Lys473, Ile474, Ser475, Ile477, Gly482, His483, Lys513, Val514, Ser515, Lys520, Arg521, Asp522, Glu610, and Lys613.

In one embodiment the antibody binds to the same or an overlapping epitope than the epitope bound by antibody having a VH domain of SEQ ID NO: 39 and a VL domain of SEQ ID NO: 30.

Screening of antibodies binding to human C3bBb was done with phage library panning and further engineering of the directly generated antibodies resulting in a plurality of antibodies that were identified to bind to a stabilized C3bBb. Amino acid sequences of VH and VL domains of the antibodies are indicated in Table D1.

TABLE D1
amino acid sequence numbers of variable heavy chain and
variable light chain domains of anti-C3bBb antibodies

| | | | VH | VL |
|---|---|---|---|---|
| | | | (SEQ | (SEQ |
| # | identifier | | ID NO:) | ID NO:) |
| 1 | P1AG9426 | IgG | 1 | 2 |
| 2 | P1AG9376 | IgG | 3 | 4 |
| 3 | P1AG9372 | IgG | 5 | 6 |
| 4 | P1AG9420 | IgG | 7 | 8 |
| 5 | P1AG9391 | IgG | 9 | 10 |
| 6 | P1AH1205 | Fab | 11 | 12 |
| 7 | P1AH1199 | Fab | 13 | 14 |
| 8 | C3bBb.P045.072 | Fab | 15 | 16 |
| 9 | C3bBb.P045.236 | Fab | 17 | 18 |
| 10 | C3bBb.P045.225 | Fab | 19 | 20 |
| 11 | C3bBb.P048.152 | Fab | 21 | 22 |
| 12 | C3bBb.P048.061 | Fab | 23 | 24 |
| 13 | C3bBb.P048.127 | Fab | 25 | 26 |
| 14 | C3bBb.P048.241 | Fab | 27 | 28 |
| 15 | C3bBb.P048.230 | Fab | 29 | 30 |
| 16 | C3bBb.P048.204 | Fab | 31 | 32 |
| 17 | C3bBb.P048.333 | Fab | 33 | 34 |
| 18 | C3bBb.P053.095 | Fab | 35 | 36 |
| 19 | C3bBb.P055.015 | Fab | 37 | 38 |
| 20 | P1AF8499 | Fab | 39 | 40 |
| 21 | GQ-5_1 | Fab | 41 | 42 |
| 22 | GQ-11_1 | Fab | 43 | 44 |
| 23 | PT5622.F19 | Fab | 45 | 46 |
| 24 | PT5624.P06 | Fab | 47 | 48 |
| 25 | PT5624.M09 | Fab | 49 | 50 |
| 26 | PT5626.M10 | Fab | 51 | 52 |
| 27 | PT5626.C10 | Fab | 53 | 54 |
| 28 | PT5628.E16 | Fab | 55 | 56 |
| 29 | PT5630.D05 | Fab | 57 | 58 |
| 30 | PT5630.A20 | Fab | 59 | 60 |
| 31 | PT5632.A13 | Fab | 61 | 62 |
| 32 | PT5634.N13 | Fab | 63 | 64 |
| 33 | PT5634.M24 | Fab | 65 | 66 |
| 34 | PT5636.P22 | Fab | 67 | 68 |
| 35 | PT5625.G07 | Fab | 69 | 70 |
| 36 | PT5627.M20 | Fab | 71 | 72 |
| 37 | PT5627.M12 | Fab | 73 | 74 |
| 38 | PT5629.F09 | Fab | 75 | 76 |
| 39 | PT5629.H08 | Fab | 77 | 78 |
| 40 | PT5631.D21 | Fab | 79 | 80 |
| 41 | PT5633.I03 | Fab | 81 | 82 |
| 42 | PT5635.C08 | Fab | 83 | 84 |
| 43 | PT5639.G15 | Fab | 85 | 86 |
| 44 | PT5622.A20 | Fab | 87 | 88 |
| 45 | PT5622.C12 | Fab | 89 | 90 |
| 46 | PT5622.G24 | Fab | 91 | 92 |
| 47 | PT5622.I20 | Fab | 93 | 94 |
| 48 | PT5622.J24 | Fab | 95 | 96 |
| 49 | PT5624.A21 | Fab | 97 | 98 |
| 50 | PT5624.B10 | Fab | 99 | 100 |
| 51 | PT5624.N05 | Fab | 101 | 102 |
| 52 | PT5624.P11 | Fab | 103 | 104 |
| 53 | PT5626.A12 | Fab | 105 | 106 |
| 54 | PT5626.N08 | Fab | 107 | 108 |
| 55 | PT5626.N18 | Fab | 109 | 110 |
| 56 | PT5626.O09 | Fab | 111 | 112 |
| 57 | PT5628.B13 | Fab | 113 | 114 |
| 58 | PT5628.C21 | Fab | 115 | 116 |
| 59 | PT5628.E12 | Fab | 117 | 118 |
| 60 | PT5628.I07 | Fab | 119 | 120 |
| 61 | PT5628.P14 | Fab | 121 | 122 |
| 62 | PT5630.G05 | Fab | 123 | 124 |
| 63 | PT5630.K04 | Fab | 125 | 126 |
| 64 | PT5630.M11 | Fab | 127 | 128 |
| 65 | PT5630.N06 | Fab | 129 | 130 |
| 66 | PT5630.O08 | Fab | 131 | 132 |
| 67 | PT5632.A10 | Fab | 133 | 134 |
| 68 | PT5632.A15 | Fab | 135 | 136 |
| 69 | PT5632.B15 | Fab | 137 | 138 |
| 70 | PT5632.D11 | Fab | 139 | 140 |
| 71 | PT5632.J23 | Fab | 141 | 142 |
| 72 | PT5634.A17 | Fab | 143 | 144 |
| 73 | PT5634.A21 | Fab | 145 | 146 |
| 74 | PT5634.K10 | Fab | 147 | 148 |
| 75 | PT5634.M16 | Fab | 149 | 150 |
| 76 | PT5634.N11 | Fab | 151 | 152 |
| 77 | PT5634.P19 | Fab | 153 | 154 |
| 78 | PT5636.A16 | Fab | 155 | 156 |
| 79 | PT5636.D21 | Fab | 157 | 158 |
| 80 | PT5636.M03 | Fab | 159 | 160 |
| 81 | PT5636.N13 | Fab | 161 | 162 |
| 82 | PT5636.P11 | Fab | 163 | 164 |
| 83 | PT5636.P15 | Fab | 165 | 166 |
| 84 | PT5638.C06 | Fab | 167 | 168 |
| 85 | PT5638.C20 | Fab | 169 | 170 |
| 86 | PT5638.E14 | Fab | 171 | 172 |
| 87 | PT5638.J23 | Fab | 173 | 174 |
| 88 | PT5638.O14 | Fab | 175 | 176 |
| 89 | PT5638.P12 | Fab | 177 | 178 |
| 90 | PT5623.E21 | Fab | 179 | 180 |
| 91 | PT5623.F17 | Fab | 181 | 182 |
| 92 | PT5623.G08 | Fab | 183 | 184 |
| 93 | PT5623.H05 | Fab | 185 | 186 |
| 94 | PT5623.008 | Fab | 187 | 188 |
| 95 | PT5625.A05 | Fab | 189 | 190 |
| 96 | PT5625.B14 | Fab | 191 | 192 |
| 97 | PT5625.C15 | Fab | 193 | 194 |
| 98 | PT5625.G05 | Fab | 195 | 196 |
| 99 | PT5625.L22 | Fab | 197 | 198 |
| 100 | PT5625.O14 | Fab | 199 | 200 |
| 101 | PT5627.121 | Fab | 201 | 202 |
| 102 | PT5629.F04 | Fab | 203 | 204 |
| 103 | PT5629.L22 | Fab | 205 | 206 |
| 104 | PT5629.L24 | Fab | 207 | 208 |
| 105 | PT5629.M15 | Fab | 209 | 210 |
| 106 | PT5631.H06 | Fab | 211 | 212 |
| 107 | PT5631.K12 | Fab | 213 | 214 |
| 108 | PT5631.N17 | Fab | 215 | 216 |
| 109 | PT5631.P07 | Fab | 217 | 218 |
| 110 | PT5633.E09 | Fab | 219 | 220 |
| 111 | PT5633.G19 | Fab | 221 | 222 |
| 112 | PT5635.F04 | Fab | 223 | 224 |
| 113 | PT5635.L21 | Fab | 225 | 226 |
| 114 | PT5635.O15 | Fab | 227 | 228 |
| 115 | PT5637.B23 | Fab | 229 | 230 |
| 116 | PT5637.C20 | Fab | 231 | 232 |
| 117 | PT5637.K09 | Fab | 233 | 234 |
| 118 | PT5637.L15 | Fab | 235 | 236 |
| 119 | PT5637.M03 | Fab | 237 | 238 |
| 120 | PT5637.P15 | Fab | 239 | 240 |
| 121 | PT5639.B15 | Fab | 241 | 242 |
| 122 | PT5639.P05 | Fab | 243 | 244 |
| 123 | PT5625.G07_22.F19_2 | Fab | 245 | 246 |
| 124 | PT5625.G07_24.M09_1 | Fab | 247 | 248 |
| 125 | PT5625.G07 24.P06_2 | Fab | 249 | 250 |
| 126 | PT5625.G07_26.C10_1 | Fab | 251 | 252 |
| 127 | PT5625.G07_26.M10_2 | Fab | 253 | 254 |
| 128 | PT5625.G07_28.E16_1 | Fab | 255 | 256 |
| 129 | PT5625.G07_30.A20_1 | Fab | 257 | 258 |
| 130 | PT5625.G07_32.A13_2 | Fab | 259 | 260 |
| 131 | PT5625.G07_34.M24_1 | Fab | 261 | 262 |
| 132 | PT5625.G07_34.N13_3 | Fab | 263 | 264 |
| 133 | PT5625.G07_36.P22_1 | Fab | 265 | 266 |
| 134 | PT5627.M20_24.M09_1 | Fab | 267 | 268 |
| 135 | PT5627.M20_24.P06_1 | Fab | 269 | 270 |
| 136 | PT5627.M20_26.C10_1 | Fab | 271 | 272 |
| 137 | PT5627.M20_26.M10_2 | Fab | 273 | 274 |
| 138 | PT5627.M20_28.E16_2 | Fab | 275 | 276 |
| 139 | PT5627.M20_30.D05_1 | Fab | 277 | 278 |
| 140 | PT5627.M20_32.A13_1 | Fab | 279 | 280 |
| 141 | PT5627.M20_34.M24_2 | Fab | 281 | 282 |
| 142 | PT5627.M20_34.N13_4 | Fab | 283 | 284 |
| 143 | PT5627.M20_36.P22_1 | Fab | 285 | 286 |
| 144 | PT5627.M12_22.F19_1 | Fab | 287 | 288 |
| 145 | PT5627.M12_24.M09_1 | Fab | 289 | 290 |
| 146 | PT5627.M12_26.M10_2 | Fab | 291 | 292 |
| 147 | PT5627.M12_32.A13_2 | Fab | 293 | 294 |
| 148 | PT5627.M12_34.M24_2 | Fab | 295 | 296 |
| 149 | PT5627.M12_34.N13_1 | Fab | 297 | 298 |
| 150 | PT5629.F09_26.C10_1 | Fab | 299 | 300 |
| 151 | PT5629.F09_28.E16_1 | Fab | 301 | 302 |
| 152 | PT5629.F09_30.A20_1 | Fab | 303 | 304 |
| 153 | PT5629.F09_30.D05_1 | Fab | 305 | 306 |
| 154 | PT5629.F09_32.A13_3 | Fab | 307 | 308 |
| 155 | PT5629.F09_36.P22_2 | Fab | 309 | 310 |
| 156 | PT5629.H08_22.F19_4 | Fab | 311 | 312 |
| 157 | PT5629.H08_24.M09_4 | Fab | 313 | 314 |
| 158 | PT5629.H08_24.P06_2 | Fab | 315 | 316 |
| 159 | PT5629.H08_26.C10_1 | Fab | 317 | 318 |
| 160 | PT5629.H08_26.M10_1 | Fab | 319 | 320 |
| 161 | PT5629.H08_28.E16_2 | Fab | 321 | 322 |
| 162 | PT5629.H08_30.A20_2 | Fab | 323 | 324 |
| 163 | PT5629.H08_30.D05_1 | Fab | 325 | 326 |
| 164 | PT5629.H08_32.A13_1 | Fab | 327 | 328 |
| 165 | PT5629.H08_34.M24_2 | Fab | 329 | 330 |
| 166 | PT5629.H08_34.N13_2 | Fab | 331 | 332 |
| 167 | PT5629.H08_36.P22_4 | Fab | 333 | 334 |
| 168 | PT5631.D21_22.F19_2 | Fab | 335 | 336 |
| 169 | PT5631.D21_24.M09_1 | Fab | 337 | 338 |
| 170 | PT5631.D21_24.P06_4 | Fab | 339 | 340 |
| 171 | PT5631.D21_26.C10_1 | Fab | 341 | 342 |
| 172 | PT5631.D21_26.M10_4 | Fab | 343 | 344 |
| 173 | PT5631.D21_28.E16_3 | Fab | 345 | 346 |
| 174 | PT5631.D21_30.A20_4 | Fab | 347 | 348 |
| 175 | PT5631.D21_30.D05 1 | Fab | 349 | 350 |
| 176 | PT5631.D21_32.A13_4 | Fab | 351 | 352 |
| 177 | PT5631.D21_34.M24_4 | Fab | 353 | 354 |
| 178 | PT5631.D21_34.N13_2 | Fab | 355 | 356 |
| 179 | PT5631.D21_36.P22_3 | Fab | 357 | 358 |
| 180 | PT5633.I03_22.F19_1 | Fab | 359 | 360 |
| 181 | PT5633.103_24.M09_1 | Fab | 361 | 362 |
| 182 | PT5633.I03_24.P06_4 | Fab | 363 | 364 |
| 183 | PT5633.I03_26.C10_1 | Fab | 365 | 366 |
| 184 | PT5633.I03_26.M10_4 | Fab | 367 | 368 |
| 185 | PT5633.103_28.E16_4 | Fab | 369 | 370 |
| 186 | PT5633.I03_30.A20_1 | Fab | 371 | 372 |
| 187 | PT5633.I03_30.D05_1 | Fab | 373 | 374 |
| 188 | PT5633.I03_32.A13_1 | Fab | 375 | 376 |
| 189 | PT5633.I03_34.M24_2 | Fab | 377 | 378 |
| 190 | PT5633.I03_34.N13_1 | Fab | 379 | 380 |
| 191 | PT5633.I03_36.P22_2 | Fab | 381 | 382 |
| 192 | PT5635.C08_22.F19_1 | Fab | 383 | 384 |
| 193 | PT5635.C08_24.M09_1 | Fab | 385 | 386 |
| 194 | PT5635.C08_24.P06_1 | Fab | 387 | 388 |
| 195 | PT5635.C08_26.C10_1 | Fab | 389 | 390 |
| 196 | PT5635.C08_26.M10_2 | Fab | 391 | 392 |
| 197 | PT5635.C08_28.E16_1 | Fab | 393 | 394 |
| 198 | PT5635.C08_30.A20_1 | Fab | 395 | 396 |
| 199 | PT5635.C08_30.D05_3 | Fab | 397 | 398 |
| 200 | PT5635.C08_32.A13_3 | Fab | 399 | 400 |
| 201 | PT5635.C08_34.M24_2 | Fab | 401 | 402 |
| 202 | PT5635.C08_34.N13_1 | Fab | 403 | 404 |
| 203 | PT5635.C08_36.P22_1 | Fab | 405 | 406 |
| 204 | PT5639.G15_22.F19_2 | Fab | 407 | 408 |
| 205 | PT5639.G15_24.P06_1 | Fab | 409 | 410 |
| 206 | PT5639.G15_24.M09_4 | Fab | 411 | 412 |
| 207 | PT5639.G15_26.M10_3 | Fab | 413 | 414 |
| 208 | PT5639.G15_28.E16_1 | Fab | 415 | 416 |
| 209 | PT5639.G15_30.A20_1 | Fab | 417 | 418 |
| 210 | PT5639.G15_30.D05_1 | Fab | 419 | 420 |
| 211 | PT5639.G15_34.N13_4 | Fab | 421 | 422 |
| 212 | PT5639.G15_34.M24_3 | Fab | 423 | 424 |
| 213 | PT5639.G15_36.P22_3 | Fab | 425 | 426 |
| 214 | PT5625.G07_30.D05_1 | Fab | 427 | 428 |
| 215 | PT5627.M12_26.C10_1 | Fab | 429 | 430 |
| 216 | PT5627.M12_30.A20_2 | Fab | 431 | 432 |
| 217 | PT5629.F09_34.N13_3 | Fab | 433 | 434 |

Antibodies Derived from Antibody Screening

Each one of the 217 antibodies disclosed herein are disclosed as an invention, being defined by their 6 CDRs as derived from the amino acid sequences of their VH domain amino acid sequence and their VL amino acid sequence as indicated in Table D1. Alternatively, the antibodies of the invention are characterized by a VH domain having the VH amino acid sequence indicated in Table D1, and a VL domain having the VL amino acid sequence as indicated in Table D1.

One aspect of the invention is an antibody that specifically binds to human C3bBb comprising a set of six CDRs of any one of the antibodies of Table D1. In one embodiment the antibody comprises a variable light chain amino acid sequence and a variable heavy chain amino acid sequence of any one of the antibodies of Table D1.

In one aspect the invention refers to an antibody selected from antibody #1 to antibody #217 as indicated in the first column of Table D1, wherein the antibody comprises a set of six CDRs as derived from the amino acid sequences of its respective VH domain amino acid sequence and its respective VL amino acid sequence as indicated in Table D1. In one embodiment, the antibody comprises a variable light chain amino acid sequence and a variable heavy chain amino acid sequence of any one of the antibodies of Table D1.

In one aspect the invention refers to an antibody selected from antibody #1 to antibody #122 as indicated in the first column of Table D1, wherein the antibody comprises a set of six CDRs as derived from the amino acid sequences of its respective VH domain amino acid sequence and its respective VL amino acid sequence as indicated in Table D1. In one embodiment, the antibody comprises a variable light chain amino acid sequence and a variable heavy chain amino acid sequence of any one of the antibodies of Table D1.

In one aspect the invention refers to an antibody selected from antibody #1 to antibody #7 and #22 to #217 as indicated in the first column of Table D1, wherein the antibody comprises a set of six CDRs as derived from the amino acid sequences of its respective VH domain amino acid sequence and its respective VL amino acid sequence as indicated in Table D1. In one embodiment, the antibody comprises a variable light chain amino acid sequence and a variable heavy chain amino acid sequence of any one of the antibodies of Table D1.

In one aspect the invention refers to an antibody selected from antibody #1 to antibody #7 and #22 to #122 as indicated in the first column of Table D1, wherein the antibody comprises a set of six CDRs as derived from the amino acid sequences of its respective VH domain amino acid sequence and its respective VL amino acid sequence as indicated in Table D1. In one embodiment, the antibody comprises a variable light chain amino acid sequence and a variable heavy chain amino acid sequence of any one of the antibodies of Table D1.

In one aspect the invention refers to an antibody selected from antibody #1 to antibody #7 as indicated in the first column of Table D1, wherein the antibody comprises a set of six CDRs as derived from the amino acid sequences of its respective VH domain amino acid sequence and its respective VL amino acid sequence as indicated in Table D1. In one embodiment, the antibody comprises a variable light chain amino acid sequence and a variable heavy chain amino acid sequence of any one of the antibodies of Table D1.

In one aspect the invention refers to an antibody derived from the antibody #22 as indicated in the first column of Table D1.

Another aspect of the invention is an antibody that specifically binds to human C3bBb comprising a set of VH and VL domains of any one of the antibodies of Table D1.

In one embodiment, the antibody comprises a set of six CDRs of any one of the antibodies of Table E4. In another embodiment, the antibody comprises a set of VH and VL domains of any one of the antibodies of Table E4.

### Individual Antibodies

### Antibody P1AG9426

In another aspect, the invention provides an antibody comprising (a) CDR-H1 comprising the amino acid sequence of SEQ ID NO:449; (b) CDR-H2 comprising the amino acid sequence of SEQ ID NO:450; (c) CDR-H3 comprising the amino acid sequence of SEQ ID NO:451; (d) CDR-L1 comprising the amino acid sequence of SEQ ID NO:452; (e) CDR-L2 comprising the amino acid sequence of SEQ ID NO:453; and (f) CDR-L3 comprising the amino acid sequence of SEQ ID NO:454 (corresponding to antibody #1, P1AG9426).

In another aspect, an anti-C3bBb antibody comprises one or more of the CDR sequences of the VH of SEQ ID NO:1. In another embodiment, an anti-C3bBb antibody comprises one or more of the CDR sequences of the VL of SEQ ID NO:2. In another embodiment, an anti-C3bBb antibody comprises the CDR sequences of the VH of SEQ ID NO:1 and the CDR sequences of the VL of SEQ ID NO:2.

In a further aspect, an anti-C3bBb antibody comprises the CDR-H1, CDR-H2 and CDR-H3 amino acid sequences of the VH domain of SEQ ID NO:1 and the CDR-L1, CDR-L2 and CDR-L3 amino acid sequences of the VL domain of SEQ ID NO:2.

In one aspect, an anti-C3bBb antibody comprises one or more of the heavy chain CDR amino acid sequences of the VH domain of SEQ ID NO:1 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 1. In one aspect, the anti-C3bBb antibody comprises the three heavy chain CDR amino acid sequences of the VH domain of SEQ ID NO:1 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:1. In one aspect, the anti-C3bBb antibody comprises the three heavy chain CDR amino acid sequences of the VH domain of SEQ ID NO:1 and a framework of at least 95% sequence identity to the framework amino acid sequence of the VH domain of SEQ ID NO:1. In another aspect, the anti-C3bBb antibody comprises the three heavy chain CDR amino acid sequences of the VH domain of SEQ ID NO:1 and a framework of at least of at least 98% sequence identity to the framework amino acid sequence of the VH domain of SEQ ID NO:1.

In one aspect, an anti-C3bBb antibody comprises one or more of the light chain CDR amino acid sequences of the VL domain of SEQ ID NO:2 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:2. In one aspect, the anti-C3bBb antibody comprises the three light chain CDR amino acid sequences of the VL domain of SEQ ID NO:2 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:2. In one aspect, the anti-C3bBb antibody comprises the three light chain CDR amino acid sequences of the VL domain of SEQ ID NO:2 and a framework of at least 95% sequence identity to the framework amino acid sequence of the VL domain of SEQ ID NO:2. In another aspect, the anti-C3bBb antibody comprises the three light chain CDR amino acid sequences of the VL domain of SEQ ID NO:2 and a framework of at least particularly of at least 98% sequence identity to the framework amino acid sequence of the VH domain of SEQ ID NO:2.

In one aspect, the anti-C3bBb antibody comprises (a) CDR-H1 comprising the amino acid sequence of SEQ ID NO:449; (b) CDR-H2 comprising the amino acid sequence of SEQ ID NO:450; (c) CDR-H3 comprising the amino acid sequence of SEQ ID NO:451; (d) CDR-L1 comprising the amino acid sequence of SEQ ID NO:452; (e) CDR-L2 comprising the amino acid sequence of SEQ ID NO:453; and (f) CDR-L3 comprising the amino acid sequence of SEQ ID NO:454, and a VH domain having at least 90%, 91%, 92%, 93%, 94%, 95%, 96%, 97%, 98%, 99%, or 100% sequence identity to the amino acid sequence of SEQ ID NO:1, and a VL domain having at least 90%, 91%, 92%, 93%, 94%, 95%, 96%, 97%, 98%, 99%, or 100% sequence identity to the amino acid sequence of SEQ ID NO:2. In one aspect, the VH domain has at least 95% sequence identity to the amino acid sequence of SEQ ID NO:1. In one aspect, the VL domain has at least 95% sequence identity to the amino acid sequence of SEQ ID NO:2.

In one aspect, the anti-C3bBb antibody comprises (a) CDR-H1 comprising the amino acid sequence of SEQ ID NO:449; (b) CDR-H2 comprising the amino acid sequence of SEQ ID NO:450; (c) CDR-H3 comprising the amino acid sequence of SEQ ID NO:451; (d) CDR-L1 comprising the amino acid sequence of SEQ ID NO:452; (e) CDR-L2 comprising the amino acid sequence of SEQ ID NO:453; and (f) CDR-L3 comprising the amino acid sequence of SEQ ID NO:454, and a VH domain having at least 90%, 91%, 92%, 93%, 94%, 95%, 96%, 97%, 98%, 99%, or 100% sequence identity to the amino acid sequence of SEQ ID NO: 1, and a VL domain having at least 90%, 91%, 92%, 93%, 94%, 95%, 96%, 97%, 98%, 99%, or 100% sequence identity to the amino acid sequence of SEQ ID NO:2; wherein the antibody specifically binds to human C3bBb. In one aspect, the VH domain has at least 95% sequence identity to the amino acid sequence of SEQ ID NO:1. In one aspect, the VL domain has at least 95% sequence identity to the amino acid sequence of SEQ ID NO:2. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:1 and a VL sequence of SEQ ID NO:2.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1. In one aspect, an anti-C3bBb antibody comprises a heavy chain variable domain (VH) sequence having at least 95%, sequence identity to the amino acid sequence of SEQ ID NO:2. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:1. In certain aspects, substitutions, insertions, or deletions occur in regions outside the CDRs (i.e., in the FRs). Optionally, the anti-C3bBb antibody comprises the VH sequence in SEQ ID NO:1, including post-translational modifications of that sequence. In a particular aspect, the VH comprises one, two or three CDRs selected from: (a) CDR-H1, comprising the amino acid sequence of SEQ ID NO:449, (b) CDR-H2, comprising the amino acid sequence of SEQ ID NO:450, and (c) CDR-H3, comprising the amino acid sequence of SEQ ID NO:451. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:2. In one aspect, an anti-C3bBb antibody comprises a light chain variable domain (VL) sequence having at least 95% sequence identity to the amino acid sequence of SEQ ID NO:2. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:2. In certain aspects, the substitutions, insertions, or deletions occur in regions outside the CDRs (i.e., in the FRs). Optionally, the anti-C3bBb antibody comprises the VL sequence in SEQ ID NO:2, including post-translational modifications of that sequence. In a particular aspect, the VL comprises one, two or three CDRs selected from: (a) CDR-L1, comprising the amino acid sequence of SEQ ID NO:452, (b) CDR-L2, comprising the amino acid sequence of SEQ ID NO:453, and (c) CDR-L3, comprising the amino acid sequence of SEQ ID NO:454.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:1 and SEQ ID NO:2, respectively, including post-translational modifications of those sequences.

In another aspect, an anti-C3bBb antibody is provided, wherein the antibody comprises a heavy chain amino acid sequence of SEQ OF NO:435 and a light chain amino acid sequence of SEQ ID NO:436.

In one aspect, an antibody is provided that comprises a VH domain as described above and the heavy chain constant domain amino acid sequences as comprised in SEQ ID NO:435, and a VL domain as described above and the light chain constant domain amino acid sequences as comprised in SEQ ID NO:436.

### Antibody P1AG9376

In another aspect, the invention provides an antibody comprising (a) CDR-H1 comprising the amino acid sequence of SEQ ID NO:455; (b) CDR-H2 comprising the amino acid sequence of SEQ ID NO:456; (c) CDR-H3 comprising the amino acid sequence of SEQ ID NO:457; (d) CDR-L1 comprising the amino acid sequence of SEQ ID NO:458; (e) CDR-L2 comprising the amino acid sequence of SEQ ID NO:459; and (f) CDR-L3 comprising the amino acid sequence of SEQ ID NO:460 (corresponding to antibody #2, P1AG9376).

In another aspect, an anti-C3bBb antibody comprises one or more of the CDR sequences of the VH of SEQ ID NO:3. In another embodiment, an anti-C3bBb antibody comprises one or more of the CDR sequences of the VL of SEQ ID NO:4. In another embodiment, an anti-C3bBb antibody comprises the CDR sequences of the VH of SEQ ID NO:3 and the CDR sequences of the VL of SEQ ID NO:4.

In a further aspect, an anti-C3bBb antibody comprises the CDR-H1, CDR-H2 and CDR-H3 amino acid sequences of the VH domain of SEQ ID NO:3 and the CDR-L1, CDR-L2 and CDR-L3 amino acid sequences of the VL domain of SEQ ID NO:4.

In one aspect, an anti-C3bBb antibody comprises one or more of the heavy chain CDR amino acid sequences of the VH domain of SEQ ID NO:3 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:3. In one aspect, the anti-C3bBb antibody comprises the three heavy chain CDR amino acid sequences of the VH domain of SEQ ID NO:3 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:3. In one aspect, the anti-C3bBb antibody comprises the three heavy chain CDR amino acid sequences of the VH domain of SEQ ID NO:3 and a framework of at least 95% sequence identity to the framework amino acid sequence of the VH domain of SEQ ID NO:3. In another aspect, the anti-C3bBb antibody comprises the three heavy chain CDR amino acid sequences of the VH domain of SEQ ID NO:3 and a framework of at least of at least 98% sequence identity to the framework amino acid sequence of the VH domain of SEQ ID NO:3.

In one aspect, an anti-C3bBb antibody comprises one or more of the light chain CDR amino acid sequences of the VL domain of SEQ ID NO:4 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:4. In one aspect, the anti-C3bBb antibody comprises the three light chain CDR amino acid sequences of the VL domain of SEQ ID NO:4 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:4. In one aspect, the anti-C3bBb antibody comprises the three light chain CDR amino acid sequences of the VL domain of SEQ ID NO:4 and a framework of at least 95% sequence identity to the framework amino acid sequence of the VL domain of SEQ ID NO:4. In another aspect, the anti-C3bBb antibody comprises the three light chain CDR amino acid sequences of the VL domain of SEQ ID NO:4 and a framework of at least particularly of at least 98% sequence identity to the framework amino acid sequence of the VH domain of SEQ ID NO:4.

In one aspect, the anti-C3bBb antibody comprises (a) CDR-H1 comprising the amino acid sequence of SEQ ID NO:455; (b) CDR-H2 comprising the amino acid sequence of SEQ ID NO:456; (c) CDR-H3 comprising the amino acid sequence of SEQ ID NO:457; (d) CDR-L1 comprising the amino acid sequence of SEQ ID NO:458; (e) CDR-L2 comprising the amino acid sequence of SEQ ID NO:459; and (f) CDR-L3 comprising the amino acid sequence of SEQ ID NO:460, and a VH domain having at least 90%, 91%, 92%, 93%, 94%, 95%, 96%, 97%, 98%, 99%, or 100% sequence identity to the amino acid sequence of SEQ ID NO:3, and a VL domain having at least 90%, 91%, 92%, 93%, 94%, 95%, 96%, 97%, 98%, 99%, or 100% sequence identity to the amino acid sequence of SEQ ID NO:4. In one aspect, the VH domain has at least 95% sequence identity to the amino acid sequence of SEQ ID NO:3. In one aspect, the VL domain has at least 95% sequence identity to the amino acid sequence of SEQ ID NO:4.

In one aspect, the anti-C3bBb antibody comprises (a) CDR-H1 comprising the amino acid sequence of SEQ ID NO:455; (b) CDR-H2 comprising the amino acid sequence of SEQ ID NO:456; (c) CDR-H3 comprising the amino acid sequence of SEQ ID NO:457; (d) CDR-L1 comprising the amino acid sequence of SEQ ID NO:458; (e) CDR-L2 comprising the amino acid sequence of SEQ ID NO:459; and (f) CDR-L3 comprising the amino acid sequence of SEQ ID NO:460, and a VH domain having at least 90%, 91%, 92%, 93%, 94%, 95%, 96%, 97%, 98%, 99%, or 100% sequence identity to the amino acid sequence of SEQ ID NO:3, and a VL domain having at least 90%, 91%, 92%, 93%, 94%, 95%, 96%, 97%, 98%, 99%, or 100% sequence identity to the amino acid sequence of SEQ ID NO:4; wherein the antibody specifically binds to human C3bBb. In one aspect, the VH domain has at least 95% sequence identity to the amino acid sequence of SEQ ID NO:3. In one aspect, the VL domain has at least 95% sequence identity to the amino acid sequence of SEQ ID NO:4. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:3 and a VL sequence of SEQ ID NO:4.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:3. In one aspect, an anti-C3bBb antibody comprises a heavy chain variable domain (VH) sequence having at least 95%, sequence identity to the amino acid sequence of SEQ ID NO:4. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:3. In certain aspects, substitutions, insertions, or deletions occur in regions outside the CDRs (i.e., in the FRs). Optionally, the anti-C3bBb antibody comprises the VH sequence in SEQ ID NO:3, including post-translational modifications of that sequence. In a particular aspect, the VH comprises one, two or three CDRs selected from: (a) CDR-H1, comprising the amino acid sequence of SEQ ID NO:455, (b) CDR-H2, comprising the amino acid sequence of SEQ ID NO:456, and (c) CDR-H3, comprising the amino acid sequence of SEQ ID NO:457. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:4. In one aspect, an anti-C3bBb antibody comprises a light chain variable domain (VL) sequence having at least 95% sequence identity to the amino acid sequence of SEQ ID NO:4. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:4. In certain aspects, the substitutions, insertions, or deletions occur in regions outside the CDRs (i.e., in the FRs). Optionally, the anti-C3bBb antibody comprises the VL sequence in SEQ ID NO:4, including post-translational modifications of that sequence. In a particular aspect, the VL comprises one, two or three CDRs selected from: (a) CDR-L1, comprising the amino acid sequence of SEQ ID NO:458, (b) CDR-L2, comprising the amino acid sequence of SEQ ID NO:459, and (c) CDR-L3, comprising the amino acid sequence of SEQ ID NO:460.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:3 and SEQ ID NO:4, respectively, including post-translational modifications of those sequences.

In another aspect, an anti-C3bBb antibody is provided, wherein the antibody comprises a heavy chain amino acid sequence of SEQ OF NO:437 and a light chain amino acid sequence of SEQ ID NO:438.

In one aspect, an antibody is provided that comprises a VH domain as described above and the heavy chain constant domain amino acid sequences as comprised in SEQ ID NO:437, and a VL domain as described above and the light chain constant domain amino acid sequences as comprised in SEQ ID NO:438.

### Antibody P1AG9372

In another aspect, the invention provides an antibody comprising (a) CDR-H1 comprising the amino acid sequence of SEQ ID NO:461; (b) CDR-H2 comprising the amino acid sequence of SEQ ID NO:462; (c) CDR-H3 comprising the amino acid sequence of SEQ ID NO:463; (d) CDR-L1 comprising the amino acid sequence of SEQ ID NO:464; (e) CDR-L2 comprising the amino acid sequence of SEQ ID NO:465; and (f) CDR-L3 comprising the amino acid sequence of SEQ ID NO:466 (corresponding to antibody #3, P1AG9372).

In another aspect, an anti-C3bBb antibody comprises one or more of the CDR sequences of the VH of SEQ ID NO:5. In another embodiment, an anti-C3bBb antibody comprises one or more of the CDR sequences of the VL of SEQ ID NO:6. In another embodiment, an anti-C3bBb antibody comprises the CDR sequences of the VH of SEQ ID NO:5 and the CDR sequences of the VL of SEQ ID NO:6.

In a further aspect, an anti-C3bBb antibody comprises the CDR-H1, CDR-H2 and CDR-H3 amino acid sequences of the VH domain of SEQ ID NO:5 and the CDR-L1, CDR-L2 and CDR-L3 amino acid sequences of the VL domain of SEQ ID NO:6.

In one aspect, an anti-C3bBb antibody comprises one or more of the heavy chain CDR amino acid sequences of the VH domain of SEQ ID NO:5 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:5. In one aspect, the anti-C3bBb antibody comprises the three heavy chain CDR amino acid sequences of the VH domain of SEQ ID NO:5 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:5. In one aspect, the anti-C3bBb antibody comprises the three heavy chain CDR amino acid sequences of the VH domain of SEQ ID NO:5 and a framework of at least 95% sequence identity to the framework amino acid sequence of the VH domain of SEQ ID NO:5. In another aspect, the anti-C3bBb antibody comprises the three heavy chain CDR amino acid sequences of the VH domain of SEQ ID NO:5 and a framework of at least of at least 98% sequence identity to the framework amino acid sequence of the VH domain of SEQ ID NO:5.

In one aspect, an anti-C3bBb antibody comprises one or more of the light chain CDR amino acid sequences of the VL domain of SEQ ID NO:6 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:6. In one aspect, the anti-C3bBb antibody comprises the three light chain CDR amino acid sequences of the VL domain of SEQ ID NO:6 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:6. In one aspect, the anti-C3bBb antibody comprises the three light chain CDR amino acid sequences of the VL domain of SEQ ID NO:6 and a framework of at least 95% sequence identity to the framework amino acid sequence of the VL domain of SEQ ID NO:6. In another aspect, the anti-C3bBb antibody comprises the three light chain CDR amino acid sequences of the VL domain of SEQ ID NO:6 and a framework of at least particularly of at least 98% sequence identity to the framework amino acid sequence of the VH domain of SEQ ID NO:6.

In one aspect, the anti-C3bBb antibody comprises (a) CDR-H1 comprising the amino acid sequence of SEQ ID NO:461; (b) CDR-H2 comprising the amino acid sequence of SEQ ID NO:462; (c) CDR-H3 comprising the amino acid sequence of SEQ ID NO:463; (d) CDR-L1 comprising the amino acid sequence of SEQ ID NO:464; (e) CDR-L2 comprising the amino acid sequence of SEQ ID NO:465; and (f) CDR-L3 comprising the amino acid sequence of SEQ ID NO:466, and a VH domain having at least 90%, 91%, 92%, 93%, 94%, 95%, 96%, 97%, 98%, 99%, or 100% sequence identity to the amino acid sequence of SEQ ID NO:5, and a VL domain having at least 90%, 91%, 92%, 93%, 94%, 95%, 96%, 97%, 98%, 99%, or 100% sequence identity to the amino acid sequence of SEQ ID NO:6. In one aspect, the VH domain has at least 95% sequence identity to the amino acid sequence of SEQ ID NO:5. In one aspect, the VL domain has at least 95% sequence identity to the amino acid sequence of SEQ ID NO:6.

In one aspect, the anti-C3bBb antibody comprises (a) CDR-H1 comprising the amino acid sequence of SEQ ID NO:461; (b) CDR-H2 comprising the amino acid sequence of SEQ ID NO:462; (c) CDR-H3 comprising the amino acid sequence of SEQ ID NO:463; (d) CDR-L1 comprising the amino acid sequence of SEQ ID NO:464; (e) CDR-L2 comprising the amino acid sequence of SEQ ID NO:465; and (f) CDR-L3 comprising the amino acid sequence of SEQ ID NO:466, and a VH domain having at least 90%, 91%, 92%, 93%, 94%, 95%, 96%, 97%, 98%, 99%, or 100% sequence identity to the amino acid sequence of SEQ ID NO:5, and a VL domain having at least 90%, 91%, 92%, 93%, 94%, 95%, 96%, 97%, 98%, 99%, or 100% sequence identity to the amino acid sequence of SEQ ID NO:6; wherein the antibody specifically binds to human C3bBb. In one aspect, the VH domain has at least 95% sequence identity to the amino acid sequence of SEQ ID NO:5. In one aspect, the VL domain has at least 95% sequence identity to the amino acid sequence of SEQ ID NO:6. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:5 and a VL sequence of SEQ ID NO:6.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:5. In one aspect, an anti-C3bBb antibody comprises a heavy chain variable domain (VH) sequence having at least 95%, sequence identity to the amino acid sequence of SEQ ID NO:6. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:5. In certain aspects, substitutions, insertions, or deletions occur in regions outside the CDRs (i.e., in the FRs). Optionally, the anti-C3bBb antibody comprises the VH sequence in SEQ ID NO:5, including post-translational modifications of that sequence. In a particular aspect, the VH comprises one, two or three CDRs selected from: (a) CDR-H1, comprising the amino acid sequence of SEQ ID NO:461, (b) CDR-H2, comprising the amino acid sequence of SEQ ID NO:462, and (c) CDR-H3, comprising the amino acid sequence of SEQ ID NO:463. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:6. In one aspect, an anti-C3bBb antibody comprises a light chain variable domain (VL) sequence having at least 95% sequence identity to the amino acid sequence of SEQ ID NO:6. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:6. In certain aspects, the substitutions, insertions, or deletions occur in regions outside the CDRs (i.e., in the FRs). Optionally, the anti-C3bBb antibody comprises the VL sequence in SEQ ID NO:6, including post-translational modifications of that sequence. In a particular aspect, the VL comprises one, two or three CDRs selected from: (a) CDR-L1, comprising the amino acid sequence of SEQ ID NO:464, (b) CDR-L2, comprising the amino acid sequence of SEQ ID NO:465, and (c) CDR-L3, comprising the amino acid sequence of SEQ ID NO:466.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:5 and SEQ ID NO:6, respectively, including post-translational modifications of those sequences.

In another aspect, an anti-C3bBb antibody is provided, wherein the antibody comprises a heavy chain amino acid sequence of SEQ OF NO:439 and a light chain amino acid sequence of SEQ ID NO:440.

In one aspect, an antibody is provided that comprises a VH domain as described above and the heavy chain constant domain amino acid sequences as comprised in SEQ ID NO:439, and a VL domain as described above and the light chain constant domain amino acid sequences as comprised in SEQ ID NO:440.

### Antibody P1AG9420

In another aspect, the invention provides an antibody comprising (a) CDR-H1 comprising the amino acid sequence of SEQ ID NO:467; (b) CDR-H2 comprising the amino acid sequence of SEQ ID NO:468; (c) CDR-H3 comprising the amino acid sequence of SEQ ID NO:469; (d) CDR-L1 comprising the amino acid sequence of SEQ ID NO:470; (e) CDR-L2 comprising the amino acid sequence of SEQ ID NO:471; and (f) CDR-L3 comprising the amino acid sequence of SEQ ID NO:472 (corresponding to antibody #4, P1AG9420).

In another aspect, an anti-C3bBb antibody comprises one or more of the CDR sequences of the VH of SEQ ID NO:7. In another embodiment, an anti-C3bBb antibody comprises one or more of the CDR sequences of the VL of SEQ ID NO:8. In another embodiment, an anti-C3bBb antibody comprises the CDR sequences of the VH of SEQ ID NO:7 and the CDR sequences of the VL of SEQ ID NO:8.

In a further aspect, an anti-C3bBb antibody comprises the CDR-H1, CDR-H2 and CDR-H3 amino acid sequences of the VH domain of SEQ ID NO:7 and the CDR-L1, CDR-L2 and CDR-L3 amino acid sequences of the VL domain of SEQ ID NO: 8.

In one aspect, an anti-C3bBb antibody comprises one or more of the heavy chain CDR amino acid sequences of the VH domain of SEQ ID NO:7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:7. In one aspect, the anti-C3bBb antibody comprises the three heavy chain CDR amino acid sequences of the VH domain of SEQ ID NO:7 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:7. In one aspect, the anti-C3bBb antibody comprises the three heavy chain CDR amino acid sequences of the VH domain of SEQ ID NO:7 and a framework of at least 95% sequence identity to the framework amino acid sequence of the VH domain of SEQ ID NO:7. In another aspect, the anti-C3bBb antibody comprises the three heavy chain CDR amino acid sequences of the VH domain of SEQ ID NO:7 and a framework of at least of at least 98% sequence identity to the framework amino acid sequence of the VH domain of SEQ ID NO:7.

In one aspect, an anti-C3bBb antibody comprises one or more of the light chain CDR amino acid sequences of the VL domain of SEQ ID NO:8 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8. In one aspect, the anti-C3bBb antibody comprises the three light chain CDR amino acid sequences of the VL domain of SEQ ID NO:8 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:8. In one aspect, the anti-C3bBb antibody comprises the three light chain CDR amino acid sequences of the VL domain of SEQ ID NO:8 and a framework of at least 95% sequence identity to the framework amino acid sequence of the VL domain of SEQ ID NO:8. In another aspect, the anti-C3bBb antibody comprises the three light chain CDR amino acid sequences of the VL domain of SEQ ID NO:8 and a framework of at least particularly of at least 98% sequence identity to the framework amino acid sequence of the VH domain of SEQ ID NO:8.

In one aspect, the anti-C3bBb antibody comprises (a) CDR-H1 comprising the amino acid sequence of SEQ ID NO:467; (b) CDR-H2 comprising the amino acid sequence of SEQ ID NO:468; (c) CDR-H3 comprising the amino acid sequence of SEQ ID NO:469; (d) CDR-L1 comprising the amino acid sequence of SEQ ID NO:470; (e) CDR-L2 comprising the amino acid sequence of SEQ ID NO:471; and (f) CDR-L3 comprising the amino acid sequence of SEQ ID NO:472, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:8. In one aspect, the VH domain has at least 95% sequence identity to the amino acid sequence of SEQ ID NO:7. In one aspect, the VL domain has at least 95% sequence identity to the amino acid sequence of SEQ ID NO:8.

In one aspect, the anti-C3bBb antibody comprises (a) CDR-H1 comprising the amino acid sequence of SEQ ID NO:467; (b) CDR-H2 comprising the amino acid sequence of SEQ ID NO:468; (c) CDR-H3 comprising the amino acid sequence of SEQ ID NO:469; (d) CDR-L1 comprising the amino acid sequence of SEQ ID NO:470; (e) CDR-L2 comprising the amino acid sequence of SEQ ID NO:471; and (f) CDR-L3 comprising the amino acid sequence of SEQ ID NO:472, and a VH domain having at least 90%, 91%, 92%, 93%, 94%, 95%, 96%, 97%, 98%, 99%, or 100% sequence identity to the amino acid sequence of SEQ ID NO:7, and a VL domain having at least 90%, 91%, 92%, 93%, 94%, 95%, 96%, 97%, 98%, 99%, or 100% sequence identity to the amino acid sequence of SEQ ID NO:8; wherein the antibody specifically binds to human C3bBb. In one aspect, the VH domain has at least 95% sequence identity to the amino acid sequence of SEQ ID NO:7. In one aspect, the VL domain has at least 95% sequence identity to the amino acid sequence of SEQ ID NO:8. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:7 and a VL sequence of SEQ ID NO:8.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:7. In one aspect, an anti-C3bBb antibody comprises a heavy chain variable domain (VH) sequence having at least 95%, sequence identity to the amino acid sequence of SEQ ID NO:8. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:7. In certain aspects, substitutions, insertions, or deletions occur in regions outside the CDRs (i.e., in the FRs). Optionally, the anti-C3bBb antibody comprises the VH sequence in SEQ ID NO:7, including post-translational modifications of that sequence. In a particular aspect, the VH comprises one, two or three CDRs selected from: (a) CDR-H1, comprising the amino acid sequence of SEQ ID NO:467, (b) CDR-H2, comprising the amino acid sequence of SEQ ID NO:468, and (c) CDR-H3, comprising the amino acid sequence of SEQ ID NO:469. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:8. In one aspect, an anti-C3bBb antibody comprises a light chain variable domain (VL) sequence having at least 95% sequence identity to the amino acid sequence of SEQ ID NO:8. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:8. In certain aspects, the substitutions, insertions, or deletions occur in regions outside the CDRs (i.e., in the FRs). Optionally, the anti-C3bBb antibody comprises the VL sequence in SEQ ID NO:8, including post-translational modifications of that sequence. In a particular aspect, the VL comprises one, two or three CDRs selected from: (a) CDR-L1, comprising the amino acid sequence of SEQ ID NO:470, (b) CDR-L2, comprising the amino acid sequence of SEQ ID NO:471, and (c) CDR-L3, comprising the amino acid sequence of SEQ ID NO:472.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:7 and SEQ ID NO:8, respectively, including post-translational modifications of those sequences.

In another aspect, an anti-C3bBb antibody is provided, wherein the antibody comprises a heavy chain amino acid sequence of SEQ OF NO:441 and a light chain amino acid sequence of SEQ ID NO:442.

In one aspect, an antibody is provided that comprises a VH domain as described above and the heavy chain constant domain amino acid sequences as comprised in SEQ ID NO:441, and a VL domain as described above and the light chain constant domain amino acid sequences as comprised in SEQ ID NO:442.

### Antibody P1AG9391

In another aspect, the invention provides an antibody comprising (a) CDR-H1 comprising the amino acid sequence of SEQ ID NO:473; (b) CDR-H2 comprising the amino acid sequence of SEQ ID NO:474; (c) CDR-H3 comprising the amino acid sequence of SEQ ID NO:475; (d) CDR-L1 comprising the amino acid sequence of SEQ ID NO:476; (e) CDR-L2 comprising the amino acid sequence of SEQ ID NO:477; and (f) CDR-L3 comprising the amino acid sequence of SEQ ID NO:478 (corresponding to antibody #5, P1AG9391).

In another aspect, an anti-C3bBb antibody comprises one or more of the CDR sequences of the VH of SEQ ID NO:9. In another embodiment, an anti-C3bBb antibody comprises one or more of the CDR sequences of the VL of SEQ ID NO:10. In another embodiment, an anti-C3bBb antibody comprises the CDR sequences of the VH of SEQ ID NO:9 and the CDR sequences of the VL of SEQ ID NO:10.

In a further aspect, an anti-C3bBb antibody comprises the CDR-H1, CDR-H2 and CDR-H3 amino acid sequences of the VH domain of SEQ ID NO:9 and the CDR-L1, CDR-L2 and CDR-L3 amino acid sequences of the VL domain of SEQ ID NO: 10.

In one aspect, an anti-C3bBb antibody comprises one or more of the heavy chain CDR amino acid sequences of the VH domain of SEQ ID NO:9 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:9. In one aspect, the anti-C3bBb antibody comprises the three heavy chain CDR amino acid sequences of the VH domain of SEQ ID NO:9 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:9. In one aspect, the anti-C3bBb antibody comprises the three heavy chain CDR amino acid sequences of the VH domain of SEQ ID NO:9 and a framework of at least 95% sequence identity to the framework amino acid sequence of the VH domain of SEQ ID NO:9. In another aspect, the anti-C3bBb antibody comprises the three heavy chain CDR amino acid sequences of the VH domain of SEQ ID NO:9 and a framework of at least of at least 98% sequence identity to the framework amino acid sequence of the VH domain of SEQ ID NO:9.

In one aspect, an anti-C3bBb antibody comprises one or more of the light chain CDR amino acid sequences of the VL domain of SEQ ID NO:10 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:10. In one aspect, the anti-C3bBb antibody comprises the three light chain CDR amino acid sequences of the VL domain of SEQ ID NO:10 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:10. In one aspect, the anti-C3bBb antibody comprises the three light chain CDR amino acid sequences of the VL domain of SEQ ID NO:10 and a framework of at least 95% sequence identity to the framework amino acid sequence of the VL domain of SEQ ID NO:10. In another aspect, the anti-C3bBb antibody comprises the three light chain CDR amino acid sequences of the VL domain of SEQ ID NO:10 and a framework of at least particularly of at least 98% sequence identity to the framework amino acid sequence of the VH domain of SEQ ID NO:10.

In one aspect, the anti-C3bBb antibody comprises (a) CDR-H1 comprising the amino acid sequence of SEQ ID NO:473; (b) CDR-H2 comprising the amino acid sequence of SEQ ID NO:474; (c) CDR-H3 comprising the amino acid sequence of SEQ ID NO:475; (d) CDR-L1 comprising the amino acid sequence of SEQ ID NO:476; (e) CDR-L2 comprising the amino acid sequence of SEQ ID NO:477; and (f) CDR-L3 comprising the amino acid sequence of SEQ ID NO:478, and a VH domain having at least 90%, 91%, 92%, 93%, 94%, 95%, 96%, 97%, 98%, 99%, or 100% sequence identity to the amino acid sequence of SEQ ID NO:9, and a VL domain having at least 90%, 91%, 92%, 93%, 94%, 95%, 96%, 97%, 98%, 99%, or 100% sequence identity to the amino acid sequence of SEQ ID NO:10. In one aspect, the VH domain has at least 95% sequence identity to the amino acid sequence of SEQ ID NO:9. In one aspect, the VL domain has at least 95% sequence identity to the amino acid sequence of SEQ ID NO:10.

In one aspect, the anti-C3bBb antibody comprises (a) CDR-H1 comprising the amino acid sequence of SEQ ID NO:473; (b) CDR-H2 comprising the amino acid sequence of SEQ ID NO:474; (c) CDR-H3 comprising the amino acid sequence of SEQ ID NO:475; (d) CDR-L1 comprising the amino acid sequence of SEQ ID NO:476; (e) CDR-L2 comprising the amino acid sequence of SEQ ID NO:477; and (f) CDR-L3 comprising the amino acid sequence of SEQ ID NO:478, and a VH domain having at least 90%, 91%, 92%, 93%, 94%, 95%, 96%, 97%, 98%, 99%, or 100% sequence identity to the amino acid sequence of SEQ ID NO:9, and a VL domain having at least 90%, 91%, 92%, 93%, 94%, 95%, 96%, 97%, 98%, 99%, or 100% sequence identity to the amino acid sequence of SEQ ID NO:10; wherein the antibody specifically binds to human C3bBb. In one aspect, the VH domain has at least 95% sequence identity to the amino acid sequence of SEQ ID NO:9. In one aspect, the VL domain has at least 95% sequence identity to the amino acid sequence of SEQ ID NO:10. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:9 and a VL sequence of SEQ ID NO:10.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:9. In one aspect, an anti-C3bBb antibody comprises a heavy chain variable domain (VH) sequence having at least 95%, sequence identity to the amino acid sequence of SEQ ID NO:10. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:9. In certain aspects, substitutions, insertions, or deletions occur in regions outside the CDRs (i.e., in the FRs). Optionally, the anti-C3bBb antibody comprises the VH sequence in SEQ ID NO:9, including post-translational modifications of that sequence. In a particular aspect, the VH comprises one, two or three CDRs selected from: (a) CDR-H1, comprising the amino acid sequence of SEQ ID NO:473, (b) CDR-H2, comprising the amino acid sequence of SEQ ID NO:474, and (c) CDR-H3, comprising the amino acid sequence of SEQ ID NO:475. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:10. In one aspect, an anti-C3bBb antibody comprises a light chain variable domain (VL) sequence having at least 95% sequence identity to the amino acid sequence of SEQ ID NO:10. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:10. In certain aspects, the substitutions, insertions, or deletions occur in regions outside the CDRs (i.e., in the FRs). Optionally, the anti-C3bBb antibody comprises the VL sequence in SEQ ID NO: 10, including post-translational modifications of that sequence. In a particular aspect, the VL comprises one, two or three CDRs selected from: (a) CDR-L1, comprising the amino acid sequence of SEQ ID NO:476, (b) CDR-L2, comprising the amino acid sequence of SEQ ID NO:477, and (c) CDR-L3, comprising the amino acid sequence of SEQ ID NO:478.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:9 and SEQ ID NO:10, respectively, including post-translational modifications of those sequences.

In another aspect, an anti-C3bBb antibody is provided, wherein the antibody comprises a heavy chain amino acid sequence of SEQ OF NO:443 and a light chain amino acid sequence of SEQ ID NO:444.

In one aspect, an antibody is provided that comprises a VH domain as described above and the heavy chain constant domain amino acid sequences as comprised in SEQ ID NO:443, and a VL domain as described above and the light chain constant domain amino acid sequences as comprised in SEQ ID NO:444.

### Antibody P1AH1205

In another aspect, the invention provides an antibody comprising (a) CDR-H1 comprising the amino acid sequence of SEQ ID NO:479; (b) CDR-H2 comprising the amino acid sequence of SEQ ID NO:480; (c) CDR-H3 comprising the amino acid sequence of SEQ ID NO:481; (d) CDR-L1 comprising the amino acid sequence of SEQ ID NO:482; (e) CDR-L2 comprising the amino acid sequence of SEQ ID NO:483; and (f) CDR-L3 comprising the amino acid sequence of SEQ ID NO:484 (corresponding to antibody #6, P1AH1205).

In another aspect, an anti-C3bBb antibody comprises one or more of the CDR sequences of the VH of SEQ ID NO:11. In another embodiment, an anti-C3bBb antibody comprises one or more of the CDR sequences of the VL of SEQ ID NO:12. In another embodiment, an anti-C3bBb antibody comprises the CDR sequences of the VH of SEQ ID NO:11 and the CDR sequences of the VL of SEQ ID NO:12.

In a further aspect, an anti-C3bBb antibody comprises the CDR-H1, CDR-H2 and CDR-H3 amino acid sequences of the VH domain of SEQ ID NO:11 and the CDR-L1, CDR-L2 and CDR-L3 amino acid sequences of the VL domain of SEQ ID NO: 12.

In one aspect, an anti-C3bBb antibody comprises one or more of the heavy chain CDR amino acid sequences of the VH domain of SEQ ID NO:11 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:11. In one aspect, the anti-C3bBb antibody comprises the three heavy chain CDR amino acid sequences of the VH domain of SEQ ID NO:11 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:11. In one aspect, the anti-C3bBb antibody comprises the three heavy chain CDR amino acid sequences of the VH domain of SEQ ID NO:11 and a framework of at least 95% sequence identity to the framework amino acid sequence of the VH domain of SEQ ID NO:11. In another aspect, the anti-C3bBb antibody comprises the three heavy chain CDR amino acid sequences of the VH domain of SEQ ID NO:11 and a framework of at least of at least 98% sequence identity to the framework amino acid sequence of the VH domain of SEQ ID NO:11.

In one aspect, an anti-C3bBb antibody comprises one or more of the light chain CDR amino acid sequences of the VL domain of SEQ ID NO:12 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:12. In one aspect, the anti-C3bBb antibody comprises the three light chain CDR amino acid sequences of the VL domain of SEQ ID NO:12 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:12. In one aspect, the anti-C3bBb antibody comprises the three light chain CDR amino acid sequences of the VL domain of SEQ ID NO:12 and a framework of at least 95% sequence identity to the framework amino acid sequence of the VL domain of SEQ ID NO:12. In another aspect, the anti-C3bBb antibody comprises the three light chain CDR amino acid sequences of the VL domain of SEQ ID NO:12 and a framework of at least particularly of at least 98% sequence identity to the framework amino acid sequence of the VH domain of SEQ ID NO:12.

In one aspect, the anti-C3bBb antibody comprises (a) CDR-H1 comprising the amino acid sequence of SEQ ID NO:479; (b) CDR-H2 comprising the amino acid sequence of SEQ ID NO:480; (c) CDR-H3 comprising the amino acid sequence of SEQ ID NO:481; (d) CDR-L1 comprising the amino acid sequence of SEQ ID NO:482; (e) CDR-L2 comprising the amino acid sequence of SEQ ID NO:483; and (f) CDR-L3 comprising the amino acid sequence of SEQ ID NO:484, and a VH domain having at least 90%, 91%, 92%, 93%, 94%, 95%, 96%, 97%, 98%, 99%, or 100% sequence identity to the amino acid sequence of SEQ ID NO:11, and a VL domain having at least 90%, 91%, 92%, 93%, 94%, 95%, 96%, 97%, 98%, 99%, or 100% sequence identity to the amino acid sequence of SEQ ID NO:12. In one aspect, the VH domain has at least 95% sequence identity to the amino acid sequence of SEQ ID NO:11. In one aspect, the VL domain has at least 95% sequence identity to the amino acid sequence of SEQ ID NO: 12.

In one aspect, the anti-C3bBb antibody comprises (a) CDR-H1 comprising the amino acid sequence of SEQ ID NO:479; (b) CDR-H2 comprising the amino acid sequence of SEQ ID NO:480; (c) CDR-H3 comprising the amino acid sequence of SEQ ID NO:481; (d) CDR-L1 comprising the amino acid sequence of SEQ ID NO:482; (e) CDR-L2 comprising the amino acid sequence of SEQ ID NO:483; and (f) CDR-L3 comprising the amino acid sequence of SEQ ID NO:484, and a VH domain having at least 90%, 91%, 92%, 93%, 94%, 95%, 96%, 97%, 98%, 99%, or 100% sequence identity to the amino acid sequence of SEQ ID NO:11, and a VL domain having at least 90%, 91%, 92%, 93%, 94%, 95%, 96%, 97%, 98%, 99%, or 100% sequence identity to the amino acid sequence of SEQ ID NO:12; wherein the antibody specifically binds to human C3bBb. In one aspect, the VH domain has at least 95% sequence identity to the amino acid sequence of SEQ ID NO:11. In one aspect, the VL domain has at least 95% sequence identity to the amino acid sequence of SEQ ID NO:12. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:11 and a VL sequence of SEQ ID NO:12.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:11. In one aspect, an anti-C3bBb antibody comprises a heavy chain variable domain (VH) sequence having at least 95%, sequence identity to the amino acid sequence of SEQ ID NO:12. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:11. In certain aspects, substitutions, insertions, or deletions occur in regions outside the CDRs (i.e., in the FRs). Optionally, the anti-C3bBb antibody comprises the VH sequence in SEQ ID NO:11, including post-translational modifications of that sequence. In a particular aspect, the VH comprises one, two or three CDRs selected from: (a) CDR-H1, comprising the amino acid sequence of SEQ ID NO:479, (b) CDR-H2, comprising the amino acid sequence of SEQ ID NO:480, and (c) CDR-H3, comprising the amino acid sequence of SEQ ID NO:481. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:12. In one aspect, an anti-C3bBb antibody comprises a light chain variable domain (VL) sequence having at least 95% sequence identity to the amino acid sequence of SEQ ID NO:12. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:12. In certain aspects, the substitutions, insertions, or deletions occur in regions outside the CDRs (i.e., in the FRs). Optionally, the anti-C3bBb antibody comprises the VL sequence in SEQ ID NO:12, including post-translational modifications of that sequence. In a particular aspect, the VL comprises one, two or three CDRs selected from: (a) CDR-L1, comprising the amino acid sequence of SEQ ID NO:482, (b) CDR-L2, comprising the amino acid sequence of SEQ ID NO:483, and (c) CDR-L3, comprising the amino acid sequence of SEQ ID NO:484.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:11 and SEQ ID NO:12, respectively, including post-translational modifications of those sequences.

In another aspect, an anti-C3bBb antibody is provided, wherein the antibody comprises a heavy chain amino acid sequence of SEQ OF NO:445 and a light chain amino acid sequence of SEQ ID NO:446.

In one aspect, an antibody is provided that comprises a VH domain as described above and the heavy chain constant domain amino acid sequences as comprised in SEQ ID NO:445, and a VL domain as described above and the light chain constant domain amino acid sequences as comprised in SEQ ID NO:446.

### Antibody P1AH1199

In another aspect, the invention provides an antibody comprising (a) CDR-H1 comprising the amino acid sequence of SEQ ID NO:485; (b) CDR-H2 comprising the amino acid sequence of SEQ ID NO:486; (c) CDR-H3 comprising the amino acid sequence of SEQ ID NO:487; (d) CDR-L1 comprising the amino acid sequence of SEQ ID NO:488; (e) CDR-L2 comprising the amino acid sequence of SEQ ID NO:489; and (f) CDR-L3 comprising the amino acid sequence of SEQ ID NO:490 (corresponding to antibody #7, P1AH1199).

In another aspect, an anti-C3bBb antibody comprises one or more of the CDR sequences of the VH of SEQ ID NO:13. In another embodiment, an anti-C3bBb antibody comprises one or more of the CDR sequences of the VL of SEQ ID NO:14. In another embodiment, an anti-C3bBb antibody comprises the CDR sequences of the VH of SEQ ID NO:13 and the CDR sequences of the VL of SEQ ID NO:14.

In a further aspect, an anti-C3bBb antibody comprises the CDR-H1, CDR-H2 and CDR-H3 amino acid sequences of the VH domain of SEQ ID NO: 13 and the CDR-L1, CDR-L2 and CDR-L3 amino acid sequences of the VL domain of SEQ ID NO: 14.

In one aspect, an anti-C3bBb antibody comprises one or more of the heavy chain CDR amino acid sequences of the VH domain of SEQ ID NO:13 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:13. In one aspect, the anti-C3bBb antibody comprises the three heavy chain CDR amino acid sequences of the VH domain of SEQ ID NO:13 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO:13. In one aspect, the anti-C3bBb antibody comprises the three heavy chain CDR amino acid sequences of the VH domain of SEQ ID NO:13 and a framework of at least 95% sequence identity to the framework amino acid sequence of the VH domain of SEQ ID NO:13. In another aspect, the anti-C3bBb antibody comprises the three heavy chain CDR amino acid sequences of the VH domain of SEQ ID NO:13 and a framework of at least of at least 98% sequence identity to the framework amino acid sequence of the VH domain of SEQ ID NO:13.

In one aspect, an anti-C3bBb antibody comprises one or more of the light chain CDR amino acid sequences of the VL domain of SEQ ID NO:14 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:14. In one aspect, the anti-C3bBb antibody comprises the three light chain CDR amino acid sequences of the VL domain of SEQ ID NO:14 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO:14. In one aspect, the anti-C3bBb antibody comprises the three light chain CDR amino acid sequences of the VL domain of SEQ ID NO:14 and a framework of at least 95% sequence identity to the framework amino acid sequence of the VL domain of SEQ ID NO:14. In another aspect, the anti-C3bBb antibody comprises the three light chain CDR amino acid sequences of the VL domain of SEQ ID NO:14 and a framework of at least particularly of at least 98% sequence identity to the framework amino acid sequence of the VH domain of SEQ ID NO:14.

In one aspect, the anti-C3bBb antibody comprises (a) CDR-H1 comprising the amino acid sequence of SEQ ID NO:485; (b) CDR-H2 comprising the amino acid sequence of SEQ ID NO:486; (c) CDR-H3 comprising the amino acid sequence of SEQ ID NO:487; (d) CDR-L1 comprising the amino acid sequence of SEQ ID NO:488; (e) CDR-L2 comprising the amino acid sequence of SEQ ID NO:489; and (f) CDR-L3 comprising the amino acid sequence of SEQ ID NO:490, and a VH domain having at least 90%, 91%, 92%, 93%, 94%, 95%, 96%, 97%, 98%, 99%, or 100% sequence identity to the amino acid sequence of SEQ ID NO:13, and a VL domain having at least 90%, 91%, 92%, 93%, 94%, 95%, 96%, 97%, 98%, 99%, or 100% sequence identity to the amino acid sequence of SEQ ID NO:14. In one aspect, the VH domain has at least 95% sequence identity to the amino acid sequence of SEQ ID NO:13. In one aspect, the VL domain has at least 95% sequence identity to the amino acid sequence of SEQ ID NO:14.

In one aspect, the anti-C3bBb antibody comprises (a) CDR-H1 comprising the amino acid sequence of SEQ ID NO:485; (b) CDR-H2 comprising the amino acid sequence of SEQ ID NO:486; (c) CDR-H3 comprising the amino acid sequence of SEQ ID NO:487; (d) CDR-L1 comprising the amino acid sequence of SEQ ID NO:488; (e) CDR-L2 comprising the amino acid sequence of SEQ ID NO:489; and (f) CDR-L3 comprising the amino acid sequence of SEQ ID NO:490, and a VH domain having at least 90%, 91%, 92%, 93%, 94%, 95%, 96%, 97%, 98%, 99%, or 100% sequence identity to the amino acid sequence of SEQ ID NO:13, and a VL domain having at least 90%, 91%, 92%, 93%, 94%, 95%, 96%, 97%, 98%, 99%, or 100% sequence identity to the amino acid sequence of SEQ ID NO:14; wherein the antibody specifically binds to human C3bBb. In one aspect, the VH domain has at least 95% sequence identity to the amino acid sequence of SEQ ID NO:13. In one aspect, the VL domain has at least 95% sequence identity to the amino acid sequence of SEQ ID NO:14. In one aspect, the antibody binds to C3bBb having a a dissociation constant (KD) that is up to 10 fold reduced or up to 10 fold increased when compared to the dissociation constant (KD) of an antibody comprising a VH sequence of SEQ ID NO:13 and a VL sequence of SEQ ID NO:14.

In another aspect, an anti-C3bBb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:13. In one aspect, an anti-C3bBb antibody comprises a heavy chain variable domain (VH) sequence having at least 95%, sequence identity to the amino acid sequence of SEQ ID NO:14. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:13. In certain aspects, substitutions, insertions, or deletions occur in regions outside the CDRs (i.e., in the FRs). Optionally, the anti-C3bBb antibody comprises the VH sequence in SEQ ID NO:13, including post-translational modifications of that sequence. In a particular aspect, the VH comprises one, two or three CDRs selected from: (a) CDR-H1, comprising the amino acid sequence of SEQ ID NO:485, (b) CDR-H2, comprising the amino acid sequence of SEQ ID NO:486, and (c) CDR-H3, comprising the amino acid sequence of SEQ ID NO:487. In another aspect, an anti-C3bBb antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO:14. In one aspect, an anti-C3bBb antibody comprises a light chain variable domain (VL) sequence having at least 95% sequence identity to the amino acid sequence of SEQ ID NO:14. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C3bBb antibody comprising that sequence retains the ability to bind to C3bBb. In certain aspects, a total of 1 to 10 amino acids have been substituted, inserted and/or deleted in SEQ ID NO:14. In certain aspects, the substitutions, insertions, or deletions occur in regions outside the CDRs (i.e., in the FRs). Optionally, the anti-C3bBb antibody comprises the VL sequence in SEQ ID NO:14, including post-translational modifications of that sequence. In a particular aspect, the VL comprises one, two or three CDRs selected from: (a) CDR-L1, comprising the amino acid sequence of SEQ ID NO:488, (b) CDR-L2, comprising the amino acid sequence of SEQ ID NO:489, and (c) CDR-L3, comprising the amino acid sequence of SEQ ID NO:490.

In another aspect, an anti-C3bBb antibody is provided, wherein the antibody comprises a VH sequence as in any of the aspects provided above, and a VL sequence as in any of the aspects provided above. In one aspect, the antibody comprises the VH and VL sequences in SEQ ID NO:13 and SEQ ID NO:14, respectively, including post-translational modifications of those sequences.

In another aspect, an anti-C3bBb antibody is provided, wherein the antibody comprises a heavy chain amino acid sequence of SEQ OF NO:447 and a light chain amino acid sequence of SEQ ID NO:448.

In one aspect, an antibody is provided that comprises a VH domain as described above and the heavy chain constant domain amino acid sequences as comprised in SEQ ID NO:447, and a VL domain as described above and the light chain constant domain amino acid sequences as comprised in SEQ ID NO:448.

### Further Aspects and Embodiments

In a further aspect of the invention, an anti-human C3bBb antibody according to any of the above aspects is a monoclonal antibody, including a chimeric, humanized or human antibody. In one aspect, an anti-human C3bBb antibody is an antibody fragment, e.g., a Fv, Fab, Fab′, scFv, diabody, or F(ab′)2 fragment.

In another aspect, the antibody is a full length antibody, e.g., an intact IgG1 antibody or other antibody class or isotype as defined herein. In one embodiment the antibody is a full length IgG1 antibody.

In one embodiment, the antibody is a monovalent antibody. In one embodiment, the antibody is a bivalent antibody. In one embodiment, the antibody is a trivalent antibody. In one embodiment, the antibody is a tetravalent antibody.

In a further aspect, an anti-human C3bBb antibody according to any of the above aspects may incorporate any of the features, singly or in combination, as described in Sections 1-8 below:

### 1. Antibody Affinity

In certain aspects, an antibody provided herein has a dissociation constant (KD) of ≤1 M, ≤100 nM, ≤10 nM, ≤1 nM, ≤0.1 nM, ≤0.01 nM, or ≤0.001 nM. In certain embodiments, an antibody provided herein has a dissociation constant (KD) 10⁻⁶ M or less, in one embodiment 10⁻⁷ M or less, in one embodiment 10⁻⁸ M or less, in one embodiment from 10⁻⁸ M to 10⁻¹³ M, in one embodiment from 10⁻⁹ M to 10⁻¹³ M.

In one embodiment the antibody binds human C3bBb at about 37° C. with an affinity of ≤300 pM as measured by SPR. In one embodiment the antibody binds human C3bBb at about 37° C. with an affinity of about 1 to 300 pM as measured by SPR. In one embodiment the antibody binds human C3bBb at about 37° C. with an affinity of about 1 to about 200 pM as measured by SPR.

In one embodiment the antibody binds human C3bBb at about 25° C. with an affinity of ≤100 pM as measured by SPR. In one embodiment the antibody binds human C3bBb at about 25° C. with an affinity of about 1 to 100 pM as measured by SPR. In one embodiment the antibody binds human C3bBb at about 25° C. with an affinity of ≤50 pM as measured by SPR.

In one aspect, KD is measured using a BIACORE® surface plasmon resonance assay. In one embodiment KD is measured as described in Example 5.

### 2. Antibody Fragments

In certain aspects, an antibody provided herein is an antibody fragment.

In one aspect, the antibody fragment is a Fab, Fab′, Fab′-SH, or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 5. Library-Derived Antibodies

In certain aspects, an antibody provided herein is derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 6. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for human C3bBb and the other specificity is for any other antigen. In certain aspects, bispecific antibodies may bind to two (or more) different epitopes of human C3bBb. Multispecific (e.g., bispecific) antibodies may also be used to localize cytotoxic agents or cells to cells which express human C3bBb. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to human C3bBb as well as another different antigen, or two different epitopes of human C3bBb (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for human C3bBb and the other is for CD3.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 7. Antibody Variants

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table D2 under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE D2
amino acid substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### b) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e.g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1 Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### d) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### e) Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 8. Immunoconjugates

The invention also provides immunoconjugates comprising an anti-human C3bBb antibody herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At211, I131, I125, Y90, Re186, Re188, Sm153, Bi212, P32, Pb212 and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-IBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### B. Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making an anti-human C3bBb antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an anti-human C3bBb antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse Sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HeLa); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR− CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### C. Assays

Anti-C3bBb antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with an antibody of the invention for binding to C3bBb. In certain aspects, such a competing antibody binds to the same epitope (e.g., a linear or a conformational epitope) that is bound by an antibody of the invention. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized C3bBb is incubated in a solution comprising a first labeled antibody that binds to C3bBb (e.g., an antibody of the invention) and a second unlabeled antibody that is being tested for its ability to compete with the first antibody for binding to C3bBb. The second antibody may be present in a hybridoma supernatant. As a control, immobilized C3bBb is incubated in a solution comprising the first labeled antibody but not the second unlabeled antibody. After incubation under conditions permissive for binding of the first antibody to C3bBb, excess unbound antibody is removed, and the amount of label associated with immobilized C3bBb is measured. If the amount of label associated with immobilized C3bBb is substantially reduced in the test sample relative to the control sample, then that indicates that the second antibody is competing with the first antibody for binding to C3bBb. See Harlow and Lane (1988) Antibodies: A Laboratory Manual ch. 14 (Cold Spring Harbor Laboratory, Cold Spring Harbor, NY).

### 2. Activity Assays

In one aspect, assays are provided for identifying anti-C3bBb antibodies thereof having biological activity. Biological activity may include, e.g., inhibition modulating the formation, stability, activity and dissociation of C3 convertase protein complex as well as influencing the interaction with further proteins (e.g. Properdin or Complement Factor I). Antibodies having such biological activity in vivo and/or in vitro are also provided.

In certain aspects, an antibody of the invention is tested for such biological activity.

### D. Methods and Compositions for Diagnostics and Detection

In certain aspects, any of the anti-C3bBb antibodies provided herein is useful for detecting the presence of C3bBb in a biological sample. The term “detecting” as used herein encompasses quantitative or qualitative detection.

In one aspect, an anti-C3bBb antibody for use in a method of diagnosis or detection is provided. In a further aspect, a method of detecting the presence of C3bBb in a biological sample is provided. In certain aspects, the method comprises contacting the biological sample with an anti-C3bBb antibody as described herein under conditions permissive for binding of the anti-C3bBb antibody to C3bBb, and detecting whether a complex is formed between the anti-C3bBb antibody and C3bBb. Such a method may be an in vitro or in vivo method. In one aspect, an anti-C3bBb antibody is used to select subjects eligible for therapy with an anti-C3bBb antibody, e.g., where C3bBb is a biomarker for selection of patients.

In certain aspects, labeled anti-C3bBb antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E. Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions (formulations) of an anti-C3bBb antibody as described herein can be prepared by combining the antibody with pharmaceutically acceptable carriers or excipients known to the skilled person. See, for example Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1st Ed., Woodhead Publishing (2015), § 4 and Falconer R. J., Biotechnology Advances (2019), 37, 107412. Exemplary pharmaceutical compositions of an anti-C3bBb antibody as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### F. Therapeutic Methods and Routes of Administration

Any of the anti-C3bBb antibodies provided herein may be used in therapeutic methods.

In one aspect, an anti-C3bBb antibody for use as a medicament is provided. In further aspects, an anti-C3bBb antibody for use in treating an ocular disease is provided. In one embodiment the ocular disease is selected from AMD (in one embodiment wet AMD, dry AMD, intermediate AMD, advanced AMD, and geographic atrophy (GA), macular degeneration, macular edema, DME (in one embodiment focal, non-center DME and diffuse, center-involved DME), retinopathy, diabetic retinopathy (DR) (in one embodiment proliferative DR (PDR), non-proliferative DR (NPDR), and high-altitude DR), other ischemia-related retinopathies, ROP, retinal vein occlusion (RVO) (in one embodiment central (CRVO) and branched (BRVO) forms), CNV (in one embodiment myopic CNV), corneal neovascularization, diseases associated with corneal neovascularization, retinal neovascularization, diseases associated with retinal/choroidal neovascularization, central serous retinopathy (CSR), pathologic myopia, von Hippel-Lindau disease, histoplasmosis of the eye, FEVR, Coats' disease, Norrie Disease, retinal abnormalities associated with osteoporosis-pseudoglioma syndrome (OPPG), subconjunctival hemorrhage, rubeosis, ocular neovascular disease, neovascular glaucoma, retinitis pigmentosa (RP), hypertensive retinopathy, retinal angiomatous proliferation, macular telangiectasia, iris neovascularization, intraocular neovascularization, retinal degeneration, cystoid macular edema (CME), vasculitis, papilloedema, retinitis, including but not limited to CMV retinitis, ocular melanoma, retinal blastoma, conjunctivitis (in one embodiment infectious conjunctivitis and non-infectious (in one embodiment allergic) conjunctivitis), Leber congenital amaurosis (also known as Leber's congenital amaurosis or LCA), uveitis (including infectious and non-infectious uveitis), choroiditis (in one embodiment multifocal choroiditis), ocular histoplasmosis, blepharitis, dry eye, traumatic eye injury, Sjögren's disease, and other ophthalmic diseases wherein the disease or disease is associated with ocular neovascularization, vascular leakage, and/or retinal edema or retinal atrophy. In one embodiment the ocular disease is selected from AMD (in one embodiment wet AMD, dry AMD, intermediate AMD, advanced AMD, and geographic atrophy (GA)), macular degeneration, macular edema, DME (in one embodiment focal, non-center DME and diffuse, center-involved DME), retinopathy, diabetic retinopathy (DR) (in one embodiment proliferative DR (PDR), non-proliferative DR (NPDR), and high-altitude DR. In one embodiment the ocular disease is from AMD, in one embodiment wet AMD.

In certain aspects, an anti-C3bBb antibody for use in a method of treatment is provided. In certain aspects, the invention provides an anti-C3bBb antibody for use in a method of treating an individual having an ocular disease comprising administering to the individual an effective amount of the anti-C3bBb antibody. In one such aspect, the method further comprises administering to the individual an effective amount of at least one additional therapeutic agent (e.g., one, two, three, four, five, or six additional therapeutic agents), e.g., as described below.

In a further aspect, the invention provides for the use of an anti-C3bBb antibody in the manufacture or preparation of a medicament. In one aspect, the medicament is for treatment of an ocular disease. In a further aspect, the medicament is for use in a method of treating an ocular disease comprising administering to an individual having an ocular disease an effective amount of the medicament. In one such aspect, the method further comprises administering to the individual an effective amount of at least one additional therapeutic agent, e.g., as described below. An “individual” according to any of the above aspects may be a human.

In a further aspect, the invention provides a method for treating an ocular disease. In one aspect, the method comprises administering to an individual having such ocular disease an effective amount of an anti-C3bBb antibody. In one such aspect, the method further comprises administering to the individual an effective amount of at least one additional therapeutic agent, as described below. An “individual” according to any of the above aspects may be a human.

In a further aspect, the invention provides pharmaceutical compositions comprising any of the anti-C3bBb antibodies provided herein, e.g., for use in any of the above therapeutic methods. In one aspect, a pharmaceutical composition comprises any of the anti-C3bBb antibodies provided herein and a pharmaceutically acceptable carrier. In another aspect, a pharmaceutical composition comprises any of the anti-C3bBb antibodies provided herein and at least one additional therapeutic agent, e.g., as described below.

Antibodies of the invention can be administered alone or used in a combination therapy. For instance, the combination therapy includes administering an antibody of the invention and administering at least one additional therapeutic agent (e.g. one, two, three, four, five, or six additional therapeutic agents). In certain aspects, the combination therapy comprises administering an antibody of the invention and administering at least one additional therapeutic agent, such as a VEGF-antagonist.

Any suitable AMD therapeutic agent can be administered as an additional therapeutic agent in combination with an antibody that binds to human C3bBb as provided herein for treatment of an ocular disorder (e.g., AMD, DME, DR, RVO, or GA), including, but not limited to, a VEGF antagonist, for example, an anti-VEGF antibody (e.g., LUCENTIS® (ranibizumab), RTH-258 (formerly ESBA-1008, an anti-VEGF single-chain antibody fragment; Novartis), or a bispecific anti-VEGF antibody (e.g., an anti-VEGF/anti-angiopoeitin 2 bispecific antibody such as faricimab or zifibancimig; Roche)), a soluble VEGF receptor fusion protein (e.g., EYLEA® (aflibercept)), an anti-VEGF DARPin® (e.g., abicipar pegol; Molecular Partners AG/Allergan), or an anti-VEGF aptamer (e.g., MACUGEN® (pegaptanib sodium)); a platelet-derived growth factor (PDGF) antagonist, for example, an anti-PDGF antibody, an anti-PDGFR antibody (e.g., REGN2176-3), an anti-PDGF-BB pegylated aptamer (e.g., FOVISTA®; Ophthotech/Novartis), a soluble PDGFR receptor fusion protein, or a dual PDGF/VEGF antagonist (e.g., a small molecule inhibitor (e.g., DE-120 (Santen) or X-82 (TyrogeneX)) or a bispecific anti-PDGF/anti-VEGF antibody)); VISUDYNE® (verteporfin) in combination with photodynamic therapy; an antioxidant; a complement system antagonist, for example, a complement factor C5 antagonist (e.g., a small molecule inhibitor (e.g., ARC-1905; Opthotech) or an anti-C5 antibody (e.g., LFG-316; Novartis), a properdin antagonist (e.g., an anti-properdin antibody, e.g., CLG-561; Alcon), or a complement factor D antagonist (e.g., an anti-complement factor D antibody, e.g., lampalizumab; Roche)); a C3 blocking peptide (e.g., APL-2, Appellis); a visual cycle modifier (e.g., emixustat hydrochloride); squalamine (e.g., OHR-102; Ohr Pharmaceutical); vitamin and mineral supplements (e.g., those described in the Age-Related Eye Disease Study 1 (AREDS1; zinc and/or antioxidants) and Study 2 (AREDS2; zinc, antioxidants, lutein, zeaxanthin, and/or omega-3 fatty acids)); a cell-based therapy, for example, NT-501 (Renexus); PH-05206388 (Pfizer), huCNS-SC cell transplantation (StemCells), CNTO-2476 (umbilical cord stem cell line; Janssen), OpRegen (suspension of RPE cells; Cell Cure Neurosciences), or MA09-hRPE cell transplantation (Ocata Therapeutics); a tissue factor antagonist (e.g., hI-con1; Iconic Therapeutics); an alpha-adrenergic receptor agonist (e.g., brimonidine tartrate; Allergan); a peptide vaccine (e.g., S-646240; Shionogi); an amyloid beta antagonist (e.g., an anti-beta amyloid monoclonal antibody, e.g., GSK-933776); an S1P antagonist (e.g., an anti-S1P antibody, e.g., iSONEP™; Lpath Inc); a ROBO4 antagonist (e.g., an anti-ROBO4 antibody, e.g., DS-7080a; Daiichi Sankyo); a lentiviral vector expressing endostatin and angiostatin (e.g., RetinoStat); and any combination thereof. In some instances, AMD therapeutic agents (including any of the preceding AMD therapeutic agents) can be co-formulated. For example, the anti-PDGFR antibody REGN2176-3 can be co-formulated with aflibercept (EYLEA®). In some instances, such a co-formulation can be administered in combination with an antibody that binds to human C3bBb of the invention. In some instances, the ocular disorder is AMD (e.g., wet AMD).

Any suitable DME and/or DR therapeutic agent can be administered in combination with an antibody that binds to human C3bBb of the invention for treatment of an ocular disorder (e.g., AMD, DME, DR, RVO, or GA), including, but not limited, to a VEGF antagonist (e.g., LUCENTIS® or EYLEA®), a corticosteroid (e.g., a corticosteroid implant (e.g., OZURDEX® (dexamethasone intravitreal implant) or ILUVIEN® (fluocinolone acetonide intravitreal implant)) or a corticosteroid formulated for administration by intravitreal injection (e.g., triamcinolone acetonide)), or combinations thereof. In some instances, the ocular disorder is DME and/or DR.

An antibody that binds to human C3bBb as provided herein may be administered in combination with a therapy or surgical procedure for treatment of an ocular disorder (e.g., AMD, DME, DR, RVO, or GA), including, for example, laser photocoagulation (e.g., panretinal photocoagulation (PRP)), drusen lasering, macular hole surgery, macular translocation surgery, implantable miniature telescopes, PHI-motion angiography (also known as micro-laser therapy and feeder vessel treatment), proton beam therapy, microstimulation therapy, retinal detachment and vitreous surgery, scleral buckle, submacular surgery, transpupillary thermotherapy, photosystem I therapy, use of RNA interference (RNAi), extracorporeal rheopheresis (also known as membrane differential filtration and rheotherapy), microchip implantation, stem cell therapy, gene replacement therapy, ribozyme gene therapy (including gene therapy for hypoxia response element, Oxford Biomedica; Lentipak, Genetix; and PDEF gene therapy, GenVec), photoreceptor/retinal cells transplantation (including transplantable retinal epithelial cells, Diacrin, Inc.; retinal cell transplant, e.g., Astellas Pharma US, Inc., ReNeuron, CHA Biotech), acupuncture, and combinations thereof.

Such combination therapies noted above encompass combined administration (where two or more therapeutic agents are included in the same or separate formulations), and separate administration, in which case, administration of the antibody that binds to human C3bBb of the invention can occur prior to, simultaneously, and/or following, administration of the additional therapeutic agent or agents. In one embodiment, administration of the antibody that binds to human C3bBb of the invention and administration of an additional therapeutic agent occur within about one, two, three, four, or five months, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. For repeated administrations over several days or longer, depending on the condition, the treatment would generally be sustained until a desired suppression of disease symptoms occurs. The progress of this therapy is easily monitored by conventional techniques and assays.

### 3. Specific Embodiments of the Invention

In the following specific embodiments of the invention are listed.
1. An antibody that specifically binds to human C3bBb.
2. An antibody that binds to human C3bBb, wherein the antibody:
   binds to wild-type human C3bBb; and/or
   binds to recombinant human C3bBb, which comprises the Bb subunit of factor B (FB) according to SEQ ID NO:491;
   binds to recombinant human C3bBb, which comprises the Bb subunit of factor B (FB) according to SEQ ID NO:492; and/or
   binds to recombinant human C3bBb, which comprises the Bb subunit of factor B (FB) according to SEQ ID NO:493; and/or
   binds to recombinant human C3bBb, which comprises the Bb subunit of a recombinant Factor B (FB) protein, which comprises a D279G mutation and optionally mutations K350N and/or a M458I;
   inhibits the alternative pathway; and/or
   is an agonist or antagonist of C3bBb activity; and/or
   specifically binds to cynomolgus C3bBb and human C3bBb, and/or
   specifically binds to African green monkey C3bBb and human C3bBb, and/or
   binds with an affinity of ≤300 pM at 37° C. as measured by SPR.
3. An antibody that specifically binds to recombinant human C3bBb comprising the Bb subunit of an FB protein with a D279G mutation and optionally further mutations K350N and/or M458I.
4. An antibody that specifically binds to recombinant human C3bBb which comprises the Bb subunit of factor B (FB) according to SEQ ID NO:491.
5. An antibody that specifically binds to recombinant human C3bBb which comprises the Bb subunit of factor B (FB) according to SEQ ID NO:492.
6. An antibody that specifically binds to recombinant human C3bBb which comprises the Bb subunit of factor B (FB) according to SEQ ID NO:493.
7. An antibody that specifically binds to an epitope of human C3bBb that includes amino acid residues of the C3b subunit and amino acid residues of the Bb subunit of human C3bBb.
8. An antibody that binds to human C3bBb that binds to an epitope comprising the following amino acid residues on C3bBb as detected by cryogenic electron microscopy:
   C3b subunit: Arg444, Lys534, Gly539, Ser540, Val524, Lys544, Gly546, Gln547, Ser548, Arg551, Gln557, Gln558, Thr560, Lys562, Glu564, Glu758, Pro759, Lys761, Asn762, Ile764, Leu768, Asn770, Asp797, and
   Bb subunit: Tyr465, Lys473, Ile474, Ser475, Ile477, Gly482, His483, Lys513, Val514, Ser515, Lys520, Arg521, Asp522, Glu610, and Lys613.
9. An antibody that binds to the same or an overlapping epitope as an antibody having a VH domain of SEQ ID NO: 39 and a VL domain of SEQ ID NO: 40 (P1AF8499).
10. An antibody that binds to human C3bBb, wherein the antibody comprises the heavy and light chain CDRs of an antibody selected from Table D1.
11. An antibody that binds to human C3bBb, wherein the antibody comprises the heavy and light chain variable domains of an antibody selected from Table D1.
12. The antibody of one of the preceding embodiments, comprising a set of six CDRs, in one embodiment a set of a VH and a VL domain, as an antibody selected from antibody #1 to antibody #217 from Table D1.
13. The antibody of one of the preceding embodiments, comprising a set of six CDRs, in one embodiment a set of a VH and a VL domain, as an antibody selected from antibody #1 to antibody #122 from Table D1.
14. The antibody of one of the preceding embodiments, comprising a set of six CDRs, in one embodiment a set of a VH and a VL domain, as an antibody selected from antibody #1 to antibody #7 and antibody #22 to antibody #217 from Table D1.
15. The antibody of one of the preceding embodiments, comprising a set of six CDRs, in one embodiment a set of a VH and a VL domain, as an antibody selected from antibody #1 to antibody #7 from Table D1.
16. The antibody of one of the preceding embodiments, comprising
   i) a heavy chain variable domain (VH) (a) CDR-H1 comprising the amino acid sequence of SEQ ID NO:449; (b) CDR-H2 comprising the amino acid sequence of SEQ ID NO:450; and (c) CDR-H3 comprising the amino acid sequence of SEQ ID NO:451, and a light chain variable domain (VL) comprising (d) CDR-L1 comprising the amino acid sequence of SEQ ID NO:452; (e) CDR-L2 comprising the amino acid sequence of SEQ ID NO:453; and (f) CDR-L3 comprising the amino acid sequence of SEQ ID NO:454 (corresponding to antibody #1, P1AG9426);
   ii) a heavy chain variable domain (VH) (a) CDR-H1 comprising the amino acid sequence of SEQ ID NO:455; (b) CDR-H2 comprising the amino acid sequence of SEQ ID NO:456; and (c) CDR-H3 comprising the amino acid sequence of SEQ ID NO:457, and a light chain variable domain (VL) comprising (d) CDR-L1 comprising the amino acid sequence of SEQ ID NO:458; (e) CDR-L2 comprising the amino acid sequence of SEQ ID NO:459; and (f) CDR-L3 comprising the amino acid sequence of SEQ ID NO:460 (corresponding to antibody #2, P1AG9376);
   iii) a heavy chain variable domain (VH) (a) CDR-H1 comprising the amino acid sequence of SEQ ID NO:461; (b) CDR-H2 comprising the amino acid sequence of SEQ ID NO:462; and (c) CDR-H3 comprising the amino acid sequence of SEQ ID NO:463, and a light chain variable domain (VL) comprising (d) CDR-L1 comprising the amino acid sequence of SEQ ID NO:464; (e) CDR-L2 comprising the amino acid sequence of SEQ ID NO:465; and (f) CDR-L3 comprising the amino acid sequence of SEQ ID NO:466 (corresponding to antibody #3, P1AG9372);
   iv) a heavy chain variable domain (VH) (a) CDR-H1 comprising the amino acid sequence of SEQ ID NO:467; (b) CDR-H2 comprising the amino acid sequence of SEQ ID NO:468; and (c) CDR-H3 comprising the amino acid sequence of SEQ ID NO:469, and a light chain variable domain (VL) comprising (d) CDR-L1 comprising the amino acid sequence of SEQ ID NO:470; (e) CDR-L2 comprising the amino acid sequence of SEQ ID NO:471; and (f) CDR-L3 comprising the amino acid sequence of SEQ ID NO:472 (corresponding to antibody #4, P1AG9420);
   v) a heavy chain variable domain (VH) (a) CDR-H1 comprising the amino acid sequence of SEQ ID NO:473; (b) CDR-H2 comprising the amino acid sequence of SEQ ID NO:474; and (c) CDR-H3 comprising the amino acid sequence of SEQ ID NO:475, and a light chain variable domain (VL) comprising (d) CDR-L1 comprising the amino acid sequence of SEQ ID NO:476; (e) CDR-L2 comprising the amino acid sequence of SEQ ID NO:477; and (f) CDR-L3 comprising the amino acid sequence of SEQ ID NO:478 (corresponding to antibody #5, P1AG9391);
   vi) a heavy chain variable domain (VH) (a) CDR-H1 comprising the amino acid sequence of SEQ ID NO:479; (b) CDR-H2 comprising the amino acid sequence of SEQ ID NO:480; and (c) CDR-H3 comprising the amino acid sequence of SEQ ID NO:481, and a light chain variable domain (VL) comprising (d) CDR-L1 comprising the amino acid sequence of SEQ ID NO:482; (e) CDR-L2 comprising the amino acid sequence of SEQ ID NO:483; and (f) CDR-L3 comprising the amino acid sequence of SEQ ID NO:484 (corresponding to antibody #6, P1AH1205); or
   vii) a heavy chain variable domain (VH) (a) CDR-H1 comprising the amino acid sequence of SEQ ID NO:485; (b) CDR-H2 comprising the amino acid sequence of SEQ ID NO:486; and (c) CDR-H3 comprising the amino acid sequence of SEQ ID NO:487, and a light chain variable domain (VL) comprising (d) CDR-L1 comprising the amino acid sequence of SEQ ID NO:488; (e) CDR-L2 comprising the amino acid sequence of SEQ ID NO:489; and (f) CDR-L3 comprising the amino acid sequence of SEQ ID NO:490 (corresponding to to antibody #7, P1AH1199).
17. The antibody of any of the preceding embodiments, comprising a sequence selected from the group consisting of
   (a) a VH sequence having at least 95% sequence identity to the amino acid sequence of SEQ ID NO:1;
   (b) a VL sequence having at least 95% sequence identity to the amino acid sequence of SEQ ID NO:2; and
   (c) a VH sequence as defined in (a) and a VL sequence as defined in (b).
18. The antibody of any of the preceding embodiments, comprising a sequence selected from the group consisting of
   (a) a VH sequence having at least 95% sequence identity to the amino acid sequence of SEQ ID NO:3;
   (b) a VL sequence having at least 95% sequence identity to the amino acid sequence of SEQ ID NO:4; and
   (c) a VH sequence as defined in (a) and a VL sequence as defined in (b).
19. The antibody of any of the preceding embodiments, comprising a sequence selected from the group consisting of
   (a) a VH sequence having at least 95% sequence identity to the amino acid sequence of SEQ ID NO:5;
   (b) a VL sequence having at least 95% sequence identity to the amino acid sequence of SEQ ID NO:6; and
   (c) a VH sequence as defined in (a) and a VL sequence as defined in (b).
20. The antibody of any of the preceding embodiments, comprising a sequence selected from the group consisting of
   (a) a VH sequence having at least 95% sequence identity to the amino acid sequence of SEQ ID NO:7;
   (b) a VL sequence having at least 95% sequence identity to the amino acid sequence of SEQ ID NO:8; and
   (c) a VH sequence as defined in (a) and a VL sequence as defined in (b).
21. The antibody of any of the preceding embodiments, comprising a sequence selected from the group consisting of
   (a) a VH sequence having at least 95% sequence identity to the amino acid sequence of SEQ ID NO:9;
   (b) a VL sequence having at least 95% sequence identity to the amino acid sequence of SEQ ID NO:10; and
   (c) a VH sequence as defined in (a) and a VL sequence as defined in (b).
22. The antibody of any of the preceding embodiments, comprising a sequence selected from the group consisting of
   (a) a VH sequence having at least 95% sequence identity to the amino acid sequence of SEQ ID NO:11;
   (b) a VL sequence having at least 95% sequence identity to the amino acid sequence of SEQ ID NO:12; and
   (c) a VH sequence as defined in (a) and a VL sequence as defined in (b).
23. The antibody of any of the preceding embodiments, comprising a sequence selected from the group consisting of
   (a) a VH sequence having at least 95% sequence identity to the amino acid sequence of SEQ ID NO:13;
   (b) a VL sequence having at least 95% sequence identity to the amino acid sequence of SEQ ID NO:14; and
   (c) a VH sequence as defined in (a) and a VL sequence as defined in (b).
24. The antibody of one of the preceding embodiments, comprising
   i) a VH domain comprising SEQ ID NO:1 and a VL domain comprising SEQ ID NO:2 (corresponding to antibody #1, P1AG9426);
   ii) a VH domain comprising SEQ ID NO:3 and a VL domain comprising SEQ ID NO:4 (corresponding to antibody #2, P1AG9376);
   iii) a VH domain comprising SEQ ID NO:5 and a VL domain comprising SEQ ID NO:6 (corresponding to antibody #3, P1AG9372);
   iv) a VH domain comprising SEQ ID NO:7 and a VL domain comprising SEQ ID NO:8 (corresponding to antibody #4, P1AG9420);
   v) a VH domain comprising SEQ ID NO:9 and a VL domain comprising SEQ ID NO:10 (corresponding to antibody #5, P1AG9391);
   vi) a VH domain comprising SEQ ID NO:11 and a VL domain comprising SEQ ID NO:12 (corresponding to antibody #6, P1AH1205); or
   vii) a VH domain comprising SEQ ID NO:13 and a VL domain comprising SEQ ID NO:14 (corresponding to to antibody #7, P1AH1199).
25. The antibody of any of the preceding embodiments comprising a heavy chain of SEQ ID NO:435 and a light chain of SEQ ID NO:436.
26. The antibody of any of the preceding embodiments comprising a heavy chain of SEQ ID NO:437 and a light chain of SEQ ID NO:438.
27. The antibody of any of the preceding embodiments comprising a heavy chain of SEQ ID NO:439 and a light chain of SEQ ID NO:440.
28. The antibody of any of the preceding embodiments comprising a heavy chain of SEQ ID NO:441 and a light chain of SEQ ID NO:442.
29. The antibody of any of the preceding embodiments comprising a heavy chain of SEQ ID NO:443 and a light chain of SEQ ID NO:444.
30. The antibody of any of the preceding embodiments comprising a heavy chain of SEQ ID NO:445 and a light chain of SEQ ID NO:446.
31. The antibody of any of the preceding embodiments comprising a heavy chain of SEQ ID NO:447 and a light chain of SEQ ID NO:447.
32. The antibody of any one of the preceding embodiments, which is a monoclonal antibody.
33. The antibody of any one of the preceding embodiments, which is a human antibody.
34. The antibody of any one of the preceding embodiments, wherein the antibody comprises a VH domain with a VH3 framework and a VL domain with a Vkappa1 framework.
35. The antibody of any one of the preceding embodiments, wherein the antibody comprises human constant regions of IgG isotype.
36. The antibody of any of the preceding embodiments, which is an antibody fragment that binds human C3bBb.
37. The antibody of any of the preceding embodiments, which is a full length IgG1 antibody.
38. The antibody of any of the preceding embodiments, wherein the antibody binds human C3bBb with an affinity of ≤300 pM as measured by SPR.
39. The antibody of any of the preceding embodiments, wherein the antibody is a multispecific antibody.
40. An antibody, which competes for binding to human C3bBb with the antibody of any one of the preceding embodiments.
41. An isolated nucleic acid encoding the antibody of any of the preceding embodiments.
42. A host cell comprising the nucleic acid of embodiment 42.
43. A method of producing an antibody that binds to human C3bBb comprising culturing the host cell of embodiment 42 under conditions suitable for the expression of the antibody.
44. The method of embodiment 43, further comprising recovering the antibody from the host cell.
45. An antibody produced by the method of embodiment 43.
46. A pharmaceutical composition comprising the antibody of any of embodiments 1 to 40 and a pharmaceutically acceptable carrier.
47. The pharmaceutical composition of embodiment 46, further comprising an additional therapeutic agent.
48. The antibody of any one of embodiments 1 to 40 or the pharmaceutical composition of any or embodiments 46 to 47 for use as a medicament.
49. The antibody of any one of embodiments 1 to 40 or the pharmaceutical composition of any of embodiments 46 to 47 for use in treating an ocular disease.
50. Use of the antibody of any one of embodiments 1 to 40 or the pharmaceutical composition of any of embodiments 46 to 47 in the manufacture of a medicament for treatment of an ocular disease.
51. A method of treating an individual having an ocular disease comprising administering to the individual an effective amount of the antibody of any one of embodiments 1 to 40 or the pharmaceutical composition of any of embodiments 46 to 47.

DESCRIPTION OF THE AMINO ACID SEQUENCES

| | # | identifier | domain | sequence |
|---|---|---|---|---|
| SEQ ID NO: 01 | 1 | P1AG9426 | VH | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 02 | 1 | P1AG9426 | VL | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 03 | 2 | P1AG9376 | VH | KVQLVESGGGLVQPGGSLRLSCAASGFK |
| | | | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 04 | 2 | P1AG9376 | VL | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 05 | 3 | P1AG9372 | VH | EVQLVESGGGLVQPGGSLRLSCAASGFRF |
| | | | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 06 | 3 | P1AG9372 | VL | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 07 | 4 | P1AG9420 | VH | RVQLVESGGGLVQPGGSLRLSCAASGFRY |
| | | | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 08 | 4 | P1AG9420 | VL | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 09 | 5 | P1AG9391 | VH | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 10 | 5 | P1AG9391 | VL | AIQMTQSPSSLSASVGDRVTITCQASYWLS |
| | | | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 11 | 6 | P1AH1205 | VH | KVQLVESGGGLVQPGGSLRLSCAASGFK |
| | | | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 12 | 6 | P1AH1205 | VL | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 13 | 7 | P1AH1199 | VH | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 14 | 7 | P1AH1199 | VL | AIQMTQSPSSLSASVGDRVTITCQASYWLS |
| | | | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 15 | 8 | C3bBb.P045.072 | VH | KLQLVESGGGLVKPGGSLRLSCAASGRRF |
| | | | | GVqDMSWVRQAPGKGLEWVGSISPKGDH |
| | | | | KYLNTKFIGRFTISRDNSKNTLYLQMNSLR |
| | | | | AEDTAVYYCARDWGYFDQWGQGTLVTV |
| | | | | SS |
| SEQ ID NO: 16 | 8 | C3bBb.P045.072 | VL | AIQMTQSPSSLSASVGDRVTITCHGSYWLS |
| | | | | SEVAWYQQKPGKAPKLLIYDGNGIIGNVP |
| | | | | SRFSGSGSHEDYTLTISSLQPEDFATYYCQ |
| | | | | QYRYHPYTFGHGTKVEIK |
| SEQ ID NO: 17 | 9 | C3bBb.P045.236 | VH | YGQLVESGGGLVKPGGSLRLSCAATGTLF |
| | | | | SKqDMSWVRQAPGKGLEWVGSISPKGDH |
| | | | | KYLNTKFIGRFTISRDNSKNTLYLQMNSLR |
| | | | | AEDTAVYYCAKDWGYFDHWGQGTLVTV |
| | | | | SS |
| SEQ ID NO: 18 | 9 | C3bBb.P045.236 | VL | AIQMTQSPSSLSASVGDRVTITCHGSYWLS |
| | | | | SEVAWYQQKPGKAPKLLIYDGNGVQGSV |
| | | | | PSRFSGSGSHEDYTLTISSLQQEDFATYYC |
| | | | | QQYRYHPYTFGHGTKVEIK |
| SEQ ID NO: 19 | 10 | C3bBb.P045.225 | VH | DPQLVESGGGLVKPGGSLRLSCAARGLHF |
| | | | | NNTDMSWVRQAPGKGLEWVGSISPKGDH |
| | | | | KYLNTKFIGRFTISRDNSKNTLYLQMNSLR |
| | | | | AEDTAVYYCARDWGYFDWWGQGTLVT |
| | | | | VSS |
| SEQ ID NO: 20 | 10 | C3bBb.P045.225 | VL | AIQMTQSPSSLSASVGDRVTITCHGSYWLS |
| | | | | SEVAWYQQKPGKAPKLLIYDGNGLKGGV |
| | | | | PSRFSGSGSHEDYTLTISSLQPEDFATYYC |
| | | | | QQYRYHPYTFGHGTKVEIK |
| SEQ ID NO: 21 | 11 | C3bBb.P048.152 | VH | GQLVESGGGLVKPGGSLRLSCKASGFYW |
| | | | | GPGAMSWVRQAPGKGLEWVGSISPKSGS |
| | | | | KGYAQNVPGRFTISRDNNQDTLYLQMNS |
| | | | | LRAEDTAVYYCARDIGFFDTWGQGTLVT |
| | | | | VSS |
| SEQ ID NO: 22 | 11 | C3bBb.P048.152 | VL | HDIQMTQSPSSLSASVGDRVTITCDADFGI |
| | | | | SNYLAWYQQKPGKAPKLLIFDAKYRAPE |
| | | | | VPSRFSGSGSGGSFTLTISSLQPEDFATYYC |
| | | | | QQYGVHPFTFGQGTKVEIK |
| SEQ ID NO: 23 | 12 | C3bBb.P048.061 | VH | GQLVESGGGLVKPGGSLRLSCKASGFYW |
| | | | | GPGAMSWVRQAPGKGLEWVGSISPKGGS |
| | | | | TDINPKIGGRFTISRDNNQDTLYLQMNSLR |
| | | | | AEDTAVYYCARDIGFFDTWGQGTLVTVS |
| | | | | S |
| SEQ ID NO: 24 | 12 | C3bBb.P048.061 | VL | AIQMTQSPSSLSASVGDRVTITCQGSWLIS |
| | | | | NYLAWYQQKPGKAPKLLIFDAKYRAPEV |
| | | | | PSRFSGSGSGTDFTLTISSLQPEDFATYYCQ |
| | | | | QYGGRLLTFGQGTKVEIK |
| SEQ ID NO: 25 | 13 | C3bBb.P048.127 | VH | GQLVESGGGLVKPGGSLRLSCKASGFYW |
| | | | | GPGAMSWVRQAPGKGLEWVGSISPKGGS |
| | | | | TEYNTKVYGRFTISRDNNQDTLYLQMNSL |
| | | | | RAEDTAVYYCARDIGFFDTWGQGTLVTV |
| | | | | SS |
| SEQ ID NO: 26 | 13 | C3bBb.P048.127 | VL | HQMTQSPSSLSASVGDRVTITCDGEWDIS |
| | | | | NYLAWYQQKPGKAPKLLIFDAKYRAPEV |
| | | | | PSRFSGSGSGRDFTLTISSLQPEDFATYYC |
| | | | | QQYNRMYFTFGQGTKVEIK |
| SEQ ID NO: 27 | 14 | C3bBb.P048.241 | VH | GQLVESGGGLVKPGGSLRLSCKASGFYW |
| | | | | GPGAMSWVRQAPGKGLEWVGSISPKGGS |
| | | | | AWYNNNFTGRFTISRDNNQDTLYLQMNS |
| | | | | LRAEDTAVYYCARDIGFFDTWGQGTLVT |
| | | | | VSS |
| SEQ ID NO: 28 | 14 | C3bBb.P048.241 | VL | HINQMTQSPSSLSASVGDRVTITCQAKWV |
| | | | | ISNYLAWYQQKPGKAPKLLIFDAKYRAPE |
| | | | | VPSRFSGSGSDGTFTLTISSLQPEDFATYYC |
| | | | | QQYDWWPYTFGQGTKVEIK |
| SEQ ID NO: 29 | 15 | C3bBb.P048.230 | VH | GQLVESGGGLVKPGGSLRLSCKASGFYW |
| | | | | GPGAMSWVRQAPGKGLEWVGSISPKGGS |
| | | | | AWYNNKFHGRFTISRDNNQDTLYLQMNS |
| | | | | LRAEDTAVYYCARDIGFFDTWGQGTLVT |
| | | | | VSS |
| SEQ ID NO: 30 | 15 | C3bBb.P048.230 | VL | HYIQMTQSPSSLSASVGDRVTITCQGNAFI |
| | | | | SNYLAWYQQKPGKAPKLLIFDAKYRAPE |
| | | | | VPSRFSGSGSGDDFTLTISSLQPEDFATYY |
| | | | | CQQYDWWPYTFGQGTKVEIK |
| SEQ ID NO: 31 | 16 | C3bBb.P048.204 | VH | GQLVESGGGLVKPGGSLRLSCKASGFYW |
| | | | | GPGAMSWVRQAPGKGLEWVGSISPKGGS |
| | | | | AWFNRNFAGRFTISRDNNQDTLYLQMNS |
| | | | | LRAEDTAVYYCARDIGFFDTWGQGTLVT |
| | | | | VSS |
| SEQ ID NO: 32 | 16 | C3bBb.P048.204 | VL | GHVQMTQSPSSLSASVGDRVTITCQAEDEI |
| | | | | SNYLAWYQQKPGKAPKLLIFDAKYRAPE |
| | | | | VPSRFSGSGSDASFTLTISSLQPEDFATYYC |
| | | | | QQYDWWPYTFGQGTKVEIK |
| SEQ ID NO: 33 | 17 | C3bBb.P048.333 | VH | GQLVESGGGLVKPGGSLRLSCKASGFYW |
| | | | | GPGAMSWVRQAPGKGLEWVGSISPKGGS |
| | | | | AWYNDKFKGRFTISRDNNQDTLYLQMNS |
| | | | | LRAEDTAVYYCARDIGFFDTWGQGTLVT |
| | | | | VSS |
| SEQ ID NO: 34 | 17 | C3bBb.P048.333 | VL | HINQMTQSPSSLSASVGDRVTITCNSTEFIS |
| | | | | NYLAWYQQKPGKAPKLLIFDAKYRAPEV |
| | | | | PSRFSGSGSDQEFTLTISSLQPEDFATYYCQ |
| | | | | QYDWWPYTFGQGTKVEIK |
| SEQ ID NO: 35 | 18 | C3bBb.P053.095 | VH | RKQLVESGGGLVQPGGSLRLSCAAHGSLV |
| | | | | SRNDMSWVRQAPGKGLEWVGSISPKGDH |
| | | | | KYYNTKFIDRFTISRDNSKNTLYLQMNSL |
| | | | | RAEDTAVYYCAKDVDYMDLWGQGTLVT |
| | | | | VSS |
| SEQ ID NO: 36 | 18 | C3bBb.P053.095 | VL | AIQMTQSPSSLSASVGDRVTITCQASYWL |
| | | | | NSELAWYQQKPGKAPKLLIFDGDQTWFG |
| | | | | VPSRFSGSGSHEDYTLTISSLQPEDFATYY |
| | | | | CQQYRYTPYTFGQGTKLEIK |
| SEQ ID NO: 37 | 19 | C3bBb.P055.015 | VH | GQLVESGGGLVKPGGSLRLSCKASGFYW |
| | | | | GPGAMSWVRQAPGKGLEWVGSISPKGGS |
| | | | | AEYASKVYGRFTISRDNNQDTLYLQMNSL |
| | | | | RAEDTAVYYCARDIGFFDTWGQGTLVTV |
| | | | | SS |
| SEQ ID NO: 38 | 19 | C3bBb.P055.015 | VL | DYQMTQSPSSLSASVGDRVTITCEAHSQIS |
| | | | | NYLAWYQQKPGKAPKLLIFDAKYRAPEV |
| | | | | PSRFSGSGSGPDFTLTISSLQPEDFATYYCQ |
| | | | | QYNDFWLTFGQGTKVEIK |
| SEQ ID NO: 39 | 20 | P1AF8499 | VH | EVQLVESGGGLVQPGGSLRLSCAASGFSW |
| | | | | EHYAMTWVRQAPGKGLEWVGSISPKGDH |
| | | | | KYYNTKFIDRFTISRDNSKNTLYLQMNSL |
| | | | | RAEDTAVYYCARDWDYFDEWGQGTLVT |
| | | | | VSS |
| SEQ ID NO: 40 | 20 | P1AF8499 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 41 | 21 | GQ-5_1 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 42 | 21 | GQ-5_1 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 43 | 22 | GQ-11_1 | VH | EVQLVESGGGLVQPGGSLRLSCAASGFSW |
| | | | | EHYAMTWVRQAPGKGLEWVGSISPKGDH |
| | | | | KYYNTKFIDRFTISRDNSKNTLYLQMNSL |
| | | | | RAEDTAVYYCARDWDYFDEWGQGTLVT |
| | | | | VSS |
| SEQ ID NO: 44 | 22 | GQ-11_1 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 45 | 23 | PT5622.F19 | VH | EVQLVESGGGLVQPGGSLRLSCAASGFRF |
| | | | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 46 | 23 | PT5622.F19 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 47 | 24 | PT5624.P06 | VH | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 48 | 24 | PT5624.P06 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 49 | 25 | PT5624.M09 | VH | KVQLVESGGGLVQPGGSLRLSCAASGFR |
| | | | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 50 | 25 | PT5624.M09 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 51 | 26 | PT5626.M10 | VH | RVQLVESGGGLVQPGGSLRLSCAASGFRY |
| | | | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 52 | 26 | PT5626.M10 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 53 | 27 | PT5626.C10 | VH | RVQLVESGGGLVQPGGSLRLSCAASGFT |
| | | | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 54 | 27 | PT5626.C10 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 55 | 28 | PT5628.E16 | VH | RVQLVESGGGLVQPGGSLRLSCAASGFTY |
| | | | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 56 | 28 | PT5628.E16 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 57 | 29 | PT5630.D05 | VH | KVQLVESGGGLVQPGGSLRLSCAASGYR |
| | | | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 58 | 29 | PT5630.D05 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 59 | 30 | PT5630.A20 | VH | KVQLVESGGGLVQPGGSLRLSCAASGFK |
| | | | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 60 | 30 | PT5630.A20 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 61 | 31 | PT5632.A13 | VH | RVQLVESGGGLVQPGGSLRLSCAASGYR |
| | | | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 62 | 31 | PT5632.A13 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 63 | 32 | PT5634.N13 | VH | KVQLVESGGGLVQPGGSLRLSCAASGFTY |
| | | | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 64 | 32 | PT5634.N13 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 65 | 33 | PT5634.M24 | VH | EVQLVESGGGLVQPGGSLRLSCAASGFQ |
| | | | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 66 | 33 | PT5634.M24 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 67 | 34 | PT5636.P22 | VH | RVQLVESGGGLVQPGGSLRLSCAASGFKF |
| | | | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 68 | 34 | PT5636.P22 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 69 | 35 | PT5625.G07 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 70 | 35 | PT5625.G07 | VL | AIQMTQSPSSLSASVGDRVTITCQAQYWL |
| | | | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 71 | 36 | PT5627.M20 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 72 | 36 | PT5627.M20 | VL | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 73 | 37 | PT5627.M12 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 74 | 37 | PT5627.M12 | VL | AIQMTQSPSSLSASVGDRVTITCQASYWLS |
| | | | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 75 | 38 | PT5629.F09 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 76 | 38 | PT5629.F09 | VL | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 77 | 39 | PT5629.H08 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 78 | 39 | PT5629.H08 | VL | AIQMTQSPSSLSASVGDRVTITCQASYWLS |
| | | | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 79 | 40 | PT5631.D21 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 80 | 40 | PT5631.D21 | VL | AIQMTQSPSSLSASVGDRVTITCQSSHWLS |
| | | | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 81 | 41 | PT5633.I03 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 82 | 41 | PT5633.I03 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 83 | 42 | PT5635.C08 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 84 | 42 | PT5635.C08 | VL | AIQMTQSPSSLSASVGDRVTITCQASYWLS |
| | | | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 85 | 43 | PT5639.G15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 86 | 43 | PT5639.G15 | VL | AIQMTQSPSSLSASVGDRVTITCQSTHWLS |
| | | | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 87 | 44 | PT5622.A20 | VH | EVQLVESGGGLVQPGGSLRLSCAASGFR |
| | | | | WDKYAMSWVRQAPGKGLEWVGEISPRG |
| | | | | DSKYYNTKFIDRFTISRDDEKNTLYLQMN |
| | | | | SLRAEDTAVYYCARDWEYFDNWGQGTL |
| | | | | VTVSS |
| SEQ ID NO: 88 | 44 | PT5622.A20 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 89 | 45 | PT5622.C12 | VH | RVQLVESGGGLVQPGGSLRLSCAASGFTY |
| | | | | EHYAMSWVRQAPGKGLEWVGSISPRGDH |
| | | | | KYYNTKFIDRFTISRDNSKNTLYLQMNSL |
| | | | | RAEDTAVYYCARDWDYFDTWGQGTLVT |
| | | | | VSS |
| SEQ ID NO: 90 | 45 | PT5622.C12 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 91 | 46 | PT5622.G24 | VH | GVQLVESGGGLVQPGGSLRLSCAASGFRY |
| | | | | DIYAMSWVRQAPGKGLEWVGEISPEGNH |
| | | | | KYYNTKFIDRFTISRDNEKNTLYLQMNSL |
| | | | | RAEDTAVYYCARDWEYFDTWGQGTLVT |
| | | | | VSS |
| SEQ ID NO: 92 | 46 | PT5622.G24 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 93 | 47 | PT5622.I20 | VH | KVQLVESGGGLVQPGGSLRLSCAASGYT |
| | | | | YDIYAMSWVRQAPGKGLEWVGEISPDGN |
| | | | | TKYYNTKFIDRFTISRDNSKNTLYLQMNS |
| | | | | LRAEDTAVYYCARDWEYFDHWGQGTLV |
| | | | | TVSS |
| SEQ ID NO: 94 | 47 | PT5622.I20 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 95 | 48 | PT5622.J24 | VH | KVQLVESGGGLVQPGGSLRLSCAASGYR |
| | | | | YEHYAMSWVRQAPGKGLEWVGSISPKGQ |
| | | | | HKYYNTKFIDRFTISRDNEKNTLYLQMNS |
| | | | | LRAEDTAVYYCARDWEYFDVWGQGTLV |
| | | | | TVSS |
| SEQ ID NO: 96 | 48 | PT5622.J24 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 97 | 49 | PT5624.A21 | VH | KVQLVESGGGLVQPGGSLRLSCAASGFRF |
| | | | | DIYAMSWVRQAPGKGLEWVGEISPKGEH |
| | | | | KYYNTKFIDRFTISRDNSKNTLYLQMNSL |
| | | | | RAEDTAVYYCARDWEYFDGWGQGTLVT |
| | | | | VSS |
| SEQ ID NO: 98 | 49 | PT5624.A21 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 99 | 50 | PT5624.B10 | VH | KVQLVESGGGLVQPGGSLRLSCAASGFHY |
| | | | | DIYAMSWVRQAPGKGLEWVGEISPDGRH |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 100 | 50 | PT5624.B10 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 101 | 51 | PT5624.N05 | VH | GVQLVESGGGLVQPGGSLRLSCAASGFKY |
| | | | | EHYAMSWVRQAPGKGLEWVGEISPKGDH |
| | | | | KYYNTKFIDRFTISRDNEKNTLYLQMNSL |
| | | | | RAEDTAVYYCARDWEYFDYWGQGTLVT |
| | | | | VSS |
| SEQ ID NO: 102 | 51 | PT5624.N05 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 103 | 52 | PT5624.P11 | VH | KVQLVESGGGLVQPGGSLRLSCAASGFR |
| | | | | WEHYAMSWVRQAPGKGLEWVGSISPRG |
| | | | | DHKYYNTKFIDRFTISRDNSKNTLYLQMN |
| | | | | SLRAEDTAVYYCARDWDYFDQWGQGTL |
| | | | | VTVSS |
| SEQ ID NO: 104 | 52 | PT5624.P11 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 105 | 53 | PT5626.A12 | VH | RVQLVESGGGLVQPGGSLRLSCAASGFK |
| | | | | WDHYAMSWVRQAPGKGLEWVGSISPDG |
| | | | | NHKYYNTKFIDRFTISRDDSKNTLYLQMN |
| | | | | SLRAEDTAVYYCARDWDYFDYWGQGTL |
| | | | | VTVSS |
| SEQ ID NO: 106 | 53 | PT5626.A12 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 107 | 54 | PT5626.N08 | VH | KVQLVESGGGLVQPGGSLRLSCAASGYK |
| | | | | YDHYAMSWVRQAPGKGLEWVGEISPEGN |
| | | | | SKYYNTKFIDRFTISRDNSKNTLYLQMNSL |
| | | | | RAEDTAVYYCARDWEYFDNWGQGTLVT |
| | | | | VSS |
| SEQ ID NO: 108 | 54 | PT5626.N08 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 109 | 55 | PT5626.N18 | VH | KVQLVESGGGLVQPGGSLRLSCAASGFKY |
| | | | | EVYAMSWVRQAPGKGLEWVGEISPKGDT |
| | | | | KYYNTKFIDRFTISRDNDKNTLYLQMNSL |
| | | | | RAEDTAVYYCARDWEYFDNWGQGTLVT |
| | | | | VSS |
| SEQ ID NO: 110 | 55 | PT5626.N18 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 111 | 56 | PT5626.009 | VH | RVQLVESGGGLVQPGGSLRLSCAASGFK |
| | | | | WEIYAMSWVRQAPGKGLEWVGDISPDGQ |
| | | | | TKYYNTKFIDRFTISRDDEKNTLYLQMNS |
| | | | | LRAEDTAVYYCARDWEYFDHWGQGTLV |
| | | | | TVSS |
| SEQ ID NO: 112 | 56 | PT5626.009 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 113 | 57 | PT5628.B13 | VH | QVQLVESGGGLVQPGGSLRLSCAASGFT |
| | | | | WDHYAMSWVRQAPGKGLEWVGEISPRG |
| | | | | DTKYYNTKFIDRFTISRDNEKNTLYLQMN |
| | | | | SLRAEDTAVYYCARDWDYFDNWGQGTL |
| | | | | VTVSS |
| SEQ ID NO: 114 | 57 | PT5628.B13 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 115 | 58 | PT5628.C21 | VH | RVQLVESGGGLVQPGGSLRLSCAASGFK |
| | | | | WEHYAMSWVRQAPGKGLEWVGSISPRG |
| | | | | DHKYYNTKFIDRFTISRDDDKNTLYLQMN |
| | | | | SLRAEDTAVYYCARDWDYFDSWGQGTL |
| | | | | VTVSS |
| SEQ ID NO: 116 | 58 | PT5628.C21 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 117 | 59 | PT5628.E12 | VH | KVQLVESGGGLVQPGGSLRLSCAASGYR |
| | | | | WDIYAMSWVRQAPGKGLEWVGSISPDGN |
| | | | | HKYYNTKFIDRFTISRDDSKNTLYLQMNS |
| | | | | LRAEDTAVYYCARDWDYFDTWGQGTLV |
| | | | | TVSS |
| SEQ ID NO: 118 | 59 | PT5628.E12 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 119 | 60 | PT5628.I07 | VH | KVQLVESGGGLVQPGGSLRLSCAASGFRY |
| | | | | DYYAMSWVRQAPGKGLEWVGSISPRGDH |
| | | | | KYYNTKFIDRFTISRDNDKNTLYLQMNSL |
| | | | | RAEDTAVYYCARDWDYFDYWGQGTLVT |
| | | | | VSS |
| SEQ ID NO: 120 | 60 | PT5628.I07 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 121 | 61 | PT5628.P14 | VH | KVQLVESGGGLVQPGGSLRLSCAASGFT |
| | | | | WEHYAMSWVRQAPGKGLEWVGSISPDG |
| | | | | QHKYYNTKFIDRFTISRDDSKNTLYLQMN |
| | | | | SLRAEDTAVYYCARDWDYFDYWGQGTL |
| | | | | VTVSS |
| SEQ ID NO: 122 | 61 | PT5628.P14 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 123 | 62 | PT5630.G05 | VH | QVQLVESGGGLVQPGGSLRLSCAASGYR |
| | | | | WDHYAMSWVRQAPGKGLEWVGSISPDG |
| | | | | RHKYYNTKFIDRFTISRDNSKNTLYLQMN |
| | | | | SLRAEDTAVYYCARDWDYFDRWGQGTL |
| | | | | VTVSS |
| SEQ ID NO: 124 | 62 | PT5630.G05 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 125 | 63 | PT5630.K04 | VH | RVQLVESGGGLVQPGGSLRLSCAASGFRF |
| | | | | DIYAMSWVRQAPGKGLEWVGEISPDGRH |
| | | | | KYYNTKFIDRFTISRDDEKNTLYLQMNSL |
| | | | | RAEDTAVYYCARDWEYFDEWGQGTLVT |
| | | | | VSS |
| SEQ ID NO: 126 | 63 | PT5630.K04 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 127 | 64 | PT5630.M11 | VH | RVQLVESGGGLVQPGGSLRLSCAASGFK |
| | | | | WEHYAMSWVRQAPGKGLEWVGEISPDG |
| | | | | RTKYYNTKFIDRFTISRDDSKNTLYLQMN |
| | | | | SLRAEDTAVYYCARDWDYFDHWGQGTL |
| | | | | VTVSS |
| SEQ ID NO: 128 | 64 | PT5630.M11 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 129 | 65 | PT5630.N06 | VH | RVQLVESGGGLVQPGGSLRLSCAASGFTY |
| | | | | DHYAMSWVRQAPGKGLEWVGEISPKGDT |
| | | | | KYYNTKFIDRFTISRDDDKNTLYLQMNSL |
| | | | | RAEDTAVYYCARDWDYFDYWGQGTLVT |
| | | | | VSS |
| SEQ ID NO: 130 | 65 | PT5630.N06 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 131 | 66 | PT5630.O08 | VH | KVQLVESGGGLVQPGGSLRLSCAASGFT |
| | | | | WDHYAMSWVRQAPGKGLEWVGSISPRG |
| | | | | DHKYYNTKFIDRFTISRDDSKNTLYLQMN |
| | | | | SLRAEDTAVYYCARDWDYFDSWGQGTL |
| | | | | VTVSS |
| SEQ ID NO: 132 | 66 | PT5630.O08 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 133 | 67 | PT5632.A10 | VH | RVQLVESGGGLVQPGGSLRLSCAASGFHF |
| | | | | DKYAMSWVRQAPGKGLEWVGSISPKGRT |
| | | | | KYYNTKFIDRFTISRDNSKNTLYLQMNSL |
| | | | | RAEDTAVYYCARDWDYFDNWGQGTLVT |
| | | | | VSS |
| SEQ ID NO: 134 | 67 | PT5632.A10 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 135 | 68 | PT5632.A15 | VH | RVQLVESGGGLVQPGGSLRLSCAASGFR |
| | | | | WDYYAMSWVRQAPGKGLEWVGSISPDG |
| | | | | QHKYYNTKFIDRFTISRDDDKNTLYLQMN |
| | | | | SLRAEDTAVYYCARDWDYFDHWGQGTL |
| | | | | VTVSS |
| SEQ ID NO: 136 | 68 | PT5632.A15 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 137 | 69 | PT5632.B15 | VH | KVQLVESGGGLVQPGGSLRLSCAASGYR |
| | | | | WDHYAMSWVRQAPGKGLEWVGEISPDG |
| | | | | QNKYYNTKFIDRFTISRDNDKNTLYLQMN |
| | | | | SLRAEDTAVYYCARDWEYFDNWGQGTL |
| | | | | VTVSS |
| SEQ ID NO: 138 | 69 | PT5632.B15 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 139 | 70 | PT5632.D11 | VH | KVQLVESGGGLVQPGGSLRLSCAASGFK |
| | | | | WEHYAMSWVRQAPGKGLEWVGSISPEG |
| | | | | NHKYYNTKFIDRFTISRDNSKNTLYLQMN |
| | | | | SLRAEDTAVYYCARDWDYFDRWGQGTL |
| | | | | VTVSS |
| SEQ ID NO: 140 | 70 | PT5632.D11 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 141 | 71 | PT5632.J23 | VH | GVQLVESGGGLVQPGGSLRLSCAASGFK |
| | | | | WEHYAMSWVRQAPGKGLEWVGEISPDG |
| | | | | RHKYYNTKFIDRFTISRDDSKNTLYLQMN |
| | | | | SLRAEDTAVYYCARDWEYFDYWGQGTL |
| | | | | VTVSS |
| SEQ ID NO: 142 | 71 | PT5632.J23 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 143 | 72 | PT5634.A17 | VH | RVQLVESGGGLVQPGGSLRLSCAASGYR |
| | | | | WDHYAMSWVRQAPGKGLEWVGSISPDG |
| | | | | RTKYYNTKFIDRFTISRDDEKNTLYLQMN |
| | | | | SLRAEDTAVYYCARDWEYFDTWGQGTL |
| | | | | VTVSS |
| SEQ ID NO: 144 | 72 | PT5634.A17 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 145 | 73 | PT5634.A21 | VH | RVQLVESGGGLVQPGGSLRLSCAASGFKF |
| | | | | DIYAMSWVRQAPGKGLEWVGEISPDGQS |
| | | | | KYYNTKFIDRFTISRDDSKNTLYLQMNSL |
| | | | | RAEDTAVYYCARDWDYFDNWGQGTLVT |
| | | | | VSS |
| SEQ ID NO: 146 | 73 | PT5634.A21 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 147 | 74 | PT5634.K10 | VH | KVQLVESGGGLVQPGGSLRLSCAASGFK |
| | | | | WEHYAMSWVRQAPGKGLEWVGEISPKG |
| | | | | DNKYYNTKFIDRFTISRDDEKNTLYLQMN |
| | | | | SLRAEDTAVYYCARDWEYFDNWGQGTL |
| | | | | VTVSS |
| SEQ ID NO: 148 | 74 | PT5634.K10 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 149 | 75 | PT5634.M16 | VH | RVQLVESGGGLVQPGGSLRLSCAASGYR |
| | | | | WDHYAMSWVRQAPGKGLEWVGEISPKG |
| | | | | DSKYYNTKFIDRFTISRDDEKNTLYLQMN |
| | | | | SLRAEDTAVYYCARDWEYFDNWGQGTL |
| | | | | VTVSS |
| SEQ ID NO: 150 | 75 | PT5634.M16 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 151 | 76 | PT5634.N11 | VH | KVQLVESGGGLVQPGGSLRLSCAASGFR |
| | | | | WEHYAMSWVRQAPGKGLEWVGEISPDG |
| | | | | QDKYYNTKFIDRFTISRDDSKNTLYLQMN |
| | | | | SLRAEDTAVYYCARDWEYFDTWGQGTL |
| | | | | VTVSS |
| SEQ ID NO: 152 | 76 | PT5634.N11 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 153 | 77 | PT5634.P19 | VH | KVQLVESGGGLVQPGGSLRLSCAASGFT |
| | | | | WDHYAMSWVRQAPGKGLEWVGSISPRG |
| | | | | NHKYYNTKFIDRFTISRDDDKNTLYLQMN |
| | | | | SLRAEDTAVYYCARDWDYFDHWGQGTL |
| | | | | VTVSS |
| SEQ ID NO: 154 | 77 | PT5634.P19 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 155 | 78 | PT5636.A16 | VH | KVQLVESGGGLVQPGGSLRLSCAASGFKY |
| | | | | DIYAMSWVRQAPGKGLEWVGDISPKGET |
| | | | | KYYNTKFIDRFTISRDNSKNTLYLQMNSL |
| | | | | RAEDTAVYYCARDWDYFDAWGQGTLVT |
| | | | | VSS |
| SEQ ID NO: 156 | 78 | PT5636.A16 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 157 | 79 | PT5636.D21 | VH | EVQLVESGGGLVQPGGSLRLSCAASGYKF |
| | | | | DKYAMSWVRQAPGKGLEWVGEISPRGD |
| | | | | HKYYNTKFIDRFTISRDDDKNTLYLQMNS |
| | | | | LRAEDTAVYYCARDWEYFDSWGQGTLV |
| | | | | TVSS |
| SEQ ID NO: 158 | 79 | PT5636.D21 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 159 | 80 | PT5636.M03 | VH | KVQLVESGGGLVQPGGSLRLSCAASGFR |
| | | | | WDIYAMSWVRQAPGKGLEWVGEISPDGR |
| | | | | TKYYNTKFIDRFTISRDDSKNTLYLQMNS |
| | | | | LRAEDTAVYYCARDWDYFDTWGQGTLV |
| | | | | TVSS |
| SEQ ID NO: 160 | 80 | PT5636.M03 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 161 | 81 | PT5636.N13 | VH | RVQLVESGGGLVQPGGSLRLSCAASGFRY |
| | | | | DHYAMSWVRQAPGKGLEWVGSISPKGN |
| | | | | HKYYNTKFIDRFTISRDNSKNTLYLQMNS |
| | | | | LRAEDTAVYYCARDWDYFDHWGQGTLV |
| | | | | TVSS |
| SEQ ID NO: 162 | 81 | PT5636.N13 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 163 | 82 | PT5636.P11 | VH | EVQLVESGGGLVQPGGSLRLSCAASGFQF |
| | | | | DHYAMSWVRQAPGKGLEWVGSISPRGDH |
| | | | | KYYNTKFIDRFTISRDNSKNTLYLQMNSL |
| | | | | RAEDTAVYYCARDWDYFDQWGQGTLVT |
| | | | | VSS |
| SEQ ID NO: 164 | 82 | PT5636.P11 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 165 | 83 | PT5636.P15 | VH | KVQLVESGGGLVQPGGSLRLSCAASGYR |
| | | | | YDYYAMSWVRQAPGKGLEWVGSISPKG |
| | | | | DHKYYNTKFIDRFTISRDNSKNTLYLQMN |
| | | | | SLRAEDTAVYYCARDWDYFDGWGQGTL |
| | | | | VTVSS |
| SEQ ID NO: 166 | 83 | PT5636.P15 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 167 | 84 | PT5638.C06 | VH | KVQLVESGGGLVQPGGSLRLSCAASGYT |
| | | | | WEHYAMSWVRQAPGKGLEWVGSISPDG |
| | | | | RHKYYNTKFIDRFTISRDDDKNTLYLQMN |
| | | | | SLRAEDTAVYYCARDWDYFDYWGQGTL |
| | | | | VTVSS |
| SEQ ID NO: 168 | 84 | PT5638.C06 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 169 | 85 | PT5638.C20 | VH | KVQLVESGGGLVQPGGSLRLSCAASGFRY |
| | | | | DHYAMSWVRQAPGKGLEWVGSISPDGD |
| | | | | HKYYNTKFIDRFTISRDDSKNTLYLQMNS |
| | | | | LRAEDTAVYYCARDWDYFDHWGQGTLV |
| | | | | TVSS |
| SEQ ID NO: 170 | 85 | PT5638.C20 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 171 | 86 | PT5638.E14 | VH | RVQLVESGGGLVQPGGSLRLSCAASGFT |
| | | | | WDHYAMTWVRQAPGKGLEWVGEISPRG |
| | | | | DSKYYNTKFIDRFTISRDNSKNTLYLQMN |
| | | | | SLRAEDTAVYYCARDWDYFDHWGQGTL |
| | | | | VTVSS |
| SEQ ID NO: 172 | 86 | PT5638.E14 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 173 | 87 | PT5638.J23 | VH | EVQLVESGGGLVQPGGSLRLSCAASGWS |
| | | | | YDHYAMSWVRQAPGKGLEWVGSISPKG |
| | | | | DHKYYNTKFIDRFTISRDNEKNTLYLQMN |
| | | | | SLRAEDTAVYYCARDWDYFDYWGQGTL |
| | | | | VTVSS |
| SEQ ID NO: 174 | 87 | PT5638.J23 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 175 | 88 | PT5638.O14 | VH | RVQLVESGGGLVQPGGSLRLSCAASGFR |
| | | | | WDYYAMSWVRQAPGKGLEWVGSISPDG |
| | | | | EHKYYNTKFIDRFTISRDDSKNTLYLQMN |
| | | | | SLRAEDTAVYYCARDWDYFDTWGQGTL |
| | | | | VTVSS |
| SEQ ID NO: 176 | 88 | PT5638.O14 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 177 | 89 | PT5638.P12 | VH | RVQLVESGGGLVQPGGSLRLSCAASGFRY |
| | | | | DYYAMSWVRQAPGKGLEWVGSISPKGDT |
| | | | | KYYNTKFIDRFTISRDDEKNTLYLQMNSL |
| | | | | RAEDTAVYYCARDWDYFDIWGQGTLVT |
| | | | | VSS |
| SEQ ID NO: 178 | 89 | PT5638.P12 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRYTPYTFGQGTKLEIK |
| SEQ ID NO: 179 | 90 | PT5623.E21 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 180 | 90 | PT5623.E21 | VL | AIQMTQSPSSLSASVGDRVTITCQASYWLS |
| | | | | NYLAWYQQKPGKAPKLLIFDGDLPYERPS |
| | | | | RFSGSGSHDEYTLTISSLQPEDFATYYCQQ |
| | | | | YRNEPYTFGQGTKLEIK |
| SEQ ID NO: 181 | 91 | PT5623.F17 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 182 | 91 | PT5623.F17 | VL | AIQMTQSPSSLSASVGDRVTITCQSQYWLS |
| | | | | NYLAWYQQKPGKAPKLLIFDGDIPYEIPSR |
| | | | | FSGSGSHEEYTLTISSLQPEDFATYYCQQY |
| | | | | RDEPYTFGQGTKLEIK |
| SEQ ID NO: 183 | 92 | PT5623.G08 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 184 | 92 | PT5623.G08 | VL | AIQMTQSPSSLSASVGDRVTITCQASYWLS |
| | | | | NYLAWYQQKPGKAPKLLIFDGDLPYEIPS |
| | | | | RFSGSGSHEEYTLTISSLQPEDFATYYCQQ |
| | | | | YREEPYTFGQGTKLEIK |
| SEQ ID NO: 185 | 93 | PT5623.H05 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 186 | 93 | PT5623.H05 | VL | AIQMTQSPSSLSASVGDRVTITCQSTLWLS |
| | | | | NYLAWYQQKPGKAPKLLIFDGDLPYEIPS |
| | | | | RFSGSGSHEEYTLTISSLQPEDFATYYCQQ |
| | | | | YKETPYTFGQGTKLEIK |
| SEQ ID NO: 187 | 94 | PT5623.O08 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 188 | 94 | PT5623.O08 | VL | AIQMTQSPSSLSASVGDRVTITCQASYWLS |
| | | | | NYLAWYQQKPGKAPKLLIFDGDIPWERPS |
| | | | | RFSGSGSHDEYTLTISSLQPEDFATYYCQQ |
| | | | | YRDTPYTFGQGTKLEIK |
| SEQ ID NO: 189 | 95 | PT5625.A05 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 190 | 95 | PT5625.A05 | VL | AIQMTQSPSSLSASVGDRVTITCQASYWLS |
| | | | | NYLAWYQQKPGKAPKLLIFDGDIPYERPS |
| | | | | RFSGSGSHEEYTLTISSLQPEDFATYYCQQ |
| | | | | YRETPYTFGQGTKLEIK |
| SEQ ID NO: 191 | 96 | PT5625.B14 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 192 | 96 | PT5625.B14 | VL | AIQMTQSPSSLSASVGDRVTITCQASYWLS |
| | | | | NYLAWYQQKPGKAPKLLIFDGDLPYERPS |
| | | | | RFSGSGSEEEYTLTISSLQPEDFATYYCQQ |
| | | | | YRNTPYTFGQGTKLEIK |
| SEQ ID NO: 193 | 97 | PT5625.C15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 194 | 97 | PT5625.C15 | VL | AIQMTQSPSSLSASVGDRVTITCQASYWLS |
| | | | | NYLAWYQQKPGKAPKLLIFDGDLPYEIPS |
| | | | | RFSGSGSHEEYTLTISSLQPEDFATYYCQQ |
| | | | | YKETPYTFGQGTKLEIK |
| SEQ ID NO: 195 | 98 | PT5625.G05 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 196 | 98 | PT5625.G05 | VL | AIQMTQSPSSLSASVGDRVTITCQASYWLS |
| | | | | NYLAWYQQKPGKAPKLLIFDGDLPYEVPS |
| | | | | RFSGSGSHDEYTLTISSLQPEDFATYYCQQ |
| | | | | YRDTPYTFGQGTKLEIK |
| SEQ ID NO: 197 | 99 | PT5625.L22 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 198 | 99 | PT5625.L22 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRETPYTFGQGTKLEIK |
| SEQ ID NO: 199 | 100 | PT5625.O14 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 200 | 100 | PT5625.O14 | VL | AIQMTQSPSSLSASVGDRVTITCQSQLWLS |
| | | | | NYLAWYQQKPGKAPKLLIFDGDIPYERPS |
| | | | | RFSGSGSHEEYTLTISSLQPEDFATYYCQQ |
| | | | | YRSEPYTFGQGTKLEIK |
| SEQ ID NO: 201 | 101 | PT5627.I21 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 202 | 101 | PT5627.I21 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YREDPYTFGQGTKLEIK |
| SEQ ID NO: 203 | 102 | PT5629.F04 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 204 | 102 | PT5629.F04 | VL | AIQMTQSPSSLSASVGDRVTITCQVSHWLS |
| | | | | NYLAWYQQKPGKAPKLLIFDGDLPYEIPS |
| | | | | RFSGSGSHDEYTLTISSLQPEDFATYYCQQ |
| | | | | YRYTPYTFGQGTKLEIK |
| SEQ ID NO: 205 | 103 | PT5629.L22 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 206 | 103 | PT5629.L22 | VL | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RqTPYTFGQGTKLEIK |
| SEQ ID NO: 207 | 104 | PT5629.L24 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 208 | 104 | PT5629.L24 | VL | AIQMTQSPSSLSASVGDRVTITCQSqFWLS |
| | | | | NYLAWYQQKPGKAPKLLIFDGDLPYERPS |
| | | | | RFSGSGSHDEYTLTISSLQPEDFATYYCQQ |
| | | | | YESEPYTFGQGTKLEIK |
| SEQ ID NO: 209 | 105 | PT5629.M15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 210 | 105 | PT5629.M15 | VL | AIQMTQSPSSLSASVGDRVTITCQASYWLS |
| | | | | NYLAWYQQKPGKAPKLLIFDGDIPYEIPSR |
| | | | | FSGSGSWDEYTLTISSLQPEDFATYYCQQ |
| | | | | YRETPYTFGQGTKLEIK |
| SEQ ID NO: 211 | 106 | PT5631.H06 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 212 | 106 | PT5631.H06 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRDTPYTFGQGTKLEIK |
| SEQ ID NO: 213 | 107 | PT5631.K12 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 214 | 107 | PT5631.K12 | VL | AIQMTQSPSSLSASVGDRVTITCQASYWLS |
| | | | | NYLAWYQQKPGKAPKLLIFDGDIPWEIPS |
| | | | | RFSGSGSHDEYTLTISSLQPEDFATYYCQQ |
| | | | | YRDTPYTFGQGTKLEIK |
| SEQ ID NO: 215 | 108 | PT5631.N17 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 216 | 108 | PT5631.N17 | VL | AIQMTQSPSSLSASVGDRVTITCQSTLWLS |
| | | | | NYLAWYQQKPGKAPKLLIFDGDIPWEIPS |
| | | | | RFSGSGSWDEYTLTISSLQPEDFATYYCQQ |
| | | | | YRYTPYTFGQGTKLEIK |
| SEQ ID NO: 217 | 109 | PT5631.P07 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 218 | 109 | PT5631.P07 | VL | AIQMTQSPSSLSASVGDRVTITCQSQYWLS |
| | | | | NYLAWYQQKPGKAPKLLIFDGDLPWEVP |
| | | | | SRFSGSGSHDEYTLTISSLQPEDFATYYCQ |
| | | | | QYKDTPYTFGQGTKLEIK |
| SEQ ID NO: 219 | 110 | PT5633.E09 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 220 | 110 | PT5633.E09 | VL | AIQMTQSPSSLSASVGDRVTITCQAqHWLS |
| | | | | NYLAWYQQKPGKAPKLLIFDGDIPYERPS |
| | | | | RFSGSGSHDEYTLTISSLQPEDFATYYCQQ |
| | | | | YRETPYTFGQGTKLEIK |
| SEQ ID NO: 221 | 111 | PT5633.G19 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 222 | 111 | PT5633.G19 | VL | AIQMTQSPSSLSASVGDRVTITCQASYWLS |
| | | | | NYLAWYQQKPGKAPKLLIFDGDIPWEVPS |
| | | | | RFSGSGSHEDYTLTISSLQPEDFATYYCQQ |
| | | | | YRSEPYTFGQGTKLEIK |
| SEQ ID NO: 223 | 112 | PT5635.F04 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 224 | 112 | PT5635.F04 | VL | AIQMTQSPSSLSASVGDRVTITCQAHYWL |
| | | | | SNYLAWYQQKPGKAPKLLIFDGDIPYEIPS |
| | | | | RFSGSGSEDEYTLTISSLQPEDFATYYCQQ |
| | | | | YRSTPYTFGQGTKLEIK |
| SEQ ID NO: 225 | 113 | PT5635.L21 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 226 | 113 | PT5635.L21 | VL | AIQMTQSPSSLSASVGDRVTITCQASYWLS |
| | | | | NYLAWYQQKPGKAPKLLIFDGDLPYEIPS |
| | | | | RFSGSGSHDEYTLTISSLQPEDFATYYCQQ |
| | | | | YRETPYTFGQGTKLEIK |
| SEQ ID NO: 227 | 114 | PT5635.O15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 228 | 114 | PT5635.O15 | VL | AIQMTQSPSSLSASVGDRVTITCQASYWLS |
| | | | | NYLAWYQQKPGKAPKLLIFDGDIPWEVPS |
| | | | | RFSGSGSHDEYTLTISSLQPEDFATYYCQQ |
| | | | | YREEPYTFGQGTKLEIK |
| SEQ ID NO: 229 | 115 | PT5637.B23 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 230 | 115 | PT5637.B23 | VL | AIQMTQSPSSLSASVGDRVTITCQSQLWLS |
| | | | | NYLAWYQQKPGKAPKLLIFDGDLPYEIPS |
| | | | | RFSGSGSHEEYTLTISSLQPEDFATYYCQQ |
| | | | | YRETPYTFGQGTKLEIK |
| SEQ ID NO: 231 | 116 | PT5637.C20 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 232 | 116 | PT5637.C20 | VL | AIQMTQSPSSLSASVGDRVTITCQASYWLS |
| | | | | NYLAWYQQKPGKAPKLLIFDGDIPYEIPSR |
| | | | | FSGSGSHDEYTLTISSLQPEDFATYYCQQY |
| | | | | REDPYTFGQGTKLEIK |
| SEQ ID NO: 233 | 117 | PT5637.K09 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 234 | 117 | PT5637.K09 | VL | AIQMTQSPSSLSASVGDRVTITCQASYWLS |
| | | | | NYLAWYQQKPGKAPKLLIFDGDIPWEVPS |
| | | | | RFSGSGSHEEYTLTISSLQPEDFATYYCQQ |
| | | | | YREEPYTFGQGTKLEIK |
| SEQ ID NO: 235 | 118 | PT5637.L15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 236 | 118 | PT5637.L15 | VL | AIQMTQSPSSLSASVGDRVTITCQSQYWLS |
| | | | | NYLAWYQQKPGKAPKLLIFDGDLPYERPS |
| | | | | RFSGSGSHDEYTLTISSLQPEDFATYYCQQ |
| | | | | YRSEPYTFGQGTKLEIK |
| SEQ ID NO: 237 | 119 | PT5637.M03 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 238 | 119 | PT5637.M03 | VL | AIQMTQSPSSLSASVGDRVTITCQASYWLS |
| | | | | NYLAWYQQKPGKAPKLLIFDGDLPYERPS |
| | | | | RFSGSGSHDEYTLTISSLQPEDFATYYCQQ |
| | | | | YRDTPYTFGQGTKLEIK |
| SEQ ID NO: 239 | 120 | PT5637.P15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 240 | 120 | PT5637.P15 | VL | AIQMTQSPSSLSASVGDRVTITCQASYWLS |
| | | | | NYLAWYQQKPGKAPKLLIFDGDIPYEIPSR |
| | | | | FSGSGSHDEYTLTISSLQPEDFATYYCQQY |
| | | | | RETPYTFGQGTKLEIK |
| SEQ ID NO: 241 | 121 | PT5639.B15 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 242 | 121 | PT5639.B15 | VL | AIQMTQSPSSLSASIGDRVTITCQSQYWLS |
| | | | | NYLAWYQQKPGKAPKLLIFDGDIPWELPS |
| | | | | RFSGSGSHDEYTLTISSLQPEDFATYYCQQ |
| | | | | YREEPYTFGQGTKLEIK |
| SEQ ID NO: 243 | 122 | PT5639.P05 | VH | GQLVESGGGLVQPGGSLRLSCAASGFTW |
| | | | | DHYAMTWVRQAPGKGLEWVGSISPKGD |
| | | | | HKYYNTKFIDRFTISRDNSKNTLYLQMNS |
| | | | | LRAEDTAVYYCARDWDYFDEWGQGTLV |
| | | | | TVSS |
| SEQ ID NO: 244 | 122 | PT5639.P05 | VL | AIQMTQSPSSLSASVGDRVTITCQASYWLS |
| | | | | NYLAWYQQKPGKAPKLLIFDGDLPYETPS |
| | | | | RFSGSGSHDEYTLTISSLQPEDFATYYCQQ |
| | | | | YRETPYTFGQGTKLEIK |
| SEQ ID NO: 245 | 123 | PT5625.G07_ | VH | EVQLVESGGGLVQPGGSLRLSCAASGFRF |
| | | 22.F19_2 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 246 | 123 | PT5625.G07_ | VL | AIQMTQSPSSLSASVGDRVTITCQAQYWL |
| | | 22.F19_2 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 247 | 124 | PT5625.G07_ | VH | KVQLVESGGGLVQPGGSLRLSCAASGFR |
| | | 24.M09_1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 248 | 124 | PT5625.G07_ | VL | AIQMTQSPSSLSASVGDRVTITCQAQYWL |
| | | 24.M09_1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 249 | 125 | PT5625.G07_ | VH | QVQLVESGGGLVQPGGSLRLSCAASGFKY |
| | | 24.P06_2 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 250 | 125 | PT5625.G07_ | VL | AIQMTQSPSSLSASVGDRVTITCQAQYWL |
| | | 24.P06_2 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 251 | 126 | PT5625.G07_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 252 | 126 | PT5625.G07_ | VL | AIQMTQSPSSLSASVGDRVTITCQAQYWL |
| | | 26.C10_1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 253 | 127 | PT5625.G07_ | VH | RVQLVESGGGLVQPGGSLRLSCAASGFRY |
| | | 26.M10_2 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 254 | 127 | PT5625.G07_ | VL | AIQMTQSPSSLSASVGDRVTITCQAQYWL |
| | | 26.M10 2 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 255 | 128 | PT5625.G07_ | VH | RVQLVESGGGLVQPGGSLRLSCAASGFTY |
| | | 28.E16_1 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 256 | 128 | PT5625.G07_ | VL | AIQMTQSPSSLSASVGDRVTITCQAQYWL |
| | | 28.E16_1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 257 | 129 | PT5625.G07_ | VH | KVQLVESGGGLVQPGGSLRLSCAASGFK |
| | | 30.A20_1 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 258 | 129 | PT5625.G07_ | VL | AIQMTQSPSSLSASVGDRVTITCQAQYWL |
| | | 30.A20 1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 259 | 130 | PT5625.G07_ | VH | RVQLVESGGGLVQPGGSLRLSCAASGYR |
| | | 32.A13_2 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 260 | 130 | PT5625.G07_ | VL | AIQMTQSPSSLSASVGDRVTITCQAQYWL |
| | | 32.A13_2 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 261 | 131 | PT5625.G07_ | VH | EVQLVESGGGLVQPGGSLRLSCAASGFQ |
| | | 34.M24_1 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 262 | 131 | PT5625.G07_ | VL | AIQMTQSPSSLSASVGDRVTITCQAQYWL |
| | | 34.M24 1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 263 | 132 | PT5625.G07_ | VH | KVQLVESGGGLVQPGGSLRLSCAASGFTY |
| | | 34.N13_3 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 264 | 132 | PT5625.G07_ | VL | AIQMTQSPSSLSASVGDRVTITCQAQYWL |
| | | 34.N13_3 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 265 | 133 | PT5625.G07_ | VH | RVQLVESGGGLVQPGGSLRLSCAASGFKF |
| | | 36.P22_1 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 266 | 133 | PT5625.G07_ | VL | AIQMTQSPSSLSASVGDRVTITCQAQYWL |
| | | 36.P22_1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 267 | 134 | PT5627.M20_ | VH | KVQLVESGGGLVQPGGSLRLSCAASGFR |
| | | 24.M09_1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 268 | 134 | PT5627.M20_ | VL | AIQMTQSPSSLSASVGDRVTITCQSQFWLS |
| | | 24.M09_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 269 | 135 | PT5627.M20_ | VH | QVQLVESGGGLVQPGGSLRLSCAASGFKY |
| | | 24.P06_1 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 270 | 135 | PT5627.M20_ | VL | AIQMTQSPSSLSASVGDRVTITCQSQFWLS |
| | | 24.P06_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 271 | 136 | PT5627.M20_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 272 | 136 | PT5627.M20_ | VL | AIQMTQSPSSLSASVGDRVTITCQSQFWLS |
| | | 26.C10_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 273 | 137 | PT5627.M20_ | VH | RVQLVESGGGLVQPGGSLRLSCAASGFRY |
| | | 26.M10_2 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 274 | 137 | PT5627.M20_ | VL | AIQMTQSPSSLSASVGDRVTITCQSQFWLS |
| | | 26.M10 2 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 275 | 138 | PT5627.M20_ | VH | RVQLVESGGGLVQPGGSLRLSCAASGFTY |
| | | 28.E16_2 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 276 | 138 | PT5627.M20_ | VL | AIQMTQSPSSLSASVGDRVTITCQSQFWLS |
| | | 28.E16_2 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 277 | 139 | PT5627.M20_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 278 | 139 | PT5627.M20_ | VL | AIQMTQSPSSLSASVGDRVTITCQSQFWLS |
| | | 30.D05_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 279 | 140 | PT5627.M20_ | VH | RVQLVESGGGLVQPGGSLRLSCAASGYR |
| | | 32.A13_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 280 | 140 | PT5627.M20_ | VL | AIQMTQSPSSLSASVGDRVTITCQSQFWLS |
| | | 32.A13_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 281 | 141 | PT5627.M20_ | VH | EVQLVESGGGLVQPGGSLRLSCAASGFQ |
| | | 34.M24_2 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 282 | 141 | PT5627.M20_ | VL | AIQMTQSPSSLSASVGDRVTITCQSQFWLS |
| | | 43.M24_2 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 283 | 142 | PT5627.M20_ | VH | KVQLVESGGGLVQPGGSLRLSCAASGFTY |
| | | 34.N13_4 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 284 | 142 | PT5627.M20_ | VL | AIQMTQSPSSLSASVGDRVTITCQSQFWLS |
| | | 34.N13_4 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 285 | 143 | PT5627.M20_ | VH | RVQLVESGGGLVQPGGSLRLSCAASGFKF |
| | | 36.P22_1 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 286 | 143 | PT5627.M20_ | VL | AIQMTQSPSSLSASVGDRVTITCQSQFWLS |
| | | 36.P22_1 | | NYLAWYQQKPGKAPKLLIFDGDLPYERPS |
| | | | | RFSGSGSHDEYTLTISSLQPEDFATYYCQQ |
| | | | | YREEPYTFGQGTKLEIK |
| SEQ ID NO: 287 | 144 | PT5627.M12_ | VH | EVQLVESGGGLVQPGGSLRLSCAASGFRF |
| | | 22.F19_1 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 288 | 144 | PT5627.M12_ | VL | AIQMTQSPSSLSASVGDRVTITCQASYWLS |
| | | 22.F19_1 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 289 | 145 | PT5627.M12_ | VH | KVQLVESGGGLVQPGGSLRLSCAASGFR |
| | | 24.M09_1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 290 | 145 | PT5627.M12_ | VL | AIQMTQSPSSLSASVGDRVTITCQASYWLS |
| | | 24.M09_1 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 291 | 146 | PT5627.M12_ | VH | RVQLVESGGGLVQPGGSLRLSCAASGFRY |
| | | 26.M10_2 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 292 | 146 | PT5627.M12_ | VL | AIQMTQSPSSLSASVGDRVTITCQASYWLS |
| | | 26.M10_2 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 293 | 147 | PT5627.M12_ | VH | RVQLVESGGGLVQPGGSLRLSCAASGYR |
| | | 32.A13_2 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 294 | 147 | PT5627.M12_ | VL | AIQMTQSPSSLSASVGDRVTITCQASYWLS |
| | | 32.A13_2 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 295 | 148 | PT5627.M12_ | VH | EVQLVESGGGLVQPGGSLRLSCAASGFQ |
| | | 34.M24_2 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 296 | 148 | PT5627.M12_ | VL | AIQMTQSPSSLSASVGDRVTITCQASYWLS |
| | | 34.M24_2 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 297 | 149 | PT5627.M12_ | VH | KVQLVESGGGLVQPGGSLRLSCAASGFTY |
| | | 34.N13_1 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 298 | 149 | PT5627.M12_ | VL | AIQMTQSPSSLSASVGDRVTITCQASYWLS |
| | | 34.N13_1 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 299 | 150 | PT5629.F09_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 300 | 150 | PT5629.F09_ | VL | AIQMTQSPSSLSASVGDRVTITCQASYWLS |
| | | 26.C10_1 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 301 | 151 | PT5629.F09_ | VH | RVQLVESGGGLVQPGGSLRLSCAASGFTY |
| | | 28.E16_1 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 302 | 151 | PT5629.F09_ | VL | AIQMTQSPSSLSASVGDRVTITCQASYWLS |
| | | 28.E16_1 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 303 | 152 | PT5629.F09_ | VH | KVQLVESGGGLVQPGGSLRLSCAASGFK |
| | | 30.A20_1 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 304 | 152 | PT5629.F09_ | VL | AIQMTQSPSSLSASVGDRVTITCQASYWLS |
| | | 30A20_1 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 305 | 153 | PT5629.F09_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 306 | 153 | PT5629.F09_ | VL | AIQMTQSPSSLSASVGDRVTITCQASYWLS |
| | | 30.D05_1 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 307 | 154 | PT5629.F09_ | VH | RVQLVESGGGLVQPGGSLRLSCAASGYR |
| | | 32.A13_3 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 308 | 154 | PT5629.F09_ | VL | AIQMTQSPSSLSASVGDRVTITCQASYWLS |
| | | 32.A13_3 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 309 | 155 | PT5629.F09_ | VH | RVQLVESGGGLVQPGGSLRLSCAASGFKF |
| | | 36.P22_2 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 310 | 155 | PT5629.F09_ | VL | AIQMTQSPSSLSASVGDRVTITCQASYWLS |
| | | 36.P22_2 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 311 | 156 | PT5629.H08_ | VH | EVQLVESGGGLVQPGGSLRLSCAASGFRF |
| | | 22.F19_4 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 312 | 156 | PT5629.H08_ | VL | AIQMTQSPSSLSASVGDRVTITCQASYWLS |
| | | 22.F19_4 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 313 | 157 | PT5629.H08_ | VH | KVQLVESGGGLVQPGGSLRLSCAASGFR |
| | | 24.M09_4 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 314 | 157 | PT5629.H08_ | VL | AIQMTQSPSSLSASVGDRVTITCQASYWLS |
| | | 24.M09_4 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 315 | 158 | PT5629.H08_ | VH | QVQLVESGGGLVQPGGSLRLSCAASGFKY |
| | | 24.P06_2 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 316 | 158 | PT5629.H08_ | VL | AIQMTQSPSSLSASVGDRVTITCQASYWLS |
| | | 24.P06_2 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 317 | 159 | PT5629.H08_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 318 | 159 | PT5629.H08_ | VL | AIQMTQSPSSLSASVGDRVTITCQASYWLS |
| | | 26.C10_1 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 319 | 160 | PT5629.H08_ | VH | RVQLVESGGGLVQPGGSLRLSCAASGFRY |
| | | 26.M10_1 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 320 | 160 | PT5629.H08_ | VL | AIQMTQSPSSLSASVGDRVTITCQASYWLS |
| | | 26.M10_1 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 321 | 161 | PT5629.H08_ | VH | RVQLVESGGGLVQPGGSLRLSCAASGFTY |
| | | 28.E16_2 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 322 | 161 | PT5629.H08_ | VL | AIQMTQSPSSLSASVGDRVTITCQASYWLS |
| | | 28.E16_2 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 323 | 162 | PT5629.H08_ | VH | KVQLVESGGGLVQPGGSLRLSCAASGFK |
| | | 30.A20_2 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 324 | 162 | PT5629.H08_ | VL | AIQMTQSPSSLSASVGDRVTITCQASYWLS |
| | | 30.A20_2 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 325 | 163 | PT5629.H08_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 326 | 163 | PT5629.H08_ | VL | AIQMTQSPSSLSASVGDRVTITCQASYWLS |
| | | 30.D05_1 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 327 | 164 | PT5629.H08_ | VH | RVQLVESGGGLVQPGGSLRLSCAASGYR |
| | | 32.A13_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 328 | 164 | PT5629.H08_ | VL | AIQMTQSPSSLSASVGDRVTITCQASYWLS |
| | | 32.A13_1 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 329 | 165 | PT5629.H08_ | VH | EVQLVESGGGLVQPGGSLRLSCAASGFQ |
| | | 34.M24_2 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 330 | 165 | PT5629.H08_ | VL | AIQMTQSPSSLSASVGDRVTITCQASYWLS |
| | | 34.M24_2 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 331 | 166 | PT5629.H08_ | VH | KVQLVESGGGLVQPGGSLRLSCAASGFTY |
| | | 34.N13_2 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 332 | 166 | PT5629.H08_ | VL | AIQMTQSPSSLSASVGDRVTITCQASYWLS |
| | | 34.N13_2 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 333 | 167 | PT5629.H08_ | VH | RVQLVESGGGLVQPGGSLRLSCAASGFKF |
| | | 36.P22_4 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 334 | 167 | PT5629.H08_ | VL | AIQMTQSPSSLSASVGDRVTITCQASYWLS |
| | | 36.P22_4 | | NYLAWYQQKPGKAPKLLIFDGDIPYEIPSR |
| | | | | FSGSGSEDEYTLTISSLQPEDFATYYCQQY |
| | | | | RETPYTFGQGTKLEIK |
| SEQ ID NO: 335 | 168 | PT5631.D21_ | VH | EVQLVESGGGLVQPGGSLRLSCAASGFRF |
| | | 22.F19_2 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 336 | 168 | PT5631.D21_ | VL | AIQMTQSPSSLSASVGDRVTITCQSSHWLS |
| | | 22.F19_2 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 337 | 169 | PT5631.D21_ | VH | KVQLVESGGGLVQPGGSLRLSCAASGFR |
| | | 24.M09 1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 338 | 169 | PT5631.D21_ | VL | AIQMTQSPSSLSASVGDRVTITCQSSHWLS |
| | | 24.M09_1 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 339 | 170 | PT5631.D21_ | VH | QVQLVESGGGLVQPGGSLRLSCAASGFKY |
| | | 24.P06_4 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 340 | 170 | PT5631.D21_ | VL | AIQMTQSPSSLSASVGDRVTITCQSSHWLS |
| | | 24.P06_4 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 341 | 171 | PT5631.D21_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 342 | 171 | PT5631.D21_ | VL | AIQMTQSPSSLSASVGDRVTITCQSSHWLS |
| | | 26.C10_1 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 343 | 172 | PT5631.D21_ | VH | RVQLVESGGGLVQPGGSLRLSCAASGFRY |
| | | 26.M10_4 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 344 | 172 | PT5631.D21_ | VL | AIQMTQSPSSLSASVGDRVTITCQSSHWLS |
| | | 26.M10_4 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 345 | 173 | PT5631.D21_ | VH | RVQLVESGGGLVQPGGSLRLSCAASGFTY |
| | | 28.E16_3 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 346 | 173 | PT5631.D21_ | VL | AIQMTQSPSSLSASVGDRVTITCQSSHWLS |
| | | 28.E16_3 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 347 | 174 | PT5631.D21_ | VH | KVQLVESGGGLVQPGGSLRLSCAASGFK |
| | | 30.A20_4 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 348 | 174 | PT5631.D21_ | VL | AIQMTQSPSSLSASVGDRVTITCQSSHWLS |
| | | 30.A20_4 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 349 | 175 | PT5631.D21_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 350 | 175 | PT5631.D21_ | VL | AIQMTQSPSSLSASVGDRVTITCQSSHWLS |
| | | 30.D05_1 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 351 | 176 | PT5631.D21_ | VH | RVQLVESGGGLVQPGGSLRLSCAASGYR |
| | | 32.A13_4 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 352 | 176 | PT5631.D21_ | VL | AIQMTQSPSSLSASVGDRVTITCQSSHWLS |
| | | 32.A13_4 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 353 | 177 | PT5631.D21_ | VH | EVQLVESGGGLVQPGGSLRLSCAASGFQ |
| | | 34.M24 4 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 354 | 177 | PT5631.D21_ | VL | AIQMTQSPSSLSASVGDRVTITCQSSHWLS |
| | | 34.M24_4 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 355 | 178 | PT5631.D21_ | VH | KVQLVESGGGLVQPGGSLRLSCAASGFTY |
| | | 34.N13_2 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 356 | 178 | PT5631.D21_ | VL | AIQMTQSPSSLSASVGDRVTITCQSSHWLS |
| | | 34.N13_2 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 357 | 179 | PT5631.D21_ | VH | RVQLVESGGGLVQPGGSLRLSCAASGFKF |
| | | 36.P22 3 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 358 | 179 | PT5631.D21_ | VL | AIQMTQSPSSLSASVGDRVTITCQSSHWLS |
| | | 36.P22_3 | | NYLAWYQQKPGKAPKLLIFDGDLPWEVP |
| | | | | SRFSGSGSEDEYTLTISSLQPEDFATYYCQ |
| | | | | QYRYTPYTFGQGTKLEIK |
| SEQ ID NO: 359 | 180 | PT5633.I03_ | VH | EVQLVESGGGLVQPGGSLRLSCAASGFRF |
| | | 22.F19_1 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 360 | 180 | PT5633.I03_ | VL | AIQMTQSPSSLSASVGDRVTITCQASYWLS |
| | | 22.F19_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 361 | 181 | PT5633.I03_ | VH | KVQLVESGGGLVQPGGSLRLSCAASGFR |
| | | 24.M09_1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 362 | 181 | PT5633.I03_ | VL | AIQMTQSPSSLSASVGDRVTITCQASYWLS |
| | | 24.M09_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 363 | 182 | PT5633.I03_ | VH | QVQLVESGGGLVQPGGSLRLSCAASGFKY |
| | | 24.P06_4 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 364 | 182 | PT5633.I03_ | VL | AIQMTQSPSSLSASVGDRVTITCQASYWLS |
| | | 24.P06_4 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 365 | 183 | PT5633.I03_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 366 | 183 | PT5633.I03_ | VL | AIQMTQSPSSLSASVGDRVTITCQASYWLS |
| | | 26.C10_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 367 | 184 | PT5633.I03_ | VH | RVQLVESGGGLVQPGGSLRLSCAASGFRY |
| | | 26.M10_4 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 368 | 184 | PT5633.I03_ | VL | AIQMTQSPSSLSASVGDRVTITCQASYWLS |
| | | 26.M10_4 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 369 | 185 | PT5633.I03_ | VH | RVQLVESGGGLVQPGGSLRLSCAASGFTY |
| | | 28.E16_4 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 370 | 185 | PT5633.I03_ | VL | AIQMTQSPSSLSASVGDRVTITCQASYWLS |
| | | 28.E16_4 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 371 | 186 | PT5633.I03_ | VH | KVQLVESGGGLVQPGGSLRLSCAASGFK |
| | | A20_1 | | WEHYAMSWVRQAPGKGLEWVGSISPRG |
| | | 30. | | DHKYYNTKFIDRFTISRDNDKNTLYLQMN |
| | | | | SLRAEDTAVYYCARDWDYFDRWGQGTL |
| | | | | VTVSS |
| SEQ ID NO: 372 | 186 | PT5633.I03_ | VL | AIQMTQSPSSLSASVGDRVTITCQASYWLS |
| | | 30.A20_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 373 | 187 | PT5633.I03_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 374 | 187 | PT5633.I03_ | VL | AIQMTQSPSSLSASVGDRVTITCQASYWLS |
| | | 30.D05_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 375 | 188 | PT5633.I03_ | VH | RVQLVESGGGLVQPGGSLRLSCAASGYR |
| | | 32.A13_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 376 | 188 | PT5633.I03_ | VL | AIQMTQSPSSLSASVGDRVTITCQASYWLS |
| | | 32.A13_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 377 | 189 | PT5633.I03_ | VH | EVQLVESGGGLVQPGGSLRLSCAASGFQ |
| | | 34.M24_2 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 378 | 189 | PT5633.I03_ | VL | AIQMTQSPSSLSASVGDRVTITCQASYWLS |
| | | 34.M24_2 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 379 | 190 | PT5633.I03_ | VH | KVQLVESGGGLVQPGGSLRLSCAASGFTY |
| | | 34.N13_1 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 380 | 190 | PT5633.I03_ | VL | AIQMTQSPSSLSASVGDRVTITCQASYWLS |
| | | 34.N13_1 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 381 | 191 | PT5633.I03_ | VH | RVQLVESGGGLVQPGGSLRLSCAASGFKF |
| | | 36.P22_2 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 382 | 191 | PT5633.I03_ | VL | AIQMTQSPSSLSASVGDRVTITCQASYWLS |
| | | 36.P22_2 | | NYLAWYQQKPGKAPKLLIFDGDIPWEVPS |
| | | | | RFSGSGSHEDYTLTISSLQPEDFATYYCQQ |
| | | | | YRNTPYTFGQGTKLEIK |
| SEQ ID NO: 383 | 192 | PT5635.C08_ | VH | EVQLVESGGGLVQPGGSLRLSCAASGFRF |
| | | 22.F19_1 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 384 | 192 | PT5635.C08_ | VL | AIQMTQSPSSLSASVGDRVTITCQASYWLS |
| | | 22.F19_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 385 | 193 | PT5635.C08_ | VH | KVQLVESGGGLVQPGGSLRLSCAASGFR |
| | | 24.M09_1 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 386 | 193 | PT5635.C08_ | VL | AIQMTQSPSSLSASVGDRVTITCQASYWLS |
| | | 24.M09_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 387 | 194 | PT5635.C08_ | VH | QVQLVESGGGLVQPGGSLRLSCAASGFKY |
| | | 24.P06_1 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 388 | 194 | PT5635.C08_ | VL | AIQMTQSPSSLSASVGDRVTITCQASYWLS |
| | | 24.P06_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 389 | 195 | PT5635.C08_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 390 | 195 | PT5635.C08_ | VL | AIQMTQSPSSLSASVGDRVTITCQASYWLS |
| | | 26.C10_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 391 | 196 | PT5635.C08_ | VH | RVQLVESGGGLVQPGGSLRLSCAASGFRY |
| | | 26.M10_2 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 392 | 196 | PT5635.C08_ | VL | AIQMTQSPSSLSASVGDRVTITCQASYWLS |
| | | 26.M10_2 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 393 | 197 | PT5635.C08_ | VH | RVQLVESGGGLVQPGGSLRLSCAASGFTY |
| | | 28.E16_1 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 394 | 197 | PT5635.C08_ | VL | AIQMTQSPSSLSASVGDRVTITCQASYWLS |
| | | 28.E16_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 395 | 198 | PT5635.C08_ | VH | KVQLVESGGGLVQPGGSLRLSCAASGFK |
| | | 30.A20_1 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 396 | 198 | PT5635.C08_ | VL | AIQMTQSPSSLSASVGDRVTITCQASYWLS |
| | | 30.A20_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 397 | 199 | PT5635.C08_ | VH | KVQLVESGGGLVQPGGSLRLSCAASGYR |
| | | 30.D05_3 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 398 | 199 | PT5635.C08_ | VL | AIQMTQSPSSLSASVGDRVTITCQASYWLS |
| | | 30.D05_3 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 399 | 200 | PT5635.C08_ | VH | RVQLVESGGGLVQPGGSLRLSCAASGYR |
| | | 32.A13_3 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDYWGQGTL |
| | | | | VTVSS |
| SEQ ID NO: 400 | 200 | PT5635.C08_ | VL | AIQMTQSPSSLSASVGDRVTITCQASYWLS |
| | | 32.A13_3 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 401 | 201 | PT5635.C08_ | VH | EVQLVESGGGLVQPGGSLRLSCAASGFQ |
| | | 34.M24_2 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 402 | 201 | PT5635.C08_ | VL | AIQMTQSPSSLSASVGDRVTITCQASYWLS |
| | | 34.M24_2 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 403 | 202 | PT5635.C08_ | VH | KVQLVESGGGLVQPGGSLRLSCAASGFTY |
| | | 34.N13_1 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 404 | 202 | PT5635.C08_ | VL | AIQMTQSPSSLSASVGDRVTITCQASYWLS |
| | | 34.N13_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 405 | 203 | PT5635.C08_ | VH | RVQLVESGGGLVQPGGSLRLSCAASGFKF |
| | | 36.P22_1 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 406 | 203 | PT5635.C08_ | VL | AIQMTQSPSSLSASVGDRVTITCQASYWLS |
| | | 36.P22_1 | | NYLAWYQQKPGKAPKLLIFDGDIPWEVPS |
| | | | | RFSGSGSHEEYTLTISSLQPEDFATYYCQQ |
| | | | | YRSTPYTFGQGTKLEIK |
| SEQ ID NO: 407 | 204 | PT5639.G15_ | VH | EVQLVESGGGLVQPGGSLRLSCAASGFRF |
| | | 22.F19_2 | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 408 | 204 | PT5639.G15_ | VL | AIQMTQSPSSLSASVGDRVTITCQSTHWLS |
| | | 22.F19_2 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 409 | 205 | PT5639.G15_ | VH | QVQLVESGGGLVQPGGSLRLSCAASGFKY |
| | | 24.P06_1 | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSS |
| SEQ ID NO: 410 | 205 | PT5639.G15_ | VL | AIQMTQSPSSLSASVGDRVTITCQSTHWLS |
| | | 24.P06_1 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 411 | 206 | PT5639.G15_ | VH | KVQLVESGGGLVQPGGSLRLSCAASGFR |
| | | 24.M09_4 | | WEHYAMSWVRQAPGKGLEWVGEISPDG |
| | | | | DTKYYNTKFIDRFTISRDDSKNTLYLQMN |
| | | | | SLRAEDTAVYYCARDWEYFDNWGQGTL |
| | | | | VTVSS |
| SEQ ID NO: 412 | 206 | PT5639.G15_ | VL | AIQMTQSPSSLSASVGDRVTITCQSTHWLS |
| | | 24.M09_4 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 413 | 207 | PT5639.G15_ | VH | RVQLVESGGGLVQPGGSLRLSCAASGFRY |
| | | 26.M10_3 | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSS |
| SEQ ID NO: 414 | 207 | PT5639.G15_ | VL | AIQMTQSPSSLSASVGDRVTITCQSTHWLS |
| | | 26M.10_3 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 415 | 208 | PT5639.G15_ | VH | RVQLVESGGGLVQPGGSLRLSCAASGFTY |
| | | 28.E16_1 | | DHYAMSWVRQAPGKGLEWVGEISPRGNT |
| | | | | KYYNTKFIDRFTISRDNSKNTLYLQMNSL |
| | | | | RAEDTAVYYCARDWEYFDYWGQGTLVT |
| | | | | VSS |
| SEQ ID NO: 416 | 208 | PT5639.G15_ | VL | AIQMTQSPSSLSASVGDRVTITCQSTHWLS |
| | | 28.E16_1 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 417 | 209 | PT5639.G15_ | VH | KVQLVESGGGLVQPGGSLRLSCAASGFK |
| | | 30.A20_1 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 418 | 209 | PT5639.G15_ | VL | AIQMTQSPSSLSASVGDRVTITCQSTHWLS |
| | | 30.A20_1 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 419 | 210 | PT5639.G15_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 420 | 210 | PT5639.G15_ | VL | AIQMTQSPSSLSASVGDRVTITCQSTHWLS |
| | | 30.D05_1 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 421 | 211 | PT5639.G15_ | VH | KVQLVESGGGLVQPGGSLRLSCAASGFTY |
| | | 34.N13_4 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 422 | 211 | PT5639.G15_ | VL | AIQMTQSPSSLSASVGDRVTITCQSTHWLS |
| | | 34.N13_4 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 423 | 212 | PT5639.G15_ | VH | EVQLVESGGGLVQPGGSLRLSCAASGFQ |
| | | 34.M24_3 | | WDHYAMSWVRQAPGKGLEWVGEISPRG |
| | | | | DTKYYNTKFIDRFTISRDDEKNTLYLQMN |
| | | | | SLRAEDTAVYYCARDWEYFDYWGQGTL |
| | | | | VTVSS |
| SEQ ID NO: 424 | 212 | PT5639.G15_ | VL | AIQMTQSPSSLSASVGDRVTITCQSTHWLS |
| | | 34.M24_3 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 425 | 213 | PT5639.G15_ | VH | RVQLVESGGGLVQPGGSLRLSCAASGFKF |
| | | 36.P22_3 | | EHYAMSWVRQAPGKGLEWVGEISPKGDH |
| | | | | KYYNTKFIDRFTISRDNEKNTLYLQMNSL |
| | | | | RAEDTAVYYCARDWEYFDRWGQGTLVT |
| | | | | VSS |
| SEQ ID NO: 426 | 213 | PT5639.G15_ | VL | AIQMTQSPSSLSASVGDRVTITCQSTHWLS |
| | | 36.P22_3 | | AYLAWYQQKPGKAPKLLIFDGDIPYERPS |
| | | | | RFSGSGSHDEYTLTISSLQPEDFATYYCQQ |
| | | | | YRNEPYTFGQGTKLEIK |
| SEQ ID NO: 427 | 214 | PT5625.G07_ | VH | KVQLVESGGGLVQPGGSLRLSCAASGYR |
| | | 30.D05_1 | | YDKYAMSWVRQAPGKGLEWVGEISPDG |
| | | | | RTKYYNTKFIDRFTISRDNDKNTLYLQMN |
| | | | | SLRAEDTAVYYCARDWEYFDNWGQGTL |
| | | | | VTVSS |
| SEQ ID NO: 428 | 214 | PT5625.G07_ | VL | AIQMTQSPSSLSASVGDRVTITCQAQYWL |
| | | 30.D05_1 | | SNYLAWYQQKPGKAPKLLIFDGDLPYEVP |
| | | | | SRFSGSGSHDEYTLTISSLQPEDFATYYCQ |
| | | | | QYRNTPYTFGQGTKLEIK |
| SEQ ID NO: 429 | 215 | PT5627.M12_ | VH | RVQLVESGGGLVQPGGSLRLSCAASGFT |
| | | 26.C10_1 | | WDHYAMSWVRQAPGKGLEWVGSISPKG |
| | | | | DHKYYNTKFIDRFTISRDNSKNTLYLQMN |
| | | | | SLRAEDTAVYYCARDWDYFDRWGQGTL |
| | | | | VTVSS |
| SEQ ID NO: 430 | 215 | PT5627.M12_ | VL | AIQMTQSPSSLSASVGDRVTITCQASYWLS |
| | | 26.C10_1 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 431 | 216 | PT5627.M12_ | VH | KVQLVESGGGLVQPGGSLRLSCAASGFK |
| | | 30.A20_2 | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSS |
| SEQ ID NO: 432 | 216 | PT5627.M12_ | VL | AIQMTQSPSSLSASVGDRVTITCQASYWLS |
| | | 30.A20_2 | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIK |
| SEQ ID NO: 433 | 217 | PT5629.F09_ | VH | KVQLVESGGGLVQPGGSLRLSCAASGFTY |
| | | 34.N13_3 | | DAYAMSWVRQAPGKGLEWVGEISPRGES |
| | | | | KYYNTKFIDRFTISRDNSKNTLYLQMNSL |
| | | | | RAEDTAVYYCARDWEYFDNWGQGTLVT |
| | | | | VSS |
| SEQ ID NO: 434 | 217 | PT5629.F09_ | VL | AIQMTQSPSSLSASVGDRVTITCQASYWLS |
| | | 34.N13_3 | | NYLAWYQQKPGKAPKLLIFDGDIPYEIPSR |
| | | | | FSGSGSHEEYTLTISSLQPEDFATYYCQQY |
| | | | | RETPYTFGQGTKLEIK |
| SEQ ID NO: 435 | 1 | P1AG9426 | HC | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSSASTKGPSVFPLAPSSKSTSGGTAALGC |
| | | | | LVKDYFPEPVTVSWNSGALTSGVHTFPAV |
| | | | | LQSSGLYSLSSVVTVPSSSLGTQTYICNVN |
| | | | | HKPSNTKVDKKVEPKSCDKTHTCPPCPAP |
| | | | | EAAGGPSVFLFPPKPKDTLMASRTPEVTC |
| | | | | VVVDVSHEDPEVKFNWYVDGVEVHNAK |
| | | | | TKPREEQYNSTYRVVSVLTVLAQDWLNG |
| | | | | KEYKCKVSNKALGAPIEKTISKAKGQPRE |
| | | | | PQVYTLPPSRDELTKNQVSLTCLVKGFYP |
| | | | | SDIAVEWESNGQPENNYKTTPPVLDSDGS |
| | | | | FFLYSKLTVDKSRWQQGNVFSCSVMHEA |
| | | | | LHNAYTQKSLSLSP |
| SEQ ID NO: 436 | 1 | P1AG9426 | LC | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RETPYTFGQGTKLEIKRTVAAPSVFIFPPSD |
| | | | | EQLKSGTASVVCLLNNFYPREAKVQWKV |
| | | | | DNALQSGNSQESVTEQDSKDSTYSLSSTL |
| | | | | TLSKADYEKHKVYACEVTHQGLSSPVTKS |
| | | | | FNRGEC |
| SEQ ID NO: 437 | 2 | P1AG9376 | HC | KVQLVESGGGLVQPGGSLRLSCAASGFK |
| | | | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSSASTKGPSVFPLAPSSKSTSGGTAAL |
| | | | | GCLVKDYFPEPVTVSWNSGALTSGVHTFP |
| | | | | AVLQSSGLYSLSSVVTVPSSSLGTQTYICN |
| | | | | VNHKPSNTKVDKKVEPKSCDKTHTCPPCP |
| | | | | APEAAGGPSVFLFPPKPKDTLMASRTPEV |
| | | | | TCVVVDVSHEDPEVKFNWYVDGVEVHN |
| | | | | AKTKPREEQYNSTYRVVSVLTVLAQDWL |
| | | | | NGKEYKCKVSNKALGAPIEKTISKAKGQP |
| | | | | REPQVYTLPPSRDELTKNQVSLTCLVKGF |
| | | | | YPSDIAVEWESNGQPENNYKTTPPVLDSD |
| | | | | GSFFLYSKLTVDKSRWQQGNVFSCSVMH |
| | | | | EALHNAYTQKSLSLSP |
| SEQ ID NO: 438 | 2 | P1AG9376 | LC | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIKRTVAAPSVFIFPPS |
| | | | | DEQLKSGTASVVCLLNNFYPREAKVQWK |
| | | | | VDNALQSGNSQESVTEQDSKDSTYSLSST |
| | | | | LTLSKADYEKHKVYACEVTHQGLSSPVT |
| | | | | KSFNRGEC |
| SEQ ID NO: 439 | 3 | P1AG9372 | HC | EVQLVESGGGLVQPGGSLRLSCAASGFRF |
| | | | | DIYAMSWVRQAPGKGLEWVGEISPDGRT |
| | | | | KYYNTKFIDRFTISRDNEKNTLYLQMNSL |
| | | | | RAEDTAVYYCARDWEYFDNWGQGTLVT |
| | | | | VSSASTKGPSVFPLAPSSKSTSGGTAALGC |
| | | | | LVKDYFPEPVTVSWNSGALTSGVHTFPAV |
| | | | | LQSSGLYSLSSVVTVPSSSLGTQTYICNVN |
| | | | | HKPSNTKVDKKVEPKSCDKTHTCPPCPAP |
| | | | | EAAGGPSVFLFPPKPKDTLMASRTPEVTC |
| | | | | VVVDVSHEDPEVKFNWYVDGVEVHNAK |
| | | | | TKPREEQYNSTYRVVSVLTVLAQDWLNG |
| | | | | KEYKCKVSNKALGAPIEKTISKAKGQPRE |
| | | | | PQVYTLPPSRDELTKNQVSLTCLVKGFYP |
| | | | | SDIAVEWESNGQPENNYKTTPPVLDSDGS |
| | | | | FFLYSKLTVDKSRWQQGNVFSCSVMHEA |
| | | | | LHNAYTQKSLSLSP |
| SEQ ID NO: 440 | 3 | P1AG9372 | LC | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIKRTVAAPSVFIFPPS |
| | | | | DEQLKSGTASVVCLLNNFYPREAKVQWK |
| | | | | VDNALQSGNSQESVTEQDSKDSTYSLSST |
| | | | | LTLSKADYEKHKVYACEVTHQGLSSPVT |
| | | | | KSFNRGEC |
| SEQ ID NO: 441 | 4 | P1AG9420 | HC | RVQLVESGGGLVQPGGSLRLSCAASGFRY |
| | | | | DHYAMSWVRQAPGKGLEWVGSISPRGNH |
| | | | | KYYNTKFIDRFTISRDDEKNTLYLQMNSL |
| | | | | RAEDTAVYYCARDWDYFDHWGQGTLVT |
| | | | | VSSASTKGPSVFPLAPSSKSTSGGTAALGC |
| | | | | LVKDYFPEPVTVSWNSGALTSGVHTFPAV |
| | | | | LQSSGLYSLSSVVTVPSSSLGTQTYICNVN |
| | | | | HKPSNTKVDKKVEPKSCDKTHTCPPCPAP |
| | | | | EAAGGPSVFLFPPKPKDTLMASRTPEVTC |
| | | | | VVVDVSHEDPEVKFNWYVDGVEVHNAK |
| | | | | TKPREEQYNSTYRVVSVLTVLAQDWLNG |
| | | | | KEYKCKVSNKALGAPIEKTISKAKGQPRE |
| | | | | PQVYTLPPSRDELTKNQVSLTCLVKGFYP |
| | | | | SDIAVEWESNGQPENNYKTTPPVLDSDGS |
| | | | | FFLYSKLTVDKSRWQQGNVFSCSVMHEA |
| | | | | LHNAYTQKSLSLSP |
| SEQ ID NO: 442 | 4 | P1AG9420 | LC | AIQMTQSPSSLSASVGDRVTITCQASYWLS |
| | | | | NYLAWYQQKPGKAPKLLIFDGDIPYEIPSR |
| | | | | FSGSGSHEEYTLTISSLQPEDFATYYCQQY |
| | | | | RETPYTFGQGTKLEIKRTVAAPSVFIFPPSD |
| | | | | EQLKSGTASVVCLLNNFYPREAKVQWKV |
| | | | | DNALQSGNSQESVTEQDSKDSTYSLSSTL |
| | | | | TLSKADYEKHKVYACEVTHQGLSSPVTKS |
| | | | | FNRGEC |
| SEQ ID NO: 443 | 5 | P1AG9391 | HC | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSSASTKGPSVFPLAPSSKSTSGGTAALGC |
| | | | | LVKDYFPEPVTVSWNSGALTSGVHTFPAV |
| | | | | LQSSGLYSLSSVVTVPSSSLGTQTYICNVN |
| | | | | HKPSNTKVDKKVEPKSCDKTHTCPPCPAP |
| | | | | EAAGGPSVFLFPPKPKDTLMASRTPEVTC |
| | | | | VVVDVSHEDPEVKFNWYVDGVEVHNAK |
| | | | | TKPREEQYNSTYRVVSVLTVLAQDWLNG |
| | | | | KEYKCKVSNKALGAPIEKTISKAKGQPRE |
| | | | | PQVYTLPPSRDELTKNQVSLTCLVKGFYP |
| | | | | SDIAVEWESNGQPENNYKTTPPVLDSDGS |
| | | | | FFLYSKLTVDKSRWQQGNVFSCSVMHEA |
| | | | | LHNAYTQKSLSLSP |
| SEQ ID NO: 444 | 5 | P1AG9391 | LC | AIQMTQSPSSLSASVGDRVTITCQASYWLS |
| | | | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIKRTVAAPSVFIFPP |
| | | | | SDEQLKSGTASVVCLLNNFYPREAKVQW |
| | | | | KVDNALQSGNSQESVTEQDSKDSTYSLSS |
| | | | | TLTLSKADYEKHKVYACEVTHQGLSSPVT |
| | | | | KSFNRGEC |
| SEQ ID NO: 445 | 6 | P1AH1205 | HC | KVQLVESGGGLVQPGGSLRLSCAASGFK |
| | | | | WEHYAMSWVRQAPGKGLEWVGSISPRG |
| | | | | DHKYYNTKFIDRFTISRDNDKNTLYLQMN |
| | | | | SLRAEDTAVYYCARDWDYFDRWGQGTL |
| | | | | VTVSSASTKGPSVFPLAPSSKSTSGGTAAL |
| | | | | GCLVKDYFPEPVTVSWNSGALTSGVHTFP |
| | | | | AVLQSSGLYSLSSVVTVPSSSLGTQTYICN |
| | | | | VNHKPSNTKVDKKVEPKSCD |
| SEQ ID NO: 446 | 6 | P1AH1205 | LC | AIQMTQSPSSLSASVGDRVTITCQSQFWLS |
| | | | | NYLAWYQQKPGKAPKLLIFDGDLPYERPS |
| | | | | RFSGSGSHDEYTLTISSLQPEDFATYYCQQ |
| | | | | YREEPYTFGQGTKLEIKRTVAAPSVFIFPPS |
| | | | | DEQLKSGTASVVCLLNNFYPREAKVQWK |
| | | | | VDNALQSGNSQESVTEQDSKDSTYSLSST |
| | | | | LTLSKADYEKHKVYACEVTHQGLSSPVT |
| | | | | KSFNRGEC |
| SEQ ID NO: 447 | 7 | P1AH1199 | HC | QVQLVESGGGLVQPGGSLRLSCAASGFKY |
| | | | | DVYAMSWVRQAPGKGLEWVGEISPRGDS |
| | | | | KYYNTKFIDRFTISRDNEKNTLYLQMNSL |
| | | | | RAEDTAVYYCARDWEYFDNWGQGTLVT |
| | | | | VSSASTKGPSVFPLAPSSKSTSGGTAALGC |
| | | | | LVKDYFPEPVTVSWNSGALTSGVHTFPAV |
| | | | | LQSSGLYSLSSVVTVPSSSLGTQTYICNVN |
| | | | | HKPSNTKVDKKVEPKSCD |
| SEQ ID NO: 448 | 7 | P1AH1199 | LC | AIQMTQSPSSLSASVGDRVTITCQASYWLS |
| | | | | NYLAWYQQKPGKAPKLLIFDGDIPWEIPS |
| | | | | RFSGSGSHEEYTLTISSLQPEDFATYYCQQ |
| | | | | YRDTPYTFGQGTKLEIKRTVAAPSVFIFPP |
| | | | | SDEQLKSGTASVVCLLNNFYPREAKVQW |
| | | | | KVDNALQSGNSQESVTEQDSKDSTYSLSS |
| | | | | TLTLSKADYEKHKVYACEVTHQGLSSPVT |
| | | | | KSFNRGEC |
| SEQ ID NO: 449 | 1 | P1AG9426 | HCDR1 | GFKYDVYAMS |
| SEQ ID NO: 450 | 1 | P1AG9426 | HCDR2 | EISPRGDSKYYNTKFID |
| SEQ ID NO: 451 | 1 | P1AG9426 | HCDR3 | DWEYFDN |
| SEQ ID NO: 452 | 1 | P1AG9426 | LCDR1 | QASYWLSNYLA |
| SEQ ID NO: 453 | 1 | P1AG9426 | LCDR2 | DGDIPY |
| SEQ ID NO: 454 | 1 | P1AG9426 | LCDR3 | QQYRETPYT |
| SEQ ID NO: 455 | 2 | P1AG9376 | HCDR1 | GFKWEHYAMS |
| SEQ ID NO: 456 | 2 | P1AG9376 | HCDR2 | SISPRGDHKYYNTKFID |
| SEQ ID NO: 457 | 2 | P1AG9376 | HCDR3 | DWDYFDR |
| SEQ ID NO: 458 | 2 | P1AG9376 | LCDR1 | QSQFWLSNYLA |
| SEQ ID NO: 459 | 2 | P1AG9376 | LCDR2 | DGDLPY |
| SEQ ID NO: 460 | 2 | P1AG9376 | LCDR3 | QQYREEPYT |
| SEQ ID NO: 461 | 3 | P1AG9372 | HCDR1 | GFRFDIYAMS |
| SEQ ID NO: 462 | 3 | P1AG9372 | HCDR2 | EISPDGRTKYYNTKFID |
| SEQ ID NO: 463 | 3 | P1AG9372 | HCDR3 | DWEYFDN |
| SEQ ID NO: 464 | 3 | P1AG9372 | LCDR1 | QSQFWLSNYLA |
| SEQ ID NO: 465 | 3 | P1AG9372 | LCDR2 | DGDLPY |
| SEQ ID NO: 466 | 3 | P1AG9372 | LCDR3 | QQYREEPYT |
| SEQ ID NO: 467 | 4 | P1AG9420 | HCDR1 | GFRYDHYAMS |
| SEQ ID NO: 468 | 4 | P1AG9420 | HCDR2 | SISPRGNHKYYNTKFID |
| SEQ ID NO: 469 | 4 | P1AG9420 | HCDR3 | DWDYFDH |
| SEQ ID NO: 470 | 4 | P1AG9420 | LCDR1 | QASYWLSNYLA |
| SEQ ID NO: 471 | 4 | P1AG9420 | LCDR2 | DGDIPY |
| SEQ ID NO: 472 | 4 | P1AG9420 | LCDR3 | QQYRETPYT |
| SEQ ID NO: 473 | 5 | P1AG9391 | HCDR1 | GFKYDVYAMS |
| SEQ ID NO: 474 | 5 | P1AG9391 | HCDR2 | EISPRGDSKYYNTKFID |
| SEQ ID NO: 475 | 5 | P1AG9391 | HCDR3 | DWEYFDN |
| SEQ ID NO: 476 | 5 | P1AG9391 | LCDR1 | QASYWLSNYLA |
| SEQ ID NO: 477 | 5 | P1AG9391 | LCDR2 | DGDIPW |
| SEQ ID NO: 478 | 5 | P1AG9391 | LCDR3 | QQYRDTPYT |
| SEQ ID NO: 479 | 6 | P1AH1205 | HCDR1 | GFKWEHYAMS |
| SEQ ID NO: 480 | 6 | P1AH1205 | HCDR2 | SISPRGDHKYYNTKFID |
| SEQ ID NO: 481 | 6 | P1AH1205 | HCDR3 | DWDYFDR |
| SEQ ID NO: 482 | 6 | P1AH1205 | LCDR1 | QSQFWLSNYLA |
| SEQ ID NO: 483 | 6 | P1AH1205 | LCDR2 | DGDLPY |
| SEQ ID NO: 484 | 6 | P1AH1205 | LCDR3 | QQYREEPYT |
| SEQ ID NO: 485 | 7 | P1AH1199 | HCDR1 | GFKYDVYAMS |
| SEQ ID NO: 486 | 7 | P1AH1199 | HCDR2 | EISPRGDSKYYNTKFID |
| SEQ ID NO: 487 | 7 | P1AH1199 | HCDR3 | DWEYFDN |
| SEQ ID NO: 488 | 7 | P1AH1199 | LCDR1 | QASYWLSNYLA |
| SEQ ID NO: 489 | 7 | P1AH1199 | LCDR2 | DGDIPW |
| SEQ ID NO: 490 | 7 | P1AH1199 | LCDR3 | QQYRDTPYT |
| SEQ ID NO: 491 | | human FactorB | | HHHHHHGSLEVLFQGPGGTPWSLARPQG |
| | | wt (FB) with | | SCSLEGVEIKGGSFRLLQEGQALEYVCPSG |
| | | His Tag and | | FYPYPVQTRTCRSTGSWSTLKTQDQKTVR |
| | | Precission site | | KAECRAIHCPRPHDFENGEYWPRSPYYNV |
| | | | | SDEISFHCYDGYTLRGSANRTCQVNGRWS |
| | | | | GQTAICDNGAGYCSNPGIPIGTRKVGSQY |
| | | | | RLEDSVTYHCSRGLTLRGSQRRTCQEGGS |
| | | | | WSGTEPSCQDSFMYDTPQEVAEAFLSSLT |
| | | | | ETIEGVDAEDGHGPGEQQKRKIVLDPSGS |
| | | | | MNIYLVLDGSDSIGASNFTGAKKCLVNLIE |
| | | | | KVASYGVKPRYGLVTYATYPKIWVKVSE |
| | | | | ADSSNADWVTKQLNEINYEDHKLKSGTN |
| | | | | TKKALQAVYSMMSWPDDVPPEGWNRTR |
| | | | | HVIILMTDGLHNMGGDPITVIDEIRDLLYI |
| | | | | GKDRKNPREDYLDVYVFGVGPLVNQVNI |
| | | | | NALASKKDNEQHVFKVKDMENLEDVFY |
| | | | | QMIDESQSLSLCGMVWEHRKGTDYHKQP |
| | | | | WQAKISVIRPSKGHESCMGAVVSEYFVLT |
| | | | | AAHCFTVDDKEHSIKVSVGGEKRDLEIEV |
| | | | | VLFHPNYNINGKKEAGIPEFYDYDVALIKL |
| | | | | KNKLKYGQTIRPICLPCTEGTTRALRLPPT |
| | | | | TTCQQQKEELLPAQDIKALFVSEEEKKLT |
| | | | | RKEVYIKNGDKKGSCERDAQYAPGYDKV |
| | | | | KDISEVVTPRFLCTGGVSPYADPNTCRGD |
| | | | | SGGPLIVHKRSRFIQVGVISWGVVDVCKN |
| | | | | QKRQKQVPAHARDFHINLFQVLPWLKEK |
| | | | | LQDEDLGFL |
| SEQ ID NO: 492 | | human FB with | | HHHHHHGSLEVLFQGPGGTPWSLARPQG |
| | | D279G with His | | SCSLEGVEIKGGSFRLLQEGQALEYVCPSG |
| | | Tag and | | FYPYPVQTRTCRSTGSWSTLKTQDQKTVR |
| | | Precission site | | KAECRAIHCPRPHDFENGEYWPRSPYYNV |
| | | | | SDEISFHCYDGYTLRGSANRTCQVNGRWS |
| | | | | GQTAICDNGAGYCSNPGIPIGTRKVGSQY |
| | | | | RLEDSVTYHCSRGLTLRGSQRRTCQEGGS |
| | | | | WSGTEPSCQDSFMYDTPQEVAEAFLSSLT |
| | | | | ETIEGVDAEDGHGPGEQQKRKIVLDPSGS |
| | | | | MNIYLVLDGSGSIGASNFTGAKKCLVNLIE |
| | | | | KVASYGVKPRYGLVTYATYPKIWVKVSE |
| | | | | ADSSNADWVTKQLNEINYEDHKLKSGTN |
| | | | | TKKALQAVYSMMSWPDDVPPEGWNRTR |
| | | | | HVIILMTDGLHNMGGDPITVIDEIRDLLYI |
| | | | | GKDRKNPREDYLDVYVFGVGPLVNQVNI |
| | | | | NALASKKDNEQHVFKVKDMENLEDVFY |
| | | | | QMIDESQSLSLCGMVWEHRKGTDYHKQP |
| | | | | WQAKISVIRPSKGHESCMGAVVSEYFVLT |
| | | | | AAHCFTVDDKEHSIKVSVGGEKRDLEIEV |
| | | | | VLFHPNYNINGKKEAGIPEFYDYDVALIKL |
| | | | | KNKLKYGQTIRPICLPCTEGTTRALRLPPT |
| | | | | TTCQQQKEELLPAQDIKALFVSEEEKKLT |
| | | | | RKEVYIKNGDKKGSCERDAQYAPGYDKV |
| | | | | KDISEVVTPRFLCTGGVSPYADPNTCRGD |
| | | | | SGGPLIVHKRSRFIQVGVISWGVVDVCKN |
| | | | | QKRQKQVPAHARDFHINLFQVLPWLKEK |
| | | | | LQDEDLGFL |
| SEQ ID NO: 493 | | human FB with | | HHHHHHGSLEVLFQGPGGTPWSLARPQG |
| | | D279G, K350N | | SCSLEGVEIKGGSFRLLQEGQALEYVCPSG |
| | | and M458I with | | FYPYPVQTRTCRSTGSWSTLKTQDQKTVR |
| | | His Tag and | | KAECRAIHCPRPHDFENGEYWPRSPYYNV |
| | | Precission site | | SDEISFHCYDGYTLRGSANRTCQVNGRWS |
| | | | | GQTAICDNGAGYCSNPGIPIGTRKVGSQY |
| | | | | RLEDSVTYHCSRGLTLRGSQRRTCQEGGS |
| | | | | WSGTEPSCQDSFMYDTPQEVAEAFLSSLT |
| | | | | ETIEGVDAEDGHGPGEQQKRKIVLDPSGS |
| | | | | MNIYLVLDGSGSIGASNFTGAKKCLVNLIE |
| | | | | KVASYGVKPRYGLVTYATYPKIWVKVSE |
| | | | | ADSSNADWVTKQLNEINYEDHKLNSGTN |
| | | | | TKKALQAVYSMMSWPDDVPPEGWNRTR |
| | | | | HVIILMTDGLHNMGGDPITVIDEIRDLLYI |
| | | | | GKDRKNPREDYLDVYVFGVGPLVNQVNI |
| | | | | NALASKKDNEQHVFKVKDIENLEDVFYQ |
| | | | | MIDESQSLSLCGMVWEHRKGTDYHKQPW |
| | | | | QAKISVIRPSKGHESCMGAVVSEYFVLTA |
| | | | | AHCFTVDDKEHSIKVSVGGEKRDLEIEVV |
| | | | | LFHPNYNINGKKEAGIPEFYDYDVALIKLK |
| | | | | NKLKYGQTIRPICLPCTEGTTRALRLPPTT |
| | | | | TCQQQKEELLPAQDIKALFVSEEEKKLTR |
| | | | | KEVYIKNGDKKGSCERDAQYAPGYDKVK |
| | | | | DISEVVTPRFLCTGGVSPYADPNTCRGDS |
| | | | | GGPLIVHKRSRFIQVGVISWGVVDVCKNQ |
| | | | | KRQKQVPAHARDFHINLFQVLPWLKEKL |
| | | | | QDEDLGFL |
| SEQ ID NO: 494 | | Complement | | ILGGREAEAHARPYMASVQLNGAHLCGG |
| | | Factor D | | VLVAEQWVLSAAHCLEDAADGKVQVLL |
| | | | | GAHSLSQPEPSKRLYDVLRAVPHPDSQPD |
| | | | | TIDHDLLLLQLSEKATLGPAVRPLPWQRV |
| | | | | DRDVAPGTLCDVAGWGIVNHAGRRPDSL |
| | | | | QHVLLPVLDRATCNRRTHHDGAITERLM |
| | | | | CAESNRRDSCKGDSGGPLVCGGVLEGVV |
| | | | | TSGSRVCGNRKKPGIYTRVASYAAWIDSV |
| | | | | L |
| SEQ ID NO: 495 | | Complement C3 | | SPMYSIITPNILRLESEETMVLEAHDAQGD |
| | | beta chain (C3b) | | VPVTVTVHDFPGKKLVLSSEKTVLTPATN |
| | | | | HMGNVTFTIPANREFKSEKGRNKFVTVQA |
| | | | | TFGTQVVEKVVLVSLQSGYLFIQTDKTIYT |
| | | | | PGSTVLYRIFTVNHKLLPVGRTVMVNIEN |
| | | | | PEGIPVKQDSLSSQNQLGVLPLSWDIPELV |
| | | | | NMGQWKIRAYYENSPQQVFSTEFEVKEY |
| | | | | VLPSFEVIVEPTEKFYYIYNEKGLEVTITAR |
| | | | | FLYGKKVEGTAFVIFGIQDGEQRISLPESL |
| | | | | KRIPIEDGSGEVVLSRKVLLDGVQNPRAE |
| | | | | DLVGKSLYVSATVILHSGSDMVQAERSGI |
| | | | | PIVTSPYQIHFTKTPKYFKPGMPFDLMVFV |
| | | | | TNPDGSPAYRVPVAVQGEDTVQSLTQGD |
| | | | | GVAKLSINTHPSQKPLSITVRTKKQELSEA |
| | | | | EQATRTMQALPYSTVGNSNNYLHLSVLR |
| | | | | TELRPGETLNVNFLLRMDRAHEAKIRYYT |
| | | | | YLIMNKGRLLKAGRQVREPGQDLVVLPLS |
| | | | | ITTDFIPSFRLVAYYTLIGASGQREVVADS |
| | | | | VWVDVKDSCVGSLVVKSGQSEDRQPVPG |
| | | | | QQMTLKIEGDHGARVVLVAVDKGVFVLN |
| | | | | KKNKLTQSKIWDVVEKADIGCTPGSGKD |
| | | | | YAGVFSDAGLTFTSSSGQQTAQRAELQCP |
| | | | | QPAARRRRSVQLTEKRMDKVGKYPKELR |
| | | | | KCCEDGMRENPMRFSCQRRTRFISLGEAC |
| | | | | KKVFLDCCNYITELRRQHARASHLGLARS |
| | | | | NLDEDIIAEENIVSRSEFPESWLWNVEDLK |
| | | | | EPPKNGISTKLMNIFLKDSITTWEILAVSM |
| | | | | SDKKGICVADPFEVTVMQDFFIDLRLPYS |
| | | | | VVRNEQVEIRAVLYNYRQNQELKVRVEL |
| | | | | LHNPAFCSLATTKRRHQQTVTIPPKSSLSV |
| | | | | PYVIVPLKTGLQEVEVKAAVYHHFISDGV |
| | | | | RKSLKVVPEGIRMNKTVAVRTLDPERLGR |
| | | | | EGVQKEDIPPADLSDQVPDTESETRILLQG |
| | | | | TPVAQMTEDAVDAERLKHLIVTPSGCGEQ |
| | | | | NMIGMTPTVIAVHYLDETEQWEKFGLEK |
| | | | | RQGALELIKKGYTQQLAFRQPSSAFAAFV |
| | | | | KRAPSTWLTAYVVKVFSLAVNLIAIDSQV |
| | | | | LCGAVKWLILEKQKPDGVFQEDAPVIHQE |
| | | | | MIGGLRNNNEKDMALTAFVLISLQEAKDI |
| | | | | CEEQVNSLPGSITKAGDFLEANYMNLQRS |
| | | | | YTVAIAGYALAQMGRLKGPLLNKFLTTA |
| | | | | KDKNRWEDPGKQLYNVEATSYALLALLQ |
| | | | | LKDFDFVPPVVRWLNEQRYYGGGYGSTQ |
| | | | | ATFMVFQALAQYQKDAPDHQELNLDVSL |
| | | | | QLPSRSSKITHRIHWESASLLRSEETKENE |
| | | | | GFTVTAEGKGQGTLSVVTMYHAKAKDQL |
| | | | | TCNKFDLKVTIKPAPETEKRPQDAKNTMI |
| | | | | LEICTRYRGDQDATMSILDISMMTGFAPD |
| | | | | TDDLKQLANGVDRYISKYELDKAFSDRNT |
| | | | | LIIYLDKVSHSEDDCLAFKVHQYFNVELIQ |
| | | | | PGAVKVYAYYNLEESCTRFYHPEKEDGK |
| | | | | LNKLCRDELCRCAEENCFIQKSDDKVTLE |
| | | | | ERLDKACEPGVDYVYKTRLVKVQLSNDF |
| | | | | DEYIMAIEQTIKSGSDEVQVGQQRTFISPIK |
| | | | | CREALKLEEKKHYLMWGLSSDFWGEKPN |
| | | | | LSYIIGKDTWVEHWPEEDECQDEENQKQC |
| | | | | QDLGAFTESMVVFGCPN |
| SEQ ID NO: 496 | | human C3a | | SVQLTEKRMDKVGKYPKELRKCCEDGMR |
| | | | | ENPMRFSCQRRTRFISLGEACKKVFLDCC |
| | | | | NYITELRRQHARASHLGLAR |
| SEQ ID NO: 497 | | Cynomolgus | | PMYSMITPNVLRLESEETVVLEAHDANGD |
| | | C3b | | VPVTVTVHDFPGKKLVLSSEKTVLTPATS |
| | | | | HMGSVTIRIPANKEFKSEKGHNKFVTVQA |
| | | | | TFGAQVVEKVVLVSLQSGYLFIQTDKTIY |
| | | | | TPGSTVLCRIFTVNHKLLPVGRTVVVNIEN |
| | | | | PDGIPVKQDSLSSQNQFGILPLSWDIPELV |
| | | | | NMGQWKIRAYYENSPQQVFSTEFEVKEY |
| | | | | VLPSFEVIVEPTEKFYYIYNQKGLEVTITA |
| | | | | RFLYGKKVEGTAFVIFGIQDGEQRISLPES |
| | | | | LKRIQIEDGSGDAVLSRKVLLDGVQNPRP |
| | | | | EDLVGKSLYVSVTVILHSGSDMVQAERSG |
| | | | | IPIVTSPYQIHFTKTPKYFKPGMPFDLMVF |
| | | | | VTNPDGSPAYRVPVAVQGEDAVQSLTQG |
| | | | | DGVAKLSINTHPSQKPLSITVRTKKRELSE |
| | | | | AEQATRTMEAQPYSTVGNSNNYLHLSVP |
| | | | | RAELRPGETLNVNFLLRMDRTQEAKIRYY |
| | | | | TYLIMNKGKLLKVGRQVREPGQDLVVLP |
| | | | | LSITTDFIPSFRLVAYYTLIGANGQREVVA |
| | | | | DSVWVDVKDSCVGSLVVKSGQSEDRQPL |
| | | | | PGQQMTLKIEGDHGARVGLVAVDKGVFV |
| | | | | LNKKNKLTQSKIWDVVEKADIGCTPGSGK |
| | | | | DYAGVFSDAGLTFASSSGQQTAQRAELQC |
| | | | | PQPATRRRRSVQLAEKRMDKVGQYPKEL |
| | | | | RKCCEHGMRENPMRFSCQRRTRYITLDEA |
| | | | | CKKAFLDCCNYITELRRQHARASHLGLAR |
| | | | | SNLDEDIIAEENIVSRSEFPESWLWKIEELK |
| | | | | EAPKNGISTKLMNIFLKDSITTWEILAVSLS |
| | | | | DKKGICVADPFEVTVMQDFFIDLRLPYSV |
| | | | | VRNEQVEIRAVLYNYRQNQELKVRVELL |
| | | | | HNPAFCSLATAKRRHQQTVTIPPKSSLSVP |
| | | | | YVIVPLKTGQQEVEVKAAVYHFFISDGVR |
| | | | | KSLKVVPEGIRMNKTVAVRTLDPERLGQE |
| | | | | GVQREDVPPADLSDQVPDTESETRILLQG |
| | | | | TPVAQMTEDAIDAERLKHLIVTPSGCGEQ |
| | | | | NMITMTPTVIAVHYLDETEQWEKFGPEKR |
| | | | | QGALELIKKGYTQQLAFRQPSSAFAAFLN |
| | | | | RAPSTWLTAYVVKVFSLAVNLIAIDSQVL |
| | | | | CGAVKWLILEKQKPDGVFQEDAPVIHQE |
| | | | | MTGGFRNTNEKDMALTAFVLISLQEAKEI |
| | | | | CEEQVNSLPGSITKAGDFLEANYMNLQRS |
| | | | | YTVAIAAYALAQMGRLKGPLLNKFLTTA |
| | | | | KDKNRWEEPGQQLYNVEATSYALLALLQ |
| | | | | LKDFDFVPPVVRWLNEQRYYGGGYGSTQ |
| | | | | ATFMVFQALAQYQKDVPDHKELNLDVSL |
| | | | | QLPSRSSKIIHRIHWESASLLRSEETKENEG |
| | | | | FTVTAEGKGQGTLSVVTMYHAKAKGQLT |
| | | | | CNKFDLKVTIKPAPETEKRPQDAKNTMIL |
| | | | | EICTRYRGDQDATMSILDISMMTGFVPDT |
| | | | | DDLKQLANGVDRYISKYELDKAFSDRNTL |
| | | | | IIYLDKVSHSEDDCIAFKVHQYFNVELIQP |
| | | | | GAVKVYAYYNLAESCTRFYHPEKEDGKL |
| | | | | NKLCRDELCRCAEENCFIQKLDDKVTLEE |
| | | | | RLDKACEPGVDYVYKTRLVKAQLSNDFD |
| | | | | EYIMAIEQIIKSGSDEVQVGQQRTFISPIKC |
| | | | | REALKLEERKHYLMWGLSSDFWGEKPNL |
| | | | | SYIIGKDTWVEHWPEEDECQDEENQKQC |
| | | | | QDLGTFTENMVVFGCPN |
| SEQ ID NO: 498 | | Cynomolgus | | ILGGREAEAHARPYMASVQVNGEHLCGG |
| | | Factor D (FD) | | VLVAEQWVLSAAHCLEDAADGKVQVLL |
| | | | | GAHSLSQPEPSKRLYDVLRAVPHPDSRPD |
| | | | | TIDHDLLLLQLSEKATLGPAVRPLPWQRV |
| | | | | DRDVEPGTLCDVAGWGIVSHAGRRPDRL |
| | | | | QHVLLPVLDRATCNRRTHHDGAITQRMM |
| | | | | CAESNRRDSCKGDSGGPLVCGGVLEGVV |
| | | | | TSGSRVCGNRKKPGIYTRVASYAAWIDSL |
| | | | | EVLFQGPGSHHHHHH |
| SEQ ID NO: 499 | | Cynomolgus | | HHHHHHGSLEVLFQGPGGTPLSSAQPQGS |
| | | Factor B WT | | CSLEGVEIKGGSFRLLQEGQALEYVCPSGF |
| | | | | YPYPVQTRTCRSTGSWSTLQTQDRKTVK |
| | | | | KAECRAIRCPRPQDFENGEYRPRSPYYNV |
| | | | | SDEISFHCYDGYTLRGSANRTCQVNGRWS |
| | | | | GQTAICDNGAGYCSNPGIPIGTRKVGSRY |
| | | | | RLEDSVTYHCSRGLTLRGSQRRTCQEGGS |
| | | | | WSGTEPSCQDSFMYDTPQEVAEAFLSSLT |
| | | | | ETIEGVDAEDGHSPGEQQKRRIILDPSGSM |
| | | | | NIYLVLDGSDSIGAGNFTGAKKCLVNLIEK |
| | | | | VASYGVKPRYALVTYATYPRIWVKVSDQ |
| | | | | ESSNADWVTKKLSEINYEDHKLKSGTNTK |
| | | | | RALQAVYSMMSWPEDIPPEGWNRTRHVII |
| | | | | LMTDGLHNMGGDPITVIDEIRDLLYIGKD |
| | | | | RKNPREDYLDVYVFGVGPLVDQVNINAL |
| | | | | ASKKDNEQHVFKVKDMENLEDVFFQMID |
| | | | | ESQSLSLCGMVWEHTTGTDYHKQPWQA |
| | | | | KISVTRPSKGHESCMGAVVSEYFVLTAAH |
| | | | | CFTVDDKEHSIKVSVGKKRDLEIEKVLFHP |
| | | | | DYNISGKKEAGIPEFYDYDVALIKLKKKL |
| | | | | NYDPTIRPICLPCTEGTTRALRLPPTTTCQQ |
| | | | | QKEELLPAQDIKALFVSEEEKKLTRKEVYI |
| | | | | KNGDKKGSCERDAQYAPGYDKVKDISEV |
| | | | | VTPRFLCTGGVSPYADPNTCRGDSGGPLI |
| | | | | VHKRSRFIQVGVISWGVVDVCKNQKRQK |
| | | | | QVPAHARDFHVNLFQVLPWLKEKLQDED |
| | | | | LGFL |
| | | | | HHHHHHGSLEVLFQGPGGTPLSSAQPQGS |
| SEQ ID NO: 500 | | Cynomolgus | | CSLEGVEIKGGSFRLLQEGQALEYVCPSGF |
| | | Factor B triple | | YPYPVQTRTCRSTGSWSTLQTQDRKTVK |
| | | mutant | | KAECRAIRCPRPQDFENGEYRPRSPYYNV |
| | | (D279G_K350N_ | | SDEISFHCYDGYTLRGSANRTCQVNGRWS |
| | | M458I) | | GQTAICDNGAGYCSNPGIPIGTRKVGSRY |
| | | | | RLEDSVTYHCSRGLTLRGSQRRTCQEGGS |
| | | | | WSGTEPSCQDSFMYDTPQEVAEAFLSSLT |
| | | | | ETIEGVDAEDGHSPGEQQKRRIILDPSGSM |
| | | | | NIYLVLDGSGSIGAGNFTGAKKCLVNLIEK |
| | | | | VASYGVKPRYALVTYATYPRIWVKVSDQ |
| | | | | ESSNADWVTKKLSEINYEDHKLNSGTNTK |
| | | | | RALQAVYSMMSWPEDIPPEGWNRTRHVII |
| | | | | LMTDGLHNMGGDPITVIDEIRDLLYIGKD |
| | | | | RKNPREDYLDVYVFGVGPLVDQVNINAL |
| | | | | ASKKDNEQHVFKVKDIENLEDVFFQMIDE |
| | | | | SQSLSLCGMVWEHTTGTDYHKQPWQAKI |
| | | | | SVTRPSKGHESCMGAVVSEYFVLTAAHCF |
| | | | | TVDDKEHSIKVSVGKKRDLEIEKVLFHPD |
| | | | | YNISGKKEAGIPEFYDYDVALIKLKKKLN |
| | | | | YDPTIRPICLPCTEGTTRALRLPPTTTCQQQ |
| | | | | KEELLPAQDIKALFVSEEEKKLTRKEVYIK |
| | | | | NGDKKGSCERDAQYAPGYDKVKDISEVV |
| | | | | TPRFLCTGGVSPYADPNTCRGDSGGPLIV |
| | | | | HKRSRFIQVGVISWGVVDVCKNQKRQKQ |
| | | | | VPAHARDFHVNLFQVLPWLKEKLQDEDL |
| | | | | GFL |
| SEQ ID NO: 501 | | Human Factor B | | TPWSLARPQGSCSLEGVEIKGGSFRLLQEG |
| | | (wt, without | | QALEYVCPSGFYPYPVQTRTCRSTGSWST |
| | | signal peptide) | | LKTQDQKTVRKAECRAIHCPRPHDFENGE |
| | | | | YWPRSPYYNVSDEISFHCYDGYTLRGSAN |
| | | | | RTCQVNGRWSGQTAICDNGAGYCSNPGIP |
| | | | | IGTRKVGSQYRLEDSVTYHCSRGLTLRGS |
| | | | | QRRTCQEGGSWSGTEPSCQDSFMYDTPQE |
| | | | | VAEAFLSSLTETIEGVDAEDGHGPGEQQK |
| | | | | RKIVLDPSGSMNIYLVLDGSDSIGASNFTG |
| | | | | AKKCLVNLIEKVASYGVKPRYGLVTYAT |
| | | | | YPKIWVKVSEADSSNADWVTKQLNEINY |
| | | | | EDHKLKSGTNTKKALQAVYSMMSWPDD |
| | | | | VPPEGWNRTRHVIILMTDGLHNMGGDPIT |
| | | | | VIDEIRDLLYIGKDRKNPREDYLDVYVFG |
| | | | | VGPLVNQVNINALASKKDNEQHVFKVKD |
| | | | | MENLEDVFYQMIDESQSLSLCGMVWEHR |
| | | | | KGTDYHKQPWQAKISVIRPSKGHESCMG |
| | | | | AVVSEYFVLTAAHCFTVDDKEHSIKVSVG |
| | | | | GEKRDLEIEVVLFHPNYNINGKKEAGIPEF |
| | | | | YDYDVALIKLKNKLKYGQTIRPICLPCTEG |
| | | | | TTRALRLPPTTTCQQQKEELLPAQDIKALF |
| | | | | VSEEEKKLTRKEVYIKNGDKKGSCERDAQ |
| | | | | YAPGYDKVKDISEVVTPRFLCTGGVSPYA |
| | | | | DPNTCRGDSGGPLIVHKRSRFIQVGVISWG |
| | | | | VVDVCKNQKRQKQVPAHARDFHINLFQV |
| | | | | LPWLKEKLQDEDLGFL |
| SEQ ID NO: 502 | | human constant | | RTVAAPSVFIFPPSDEQLKSGTASVVCLLN |
| | | light chain | | NFYPREAKVQWKVDNALQSGNSQESVTE |
| | | domain CL | | QDSKDSTYSLSSTLTLSKADYEKHKVYAC |
| | | | | EVTHQGLSSPVTKSFNRGEC |
| SEQ ID NO: 503 | | human constant | | ASTKGPSVFPLAPSSKSTSGGTAALGCLVK |
| | | heavy chain | | DYFPEPVTVSWNSGALTSGVHTFPAVLQS |
| | | domain CH1 | | SGLYSLSSVVTVPSSSLGTQTYICNVNHKP |
| | | | | SNTKVDKKVEPKSCD |
| SEQ ID NO: 504 | | Human Factor B | | MGSNLSPQLCLMPFILGLLSGGVTTTPWS |
| | | (wt, with signal | | LARPQGSCSLEGVEIKGGSFRLLQEGQALE |
| | | peptide) | | YVCPSGFYPYPVQTRTCRSTGSWSTLKTQ |
| | | | | DQKTVRKAECRAIHCPRPHDFENGEYWP |
| | | | | RSPYYNVSDEISFHCYDGYTLRGSANRTC |
| | | | | QVNGRWSGQTAICDNGAGYCSNPGIPIGT |
| | | | | RKVGSQYRLEDSVTYHCSRGLTLRGSQRR |
| | | | | TCQEGGSWSGTEPSCQDSFMYDTPQEVAE |
| | | | | AFLSSLTETIEGVDAEDGHGPGEQQKRKIV |
| | | | | LDPSGSMNIYLVLDGSDSIGASNFTGAKK |
| | | | | CLVNLIEKVASYGVKPRYGLVTYATYPKI |
| | | | | WVKVSEADSSNADWVTKQLNEINYEDHK |
| | | | | LKSGTNTKKALQAVYSMMSWPDDVPPEG |
| | | | | WNRTRHVIILMTDGLHNMGGDPITVIDEIR |
| | | | | DLLYIGKDRKNPREDYLDVYVFGVGPLV |
| | | | | NQVNINALASKKDNEQHVFKVKDMENLE |
| | | | | DVFYQMIDESQSLSLCGMVWEHRKGTDY |
| | | | | HKQPWQAKISVIRPSKGHESCMGAVVSEY |
| | | | | FVLTAAHCFTVDDKEHSIKVSVGGEKRDL |
| | | | | EIEVVLFHPNYNINGKKEAGIPEFYDYDVA |
| | | | | LIKLKNKLKYGQTIRPICLPCTEGTTRALR |
| | | | | LPPTTTCQQQKEELLPAQDIKALFVSEEEK |
| | | | | KLTRKEVYIKNGDKKGSCERDAQYAPGY |
| | | | | DKVKDISEVVTPRFLCTGGVSPYADPNTC |
| | | | | RGDSGGPLIVHKRSRFIQVGVISWGVVDV |
| | | | | CKNQKRQKQVPAHARDFHINLFQVLPWL |
| | | | | KEKLQDEDLGFL |

### EXAMPLES

The following examples are provided to aid the understanding of the present invention, the true scope of which is set forth in the appended claims. It is understood that modifications can be made in the procedures set forth without departing from the spirit of the invention.

### Example 1

### Stabilization of Human C3bBb Complex

Wild-type C3bBb is a rapidly decaying complex unsuitable for antibody discovery purposes. In order to assess the influence of different mutations within the factor B (FB) subunit on the stability of C3bBb the following mutations were introduced in the FB protein: D279G, K350N and M458I. Unmutated FB is indicated in SEQ ID NO: 491, FB with a D279G mutation is indicated in SEQ ID NO: 492 and FB with D279G, K350N and M458I mutations is indicated in SEQ ID NO: 493. Numbering of the mutations is based on the amino acid sequence of human Factor B with signal peptide (SEQ ID NO: 504). Assembly and decay of the different described C3bBb variants was assessed by Bio Layer Interferometry (BLI) analysis on a sensor coated with C3b.

Stability Detection with BLI:

### a) C3bBb Assembly in Presence of Mg-Ions

To analyze stability of the so formed C3bBb complexes having the different Bb subunits, C3b was biotinylated at cysteine 1010 by incubating plasma purified C3b (Complement Technology) overnight at 4° C. in presence of 5 molar excess Maleimide-PEG3-biotin (Thermo Fisher Scientific) followed by purification on a Zeba spin column 40K (Thermo Fisher Scientific). This material was used to load streptavidin functionalized BLI sensors (SA sensors; Fortebio) in an Octet RED 384 device (Fortebio) at high density (5 nm BLI signal). C3bBb was assembled by exposing the C3b loaded sensors to different recombinant factor B variants (i.e. unmutated SEQ ID NO: 491; D279G variant of SEQ ID NO: 492; D279G K350N M458I triple variant of SEQ ID NO: 493) in presence of 54 nM plasma purified factor D (Complement Technology) in HBS-T(Mg) buffer (150 mM NaCl, 10 mM HEPES, 1 mM MgCl₂, 0.005% Tween20. pH 7.4) at 22° C. After allowing the convertase to reach equilibrium for 12 minutes, sensors were transferred to 200 pl HBS-T(Mg) buffer initiating dissociation of the assembled C3bBb. Convertase decay was followed for three hours.

After subtraction of the signal recorded from parallel reference sensors omitting the C3b loading step and subtraction of a control sensor omitting factor B and factor D, data of the dissociation phase was fitted to the double exponential ((A*exp(((−1)*B)*x)))+(C*exp(((−1)*D)*x)) using XLfit (IDBS software), allowing for heterogeneity in the assembled convertase population. Half life (=LN(2)/B) and relative importance of the main species are reported in Table E1.

### b) C3bBb Assembly in Presence of Ni-Ions

In another experiment, the structural Mg²⁺ ion coordinated in factor B VWA was substituted for Ni²⁺ in order to further stabilize the C3 convertase. The impact on C3bBb complex stability was explored using a setup that was identical to the precedent experiment, except that HBS-T(Ni) buffer (150 mM NaCl, 10 mM HEPES, 1 mM NiCl₂, 0.005% Tween20. pH 7.4) was used instead of HBS-T(Mg).

Correction of the BLI trace was identical to the experiment in presence of magnesium. Different from the Mg²⁺ containing C3bBb complexes, only one species was detected during dissociation and a single exponential decay (A*exp(((−1)*B)*x)) was used for analysis. The influence of the addition of Ni²⁺ ions during assembly of C3bBb using the different Bb proteins, i.e. wild type Bb, single mutated Bb D279G and triple mutated Bb D279G, K350N and M458I for the formation of the C3bBb complex are shown in Table E1.

TABLE E1
t½ of C3bBb complex formed using different
Bb proteins as measured by BLI

| | | prevalence of main |
|---|---|---|
| Bb comprised in C3bBb complex | t ½ | species |
| wt FB, Mg²⁺ | 4 min 46 s | 93% |
| FB (D279G), Mg²⁺ | 1 h 03 min | 97% |
| FB (D279G, K350N and M458I), Mg²⁺ | 1 h 59 min | 93% |
| wt FB, Ni²⁺ | 0 h 29 min | 100% |
| FB (D279G), Ni²⁺ | 3 h 18 min | 100% |
| FB (D279G, K350N and M458I), Ni²⁺ | 11 h 18 min | 100% |

While wild type C3bBb complex formed in the presence of magnesium ions had a half-life of less than five minutes, stabilization by mutations within the Bb subunit of FB and further stabilizing C3bBb by formation in presence of Ni²⁺ ions results in a half life of up to 11 hours and 15 minutes (equaling 675 minutes, which equals a more than 135-fold increase in C3bBb half-life).

### Assembly and Purification of Stabilized C3bBb Complex for Antibody Discovery

C3b was biotinylated at cysteine 1010 by incubating plasma purified C3b (Complement Technology) 2 h at room temperature in presence of 0.4 mM EZ-Link HPDP-Biotin (Thermo Fisher Scientific) followed by purification on a NAP-10 Column (Thermo Fisher Scientific). The biotinylated C3b was mixed in a molar ratio of 1:1 with hu Factor B (i.e. unmutated SEQ ID NO: 491; D279G variant of SEQ ID NO: 492; D279G K350N M458I triple variant of SEQ ID NO: 493) additional added NiSO₄ to a final concentration of 5 mM. After 10 minutes of pre incubation at room temperature FactorD (Complement Technology) added in a molar ratio of 1 (C3b): 1 (Factor B): 0.02 (Factor D) and incubated for another 10 min at room temperature in the reaction buffer (20 mM Hepes, 150 mM NaCl, 5 mM NiSO₄ pH 7.4). The purification by size exclusion chromatography Superdex200 (Cytavia) follows immediately at room temperature in storage buffer (20 mM Hepes, 150 mM NaCl, 1 mM NiSO₄ pH 7.4).

### Example 2

### Antibody Screening Using Stabilized C3bBb Complex

Antibodies specifically binding to the stabilized C3bBb complex were screened as follows:

### Panning

For naïve selection, phage library panning was performed in 4 rounds, wherein the first round was performed with 800 nM of biotinylated C3bBb triple mutant-HPDP (wherein the C3bBb was formed in the presence of nickel ions using FB of SEQ ID NO: 493) pre-immobilized on Dynabeads M-280 Streptavidin (Thermofisher catalog number 11206D). Panning rounds 2-4 were performed with 150 nM of biotinylated target in solution, followed by capture of Fab-on-phage/target complexes on SpeedBead Magnetic Neutravidin Coated Particles (Cytiva, catalog number 78152104010350, round 2 and 4) or Dynabeads M-280 Streptavidin (round 3). In brief, in the first panning round the phage/target/bead complex were washed 2× with HBS-NT+NiSO₄ (0.01M HEPES pH7.4; 0.15M NaCl; Cytiva catalog BR-1006-70 supplemented with 0.1% Tween-20 and 1 mM NiSO4) and 1× with HBS-N+NiSO₄ (0.01M HEPES pH7.4; 0.15M NaCl; Cytiva catalog BR-1006-70 supplemented with 1 mM NiSO₄). In panning rounds 2, 3 and 4 the phage/target/bead complex was washed 5× with HBS-NT+NiSO₄ and 2× with 1×HBS-N+NiSO₄. Captured phage clones bearing target-specific Fabs were eluted from the M-280 beads using 100 mM DTT, used for infection of log-phase TG1 E. coli cells, and rescued using M13 K07 helper phage, according to standard protocols.

### Screening

For screening of selection outputs, a polyclonal plasmid miniprep of the respective selection round was prepared from the infected TG1 E. coli cells. Plasmids were reformatted to produce soluble Fab in E. coli supernatants with a T7 tag at the C-terminus of the Fab CH1 domain. The ligated polyclonal plasmids encoding T7-tagged Fabs were transformed into TG1 E. coli cells (Zymo Research catalog number T3017), and single colonies were picked into microtiter plates. Soluble Fabs were expressed in microtiter plates and supernatants were clarified by centrifugation.

Binding of the antibodies to C3bBb was analyzed by different ELISA assays to analyze binding towards C3bBb formed from Factor B with three mutations D279G, K350N and M458I (herein referred to as “C3bBb triple mutant Elisa”) as well as controls assessing binding to different related components of the complement system, i.e. C3, C3a, C3b and Factor B, Ba, Bb. Briefly, the following protocols were used:

### C3bBb Triple Mutant Elisa

### Buffer:

50 mM Hepes pH 7.4 containing 150 mM NaCl, 0.5% BSA and 0.1% Tween20 was used as diluent for the ELISA. PBS containing 0.1% Tween20 was used as a washing buffer.

### Method:

20 μl of a mixture of 500 ng/ml human C3bBb-Human Factor B Triple mutant (D279G_M350N_M458I)-Cys-HPDP-Biotin, 1:3000 Mouse anti Human kappa HRP (Southern Biotek 9230-05) and 1:2000 Mouse IgG anti T7 Tag HRP (Novagen 69048-3) was added to a Microcoat Streptavidine coated 384 well Microplate (Microcoat 11974998001). 25 pl sample was added to the plate and incubated at room temperature for 1 hour. After incubation plates were washed with 90 pl/well washing buffer using a Biotek EL406 Washer and 30 pl TMB (3,3′,5,5′-3,3,5,5-Tetramethylbenzidine, Roche 11835033001) was added. After a 5 Minute incubation at room temperature the optical density (OD) was measured at 370 nm using an EnVision Multimode Plate Reader (Perkin Elmer).

### C3, C3a, C3b Counterscreening Elisa

### Buffer:

50 mM Hepes pH 7.4 with 150 mM NaCl was used as coating buffer. 2% BSA in 50 mM Hepes pH 7.4 containing 150 mM NaCl, 0.5% BSA and 0.1% Tween20 was used as blocking buffer. 50 mM Hepes pH 7.4 containing 150 mM NaCl, 0.5% BSA and 0.1% Tween20 was used as diluent for the ELISA. PBS containing 0.1% Tween20 was used as a washing buffer.

### Method:

A clear 384 well Nunc MaxiSorp Plate (Nunc, 464718) was coated with 25 μl of a mixture of 5 nM human C3 (Complement technology, A113), 5 nM human C3a (Complement technology, Al 18) and 2.5 nM human C3b (Complement technology, A114) in coating buffer and incubated at room temperature for 1 hour. After a single wash with 90 pl/well washing buffer on a EL406 Biotek washer, 90 pl blocking buffer was added and incubated for 30 Minutes at room temperature. After washing plates 3 times with 90 pl/well washing buffer on a EL406 Biotek washer 25 pl Sample was added to the plate and incubated at room temperature for 1 hour. After washing the plates as described above 25 pl Detection Mix (1:3000 Mouse anti Human kappa HRP (Southern Biotek 9230-05) and 1:2000 Mouse IgG anti T7 Tag HRP (Novagen 69048-3) was added. After a 1 hour incubation at room temperature plates were washed 6 times with 90 pl/well washing buffer using a Biotek EL406 Washer and 30 pl TMB (3,3′,5,5′-3,3,5,5-Tetramethylbenzidine, Roche 11835033001) was added. After a 5 Minute incubation at room temperature the optical density (OD) was measured at 370 nm using a EnVision Multimode Plate Reader (Perkin Elmer).

### Factor B, Ba, Bb Counterscreening Elisa

### Buffer:

50 mM Hepes pH 7.4 with 150 mM NaCl was used as coating buffer. 2% BSA in 50 mM Hepes pH 7.4 containing 150 mM NaCl, 0.5% BSA and 0.1% Tween20 was used as blocking buffer. 50 mM Hepes pH 7.4 containing 150 mM NaCl, 0.5% BSA and 0.1% Tween20 was used as diluent for the ELISA. PBS containing 0.1% Tween20 was used as a washing buffer.

### Method:

A clear 384 well Nunc MaxiSorp Plate (Nunc, 464718) was coated with 25 μl of a mixture of 10 nM human Factor B (Complement technology, A135), 5 nM human Ba (Complement technology, A154) and 10 nM human Bb (Complement technology, A155) in coating buffer and incubated at room temperature for 1 hour. After a single wash with 90 pl/well washing buffer on a EL406 Biotek washer, 90 pl blocking buffer was added and incubated for 30 Minutes at room temperature. After washing plates 3 times with 90 pl/well washing buffer on a EL406 Biotek washer 25 pl Sample was added to the plate and incubated at room temperature for 1 hour. After washing the plates as described above 25 pl Detection Mix (1:3000 Mouse anti Human kappa HRP (Southern Biotek 9230-05) and 1:2000 Mouse IgG anti T7 Tag HRP (Novagen 69048-3) was added in the sample area. After a 1 hour incubation at room temperature plates were washed 6 times with 90 pl/well washing buffer using a Biotek EL406 Washer and 30 pl TMB (3,3′,5,5′-3,3,5,5-Tetramethylbenzidine, Roche 11835033001) was added. After a 5 Minute incubation at room temperature the optical density (OD) was measured at 370 nm using a EnVision Multimode Plate Reader (Perkin Elmer).

### Example 3

### Identification and Antigen Binding of C3bBb Antibodies

A variety of antibody molecules that showed activity in binding to C3bBb were identified from panning and screening as described in Example 2 and optionally, further engineering of the so-derived antibody molecules was conducted.

All antibodies were initially expressed as Fab fragments comprising a constant light chain domain of SEQ ID NO:502 and a constant heavy chain domain of SEQ ID NO:503. Table E2 includes an overview over VH and VL amino acid sequences.

TABLE E2
amino acid sequences of antibodies identified in Example 2.

| | | VH amino acid | VL amino acid |
|---|---|---|---|
| | | sequence | sequence |
| # | identifier | (SEQ ID NO:) | (SEQ ID NO:) |
| 1 | P1AG9426 | 1 | 2 |
| 2 | P1AG9376 | 3 | 4 |
| 3 | P1AG9372 | 5 | 6 |
| 4 | P1AG9420 | 7 | 8 |
| 5 | P1AG9391 | 9 | 10 |
| 6 | P1AH1205 | 11 | 12 |
| 7 | P1AH1199 | 13 | 14 |
| 8 | C3bBb.P045.072 | 15 | 16 |
| 9 | C3bBb.P045.236 | 17 | 18 |
| 10 | C3bBb.P045.225 | 19 | 20 |
| 11 | C3bBb.P048.152 | 21 | 22 |
| 12 | C3bBb.P048.061 | 23 | 24 |
| 13 | C3bBb.P048.127 | 25 | 26 |
| 14 | C3bBb.P048.241 | 27 | 28 |
| 15 | C3bBb.P048.230 | 29 | 30 |
| 16 | C3bBb.P048.204 | 31 | 32 |
| 17 | C3bBb.P048.333 | 33 | 34 |
| 18 | C3bBb.P053.095 | 35 | 36 |
| 19 | C3bBb.P055.015 | 37 | 38 |
| 20 | P1AF8499 | 39 | 40 |
| 21 | GQ-5_1 | 41 | 42 |
| 22 | GQ-11_1 | 43 | 44 |
| 23 | PT5622.F19 | 45 | 46 |
| 24 | PT5624.P06 | 47 | 48 |
| 25 | PT5624.M09 | 49 | 50 |
| 26 | PT5626.M10 | 51 | 52 |
| 27 | PT5626.C10 | 53 | 54 |
| 28 | PT5628.E16 | 55 | 56 |
| 29 | PT5630.D05 | 57 | 58 |
| 30 | PT5630.A20 | 59 | 60 |
| 31 | PT5632.A13 | 61 | 62 |
| 32 | PT5634.N13 | 63 | 64 |
| 33 | PT5634.M24 | 65 | 66 |
| 34 | PT5636.P22 | 67 | 68 |
| 35 | PT5625.G07 | 69 | 70 |
| 36 | PT5627.M20 | 71 | 72 |
| 37 | PT5627.M12 | 73 | 74 |
| 38 | PT5629.F09 | 75 | 76 |
| 39 | PT5629.H08 | 77 | 78 |
| 40 | PT5631.D21 | 79 | 80 |
| 41 | PT5633.I03 | 81 | 82 |
| 42 | PT5635.C08 | 83 | 84 |
| 43 | PT5639.G15 | 85 | 86 |
| 44 | PT5622.A20 | 87 | 88 |
| 45 | PT5622.C12 | 89 | 90 |
| 46 | PT5622.G24 | 91 | 92 |
| 47 | PT5622.I20 | 93 | 94 |
| 48 | PT5622.J24 | 95 | 96 |
| 49 | PT5624.A21 | 97 | 98 |
| 50 | PT5624.B10 | 99 | 100 |
| 51 | PT5624.N05 | 101 | 102 |
| 52 | PT5624.P11 | 103 | 104 |
| 53 | PT5626.A12 | 105 | 106 |
| 54 | PT5626.N08 | 107 | 108 |
| 55 | PT5626.N18 | 109 | 110 |
| 56 | PT5626.O09 | 111 | 112 |
| 57 | PT5628.B13 | 113 | 114 |
| 58 | PT5628.C21 | 115 | 116 |
| 59 | PT5628.E12 | 117 | 118 |
| 60 | PT5628.I07 | 119 | 120 |
| 61 | PT5628.P14 | 121 | 122 |
| 62 | PT5630.G05 | 123 | 124 |
| 63 | PT5630.K04 | 125 | 126 |
| 64 | PT5630.M11 | 127 | 128 |
| 65 | PT5630.N06 | 129 | 130 |
| 66 | PT5630.O08 | 131 | 132 |
| 67 | PT5632.A10 | 133 | 134 |
| 68 | PT5632.A15 | 135 | 136 |
| 69 | PT5632.B15 | 137 | 138 |
| 70 | PT5632.D11 | 139 | 140 |
| 71 | PT5632.J23 | 141 | 142 |
| 72 | PT5634.A17 | 143 | 144 |
| 73 | PT5634.A21 | 145 | 146 |
| 74 | PT5634.K10 | 147 | 148 |
| 75 | PT5634.M16 | 149 | 150 |
| 76 | PT5634.N11 | 151 | 152 |
| 77 | PT5634.P19 | 153 | 154 |
| 78 | PT5636.A16 | 155 | 156 |
| 79 | PT5636.D21 | 157 | 158 |
| 80 | PT5636.M03 | 159 | 160 |
| 81 | PT5636.N13 | 161 | 162 |
| 82 | PT5636.P11 | 163 | 164 |
| 83 | PT5636.P15 | 165 | 166 |
| 84 | PT5638.C06 | 167 | 168 |
| 85 | PT5638.C20 | 169 | 170 |
| 86 | PT5638.E14 | 171 | 172 |
| 87 | PT5638.J23 | 173 | 174 |
| 88 | PT5638.O14 | 175 | 176 |
| 89 | PT5638.P12 | 177 | 178 |
| 90 | PT5623.E21 | 179 | 180 |
| 91 | PT5623.F17 | 181 | 182 |
| 92 | PT5623.G08 | 183 | 184 |
| 93 | PT5623.H05 | 185 | 186 |
| 94 | PT5623.O08 | 187 | 188 |
| 95 | PT5625.A05 | 189 | 190 |
| 96 | PT5625.B14 | 191 | 192 |
| 97 | PT5625.C15 | 193 | 194 |
| 98 | PT5625.G05 | 195 | 196 |
| 99 | PT5625.L22 | 197 | 198 |
| 100 | PT5625.O14 | 199 | 200 |
| 101 | PT5627.I21 | 201 | 202 |
| 102 | PT5629.F04 | 203 | 204 |
| 103 | PT5629.L22 | 205 | 206 |
| 104 | PT5629.L24 | 207 | 208 |
| 105 | PT5629.M15 | 209 | 210 |
| 106 | PT5631.H06 | 211 | 212 |
| 107 | PT5631.K12 | 213 | 214 |
| 108 | PT5631.N17 | 215 | 216 |
| 109 | PT5631.P07 | 217 | 218 |
| 110 | PT5633.E09 | 219 | 220 |
| 111 | PT5633.G19 | 221 | 222 |
| 112 | PT5635.F04 | 223 | 224 |
| 113 | PT5635.L21 | 225 | 226 |
| 114 | PT5635.O15 | 227 | 228 |
| 115 | PT5637.B23 | 229 | 230 |
| 116 | PT5637.C20 | 231 | 232 |
| 117 | PT5637.K09 | 233 | 234 |
| 118 | PT5637.L15 | 235 | 236 |
| 119 | PT5637.M03 | 237 | 238 |
| 120 | PT5637.P15 | 239 | 240 |
| 121 | PT5639.B15 | 241 | 242 |
| 122 | PT5639.P05 | 243 | 244 |
| 123 | PT5625.G07_22.F19_2 | 245 | 246 |
| 124 | PT5625.G07_24.M09_1 | 247 | 248 |
| 125 | PT5625.G07_24.P06_2 | 249 | 250 |
| 126 | PT5625.G07_26.C10_1 | 251 | 252 |
| 127 | PT5625.G07_26.M10_2 | 253 | 254 |
| 128 | PT5625.G07_28.E16_1 | 255 | 256 |
| 129 | PT5625.G07_30.A20_1 | 257 | 258 |
| 130 | PT5625.G07_32.A13_2 | 259 | 260 |
| 131 | PT5625.G07_34.M24_1 | 261 | 262 |
| 132 | PT5625.G07_34.N13_3 | 263 | 264 |
| 133 | PT5625.G07_36.P22_1 | 265 | 266 |
| 134 | PT5627.M20_24.M09_1 | 267 | 268 |
| 135 | PT5627.M20_24.P06_1 | 269 | 270 |
| 136 | PT5627.M20_26.C10_1 | 271 | 272 |
| 137 | PT5627.M20_26.M10_2 | 273 | 274 |
| 138 | PT5627.M20_28.E16_2 | 275 | 276 |
| 139 | PT5627.M20_30.D05_1 | 277 | 278 |
| 140 | PT5627.M20_32.A13_1 | 279 | 280 |
| 141 | PT5627.M20_34.M24_2 | 281 | 282 |
| 142 | PT5627.M20_34.N13_4 | 283 | 284 |
| 143 | PT5627.M20_36.P22_1 | 285 | 286 |
| 144 | PT5627.M12_22.F19_1 | 287 | 288 |
| 145 | PT5627.M12_24.M09_1 | 289 | 290 |
| 146 | PT5627.M12_26.M10_2 | 291 | 292 |
| 147 | PT5627.M12_32.A13_2 | 293 | 294 |
| 148 | PT5627.M12_34.M24_2 | 295 | 296 |
| 149 | PT5627.M12_34.N13_1 | 297 | 298 |
| 150 | PT5629.F09_26.C10_1 | 299 | 300 |
| 151 | PT5629.F09_28.E16_1 | 301 | 302 |
| 152 | PT5629.F09_30.A20_1 | 303 | 304 |
| 153 | PT5629.F09_30.D05_1 | 305 | 306 |
| 154 | PT5629.F09_32.A13_3 | 307 | 308 |
| 155 | PT5629.F09_36.P22_2 | 309 | 310 |
| 156 | PT5629.H08_22.F19_4 | 311 | 312 |
| 157 | PT5629.H08_24.M09_4 | 313 | 314 |
| 158 | PT5629.H08_24.P06_2 | 315 | 316 |
| 159 | PT5629.H08_26.C10_1 | 317 | 318 |
| 160 | PT5629.H08_26.M10_1 | 319 | 320 |
| 161 | PT5629.H08_28.E16_2 | 321 | 322 |
| 162 | PT5629.H08_30.A20_2 | 323 | 324 |
| 163 | PT5629.H08_30.D05_1 | 325 | 326 |
| 164 | PT5629.H08_32.A13_1 | 327 | 328 |
| 165 | PT5629.H08_34.M24_2 | 329 | 330 |
| 166 | PT5629.H08_34.N13_2 | 331 | 332 |
| 167 | PT5629.H08_36.P22_4 | 333 | 334 |
| 168 | PT5631.D21_22.F19_2 | 335 | 336 |
| 169 | PT5631.D21_24.M09_1 | 337 | 338 |
| 170 | PT5631.D21_24.P06_4 | 339 | 340 |
| 171 | PT5631.D21_26.C10_1 | 341 | 342 |
| 172 | PT5631.D21_26.M10_4 | 343 | 344 |
| 173 | PT5631.D21_28.E16_3 | 345 | 346 |
| 174 | PT5631.D21_30.A20_4 | 347 | 348 |
| 175 | PT5631.D21_30.D05_1 | 349 | 350 |
| 176 | PT5631.D21_32.A13_4 | 351 | 352 |
| 177 | PT5631.D21_34.M24_4 | 353 | 354 |
| 178 | PT5631.D21_34.N13_2 | 355 | 356 |
| 179 | PT5631.D21_36.P22_3 | 357 | 358 |
| 180 | PT5633.I03_22.F19_1 | 359 | 360 |
| 181 | PT5633.I03_24.M09_1 | 361 | 362 |
| 182 | PT5633.I03_24.P06_4 | 363 | 364 |
| 183 | PT5633.I03_26.C10_1 | 365 | 366 |
| 184 | PT5633.I03_26.M10_4 | 367 | 368 |
| 185 | PT5633.I03_28.E16_4 | 369 | 370 |
| 186 | PT5633.I03_30.A20_1 | 371 | 372 |
| 187 | PT5633.I03_30.D05_1 | 373 | 374 |
| 188 | PT5633.I03_32.A13_1 | 375 | 376 |
| 189 | PT5633.I03_34.M24_2 | 377 | 378 |
| 190 | PT5633.I03_34.N13_1 | 379 | 380 |
| 191 | PT5633.I03_36.P22_2 | 381 | 382 |
| 192 | PT5635.C08_22.F19_1 | 383 | 384 |
| 193 | PT5635.C08_24.M09_1 | 385 | 386 |
| 194 | PT5635.C08_24.P06_1 | 387 | 388 |
| 195 | PT5635.C08_26.C10_1 | 389 | 390 |
| 196 | PT5635.C08_26.M10_2 | 391 | 392 |
| 197 | PT5635.C08_28.E16_1 | 393 | 394 |
| 198 | PT5635.C08_30.A20_1 | 395 | 396 |
| 199 | PT5635.C08_30.D05_3 | 397 | 398 |
| 200 | PT5635.C08_32.A13_3 | 399 | 400 |
| 201 | PT5635.C08_34.M24_2 | 401 | 402 |
| 202 | PT5635.C08_34.N13_1 | 403 | 404 |
| 203 | PT5635.C08_36.P22_1 | 405 | 406 |
| 204 | PT5639.G15_22.F19_2 | 407 | 408 |
| 205 | PT5639.G15_24.P06_1 | 409 | 410 |
| 206 | PT5639.G15_24.M09_4 | 411 | 412 |
| 207 | PT5639.G15_26.M10_3 | 413 | 414 |
| 208 | PT5639.G15_28.E16_1 | 415 | 416 |
| 209 | PT5639.G15_30.A20_1 | 417 | 418 |
| 210 | PT5639.G15_30.D05_1 | 419 | 420 |
| 211 | PT5639.G15_34.N13_4 | 421 | 422 |
| 212 | PT5639.G15_34.M24_3 | 423 | 424 |
| 213 | PT5639.G15_36.P22_3 | 425 | 426 |
| 214 | PT5625.G07_30.D05_1 | 427 | 428 |
| 215 | PT5627.M12_26.C10_1 | 429 | 430 |
| 216 | PT5627.M12_30.A20_2 | 431 | 432 |
| 217 | PT5629.F09_34.N13_3 | 433 | 434 |

From the antibodies identified during screening, candidate P1AF8499 (#20 in Table E2, comprising VH SEQ ID NO: 39 and VL SEQ ID NO: 40) was selected based on its selectivity, convertase inhibition and impact on convertase stability profile for further optimization.

To this end two phage libraries were created containing diversification of the parental candidate antibody either in the heavy chain CDRs, or in the light chain CDRs. The libraries were panned and screened against the target as described in Example 2.

Candidates with improved properties compared to the parental molecule were selected and heavy and light chains of individual molecules were combined into single molecules in a combinatorial manner. Improved versions of the parental antibody candidate are antibodies #1-#7 as well as antibodies #23 to #217 as indicated in Table E2 further below.

Binding of the antibodies to human C3bBb was analyzed by different ELISA assays, as described in Example 2 to analyze binding towards human C3bBb formed from Factor B with three mutations D279G, K350N and M458I (herein referred to as “C3bBb triple mutant ELISA”) as well as controls assessing binding to different related components of the complement system, i.e. C3, C3a, C3b and Factor B, Ba, Bb. Results of the different ELISA assays for the antibodies as defined in Table E2 are shown in Table E3.

TABLE E3
Antigen binding of C3bBb complex of antibodies identified
in Table E2 using C3bBb triple mutant ELISA

| | | C3bBb | | | | |
|---|---|---|---|---|---|---|
| | | (D279G/ | | C3/ | | |
| | | 350N/ | C3bBb | C3a/ | CFB/ | negative |
| # | identifier | M458I) | (D279G) | C3b | Ba/Bb | control |
| 8 | C3bBb.P045.072 | 2.904 | 0.023 | −0.005 | −0.010 | −0.026 |
| 9 | C3bBb.P045.236 | 1.815 | −0.003 | 0.005 | 0.000 | −0.024 |
| 10 | C3bBb.P045.225 | 2.615 | 0.017 | 0.027 | −0.021 | −0.003 |
| 11 | C3bBb.P048.152 | 0.405 | −0.035 | −0.007 | −0.005 | −0.025 |
| 12 | C3bBb.P048.061 | 1.899 | 0.004 | 0.001 | 0.006 | 0.015 |
| 13 | C3bBb.P048.127 | 0.187 | 2.423 | −0.006 | 0.002 | −0.010 |
| 14 | C3bBb.P048.241 | 2.384 | 2.573 | 0.170 | 0.037 | 0.051 |
| 15 | C3bBb.P048.230 | 2.692 | 2.670 | 0.210 | 0.021 | 0.018 |
| 16 | C3bBb.P048.204 | 1.341 | 1.943 | 0.126 | 0.015 | −0.014 |
| 17 | C3bBb.P048.333 | 2.786 | 2.758 | 0.274 | 0.034 | 0.008 |
| 18 | C3bBb.P053.095 | −0.006 | 0.353 | −0.001 | 0.003 | 0.008 |
| 19 | C3bBb.P055.015 | −0.010 | 0.860 | −0.014 | 0.004 | −0.014 |
| 23 | PT5622.F19 | 2.7865 | 2.8105 | −0.002 | −0.0145 | −0.0955 |
| 24 | PT5624.P06 | 2.93375 | 2.76175 | 0.0175 | 0.05575 | −0.02825 |
| 25 | PT5624.M09 | 2.88075 | 2.72575 | −0.0035 | −0.01025 | −0.07625 |
| 26 | PT5626.M10 | 2.7295 | 2.4185 | −0.00175 | −0.0075 | −0.05975 |
| 27 | PT5626.C10 | 2.4645 | 2.1365 | −0.01175 | −0.0235 | −0.07275 |
| 28 | PT5628.E16 | 2.3335 | 2.4705 | 0.013 | −0.0215 | −0.087 |
| 29 | PT5630.D05 | 2.41075 | 2.42475 | −0.006 | −0.01625 | −0.10825 |
| 30 | PT5630.A20 | 2.14975 | 2.28075 | 0.013 | −0.01925 | −0.07925 |
| 31 | PT5632.A13 | 2.34725 | 2.41225 | 0.012 | −0.02675 | −0.079 |
| 32 | PT5634.N13 | 2.58075 | 2.25775 | 0.025 | 0.02175 | −0.06425 |
| 33 | PT5634.M24 | 2.08675 | 1.68075 | 0.027 | 0.02075 | −0.04625 |
| 34 | PT5636.P22 | 2.7385 | 2.2665 | 0.049 | 0.0165 | 0.0055 |
| 35 | PT5625.G07 | 2.26575 | 1.43375 | −0.006 | −0.01225 | −0.0875 |
| 36 | PT5627.M20 | 2.09725 | 1.48025 | 0.00975 | 0.01525 | −0.01875 |
| 37 | PT5627.M12 | 1.96125 | 1.16125 | 0.00875 | −0.00175 | −0.05675 |
| 38 | PT5629.F09 | 2.172 | 1.536 | −0.003 | −0.015 | −0.07725 |
| 39 | PT5629.H08 | 1.893 | 0.93 | 0.003 | −0.003 | −0.06825 |
| 40 | PT5631.D21 | 1.597 | 0.671 | −0.018 | −0.032 | −0.087 |
| 41 | PT5633.I03 | 0.9495 | 0.2615 | 0.008 | −0.0175 | −0.09175 |
| 42 | PT5635.C08 | 1.646 | 0.676 | −0.004 | −0.017 | −0.07625 |
| 43 | PT5639.G15 | 1.25525 | 0.43425 | −0.014 | −0.02575 | −0.05875 |
| 44 | PT5622.A20 | 2.4135 | 2.2785 | −0.014 | −0.0365 | −0.0745 |
| 45 | PT5622.C12 | 2.5055 | 2.5475 | −0.007 | −0.0235 | −0.1035 |
| 46 | PT5622.G24 | 2.7145 | 2.3815 | 0.007 | 0.0025 | −0.0715 |
| 47 | PT5622.I20 | 2.6745 | 2.5155 | 0.005 | 0.0095 | −0.0945 |
| 48 | PT5622.J24 | 2.6815 | 2.5955 | 0.031 | 0.0085 | 0.0195 |
| 49 | PT5624.A21 | 2.36775 | 2.72575 | −0.0105 | −0.02625 | −0.06325 |
| 50 | PT5624.B10 | 2.68475 | 2.79175 | −0.0095 | −0.02525 | −0.09025 |
| 51 | PT5624.N05 | 2.59275 | 2.51675 | 0.0105 | 0.07275 | −0.06425 |
| 52 | PT5624.P11 | 2.67675 | 2.78175 | 0.0235 | 0.02175 | −0.00625 |
| 53 | PT5626.A12 | 2.5245 | 2.3225 | 0.03925 | −0.0205 | −0.04575 |
| 54 | PT5626.N08 | 2.9095 | 2.6135 | 0.01525 | 0.0095 | −0.04375 |
| 55 | PT5626.N18 | 2.7745 | 2.8355 | 0.01125 | 0.0015 | −0.04475 |
| 56 | PT5626.O09 | 2.6955 | 2.4135 | 0.00025 | 0.0065 | −0.04975 |
| 57 | PT5628.B13 | 2.3475 | 2.0165 | 0.038 | −0.0255 | −0.087 |
| 58 | PT5628.C21 | 2.5615 | 2.8435 | −0.007 | −0.0285 | −0.09 |
| 59 | PT5628.E12 | 2.4015 | 2.3075 | 0.008 | −0.0185 | −0.093 |
| 60 | PT5628.I07 | 2.3375 | 2.5365 | 0.014 | −0.0115 | −0.084 |
| 61 | PT5628.P14 | 2.4965 | 2.3855 | 0.033 | 0.0185 | −0.024 |
| 62 | PT5630.G05 | 2.34275 | 2.11075 | −0.009 | −0.01225 | −0.10725 |
| 63 | PT5630.K04 | 1.96875 | 1.56875 | −0.006 | −0.01325 | −0.10625 |
| 64 | PT5630.M11 | 2.53775 | 2.35075 | 0.009 | 0.00575 | −0.09325 |
| 65 | PT5630.N06 | 2.33875 | 1.97975 | 0.012 | 0.00875 | −0.08625 |
| 66 | PT5630.O08 | 2.55475 | 2.22675 | 0.003 | 0.00475 | −0.08125 |
| 67 | PT5632.A10 | 2.11325 | 1.10025 | 0.009 | −0.03875 | −0.081 |
| 68 | PT5632.A15 | 2.44325 | 2.20125 | 0.008 | −0.04475 | −0.081 |
| 69 | PT5632.B15 | 2.41625 | 2.42125 | −0.049 | −0.04375 | −0.09 |
| 70 | PT5632.D11 | 2.47725 | 2.01625 | 0.01 | −0.02275 | −0.081 |
| 71 | PT5632.J23 | 2.17625 | 2.17325 | 0.047 | −0.01075 | −0.067 |
| 72 | PT5634.A17 | 2.38575 | 1.77875 | −0.014 | −0.03525 | −0.06325 |
| 73 | PT5634.A21 | 2.38175 | 2.40675 | −0.001 | −0.02225 | −0.07825 |
| 74 | PT5634.K10 | 2.55575 | 2.82475 | −0.009 | −0.02825 | −0.10725 |
| 75 | PT5634.M16 | 2.32575 | 2.51775 | 0.001 | −0.00225 | −0.09525 |
| 76 | PT5634.N11 | 2.46075 | 2.31875 | 0.017 | 0.00575 | −0.07625 |
| 77 | PT5634.P19 | 2.16375 | 1.91875 | 0.051 | 0.05675 | 0.00975 |
| 78 | PT5636.A16 | 2.4105 | 2.2515 | −0.006 | −0.0325 | −0.0645 |
| 79 | PT5636.D21 | 2.0895 | 1.5595 | −0.002 | −0.0195 | −0.0705 |
| 80 | PT5636.M03 | 2.3435 | 2.0835 | 0 | −0.0195 | −0.0665 |
| 81 | PT5636.N13 | 2.5165 | 2.3515 | 0.038 | 0.0165 | −0.0215 |
| 82 | PT5636.P11 | 2.0955 | 1.1785 | 0.034 | 0.0315 | −0.0105 |
| 83 | PT5636.P15 | 2.3905 | 2.0715 | 0.04 | 0.0275 | 0.0115 |
| 84 | PT5638.C06 | 1.8775 | 1.5045 | 0.021 | −0.0275 | −0.10825 |
| 85 | PT5638.C20 | 1.8975 | 1.6935 | −0.006 | −0.0305 | −0.10425 |
| 86 | PT5638.E14 | 2.0825 | 1.6525 | 0.008 | −0.0225 | −0.10325 |
| 87 | PT5638.J23 | 1.5375 | 0.7815 | 0.055 | 0.0885 | −0.07825 |
| 88 | PT5638.O14 | 2.1635 | 1.3795 | 0.108 | 0.0135 | −0.06925 |
| 89 | PT5638.P12 | 2.1835 | 1.3065 | 0.098 | 0.0375 | −0.02925 |
| 90 | PT5623.E21 | 2.1255 | 0.9965 | −0.0105 | −0.0165 | −0.09625 |
| 91 | PT5623.F17 | 2.1345 | 1.1215 | −0.0075 | −0.0205 | −0.08725 |
| 92 | PT5623.G08 | 2.0515 | 1.2285 | 0.0165 | −0.0125 | −0.09225 |
| 93 | PT5623.H05 | 1.7455 | 0.8205 | 0.0005 | −0.0135 | −0.09125 |
| 94 | PT5623.O08 | 2.0355 | 0.6395 | −0.0025 | 0.0155 | −0.07525 |
| 95 | PT5625.A05 | 2.06975 | 1.07475 | −0.011 | −0.02125 | −0.0585 |
| 96 | PT5625.B14 | 1.60275 | 0.60975 | −0.008 | −0.01525 | −0.0835 |
| 97 | PT5625.C15 | 1.85375 | 0.84275 | −0.014 | −0.02225 | −0.0905 |
| 98 | PT5625.G05 | 1.84375 | 0.88175 | 0.007 | −0.01425 | −0.0855 |
| 99 | PT5625.L22 | 1.38775 | 0.45375 | 0.003 | −0.00125 | −0.0685 |
| 100 | PT5625.O14 | 1.89675 | 0.71975 | 0.016 | 0.02175 | −0.0575 |
| 101 | PT5627.I21 | 1.11525 | 0.42125 | 0.00475 | −0.00375 | −0.05775 |
| 102 | PT5629.F04 | 2.073 | 1.086 | −0.007 | 0.027 | −0.06525 |
| 103 | PT5629.L22 | 1.925 | 0.996 | 0.004 | −0.007 | −0.06325 |
| 104 | PT5629.L24 | 1.929 | 0.724 | 0.001 | −0.004 | −0.04625 |
| 105 | PT5629.M15 | 2.284 | 1.287 | 0.005 | 0.002 | −0.07025 |
| 106 | PT5631.H06 | 1.235 | 0.368 | 0 | −0.014 | −0.081 |
| 107 | PT5631.K12 | 1.905 | 1.014 | −0.005 | −0.017 | −0.091 |
| 108 | PT5631.N17 | 1.825 | 0.872 | 0.015 | 0.013 | −0.046 |
| 109 | PT5631.P07 | 1.858 | 0.598 | 0.004 | −0.001 | −0.007 |
| 110 | PT5633.E09 | 0.6525 | 0.2785 | −0.013 | −0.0345 | −0.09775 |
| 111 | PT5633.G19 | 0.9495 | 0.2375 | 0.016 | −0.0135 | −0.08475 |
| 112 | PT5635.F04 | 1.671 | 0.649 | −0.013 | −0.022 | −0.07625 |
| 113 | PT5635.L21 | 1.896 | 1.355 | 0.01 | −0.001 | −0.05725 |
| 114 | PT5635.O15 | 1.647 | 0.674 | 0 | 0.014 | −0.05125 |
| 115 | PT5637.B23 | 2.044 | 1.284 | 0.018 | −0.047 | −0.1105 |
| 116 | PT5637.C20 | 1.972 | 1.224 | −0.011 | −0.034 | −0.1155 |
| 117 | PT5637.K09 | 1.716 | 0.708 | −0.001 | −0.023 | −0.1165 |
| 118 | PT5637.L15 | 1.975 | 1.252 | 0.025 | −0.012 | −0.0965 |
| 119 | PT5637.M03 | 2.026 | 1.181 | 0.022 | −0.028 | −0.1155 |
| 120 | PT5637.P15 | 2.142 | 1.292 | 0.037 | 0.019 | −0.0185 |
| 121 | PT5639.B15 | 1.68625 | 0.80525 | −0.018 | −0.02675 | −0.06275 |
| 122 | PT5639.P05 | 0.94025 | 0.34925 | 0.004 | 0.00825 | −0.01175 |

Seven candidate antibodies were generated and further characterized with respect to their potential for therapeutic application, five antibodies were generated as a full length IgG1 (Mab) and two antibodies were generated as Fab fragments (see Table E4).

All Mabs and Fabs were produced in a serum-free medium, with the ExpiCHO Expression System (Thermo Fisher Scientific). For cultivation of the ExpiCHO cells the corresponding High-Titer Protocol was used. The Molar Plasmid Ratio was target dependent 1:1 or 1:3 Light Chain to Heavy Chain.

The purification of antibodies performed by Kappa Select (Cytavia) followed by size exclusion chromatography Superdex200 (Cytavia) in 20 mM Histidine, 140 mM NaCl pH 6.0. The purification of Fab's started by Kappa Select (Cytavia) followed by a CaptureSelect CH1-XL (Thermo Fisher Scientific) Step and finalized by Superdex200 (Cytavia) in 20 mM Histidine, 140 mM NaCl pH 6.0.

TABLE E4
amino acid sequences of clinical candidate
antibodies identified in Example 2.

| | | VH amino | VL amino | HC amino | LC amino |
|---|---|---|---|---|---|
| | | acid | acid | acid | acid |
| # | identifier | sequence | sequence | sequence | sequence |
| 1 | P1AG9426 (Mab) | 1 | 2 | 435 | 436 |
| 2 | P1AG9376 (Mab) | 3 | 4 | 437 | 438 |
| 3 | P1AG9372 (Mab) | 5 | 6 | 439 | 440 |
| 4 | P1AG9420 (Mab) | 7 | 8 | 441 | 442 |
| 5 | P1AG9391 (Mab) | 9 | 10 | 443 | 444 |
| 6 | P1AH1205 (Fab) | 11 | 12 | 445 | 446 |
| 7 | P1AH1199 (Fab) | 13 | 14 | 447 | 448 |

### Example 4

### Trimeric Complex Formed by C3bBb and a Fab Fragment of the Invention Analyzed by Cryo Electron Microscopy

A trimeric complex of parental antibody P1AF8499 (#20) with the C3bBb complex was analyzed by electron microscopy. Briefly, the following protocol was adhered to:

Cryo-EM grid preparation. Purified recombinant complex of human C3bBb triple mutant+Fab P1AF8499 in 20 mM HEPES, 150 mM NaCl, 1 mM NiSO4, pH 7.4 were frozen in liquid nitrogen (0.15 mg/ml, 0.7 pM). After thawing, 3 pl were applied to freshly glow-discharged Quantifoil R1.2/1.3 400-mesh copper grids (Plano). Grids were blotted (Whatman filter paper, Grade 1) for 4 s at 20° C. under 95% chamber humidity and plunge frozen in liquid ethane using a EM GP2 plunge freezer (Leica).

Cryo-EM data acquisition. Two datasets were collected on an FEI Titan Krios (Thermo Fisher Scientific, Waltham, MA, USA) at C-CINA, University of Basel, operating at 300 keV and equipped with a Gatan Quantum-LS imaging energy filter (GIF, 20 eV energy-loss window; Gatan Inc, Pleasanton, CA, USA). Altogether, 7550 movies of 40 frames were acquired with a K2 Summit direct electron detector (Gatan Inc) in the counted mode at a physical pixel size of 0.82 Å and a total electron dose of 56 e−/Å2 with the SerialEM software [LK1]. Initial quality check of captured images was performed by the FOCUS programme [LK3] that employed the MotionCor2 programme [LK2] for correction of frame movements and their dose-weighting, and the CTFFIND4 programme [LK4] for the estimation of CTF parameters. Images displaying a resolution lower than 7 Å or average drifts higher than 2 Å per frame were excluded from the analysis.

Cryo-EM data processing. Image stacks were processed using cryoSPARC v3.3 (Structura Biotechnology). Frame motion was corrected and contrast-transfer function (CTF) parameters were fit from movies using the 25-4 Å band of the spectrum. Images with CTF fits to 4.5 Å or better were selected for particle picking using a spherical blob picker with maximum and characteristic diameters of 100 and 200 Å, respectively. Three rounds of 2D classification with 150 classes and a box size of 384 pixels were performed to sort C3bBb-P1AF8499 protein particles from debris and other false positives. The remaining particles were subjected to ab initio reconstruction and homogenous refinement to obtain a preliminary 3D reconstruction, which was used to create low-pass filtered templates for another round of particle picking. The picking and classification procedure was repeated to obtain a particle selection of 497884 particles. Reference volumes for 3D heterogeneous refinement were obtained by ab initio reconstruction into 4 classes, and particles were further classified using 2500 batch size and 4 full iterations. One class with the best structural features containing 172991 particles was selected for non-uniform refinement. For model building and figure preparation, sharpened maps were used. Maps were filtered according to the local resolution using the implementation of blocres within cryoSPARC.

Model building. The structure of C3bBb (PDB 2WIN) was deconstructed into four separate models (TED-CUB, B2-MG, C345C and cleaved factor B). These models were fitted as rigid bodies into the cryo-EM map. After modelling P1AF8499 and performing manual adjustments mostly pertaining to P1AF8499 CDRs, a single round of real space refinement using the phenix.real_space_refinement tool with tight secondary structure restraints (TC1) was used to correct global structural differences between the initial model and the map. The model was further manually adjusted in Coot (TC2) through iterative rounds of model building and real space refinement. Phenix.mtriage was used to compute model vs map FSC curves that are consistent with our resolution estimate from the half-map FSC measurement.

Results are shown in FIG. 1. The left image shows the trimeric complex formed between P1AF8499 Fab fragment and C3bBb. The epitope bound by P1AF8499 comprises amino acids from both components, C3b and Bb (FIG. 1, right image). Hence, crossreactivity with individual subunits of C3bBb should be minimal or even excluded. The following epitope bound by P1AF8499 Fab fragment was identified by defining interaction of amino acid residues within 5 Å of P1AF8499:

TABLE E5
epitope bound by parental antibody P1AF8499 Fab fragment

| | SEQ ID | |
|---|---|---|
| | NO: of | |
| Subunit of C3bBb | subunit | amino acid residues within subunit |
| C3b | 495 | Arg444, Lys534, Gly539, Ser540, Val524, |
| | | Lys544, Gly546, Gln547, Ser548, Arg551, |
| | | Gln557, Gln558, Thr560, Lys562, Glu564, |
| | | Glu758, Pro759, Lys761, Asn762, Ile764, |
| | | Leu768, Asn770, Asp797 |
| Bb subunit of | 501 | Tyr465, Lys473, Ile474, Ser475, Ile477, |
| human FB | | Gly482, His483, Lys513, Val514, Ser515, |
| | | Lys520, Arg521, Asp522, Glu610, Lys613 |

### Example 5

### Antigen Binding to Human and Cynomolgus C3bBb Complex as Analyzed by SPR

Antigen binding of the antibodies of Example 3 to C3bBb from different species was assessed by SPR, using the following setup:

A Biacore T200 system was primed with 1×HBS-P+ buffer (10 mM HEPES, 150 mM NaCl and 0.05% v/v Surfactant P20), prepared from a 10× stock (Cytiva BR100671). A CAP chip (part of the Biotin CAPture KIT—Cytiva 28920233) was docked to the system and equilibrated overnight.

### Human C3bBb Binding Characteristics

In the following, the measurement on human C3bBb complex is described. The running buffer was changed to HBS-P+ supplemented with 5 mM NiSO₄, to obtain stabilization of the C3bBb complex, which was built up on the chip surface from scratch in every cycle. Flow cells (FCs) 1 and 2 were used as active FCs. The sample compartment temperature was set to 12° C., while the analysis temperature was defined to 25° C. and 37° C., respectively, dependent on the assay step. Each analysis cycle started with an injection of the undiluted CAP reagent (part of the Biotin CAPture KIT—Cytiva 28920233) for 300 s at 5 pl/min passing over both FCs. Biotinylated-C3b was injected to FC 1 and 2 at 9 nM and 10 pl/min for 30 s (IgG analysis) and 60 s (Fab analysis), respectively. The injection was done separately on each FC to obtain comparable levels on both FCs. D-biotin (Invitrogen B20656) was injected at 50 pM and 10 pl/min for 60 s to both FCs, to block the remaining biotin binding sites. The FB triple mutant was injected to FC 2 at 100 nM and 5 pl/min for 300s to build the intermediate C3bB on FC2. Subsequently, FD was injected to FC 2 at 100 nM and 5 pl/min for 300s to build the C3bBb complex. After a stabilization period of 60 s, the sample injection for a single cycle kinetic was started. The sample was injected for 60 s at 30 pl/min to both FCs with five increasing concentrations of a 3-fold dilution series. The final dissociation time was set to 1200 s at 30 pl/min, due to the highly stable interaction. To regenerate the surface for the next cycle, the standard CAP chip regeneration, 8 M Gua-NaOH, was injected for 120s to both FCs. Finally, running buffer was injected for 60 s at 30 pl/min to both FCs with a final stabilization period of 60 s.

For the analysis, a double referencing, with subtraction of the reference flow cell (FC1) signals and the bracketing reference blank cycles with buffer injection only, was executed. The curves were fitted in the BIAevaluation Software using a 1:1 fit model for Fabs and the heterogeneous ligand model for IgGs to separate affin and avid binding, respectively.

Results of the interaction to human C3bBb are shown in Table E6.

TABLE E6
human C3bBb binding of clinical candidate antibodies of Example 3.

| # | identifier | KD 37° C. | KD 25° C. |
|---|---|---|---|
| 1 | P1AG9426 (Mab) | 14 pM | <1 pM |
| 2 | P1AG9376 (Mab) | <1 pM | <1 pM |
| 3 | P1AG9372 (Mab) | 11.6 pM | <1 pM |
| 4 | P1AG9420 (Mab) | 11 pM | <1 pM |
| 5 | P1AG9391 (Mab) | 1.6 pM | <1 pM |
| 6 | P1AH1205 (Fab) | 95 pM | 16 pM |
| 7 | P1AH1199 (Fab) | 200 pM | 30 pM |

### Cynomolgus C3bBb Binding Characteristics

In order to measure the kinetic to cynomolgus C3bBb, the setup described above was adopted. To built cynomolgus C3bBb, the respective cynomolgus components were used: biotinylated cyno C3b (SEQ ID NO: 497), cyno FB (triple mutant, SEQ ID NO: 500) and cyno FD (SEQ ID NO: 498). Concentrations and injection times of the three components were changed as following: Injection of biotinylated cyno C3b at 60 nM and 10 pl/min for 180 s (IgG analysis) and 300 s (Fab analysis), respectively; Injection of cyno FB triple mutant at 400 nM and 5 pl/min for 400 s; Injection of cyno FD at 600 nM and 5 pl/min for 400 s. Each concentration of the analytes was injected for 75 s and the dissociation time was shortened to 300 s. All remaining parameters remained as described for the measurement on the human C3bBb complex.

Results are shown in Table E7.

TABLE E7
cynomolgus C3bBb binding of clinical candidate antibodies in Example 3.

| # | identifier | KD 37° C. | KD 25° C. |
|---|---|---|---|
| 1 | P1AG9426 (Mab) | 197 nM | 206 nM |
| 2 | P1AG9376 (Mab) | 26 nM | 50 nM |
| 3 | P1AG9372 (Mab) | 260 nM | 207 nM |
| 4 | P1AG9420 (Mab) | 277 nM | 117 nM |
| 5 | P1AG9391 (Mab) | 150 nM | 130 nM |
| 6 | P1AH1205 (Fab) | 126 nM | 65 nM |
| 7 | P1AH1199 (Fab) | 460 nM | 245 nM |

### Example 6

### Assessment of Crossreactivity Towards Other Components of the Alternative Pathway as Analyzed by SPR

In order to show that the binders are specific for the C3bBb complex the binding against individual components as well as precursor and breakdown products of the complex was tested in Biacore (also confirmed in ELISA and BLI). Proteins tested were: C3b (component of the complex), C3 (precursor of C3b), C3d, iC3b, C3a (breakdown products of C3b), as well as Bb (component of the complex), FB (precursor of Bb) and Ba (breakdown product of FB).

The off-target SPR assay was set up as described below.

A Biacore T200 system was equipped with a CM5 chip and primed with 1×HBS-N(10 mM HEPES, 150 mM NaCl), prepared from a 10× stock solution (Cytiva BR100670). An anti-human Fab antibody (Thermo Fisher Scientific 7103082100) was immobilized to all flow cells (FC1-4) in an amine coupling with default settings and a capture antibody injection time of 420 s at 10 pl/min and 30 μg/ml in 10 mM Acetate pH 5.

For the following analysis, the system was primed with HBS-P+1 mM NiSO₄. After at least three initial conditioning cycles with capturing and regeneration, three antibodies were captured each cycle on FC 2, 3 and 4, respectively, at 10 μg/ml for 20 s. The off-target was injected at 200 nM for 60 s and the dissociation was followed for 60 s at a flow rate of 10 pl/min. Each cycle finished with a regeneration using 10 mM Glycine pH 2.1 for 70 s at 30 pl/min and a final stabilization time of 120 s. For each antibody, the different off-target injection cycles were bracketed by buffer injection cycles for double referencing purposes.

In order to analyze the potential off-target binding, the default binding and stability report points were analyzed. The RUs at the two report points were extracted after double referencing the curves (active flow cell subtracted by blank flow cell FC1 and the respective blank cycles). The RU values were normalized to binding [%] and stability [%], respectively, using the following equation:

$$binding [ \% ] or stability [ \% ] = ( double ref . binding or stability [ RU ] / capture level [ RU ] ) * ( capture molecule [ MW ] / analyte [ MW ] ) * 100$$

Results are shown in Table E8, which reports response levels directly after binding and after reaching stability (calculated as theoretical Rmax in %)

TABLE E8
Crossreactivity of indicated antibodies of Example
3 to components of the alternative pathway

| | P1AG9376 | P1AG9372 | P1AG9426 | P1AG9391 | P1AH1205 | P1AH1199 |
|---|---|---|---|---|---|---|
| # | 2 | 3 | 1 | 5 | 6 | 7 |
| binding | | | | | | |
| C3b | 0.06 | 0.06 | 0.32 | 0.49 | 0 | 0.39 |
| C3 | 0.14 | 0.72 | 2.01 | 2.61 | 0.01 | 2.39 |
| C3d | 0 | 0 | 0 | 0 | 0 | 0.53 |
| iC3b | 0 | 0.04 | 0.34 | 0.44 | 0 | 0.36 |
| C3a | 0 | 5.67 | 6.13 | 14.53 | 1.33 | 18.94 |
| Bb | 0 | 0 | 0 | 0 | 0 | 0.29 |
| FB | 0 | 0.11 | 0.16 | 0.20 | 0 | 0.53 |
| Ba | 0 | 0 | 0 | 0 | 0 | 0.72 |
| stability | | | | | | |
| C3b | 0.04 | 0.01 | 0.08 | 0.15 | 0.01 | 0.18 |
| C3 | 0.02 | 0.24 | 1.07 | 1.72 | 0.01 | 1.65 |
| C3d | 0 | 0 | 0 | 0 | 0 | 0.48 |
| iC3b | 0 | 0.02 | 0.21 | 0.26 | 0.00 | 0.25 |
| C3a | 0 | 1.45 | 0.53 | 3.87 | 0.65 | 7.40 |
| Bb | 0 | 0 | 0 | 0 | 0 | 0.27 |
| FB | 0 | 0.06 | 0.07 | 0.14 | 0 | 0.46 |
| Ba | 0 | 0 | 0 | 0 | 0 | 0.50 |

### Example 7

### Further Characteristics of Antibodies of the Invention

Thermal stability of antibodies of Example 3 was assessed by exposing the antibody to increasing temperature in a controlled gradient and structural unfolding (Tₘ by intrinsic fluorescence) and/or aggregation propensity (T_{agg} by SLS) are determined.

Apparent hydrophobicity was evaluated by determining the retention time on a hydrophobic HPLC column in comparison with known high and low hydrophobicity standard molecules.

FcRn affinity was assessed as follows: Interaction with immobilized FcRn was measured via SPR as no specific interaction is expected: biotinylated scFcRn was immobilized to a Series S Sensor Chip SA (500-600 RU). The antibodies were injected at 10 pl/min for 60 s at 5 nM. The dissociation was followed for 90 s. The run was performed at 25° C. in PBS-P pH 6 and the surface was regenerated using PBS-P pH 8 for 60s at 30 pl/min. The sensorgrams of the double referenced signals were compared to FcRn binding competent (Herceptin, wt FC part) and incompetent (Faricimab, Triple A FC part) controls. The positive control was diluted in a two fold dilution series down to 3.125% of 5 nM. The results were double referenced (control flow cell and buffer cycle) using the Biacore T200 Evaluation Software 3.0. The binding response (RU) of the positive control at different concentrations was used to create a standard curve. The remaining binding % (derived from the binding RUs) of all candidates at 100% concentration (5 nM) were calculated. All candidates indicated very low (<1 or 5%) FcRn surface interaction compared to the positive control. The negative control (Faricimab 3A) showed similar signals as P1AH1205 and P1AH1199.

Heparin affinity chromatography was done on specific HPLC affinity columns. The corresponding retention times are compared to established thresholds based on known molecules with good and bad PK.

Quality of antibody integrity was measured monitoring high molecular weight species with retention times shorter than the main species. This was done by using a size exclusion HPLC setup with UV A280 nm detection. Obtained peaks were integrated using a custom baseline and calculating the area under the curve. Relative areas to the total curve integral are displayed. Conditions for this chromatography were selected based on molecule size and the manufacturer's recommendation for protein analysis. Fragmentation of molecules is assessed using reduced and untreated capillary gel electrophoresis with intrinsic protein fluorescence detection. The low molecular weight species with shorter migration times than the main species are monitored and peak areas under the curve are integrated. Relative areas to the total curve integral are displayed. Functional integrity of the molecules is assessed with a binding assay specific for the target of the molecule (as described in example 5). Relative active concentrations were determined in cases of thermally stressed material. Putative asparagine and aspartic acid degradation hotspots are experimentally confirmed or excluded by tryptic Peptide Mapping MS analysis. Integration of the peak area of extracted ion chromatograms (XIC) was performed for wildtype and modified peptides. The ratio of modified peptides XIC areas relative to the wildtype XIC areas were taken into account when evaluating a potential modification site in the predicted complementarity determining regions.

Viscosity was assessed at different concentrations of the molecules of interest in a salt free generic buffer system. This is done by co incubating the molecule of interest at different concentrations with beads and detection by dynamic light scattering. The maximum feasible concentration is defined by a viscosity limit relevant for the target product profile and determined by fitting the available data to an appropriate model and applying the relevant threshold.

TABLE E9
Further characteristics of indicated antibodies

| | P1AG9376 | | P1AG9372 | | P1AG9426 | | P1AG9391 | | P1AH1205 | | P1AH1199 | |
|---|---|---|---|---|---|---|---|---|---|---|---|---|
| # | 2 | | 3 | | 1 | | 5 | | 6 | | 7 | |
| Thermal stability | 56 | | 62 | | 63 | | 59 | | 68 | | 67 | |
| (DLS T_{agg}/DLS | | | | | | | | | | | | |
| Tₘ) | | | | | | | | | | | | |
| Apparent | 0 | | 0.1 | | 0.2 | | 0.1 | | −0.4 | | −0.2 | |
| hydrophobicity | | | | | | | | | | | | |
| (HIC) | | | | | | | | | | | | |
| FcRn affinity on | No | | No | | No | | No | | No | | No | |
| biacore | interaction | | interaction | | interaction | | interaction | | interaction | | interaction | |
| Heparin affinity | 0.87 | | 0.28 | | 0.23 | | 0.26 | | 0.81 | | 0.12 | |
| chromatography | | | | | | | | | | | | |
| Titer in CHO | 3.4 | g/l | 1.6 | g/l | 6.0 | g/l | 3.3 | g/l | 3.0 | g/l | 2.6 | g/l |

| pools | | | | | | |
|---|---|---|---|---|---|---|
| Quality | >70% | >70% | >70% | >70% | >70% | >70% |
| max feasible conz | 29.9 | 96.5 | 81.8 | 85.7 | 220.2 | 223.2 |
| below viscosity | | | | | | |
| limit (mg/ml) | | | | | | |

### Example 8

### Integrity of Antibodies of the Invention Under Thermal Stress

Stability of antibodies of Example 3 under different stress conditions was assessed as follows: molecules were stored in solution in polypropylene vials with screw caps under conditions mimicking both physiological (phosphate buffered saline pH 7.4) and shelf life indicating conditions (Histidine/HCl, sodium chloride buffer pH 6.0). The molecule solutions were stored for prolonged periods of time at relevant temperatures (physiological: 37° C. and elevated stressed conditions: 40° C.). Subsequent analysis focuses on changes observed in the stored samples compared to an untreated control.

The changes were analyzed with the methods for integrity determination in Example 7.

TABLE E10
Residual activity of antibodies treated under stress conditions [%]

| | | 40° C. HisNaCl 37° C. | 40° C. HisNaCl 37° C. |
|---|---|---|---|
| | | PBS | PBS |
| # | identifier | 2 weeks | 4 weeks |
| 3 | P1AG9372 (Mab) | 98 | 97 |
| 2 | P1AG9376 (Mab) | 100 | — |
| 5 | P1AG9391 (Mab) | 99 | 97 |
| 1 | P1AG9426 (Mab) | 99 | 98 |
| 6 | P1AH1205 (Fab) | 99 | 98 |
| 7 | P1AH1199 (Fab) | 96 | 97 |

### Example 9

### Potency of Antibodies of the Invention (Wieslab Assay and Hemolysis Assay)

Wieslab assay: Potency of antibodies of Example 3 was assessed by the Wieslab Complement system Alternative pathway (AP) kit. This kit is an enzyme immunoassay for the determination of functional alternative complement pathway in human serum.

It combines principles of a hemolytic assay for complement activation with the use of specific antibodies to detect the generated neoantigen, which is proportional to the functional activity of the complement pathway. In brief, 2-fold dilution series of the antibody candidates and control compounds Pegcetacoplan (APL-2, a C3-inhibitor, Eur J Clin Invest. 2015 April; 45(4): 423-440. doi:10.1111/eci.12419) and Iptacopan (LNP023, an FB-inhibitor, Proc Natl Acad Sci USA. 2019 Apr. 16; 116(16):7926-7931. doi: 10.1073/pnas.1820892116. Epub 2019 Mar. 29) were provided in Histidine-buffer and then pre-incubated on ice for 15 min with human serum. The actual serum concentration at this point was 90%. This mixture was diluted 1:18 in a diluent with a specific blocker (so that only AP will be activated) and incubated for another 15 min at RT. Solution was added to a 96-well plate specifically coated with activators of the AP and incubated for 60 min at 37° C. Wells were washed and C5b-9 detected following the protocol. After 30 min the absorbance was measured at 405 nm and the amount of complement activation calculated using Xlfit.

TABLE E11
IC50 of indicated antibodies in Wieslab AP functional assay

| # | identifier | IC50 (nM) | 95conf_i |
|---|---|---|---|
| 3 | P1AG9372 (Mab) | 36.7 | 8.8 |
| 2 | P1AG9376 (Mab) | 35.7 | 9.7 |
| 5 | P1AG9391 (Mab) | 39.1 | 9.2 |
| 4 | P1AG9420 (Mab) | 51.8 | 11.2 |
| 1 | P1AG9426 (Mab) | 46.4 | 7.4 |
| 6 | P1AH1205 (Fab) | 71.6 | 13.0 |
| 7 | P1AH1199 (Fab) | 115.4 | 24.3 |

Hemolysis assay: In another experiment, the potency of the antibodies was assessed via an alternative pathway hemolysis assay. In brief, 2-fold dilution series of the antibody candidates and control compounds APL-2 (C3-inhibitor) and LNP023 (FB-inhibitor) were provided in Histidine-buffer. Samples were prepared at 4° C. or kept on ice so that the complement cascade would not start. Washed Rabbit Erythrocytes (RBC) were added to a V-shaped 96-well plate together with the dilution series of antibodies in a buffer appropriate for the alternative pathway. Normal human serum was added at a final assay concentration of 6.7% Complement activation was started by incubating the plates at 37° C. Every 10 min the plates were shaken for 1 min with 800 rpm to prevent the total sedimentation of the RBCs. Hemolysis reaction was stopped after 60 min with the addition of ice-cold NaCl-solution containing 10 mM EDTA. Plates were centrifuged for 3 min at 1250×g at 4° C. to pellet the non-lysed RBCs. Supernatant was carefully transferred and the absorbance was measured at 405 nm. Percentage of hemolysis was calculated: (OD test sample−OD BG or blank)/(OD full lysis−OD BG or blank)×100. IC50 values were calculated using Xlfit,

TABLE E12
IC50 of indicated antibodies in hemolysis assay

| # | identifier | IC50 (nM) | 95conf_i |
|---|---|---|---|
| 3 | P1AG9372 (Mab) | 11.2 | 1.8 |
| 2 | P1AG9376 (Mab) | 10.4 | 2.9 |
| 5 | P1AG9391 (Mab) | 13.0 | 2.0 |
| 4 | P1AG9420 (Mab) | 16.3 | 3.5 |
| 1 | P1AG9426 (Mab) | 12.8 | 1.8 |
| 6 | P1AH1205 (Fab) | 23.8 | 1.1 |
| 7 | P1AH1199 (Fab) | 48.1 | 2.8 |

In another experiment, the potency of the antibodies was assessed via a BLI based C3b deposition assay.

BLI based C3b deposition assay: The activated carbonyl group created upon C3 cleavage by the C3 convertase reacts and forms covalent adducts with nucleophiles in close proximity to the enzyme. We utilized this property to measure convertase activity by quantifying the C3b mass deposited on a BLI sensor loaded with C3bBb and exposed to C3.

To this end, biotinylated C3b was loaded with low density on a streptavidin functionalized SAX BLI sensor (Sartorius) in an Octet red 384 device, reaching a signal of 1 nm. The convertase was assembled by exposing it to 270 nM plasma purified factor B and 27 nM plasma purified factor D in HBS-T buffer (150 mM NaCl, 10 mM HEPES, 1 mM MgCl₂, 0.005% Tween20, pH 7.4) at 30° C. for 5 min, at which time a dynamic equilibrium between convertase assembly and dissociation was reached. Active sensors were then exposed to C3 (500 nM) in presence of test items at different concentrations and control sensors to test item alone, for 15 min, at which time convertase was considered completely dissociated. After a further 5 min exposition to buffer to allow the release of non-covalently associated material, deposited C3b was calculated from the signal difference between active and control sensor and used as index for activity and IC50 calculation.

TABLE E13
IC50 of indicated antibodies in BLI assay

| # | identifier | IC50 (nM) | 95conf_i |
|---|---|---|---|
| 3 | P1AG9372 (Mab) | 1.5 | 0.11 |
| 2 | P1AG9376 (Mab) | 2.2 | 0.30 |
| 5 | P1AG9391 (Mab) | 1.6 | 0.11 |
| 4 | P1AG9420 (Mab) | 1.4 | 0.23 |
| 1 | P1AG9426 (Mab) | 1.6 | 0.20 |
| 6 | P1AH1205 (Fab) | 4.5 | 0.27 |
| 7 | P1AH1199 (Fab) | 6.9 | 0.38 |

### Example 10

### Potency of Antibodies of the Invention Compared to Prior Art Clinical Candidates Targeting the Alternative Pathway

In a control experiment, the potency of prior art clinical candidates targeting the alternative pathways was compared using the assays described in the previous examples. As controls, APL-2 peptide, which binds to C3 (Eur J Clin Invest. 2015 April; 45(4): 423-440. doi:10.1111/eci.12419), and LNP023, which inhibits FB, (Proc Natl Acad Sci USA. 2019 Apr. 16; 116(16):7926-7931. doi: 10.1073/pnas.1820892116. Epub 2019 Mar. 29) were used.

TABLE E14
IC50 of indicated comparative compounds

| | Wieslab assay | | Hemolysis assay | | BLI assay | |
|---|---|---|---|---|---|---|
| | IC50 | | IC50 | | IC50 | |
| identifier | (nM) | 95conf_i | (nM) | 95conf_i | (nM) | 95conf_i |
| APL-2 | 274.2 | 10.9 | 274.2 | 10.9 | 200 | 37.6 |
| LNP023 | 161.4 | 15.2 | 161.4 | 15.2 | 14 | 1.17 |

### Example 11

Cross-Reactivity of Antibodies of the Invention with Different Monkey Species

Cross-reactivity of antibodies of the invention towards C3bBb from different monkey species, i.e. Macaca Fascicularis (Cynomolgus) and Chlorocebus sabaeus (African Green Monkey), was assessed using the potency assays (hemolysis assay) which varied slightly to the one described in the previous examples depending on the species.

In brief, 2-fold dilution series of the antibody candidates and control compounds APL-2 (C3-inhibitor) and LNP023 (FB-inhibitor) were provided in Histidine-buffer. Samples were prepared at 4° C. or kept on ice so that the complement cascade would not start. Washed Rabbit Erythrocytes (RBC) were added to a V-shaped 96-well plate together with the dilution series of antibodies in a buffer appropriate for the alternative pathway.

For Cynomolgus normal human serum was added at a final assay concentration of 500 (serum pool of 2 individuals). The complement activation started with the incubation of the plates at 37° C. Every 10 min the plates were shaken for 1 min with 800 rpm to prevent the total sedimentation of the RBCs. Hemolysis reaction was stopped after 20 min with the addition of ice-cold NaCl-solution containing 10 mM EDTA. Plates were centrifuged for 3 min at 1000×g at 4° C. to pellet the non-lysed RBCs. Supernatant was carefully transferred and the absorbance was measured at 405 nm. Percentage of hemolysis was calculated: (GD test sample−GD BG or blank)/(OD full lysis−GD BG or blank)×100. IC50 values were calculated using Xlfit.

For African Green Monkey (AGM) normal human serum was added at a final assay concentration varying between 4.200 and 6.7% depending on the individual animal. These concentrations were determined in a previous experiment as well with the incubation time and temperature to have the appropriate level of hemolysis. The biggest difference to the ‘regular’ hemolysis assay was that the plates were incubated at RT and the activation was stopped after 20 min and the plates were further processed as described above.

TABLE E15
Cross-reactivity of indicated compounds in hemolysis assay

| | | Macaca Fascicularis | | Chlorocebus sabaeus | |
|---|---|---|---|---|---|
| identifier | # | IC50 (nM) | 95conf_i | IC50 (nM) | 95conf_i |
| P1AG9372 | 3 | 1115 | 176.2 | 111.8 | 30.4 |
| (Mab) | | | | | |
| P1AG9376 | 2 | 802 | 1108.9 | 27.6 | 6.4 |
| (Mab) | | | | | |
| P1AG9391 | 5 | 981 | 445.5 | 74.8 | 27.3 |
| (Mab) | | | | | |
| P1AG9420 | 4 | >578 | | >578 | |
| (Mab) | | | | | |
| P1AG9426 | 1 | >2000 | 584.3 | 139.9 | 42.2 |
| (Mab) | | | | | |
| P1AH1205 | 6 | 3059 | 600.8 | 445.9 | 149.2 |
| (Fab) | | | | | |
| P1AH1199 | 7 | >6000 | 2246.5 | 1306.5 | 248.4 |
| (Fab) | | | | | |
| APL-2 | | 316 | 30.3 | 147.4 | 16.3 |
| LNP023 | | 73.2 | 7.1 | 113.0 | 11.3 |

### Example 12

### Impact of Antibodies of the Invention on Convertase Dissociation

### Impact of Different Anti-C3bBb Antibodies (Inhibitors) on Half-Life of Tripartite Complex Comprising C3bBb Inhibitors and C3bBb

Impact of C3bBb inhibitors (Inh) on the convertase stability was investigated by analyzing the decay of C3bBb:Inh tripartite complex assembled on a Bio Layer interferometry sensor.

To this end, biotinylated C3b was loaded at low density on a streptavidin functionalized SAX BLI sensor on an Octet red384 device (Sartorius), reaching a signal of 1.5 nm. The convertase was assembled by exposing the sensor to 270 nM plasma buffer (150 mM NaCl, 10 mM HEPES, 1 mM MgCl₂, 0.005% Tween20. pH 7.4)) at 30° C. for 5 min, at which time a dynamic equilibrium between convertase assembly and dissociation was reached. The convertase loaded sensors were then exposed to 500 nM test items in continued presence of FB and FD for 10 min allowing the inhibitor to reach binding equilibrium with the convertase.

Signal decay of the C3bBb:Inh tripartite complex was followed on the control sensor and fitted to an exponential function using XL fit (IDBS software) to calculate the tripartite complex half-life. Data were compared to a parallel assay performed in absence of inhibitor, revealing that some of the explored C3bBb specific binders strongly stabilized the convertase, a probable effect of C3b-Bb interface binding, whereas other inhibitors did not stabilize the convertase.

TABLE E16a

| | | | Initial | | | | |
|---|---|---|---|---|---|---|---|
| | | | deposition | C3b | | | |
| | | | rate | deposition | C3bBb | | |
| | | Concentration | inhibition | inhibition | t½ | C3bBb:Inh | Increase |
| # | identifier | (nM) | (%) | (%) | (s) | t½ (s) | (%) |
| 8 | P045.072 | 500 | 87 | Not | 207 | 2453 | 1085 |
| | (Fab) | | | determined | | | |
| 10 | P045.225 | 500 | 82 | Not | 207 | 1105 | 434 |
| | (Fab) | | | determined | | | |
| 9 | P045.236 | 500 | 58 | Not | 207 | 484 | 134 |
| | (Fab) | | | determined | | | |
| 20 | P046.185 | 500 | 88 | Not | 207 | 229 | 11 |
| | (Fab) | | | determined | | | |
| 13 | P046.127 | 500 | 100 | Not | 207 | 4120 | 1890 |
| | (Fab) | | | determined | | | |
| 19 | P055.015 | 500 | 90 | Not | 204 | 628 | 207 |
| | (Fab) | | | determined | | | |

In another experiment, the impact of selected inhibitors on the convertase half-life was quantitatively examined in conditions ensuring that the convertase was saturated with tested compounds. Design similar to the previous experiment was implemented, except that C3b loading was limited to 1 nm BLI signal. Compounds were tested over a 316-fold dilution range, equilibration with the convertase extended to 15 m and exposure to C3 (paralleling convertase decay on the control sensor) limited to 15 m(.

At the highest concentration tested, 31.6 nM, 63.2 nM, 320 nM and 5 μM for respectively Mabs, and Fabs, convertase activity was essentially fully suppressed suggesting that, in the experimental condition, convertase occupancy was close to 100%. Furthermore analysis of the BLI signal shift associated to Mab and Fab binding confirmed that saturation was achieved. Therefore the reported half-life refers to the tripartite complex and not a bound/unbound convertase mixture.

TABLE E16b

| | | | Initial | | | | |
|---|---|---|---|---|---|---|---|
| | | | deposition | C3b | | | |
| | | | rate | deposition | C3bBb | C3bBb:Inh | t½ |
| | | Concentration | inhibition | inhibition | t½ | t½ | increase |
| # | Identifier | (nM) | (%) | (%) | (s) | (s) | (%) |
| 3 | P1AG9372 | 31.6 | 100 | 97 | 229 | 318 | 39 |
| | (Mab) | | | | | | |
| 2 | P1AG9376 | 31.6 | 99 | 99 | 197 | 299 | 52 |
| | (Mab) | | | | | | |
| 5 | P1AG9391 | 31.6 | 100 | 99 | 231 | 313 | 35 |
| | (Mab) | | | | | | |
| 4 | P1AG9420 | 31.6 | 99 | 96 | 188 | 215 | 14 |
| | (Mab) | | | | | | |
| 1 | P1AG9426 | 31.6 | 100 | 99 | 257 | 326 | 27 |
| | (Mab) | | | | | | |
| 6 | P1AH1205 | 63.2 | 97 | 96 | 184 | 207 | 14 |
| | (Fab) | | | | | | |
| 7 | P1AH1199 | 63.2 | 94 | 93 | 204 | 219 | 7.3 |
| | (Fab) | | | | | | |

### Example 13

### Antibodies of the Invention are Selective Inhibitors of the Alternative Pathway and do not Inhibit the Classical Pathway and Lectin Pathway

In order to confirm that the antibodies of the invention are selective inhibitors of the alternative pathway, a classical pathway hemolysis assay was performed as follows

In brief, 2-fold dilution series of the antibody candidates and control compounds APL-2 (C3-inhibitor) and LNP023 (FB-inhibitor) were provided in Histidine-buffer. Everything was prepared at RT except for the serum which was kept on ice so that the complement cascade would not start. Washed Ab-sensitized sheep erythrocytes (EA) were added to a V-shaped 96-well plate together with the dilution series of antibodies in a buffer appropriate for the classical pathway. Normal human serum was added at a final assay concentration of 2% and mixed gently with the other components. Complement activation was started by incubating the plates at 37° C. shaking continuously at 600 rpm to prevent the total sedimentation of the EAs. Hemolysis reaction was stopped after 30 min with the addition of ice-cold NaCl-solution containing 10 mM EDTA. Plates were centrifuged for 2 min at 1000×g at 4° C. to pellet the non-lysed EAs. Supernatant was carefully transferred and the absorbance was measured at 405 nm. Percentage of hemolysis was calculated: (OD test sample−OD BG or EDTA ctrl)/(OD full lysis−OD BG or EDTA ctrl)×100. IC50 values were calculated using Xlfit.

TABLE E17
IC50 of indicated molecules assessed by classical pathway hemolysis assay

| # | identifier | IC50 (nM) | 95conf_i |
|---|---|---|---|
| 3 | P1AG9372 (Mab) | >2000 | |
| 2 | P1AG9376 (Mab) | >2000 | |
| 5 | P1AG9391 (Mab) | >2000 | |
| 4 | P1AG9420 (Mab) | >578 | |
| 1 | P1AG9426 (Mab) | >2000 | |
| 6 | P1AH1205 (Fab) | >6000 | |
| 7 | P1AH1199 (Fab) | >6000 | |
| | APL-2 | 1022 | 170.7 |
| | LNP023 | >20.000 | |

As expected, LNP023, a Factor B-inhibitor does not inhibit the classical pathway. It is specific for the alternative pathway only.

### Example 14

### Binding to Wild-Type Human C3bBb

Specific binding of antibody P1AG9426 (#1) of the invention of the wild-type human C3bBb complex was analyzed via SPR analysis.

### Wild-Type Human C3bBb SPR Assay

A Biacore T200 system was equipped with a CM5 chip and primed with 1×HBS-N(10 mM HEPES, 150 mM NaCl), prepared from a 10× stock solution (Cytiva BR100670). An anti-human Fab antibody (Thermo Fisher Scientific 7103082100) was immobilized to all flow cells (FC1-4) in an amine coupling with default settings and a capture antibody injection time of 420 s at 10 pl/min and 30 μg/ml in 10 mM Acetate pH 5.

The following method was run in HBS-P+1 mM NiSO₄. If not stated differently, the samples were diluted in the running buffer. The antibody was captured for 60 s at 10 pl/min (diluted in HBS-N). Afterwards the prebuilt C3bBb wt complex was injected for 120 s at 10 pl/min with 600 s dissociation. The complex was prebuilt by incubating 300 nM human C3b, 310 nM human wt Factor B and 40 nM human Factor D for 5 min at RT. The surface was regenerated by injecting 10 mM Glycine pH 2.1 for 60 s at 30 pl/min. The data were analyzed by double-referencing the signals on FC2 to FC1 and a buffer cycle, injecting running buffer instead of C3bBb wt complex.

### Example 15

### C5 Convertase Inhibition

Hemolysis assay: The standard hemolysis assay was modified in order to assess the inhibitory potential of the antibodies specifically at the level of the alternative pathway C5-convertase. Samples were prepared at 4° C. or kept on ice so that the complement cascade would not start. Washed Rabbit Erythrocytes (RBC) were added to a V-shaped 96-well plate in a low salt (60 mM NaCl) buffer appropriate for the alternative pathway, but containing also 200 nM C5-inhibitor (Crovalimab) to prevent MAC formation. Cells were pre-heated for 10 min at 37° C. followed by the addition of ice-cold normal human serum at a final assay concentration of 6.7% and incubated for additional 10 minutes with constant shaking enabling the formation of C3- and C5-convertases, but no C5 cleavage. The formation of further convertases was stopped by adding a buffer containing 40 mM EDTA, cells were spin down, and the pellet was resuspended in 2 consecutive steps: first with a buffer containing final 1.25% guinea pig serum (as a source of the complement proteins C5-9 needed for MAC formation), EDTA and the antibodies (see Table E18 for concentrations), followed by a buffer containing Mg-EGTA. Under these conditions, there is no de novo convertase formation and the inhibition of hemolysis is a direct consequence of the inhibition of C5-convertases formed earlier. Hemolysis was measured after incubating the cells for 1 h at 37° C. with constant shaking.

TABLE E18
C5 convertase inhibition of indicated antibodies

| | | Concentration | Level of inhibition |
|---|---|---|---|
| # | identifier | (nM) | [%] |
| 3 | P1AG9372 (Mab) | 21 | 68 |
| 2 | P1AG9376 (Mab) | 58 | 70 |
| 5 | P1AG9391 (Mab) | 52 | 73 |
| 4 | P1AG9420 (Mab) | 165 | 63 |
| 1 | P1AG9426 (Mab) | 276 | 63 |
| 6 | P1AH1205 (Fab) | 520 | 74 |
| 20 | P1AF8499 | 1000 | 70 |
| 153 | PT5629.F09_30.A20_1 | 839 | 73 |
| | LNP023 | 20000 | 70 |

1. An antibody that binds to human C3bBb, comprising
i) a heavy chain variable domain (VH) comprising (a) a CDR-H1 comprising the amino acid sequence of SEQ ID NO:449; (b) a CDR-H2 comprising the amino acid sequence of SEQ ID NO:450; and (c) a CDR-H3 comprising the amino acid sequence of SEQ ID NO:451, and a light chain variable domain (VL) comprising (d) a CDR-L1 comprising the amino acid sequence of SEQ ID NO:452; (e) a CDR-L2 comprising the amino acid sequence of SEQ ID NO:453; and (f) a CDR-L3 comprising the amino acid sequence of SEQ ID NO:454;
ii) a heavy chain variable domain (VH) comprising (a) a CDR-H1 comprising the amino acid sequence of SEQ ID NO:455; (b) a CDR-H2 comprising the amino acid sequence of SEQ ID NO:456; and (c) a CDR-H3 comprising the amino acid sequence of SEQ ID NO:457, and a light chain variable domain (VL) comprising (d) a CDR-L1 comprising the amino acid sequence of SEQ ID NO:458; (e) a CDR-L2 comprising the amino acid sequence of SEQ ID NO:459; and (f) a CDR-L3 comprising the amino acid sequence of SEQ ID NO:460;
iii) a heavy chain variable domain (VH) comprising (a) a CDR-H1 comprising the amino acid sequence of SEQ ID NO:461; (b) a CDR-H2 comprising the amino acid sequence of SEQ ID NO:462; and (c) a CDR-H3 comprising the amino acid sequence of SEQ ID NO:463, and a light chain variable domain (VL) comprising (d) a CDR-L1 comprising the amino acid sequence of SEQ ID NO:464; (e) a CDR-L2 comprising the amino acid sequence of SEQ ID NO:465; and (f) a CDR-L3 comprising the amino acid sequence of SEQ ID NO:466;
iv) a heavy chain variable domain (VH) comprising (a) a CDR-H1 comprising the amino acid sequence of SEQ ID NO:467; (b) a CDR-H2 comprising the amino acid sequence of SEQ ID NO:468; and (c) a CDR-H3 comprising the amino acid sequence of SEQ ID NO:469, and a light chain variable domain (VL) comprising (d) a CDR-L1 comprising the amino acid sequence of SEQ ID NO:470; (e) a CDR-L2 comprising the amino acid sequence of SEQ ID NO:471; and (f) a CDR-L3 comprising the amino acid sequence of SEQ ID NO:472;
v) a heavy chain variable domain (VH) comprising (a) a CDR-H1 comprising the amino acid sequence of SEQ ID NO:473; (b) a CDR-H2 comprising the amino acid sequence of SEQ ID NO:474; and (c) a CDR-H3 comprising the amino acid sequence of SEQ ID NO:475, and a light chain variable domain (VL) comprising (d) a CDR-L1 comprising the amino acid sequence of SEQ ID NO:476; (e) a CDR-L2 comprising the amino acid sequence of SEQ ID NO:477; and (f) a CDR-L3 comprising the amino acid sequence of SEQ ID NO:478 (corresponding to antibody #5, P1AG9391);
vi) a heavy chain variable domain (VH) comprising (a) a CDR-H1 comprising the amino acid sequence of SEQ ID NO:479; (b) a CDR-H2 comprising the amino acid sequence of SEQ ID NO:480; and (c) a CDR-H3 comprising the amino acid sequence of SEQ ID NO:481, and a light chain variable domain (VL) comprising (d) a CDR-L1 comprising the amino acid sequence of SEQ ID NO:482; (e) a CDR-L2 comprising the amino acid sequence of SEQ ID NO:483; and (f) a CDR-L3 comprising the amino acid sequence of SEQ ID NO:484; or
vii) a heavy chain variable domain (VH) comprising (a) a CDR-H1 comprising the amino acid sequence of SEQ ID NO:485; (b) a CDR-H2 comprising the amino acid sequence of SEQ ID NO:486; and (c) a CDR-H3 comprising the amino acid sequence of SEQ ID NO:487, and a light chain variable domain (VL) comprising (d) a CDR-L1 comprising the amino acid sequence of SEQ ID NO:488; (e) a CDR-L2 comprising the amino acid sequence of SEQ ID NO:489; and (f) a CDR-L3 comprising the amino acid sequence of SEQ ID NO:490.

2. The antibody of claim 1, wherein the antibody comprises a VH domain with a VH3 framework and a VL domain with a Vkappa1 framework.

3. The antibody of claim 1, comprising a sequence selected from the group consisting of:
i) a VH domain comprising SEQ ID NO:1 and a VL domain comprising SEQ ID NO:2;
ii) a VH domain comprising SEQ ID NO:3 and a VL domain comprising SEQ ID NO:4;
iii) a VH domain comprising SEQ ID NO:5 and a VL domain comprising SEQ ID NO:6;
iv) a VH domain comprising SEQ ID NO:7 and a VL domain comprising SEQ ID NO:8;
v) a VH domain comprising SEQ ID NO:9 and a VL domain comprising SEQ ID NO:10;
vi) a VH domain comprising SEQ ID NO:11 and a VL domain comprising SEQ ID NO:12; or
vii) a VH domain comprising SEQ ID NO:13 and a VL domain comprising SEQ ID NO:14.

4. The antibody of claim 1, comprising a sequence selected from the group consisting of:
viii) a heavy chain comprising the amino acid sequence of SEQ ID NO:435 and a light chain comprising the amino acid sequence of SEQ ID NO:436;
ix) a heavy chain comprising the amino acid sequence of SEQ ID NO:437 and a light chain comprising the amino acid sequence of SEQ ID NO:438;
x) a heavy chain comprising the amino acid sequence of SEQ ID NO:439 and a light chain comprising the amino acid sequence of SEQ ID NO:440;
xi) a heavy chain comprising the amino acid sequence of SEQ ID NO:441 and a light chain comprising the amino acid sequence of SEQ ID NO:442;
xii) a heavy chain comprising the amino acid sequence of SEQ ID NO:443 and a light chain comprising the amino acid sequence of SEQ ID NO:444;
xiii) a heavy chain comprising the amino acid sequence of SEQ ID NO:445 and a light chain comprising the amino acid sequence of SEQ ID NO:446; or
xiv) a heavy chain comprising the amino acid sequence of SEQ ID NO:447 and a light chain comprising the amino acid sequence of SEQ ID NO:448.

5. An isolated nucleic acid encoding the antibody of claim 1.

6. A pharmaceutical composition comprising the antibody of claim 1 and a pharmaceutically acceptable carrier.

7. A method of treating an individual having an ocular disease, comprising administering to the individual an effective amount of the antibody of claim 1.
